(19) United States
(12) Patent Application Publication
(10) Pub. No.: US 2017/0166972 A1
(43) Pub. Date: Jun. 15, 2017

(54) LONG NON-CODING RNA AS A DIAGNOSTIC AND THERAPEUTIC AGENT

(71) Applicant: SANFORD-BURNHAM MEDICAL RESEARCH INSTITUTE, La Jolla, CA (US)

(72) Inventor: Ranjan Perera, La Jolla, CA (US)

(73) Assignee: SANFORD-BURNHAM MEDICAL RESEARCH INSTITUTE, La Jolla, CA (US)

(21) Appl. No.: 15/039,029

(22) PCT Filed: Nov. 26, 2014
(86) PCT No.: PCT/US2014/067747

§ 371 Date: May 24, 2016

Related U.S. Application Data
(60) Provisional application No. 61/909,319, filed on Nov. 26, 2013.
(60) Provisional application No. 61/920,318, filed on Dec. 23, 2013.

(51) Int. Cl. C12Q 1/68; C12N 15/113

(52) U.S. Cl. CPC C12Q 1/6886; C12N 15/113; C12Q 2600/158; C12Q 2600/178; C12Q 2600/106; C12Q 2600/112; C12N 2310/14; C12N 2310/11

(57) ABSTRACT

Provided herein are methods for the diagnosis of cancer by comparison of a quantification of long non-coding RNA with the same measurement taken in a reference sample from a healthy patient. Further provided herein are methods of anticipating the likelihood that such a disease will develop, and methods of treatment in the event of such development.

### CROSS-REFERENCE TO RELATED APPLICATIONS

This application claims priority to and benefit of U.S. Provisional Patent Application Ser. No. 61/909,319, filed Nov. 26, 2013 and U.S. Provisional Patent Application Ser. No. 61/920,318, filed Dec. 23, 2013, the disclosures of which are incorporated herein by reference in their entirety for all purposes.

### FIELD OF THE INVENTION

The present technology relates to methods of diagnosing and treating human cancers, e.g., prostate cancer.

### BACKGROUND

The following discussion of the background of the invention is merely provided to aid the reader in understanding the invention and is not admitted to describe or constitute prior art to the present invention.

There is considerable interest in understanding the function of RNA transcripts that do not code for proteins in eukaryotic cells. As evidenced by cDNA cloning projects and genomic tiling arrays, more than 90% of the human genome undergoes transcription but does not code for proteins. These transcriptional products are referred to as non-protein coding RNAs (ncRNAs). A variety of ncRNA transcripts, such as ribosomal RNAs, transfer RNAs, and spliceosomal RNAs, are essential for cell function. Similarly, a large number of short ncRNAs such as micro-RNAs (miRNAs), endogenous short interfering RNAs (siRNAs), PIWI-interacting RNAs (piRNAs) and small nucleolar RNAs (snoRNAs) are also known to play important regulatory roles in eukaryotic cells. Recent studies have demonstrated a group of long ncRNA (lncRNA) transcripts that exhibit cell type-specific expression and localize into specific subcellular compartments. lncRNAs are also known to play an important roles during cellular development and differentiation supporting the view that they have been selected during the evolutionary process.

LncRNAs appear to have many different functions. In many cases, they seem to play a role in regulating the activity or localization of proteins, or serve as organizational frameworks for subcellular structures. In other cases, lncRNAs are processed to yield multiple small RNAs or they may modulate how other RNAs are processed.

Interestingly, lncRNAs can influence the expression of specific target proteins at specific genomic loci, modulate the activity of protein binding partners, direct chromatin-modifying complexes to their sites of action, and are post-transcriptionally processed to produce numerous 5′-capped small RNAs. Epigenetic pathways can also regulate the differential expression of lncRNAs. lncRNAs are misregulated in various diseases, including ischaemia, heart disease, Alzheimer's disease, psoriasis, and spinocerebellar ataxia type 8. This misregulation has also been shown in various types of cancers, such as breast cancer, colon cancer, prostate cancer, hepatocellular carcinoma and leukemia. One such lncRNA, DD3 (also known as PCA3), is listed as a specific prostate cancer biomarker. Recent studies have revealed the contribution of ncRNAs as proto-oncogenes, e.g. GAGE6, as tumor suppressor genes in tumorigenesis, and as drivers of metastatic transformation, e.g. HOTAIR in breast cancer.

Prostate cancer (PCa) is one of the leading causes of cancer deaths among American men. According to 2013 National Cancer Institute estimates, there will be 238,590 new prostate cancer diagnoses this year; for 29,720 men this is likely to be fatal. Although the incidence of prostate cancer has been steadily rising [2], with a concurrent increase in aggressive surgical management [3], most men have indolent disease for which conservative therapy or an active surveillance approach would be more appropriate and result in less treatment-related morbidity [1]. A contributing problem has been the widespread use of prostate specific antigen (PSA) testing, which has low specificity for cancer and cannot differentiate indolent and aggressive cancers; this has resulted in large numbers of unnecessary biopsies and overtreatment. There is therefore an urgent unmet need for a specific prognostic biomarker that can refine existing diagnostic methods.

### SUMMARY OF THE INVENTION

The present technology is based on the discovery of the biomarkers for the early detection of prostate cancer to reduce over-treatment and accompanying morbidity.

In one aspect, the present technology provides for a method for accessing the progression of prostate cancer in a subject who is undergoing treatment for prostate cancer, which method comprises: (i) assessing the expression level of a long noncoding RNA in a biological sample obtained from the subject; (ii) comparing the expression level of the long noncoding RNA in the sample to a reference derived from the expression level of the long noncoding RNA in samples obtained from healthy subjects and determining the current condition of the subject; and (iii) for the subject determined to suffer from prostate cancer periodically repeating steps (i) and (ii) during treatment as a basis to determine the efficacy of said treatment by assessing whether the expression level of the long noncoding RNA in the subject is up-regulated or down-regulated, wherein a down-regulation in the expression level of the long noncoding RNA correlates to an improvement in the subject's condition.

In some embodiments, the long noncoding RNA is selected from the group consisting of SEQ ID NOs: 2-76. In some embodiments, the method further comprises assessing the expression level of SPRY4-IT1 (SEQ ID NO: 1).

In some embodiments, the expression level of the long noncoding RNA is assessed by evaluating the amount of the long noncoding RNA using a probe. In some embodiments, the biological sample comprises a tissue sample. In some embodiments, the tissue sample is a prostatic adenocarcinoma tissue sample. In some embodiments, the prostate cancer is early stage prostate cancer.

In some embodiments, the long noncoding RNA is XLOC_007697 (SEQ ID NO: 2). In some embodiments, the long noncoding RNA is XLOC_009911 (SEQ ID NO: 3). In some embodiments, the long noncoding RNA is XLOC_008559 (SEQ ID NO: 4). In some embodiments, the long noncoding RNA is XLOC_005327 (SEQ ID NO: 5). In some embodiments, the long noncoding RNA is LOC100287482 (SEQ ID NO: 6).

In another aspect, the present technology provides for a method for treating prostate cancer in a patient diagnosed as having prostate cancer comprising administering to the patient an effective amount of a therapeutic agent that reduces or down-regulates the expression level of a long noncoding RNA.

In some embodiments, the long noncoding RNA is selected from the group consisting of SEQ ID NOs: 2-76. In some embodiments, the long noncoding RNA expression is reduced or down-regulated in prostate cancer cells. In some embodiments, the long noncoding RNA expression is reduced by at least about 50%, 60%, 70%, 80% or 90%. In some embodiments, the therapeutic agent is an siRNA. In some embodiments, the therapeutic agent is contained within a liposome.

In yet another aspect, the present technology provides for a method for determining a treatment regimen for a patient with prostate cancer which method comprises: identifying whether said cancer is aggressive or indolent by identifying one or more of markers for aggressive prostate cancer said marker is one or more of PSA isoforms, kallikreins, GSTP1, AMACR, ERG, gene fusions involving ETS-related genes, PCA3, or a combination thereof; treating said cancer with a regimen consistent with whether the cancer is aggressive or indolent.

In some embodiments, the progress of said treatment regimen is monitored by further evaluating the presence and quantity of one or more of said markers in said patient and optionally adjusting the treatment protocol based on said evaluation.

In some embodiments, the treatment regimen is one or more of open prostatectomy, minimally invasive laparoscopic robotic surgery, intensity modulated radiation therapy (IMRT), proton therapy, brachytherapy, cryotherapy, molecular-targeted therapy, vaccine therapy and gene therapy, hormone therapy, active surveillance, or a combination thereof.

In yet another aspect, the present technology provides for a method for detecting prostate cancer in a patient suspected of having prostate cancer, which method comprises: (i) assessing the expression level of a long noncoding RNA in a biological sample obtained from said patient; (ii) comparing the expression level of the long noncoding RNA in the sample to a reference derived from the expression level of the long noncoding RNA in samples obtained from healthy subjects; (iii) identifying said patient as having prostate cancer when the expression level of the long noncoding RNA in said patient is greater than the reference or identifying said patient as not having prostate cancer when the expression level of the long noncoding RNA is equal or less than the reference.

In some embodiments, the patient is suspected of prostate cancer based on the patient's prostate specific antigen (PSA) Score, the Myriad Prolaris Assay (MPA) Score, the Oncotype DX Genomic Prostate Score (GPS), or the Cancer of the Prostate Risk Assessment (CAPRA) Score.

In yet another aspect, the present technology provides for a method for differentiating indolent and aggressive prostate cancer, which method comprises: identifying the aggressive prostate cancer based on the expression of one or more of aggressive tumor-predictive genes associated with the aggressive prostate cancer; and identifying the indolent prostate cancer based on the lack of the expression or the low expression of one or more of aggressive tumor-predictive genes associated, and wherein the expression of aggressive tumor-predictive genes is determined by one or more of prostate specific antigen (PSA) Score, the Myriad Prolaris Assay (MPA) Score, the Oncotype DX Genomic Prostate Score (GPS), the Cancer of the Prostate Risk Assessment (CAPRA) Score, or a combination thereof.

In yet another aspect, the present technology provides for a kit comprising a composition comprising a long noncoding RNA, and instructions for use, wherein the long noncoding RNA is selected from the group consisting of SEQ ID NOs: 2-76.

### BRIEF DESCRIPTION OF THE FIGURES

FIG. 1 depicts screening of prostate cancer related IncRNA expression using microarrays. Alterations in IncRNA expression profiles between FIG. 1A prostatic epithelial cells and PC3 and FIG. 1B between prostate epithelial cells, PC3, and LNCaP cells. Hierarchical clustering shows distinguishable IncRNA expression profiles. Red indicates high relative expression and green indicates low relative expression.

FIG. 2 depicts the expression of the IncRNAs XLOC-007697, LOC100506411, LOC100287482, SPRY4-IT1, and the mRNA of SPRY4 in prostate cancer cell lines and prostatic epithelial cells. Expression of three IncRNAs (XLOC-007697 as shown in FIG. 2A, LOC100506411 as shown in FIG. 2B, and LOC100287482 as shown in FIG. 2C) as measured by qRT-PCR in five prostate cancer cell lines (PPC1, 22Rv1, DU-145, LNCaP, and PC3) using prostatic epithelial cells as a reference. Experiment performed in triplicate. FIG. 2D depicts the expression of SPRY4-IT, and FIG. 2E depicts the expression of SPRY4 as measured by qRT-PCR in the same samples as in FIG. 2A-C. Experiment performed in triplicate. FIG. 2F depicts the expression of SPRY4-IT1 and SPRY4 by RNA-FISH staining of prostatic epithelial, LNCaP, and PC3 cells. SPRY4-IT1 staining is in green (FITC), SPRY4 staining is in red (Alexa 590), and nuclei are stained in blue (DAPI).

FIG. 3 depicts the methylation of an upstream CpG Island can simultaneously regulate both SPRY4 and SPRY4-IT1. FIG. 3A is a map illustrating the genomic position of the SPRY4 ORF, promoter, and upstream CpG island at the SPRY4 locus. FIG. 3B is an illustration and examination of the methylation state of the CpG Island upstream of SPRY4 in LNCaP cells before and after treatment with 5-aza-2′-deoxycytidine. Six clones of each were sequenced and annotated, and the total numbers of methylated sites for each clone are indicated on the far right. FIG. 3C depicts the expression of the mRNA of SPRY4 as measured by qRT-PCR in LNCaP cells before and after treatment with 5-aza-2′-deoxycytidine. Experiment performed in triplicate. FIG. 3D depicts the expression of the IncRNA SPRY4-IT1 by qRT-PCR in LNCaP cells, as performed in FIG. 2F. Experiment performed in triplicate.

FIG. 4 depicts the differential expression of the IncRNAs in human prostatic adenocarcinoma. FIG. 4A depicts a heat map showing differential IncRNA expression between prostate tumor samples and adjacent normal tissues. FIG. 4B depicts four IncRNAs (XLOC-009911, XLOC-008559, XLOC-005327, and XLOC-001699) were selected on the basis of the microarray results performed with patient samples. The expression level was measured in 15 matched normal versus prostate tumor samples by qRT-PCR. The box plot indicates fold changes (ΔCt) in tumor tissues relative to adjacent normal tissues. Expression is normalized to 0 in matched normal tissues. FIG. 4C depicts the expression level of three IncRNAs (XLOC-007697, LOC100506411, and LOC100287482) was measured in 12 matched normal versus tumor prostate tissue samples by qRT-PCR. The box plot indicates fold changes (ΔCt) in tumor tissues relative to adjacent normal tissues. Expression is normalized to 0 in matched normal tissues. FIG. 4D depicts the expression level of SPRY4-IT1 was measured by qRT-PCR in 18 paired prostate tumor and normal samples. FIG. 4E depicts the correlation between SPRY4-IT1 and SPRY4 expression in patient samples. The correlation between gene expression data was calculated using linear regression analysis. The number of analyzed samples was 11. FIG. 4F depicts the expression level of SPRY4-IT1 in patient samples measured by droplet digital PCR (ddPCR). SPRY4-IT1 expression was measured using TaqMan assays, Hs03865501_s1 for SPRY4-IT1 and Hs02758991_g1 for GAPDH, in 18 paired patient samples. The relative expression in tumor tissues is normalized to that of matched normal tissues.

FIG. 5 depicts the RNA-CISH analysis of SPRY4-IT1. FIG. 5A depicts the RNA-CISH staining of SPRY4-IT1 in matched normal and tumor samples. Expression is visualized using alkaline phosphatase labeled probes. (Scale bar: 100 μm). FIG. 5B depicts the qRT-PCR for SPRY-IT1 expression in matched normal and tumor samples stained in 5A. FIG. 5C depicts the RNA-CISH staining for SPRY4-IT1 expression in a human prostate cancer tissue array. Tissue samples include normal prostate, adjacent normal, and prostate cancer samples indicated by Gleason scores: 6 (3+3), 7 (3+4), 8 (4+4), 9 (5+4 & 4+5), and 10 (5+5). Expression is visualized using alkaline phosphatase labeled probes.

FIG. 6 depicts the examination of the physiological impact of SPRY4-IT1 knockdown on prostate cancer cells. FIG. 6A depicts the efficiency of knockdown of SPRY4-IT1 in PC3 cells using siRNA after 48 hours transient transfection, as measured by qRT-PCR. FIG. 6B depicts the MTT assay measuring cell viability after 48 hours transient transfection with siRNA in PC3 cells. FIG. 6C depicts an invasion assay after 48 hours transfection with siRNA in PC3 cells. FIG. 6D depicts the staining of PC3 cells (crystal violet) after 48 hours transfection with SPRY4-IT1 siRNA. FIG. 6E depicts the apoptosis measured by caspase 3/7 activity in PC3 cells 48 hours after transfection with SPRY4-IT1 siRNA. All experiments performed in triplicate.

FIG. 7 depicts the putative prostate biomarker expression in urine samples. Expression of eight lncRNAs (SPRY4-IT1, XLOC-007697, LOC100506411, LOC100287482, XLOC-009911, XLOC-008559, XLOC-005327, and XLOC-001699) and PCA3 was measured by qRT-PCR in one normal and three prostate cancer patients. The relative expression to normal control is presented as fold change for each gene. The expression of all eight lncRNAs and PCA3 was significantly higher in prostate cancer patients.

FIG. 8 depicts the probe and LncRNA sequence alignment: Probe ID (A_21_P0006269), Gene Name (XLOC_007697; SEQ ID NO: 2) and Accession # (TCONS_00016323.1).

FIG. 9 depicts the probe and LncRNA sequence alignment: Probe ID (A_19_P00802433), Gene Name (XLOC_005327; SEQ ID NO: 5) and Accession # (ENST00000448327.1).

FIG. 10 depicts the probe and LncRNA sequence alignment: Probe ID (A_21_P0007070), Gene Name (XLOC_008559; SEQ ID NO: 4) and Accession # (TCONS_00018783.1).

FIG. 11 depicts the probe and LncRNA sequence alignment: Probe ID (A_21_P0007854), Gene Name (XLOC_009911; SEQ ID NO: 3) and Accession # (TCONS_00021223.1).

FIG. 12 depicts the probe and LncRNA sequence alignment: Probe ID (A_21_P0000125) and Gene Name (LOC100287482; SEQ ID NO: 6).

### DETAILED DESCRIPTION

The present invention relates generally to identifying and characterizing long non-coding RNAs (“lncRNAs”) that are differentially expressed in cancer cells, particularly in prostate cancer, as compared to normal tissue. The identification of cancer-associated lncRNAs and the investigation of their molecular and biological functions aids in understanding the molecular etiology of cancer and its progression.

### DEFINITION

As used herein, the term “nucleic acid molecule” or “nucleic acid” refer to an oligonucleotide, nucleotide or polynucleotide. A nucleic acid molecule may include deoxyribonucleotides, ribonucleotides, modified nucleotides or nucleotide analogs in any combination.

As used herein, the term “nucleotide” refers to a chemical moiety having a sugar (modified, unmodified, or an analog thereof), a nucleotide base (modified, unmodified, or an analog thereof), and a phosphate group (modified, unmodified, or an analog thereof). Nucleotides include deoxyribonucleotides, ribonucleotides, and modified nucleotide analogs including, for example, locked nucleic acids (“LNAs”), peptide nucleic acids (“PNAs”), L-nucleotides, ethylene-bridged nucleic acids (“ENAs”), arabinoside, and nucleotide analogs (including abasic nucleotides).

As used herein, the term “short interfering nucleic acid” or “siNA” refers to any nucleic acid molecule capable of down regulating (i.e., inhibiting) gene expression in a mammalian cells (preferably a human cell). siNA includes without limitation nucleic acid molecules that are capable of mediating sequence specific RNAi, for example short interfering RNA (siRNA), double-stranded RNA (dsRNA), micro-RNA (miRNA), and short hairpin RNA (shRNA).

As used herein, the term “sense region” refers to a nucleotide sequence of a siNA molecule complementary (partially or fully) to an antisense region of the siNA molecule. Optionally, the sense strand of a siNA molecule may also include additional nucleotides not complementary to the antisense region of the siNA molecule.

As used herein, the term “ectopic expression” refers to the occurrence of gene expression or the occurrence of a level of gene expression in a tissue in which it is not generally expressed, or not generally expressed at such a level.

As used herein, the term “antisense region” refers to a nucleotide sequence of a siNA molecule complementary (partially or fully) to a target nucleic acid sequence. Optionally, the antisense strand of a siNA molecule may include additional nucleotides not complementary to the sense region of the siNA molecule.

As used herein, the term “duplex region” refers to the region in two complementary or substantially complementary oligonucleotides that form base pairs with one another that allows for a duplex between oligonucleotide strands that are complementary or substantially complementary. For example, an oligonucleotide strand having 21 nucleotide units can base pair with another oligonucleotide of 21 nucleotide units, yet only 19 bases on each strand are complementary or substantially complementary, such that the “duplex region” consists of 19 base pairs. The remaining base pairs may, for example, exist as 5′ and/or 3′ overhangs.

An “abasic nucleotide” conforms to the general requirements of a nucleotide in that it contains a ribose or deoxyribose sugar and a phosphate but, unlike a normal nucleotide, it lacks a base (i.e., lacks an adenine, guanine, thymine, cytosine, or uracil). Abasic deoxyribose moieties include, for example, abasic deoxyribose-3′-phosphate; 1,2-dideoxy-D-ribofuranose-3-phosphate; 1,4-anhydro-2-deoxy-D-ribitol-3-phosphate.

As used herein, the term “inhibit”, “down-regulate”, or “reduce,” with respect to gene expression, means that the level of RNA molecules encoding one or more proteins or protein subunits (e.g., mRNA) is reduced below that observed in the absence of the inhibitor. Expression may be reduced by at least 90%, 80%, 70%, 60%, 50%, 40%, 30%, 20%, 10%, 5% or below the expression level observed in the absence of the inhibitor.

A group of differentially expressed long noncoding RNAs (IncRNAs) are identified in prostate cancer cell lines and patient samples using DNA microarrays, and performed confirmatory analysis using qRT-PCR and RNA-FISH. Several highly upregulated IncRNAs were further tested in prostatic adenocarcinoma tissue samples (Gleason score >6.0) and compared to matched normal tissues. AK024556, XLOC-007697, LOC100506411, LOC100287482, XLOC-001699, XLOC-005327, XLOC-008659, and XLOC-009911 were confirmed as significantly upregulated in patient samples,

In some embodiments, the IncRNA that is significantly upregulated in prostate cancer cells comparing to a reference level determined in a healthy subject is one or more of SEQ ID NOs: 1-76, or a combination thereof. In some embodiments, the IncRNA that is significantly upregulated in prostate cancer cells is XLOC_007697 (SEQ ID NO: 2). In some embodiments, the IncRNA that is significantly upregulated in prostate cancer cells is XLOC_009911 (SEQ ID NO: 3). In some embodiments, the IncRNA that is significantly upregulated in prostate cancer cells is XLOC_008559 (SEQ ID NO: 4). In some embodiments, the IncRNA that is significantly upregulated in prostate cancer cells is XLOC_005327 (SEQ ID NO: 5). In some embodiments, the IncRNA that is significantly upregulated in prostate cancer cells is LOC100287482 (SEQ ID NO: 6).

AK024556, also known as SPRY4-IT1, is an intronic IncRNA originating from the first intron of the SPRY4 gene) was previously reported to be upregulated in primary human melanomas and cell lines. SPRY4-IT1 was not expressed in LNCaP cells due to the epigenetic modification of the SPRY4 promoter by CpG island methylation. Furthermore, epigenetic silencing was reversed by treatment with 5-aza-2′-deoxycytidine (a DNA methyltransferase inhibitor) and resulted in upregulation of SPRY4 and SPRY4-IT1, indicating that SPRY4 and SPRY4-IT1 are epigenetically co-regulated. siRNA knockdown of SPRY4-IT1 inhibited proliferation and invasion, and increased apoptosis, in PC3 cells. Chromogenic in situ hybridization (CISH) assay was developed to detect SPRY4-IT1 in patient samples. The present technology is useful for prostate cancer diagnosis in a clinical setting. Results are reported here to support the notion that IncRNAs are potential diagnostic biomarkers for prostate cancers with have a role in prostate carcinogenesis.

To address the need for a specific prognostic biomarker that can refine existing diagnostic methods, several diagnostic and predictive biomarkers are being actively investigated or are in clinical use [4], including the use of PSA isoforms, kallikreins, and measurement of the expression of genes that are associated with prostate cancer (such as GSTP1, AMACR, ERG, and gene fusions involving ETS-related genes). In particular, PCA3, a long non-coding RNA (IncRNA), has shown promise for the urinary detection of prostate cancer with superior specificity to PSA [42].

LncRNAs are RNA transcripts >200 nucleotides in length [5, 6], many of which exhibit cell type-specific expression [7-9] and are localized to specific subcellular compartments [10-14]. A number of IncRNAs are known to play important roles during cellular development and differentiation [15-17], supporting the view that they are under evolutionary selection [18-21].

LncRNAs can influence the expression of target proteins at specific genomic loci [22-25], modulate the activity of protein binding partners [26-28], direct chromatin-modifying complexes to their sites of action, and undergo post-transcriptional processing to produce numerous 5′-capped small RNAs [10, 29]. Like microRNAs (miRNAs), IncRNAs are dysregulated in various diseases, including ischemia, heart disease [30, 31], Alzheimer's disease [32], psoriasis [33], spinocerebellar ataxia type 8 [34, 35], and several cancers such as breast cancer [16, 36, 37], colon cancer [38], prostate cancer [39], hepatocellular carcinoma [40, 41], and leukemia [40].

SPRY4-IT1 is upregulated in human melanomas, and siRNA-mediated knockdown of SPRY4-IT1 in melanoma cells alters cellular growth and differentiation and increases the rate of apoptosis [43]. The differential expression of several prostate cancer specific IncRNAs and their expression are investigated in prostate cancer cell lines, normal epithelial cells, and prostate cancer patient samples matched with normal tissues, and explore the molecular function of the IncRNA SPRY4-IT1 in prostate cancer cells using siRNA knockdown and cellular assays.

In some embodiments, the reduction or inhibition or down-regulation of one or more of the IncRNAs (i.e., SEQ ID NOs: 1-76, or a combination thereof) that are significantly upregulated in prostate cancer cells influence the expression of target proteins at specific genomic loci. In some embodiments, the reduction or inhibition or down-regulation of one or more of the IncRNAs (i.e., SEQ ID NOs: 1-76, or a combination thereof) that are significantly upregulated in prostate cancer cells modulate the activity of protein binding partners. In some embodiments, the reduction or inhibition or down-regulation of one or more of the IncRNAs (i.e., SEQ ID NOs: 1-76, or a combination thereof) that are significantly upregulated in prostate cancer cells direct chromatin-modifying complexes to their sites of action. In some embodiments, the reduction or inhibition or down-regulation of one or more of the IncRNAs (i.e., SEQ ID NOs: 1-76, or a combination thereof) that are significantly upregulated in prostate cancer cells undergo post-transcriptional processing to produce 5′-capped small RNAs. In some embodiments, the IncRNA is XLOC_007697 (SEQ ID NO: 2). In some embodiments, the IncRNA is XLOC_009911 (SEQ ID NO: 3). In some embodiments, the IncRNA is XLOC_008559 (SEQ ID NO: 4). In some embodiments, the IncRNA is XLOC_005327 (SEQ ID NO: 5). In some embodiments, the IncRNA is LOC100287482 (SEQ ID NO: 6).

RNA Interference and siNA

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). Post-transcriptional gene silencing is believed to be an evolutionarily-conserved cellular mechanism for preventing expression of foreign genes that may be introduced into the host cell (Fire et al., 1999, Trends Genet., 15, 358). Post-transcriptional gene silencing may be an evolutionary response to the production of double-stranded RNAs (dsRNAs) resulting from viral infection or from the random integration of transposable elements (transposons) into a host genome. The presence of dsRNA in cells triggers the RNAi response that appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. No. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of dicer, a ribonuclease III enzyme (Bass, 2000, Cell, 101, 235; Zamore et al., 2000, Cell, 101, 25-33; Hammond et al., 2000, Nature, 404, 293). Dicer processes long dsRNA into double-stranded short interfering RNAs (siRNAs) which are typically about 21 to about 23 nucleotides in length and include about 19 base pair duplexes (Zamore et al., 2000, Cell, 101, 25-33; Bass, 2000, Cell, 101, 235; Elbashir et al., 2001, Genes Dev., 15, 188).

Single-stranded RNA, including the sense strand of siRNA, trigger an RNAi response mediated by an endonuclease complex known as an RNA-induced silencing complex (RISC). RISC mediates cleavage of this single-stranded RNA in the middle of the siRNA duplex region (i.e., the region complementary to the antisense strand of the siRNA duplex) (Elbashir et al., 2001, Genes Dev., 15, 188).

In certain embodiments, the siNAs may be a substrate for the cytoplasmic Dicer enzyme (i.e., a “Dicer substrate”) which is characterized as a double stranded nucleic acid capable of being processed in vivo by Dicer to produce an active nucleic acid molecules. The activity of Dicer and requirements for Dicer substrates are described, for example, U.S. 2005/0244858. Briefly, it has been found that dsRNA, having about 25 to about 30 nucleotides, effective result in a down-regulation of gene expression. Without wishing to be bound by any theory, it is believed that Dicer cleaves the longer double stranded nucleic acid into shorter segments and facilitates the incorporation of the single-stranded cleavage products into the RNA-induced silencing complex (RISC complex). The active RISC complex, containing a single-stranded nucleic acid cleaves the cytoplasmic RNA having complementary sequences.

It is believed that Dicer substrates must conform to certain general requirements in order to be processed by Dicer. The Dicer substrates must of a sufficient length (about 25 to about 30 nucleotides) to produce an active nucleic acid molecule and may further include one or more of the following properties: (i) the dsRNA is asymmetric, e.g., has a 3′ overhang on the first strand (antisense strand) and (ii) the dsRNA has a modified 3′ end on the antisense strand (sense strand) to direct orientation of Dicer binding and processing of the dsRNA to an active siRNA. The Dicer substrates may be symmetric or asymmetric. For example, Dicer substrates may have a sense strand includes 22-28 nucleotides and the antisense strand may include 24-30 nucleotides, resulting in duplex regions of about 25 to about 30 nucleotides, optionally having 3′-overhangs of 1-3 nucleotides.

Dicer substrates may have any modifications to the nucleotide base, sugar or phosphate backbone as known in the art and/or as described herein for other nucleic acid molecules (such as siNA molecules).

The RNAi pathway may be induced in mammalian and other cells by the introduction of synthetic siRNAs that are 21 nucleotides in length (Elbashir et al., 2001, Nature, 411, 494 and Tuschl et al., WO 01/75164; incorporated by reference in their entirety). Other examples of the requirements necessary to induce the down-regulation of gene expression by RNAi are described in Zamore et al., 2000, Cell, 101, 25-33; Bass, 2001, Nature, 411, 428-429; Kreutzer et al., WO 00/44895; Zernicka-Goetz et al., WO 01/36646; Fire, WO 99/32619; Plaetinck et al., WO 00/01846; Mello and Fire, WO 01/29058; Deschamps-Depaillette, WO 99/07409; and Li et al., WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831; each of which is hereby incorporated by reference in its entirety.

Briefly, an siNA nucleic acid molecule can be assembled from two separate polynucleotide strands (a sense strand and an antisense strand) that are at least partially complementary and capable of forming stable duplexes. The length of the duplex region may vary from about 15 to about 49 nucleotides (e.g., about 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides). Typically, the antisense strand includes nucleotide sequence that is complementary to nucleotide sequence in a target nucleic acid molecule. The sense strand includes nucleotide sequence corresponding to the target nucleic acid sequence which is therefore at least substantially complementary to the antisense stand. Optionally, an siNA is “RISC length” and/or may be a substrate for the Dicer enzyme. Optionally, an siNA nucleic acid molecule may be assembled from a single polynucleotide, where the sense and antisense regions of the nucleic acid molecules are linked such that the antisense region and sense region fold to form a duplex region (i.e., forming a hairpin structure).

### 5′ Ends, 3′ Ends and Overhangs

siNAs may be blunt-ended on both sides, have overhangs on both sides or a combination of blunt and overhang ends. Overhangs may occur on either the 5′- or 3′-end of the sense or antisense strand. Overhangs typically consist of 1-8 nucleotides (e.g., 1, 2, 3, 4, 5, 6, 7, or 8 nucleotides each) and are not necessarily the same length on the 5′- and 3′-end of the siNA duplex. The nucleotide(s) forming the overhang need not be of the same character as those of the duplex region and may include deoxyribonucleotide(s), ribonucleotide(s), natural and non-natural nucleobases or any nucleotide modified in the sugar, base or phosphate group such as disclosed herein.

The 5′- and/or 3′-end of one or both strands of the nucleic acid may include a free hydroxyl group or may contain a chemical modification to improve stability. Examples of end modifications (e.g., terminal caps) include, but are not limited to, abasic, deoxy abasic, inverted (deoxy) abasic, glyceryl, dinucleotide, acyclic nucleotide, amino, fluoro, chloro, bromo, CN, CF, methoxy, imidazole, carboxylate, thioate, C1 to C10 lower alkyl, substituted lower alkyl, alkaryl or aralkyl, OCF3, OCN, O-, S-, or N-alkyl; O-, S-, or N-alkenyl; SOCH3; SO2CH3; ONO2; NO2, N3; heterocycloalkyl; heterocycloalkaryl; aminoalkylamino; polyalkylamino or substituted silyl, as, among others, described in European patents EP 586,520 and EP 618,925.

### Chemical Modifications

siNA molecules optionally may contain one or more chemical modifications to one or more nucleotides. There is no requirement that chemical modifications are of the same type or in the same location on each of the siNA strands. Thus, each of the sense and antisense strands of an siNA may contain a mixture of modified and unmodified nucleotides. Modifications may be made for any suitable purpose including, for example, to increase RNAi activity, increase the in vivo stability of the molecules (e.g., when present in the blood), and/or to increase bioavailability.

Suitable modifications include, for example, internucleotide or internucleoside linkages, dideoxyribonucleotides, 2′-sugar modification including amino, fluoro, methoxy, alkoxy and alkyl modifications; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidi-ne (d4T) and the monophosphate nucleotides of 3′-azido-3′-deoxythymidine (AZT), 2′,3′-dideoxy-3′-thiacytidine (3TC) and 2′,3′-didehydro-2′,3′-dide-oxythymidine (d4T).

Other suitable modifications include, for example, locked nucleic acid (LNA) nucleotides (e.g., 2′-0, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides (WO 00/47599, WO 99/14226, WO 98/39352, and WO 2004/083430).

Chemical modifications also include terminal modifications on the 5′ and/or 3′ part of the oligonucleotides and are also known as capping moieties. Such terminal modifications are selected from a nucleotide, a modified nucleotide, a lipid, a peptide, and a sugar.

Chemical modifications also include L-nucleotides. Optionally, the L-nucleotides may further include at least one sugar or base modification and/or a backbone modification as described herein.

### Delivery of Nucleic Acid-Containing Pharmaceutical Formulations

Nucleic acid molecules disclosed herein (including siNAs and Dicer substrates) may be administered with a carrier or diluent or with a delivery vehicle which facilitate entry to the cell. Suitable delivery vehicles include, for example, viral vectors, viral particles, liposome formulations, and lipofectin.

Methods for the delivery of nucleic acid molecules are described in Akhtar et al., Trends Cell Bio., 2: 139 (1992); Delivery Strategies for Antisense Oligonucleotide Therapeutics, ed. Akhtar, (1995), Maurer et al., Mol. Membr. Biol., 16: 129-140 (1999); Hofland and Huang, Handb. Exp. Pharmacol., 137: 165-192 (1999); and Lee et al., ACS Symp. Ser., 752: 184-192 (2000); U.S. Pat. Nos. 6,395,713; 6,235,310; 5,225,182; 5,169,383; 5,167,616; 4,959217; 4.925,678; 4,487,603; and 4,486,194; WO 94/02595; WO 00/03683; WO 02/08754; and U.S. 2003/077829.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see e.g., Gonzalez et al., Bioconjugate Chem., 10: 1068-1074 (1999); WO 03/47518; and WO 03/46185), poly(lactic-co-glycolic)acid (PLGA) and PLCA microspheres (see for example U.S. Pat. No. 6,447,796 and U.S. 2002/130430), biodegradable nanocapsules, and bioadhesive microspheres, or by proteinaceous vectors (WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5: 2330-2337 (1999) and WO 99/31262. The molecules of the instant invention can be used as pharmaceutical agents.

Nucleic acid molecules may be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly (ethylene glycol) lipids (PEG-modified, or long-circulating liposomes or stealth liposomes).

Nucleic acid molecules may be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof (see, for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; U.S. Pat. No. 6,586,524 and U.S. 2003/0077829).

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA, the neutral lipid DOPE (GIBCO BRL) and Di-Alkylated Amino Acid (DiLA2).

Therapeutic nucleic acid molecules may be expressed from transcription units inserted into DNA or RNA vectors. Recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors are capable of expressing the nucleic acid molecules either permanently or transiently in target cells. Delivery of nucleic acid molecule expressing vectors can be systemic, such as by intravenous, subcutaneous, or intramuscular administration.

Expression vectors may include a nucleic acid sequence encoding at least one nucleic acid molecule disclosed herein, in a manner which allows expression of the nucleic acid molecule. For example, the vector may contain sequence(s) encoding both strands of a nucleic acid molecule that include a duplex. The vector can also contain sequence(s) encoding a single nucleic acid molecule that is self-complementary and thus forms a nucleic acid molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine. An expression vector may encode one or both strands of a nucleic acid duplex, or a single self-complementary strand that self hybridizes into a nucleic acid duplex. The nucleic acid sequences encoding nucleic acid molecules can be operably linked to a transcriptional regulatory element that results expression of the nucleic acid molecule in the target cell. Transcriptional regulatory elements may include one or more transcription initiation regions (e.g., eukaryotic pol I, II or III initiation region) and/or transcription termination regions (e.g., eukaryotic pol I, II or III termination region). The vector can optionally include an open reading frame (ORF) for a protein operably linked on the 5′ side or the 3′-side of the sequence encoding the nucleic acid molecule; and/or an intron (intervening sequences).

The nucleic acid molecules or the vector construct can be introduced into the cell using suitable formulations. One preferable formulation is with a lipid formulation such as in Lipofectamine™ 2000 (Invitrogen, CA, USA), vitamin A coupled liposomes (Sato et al. Nat Biotechnol 2008; 26:431-442, PCT Patent Publication No. WO 2006/068232). Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA in a buffer or saline solution and directly inject the formulated dsRNA into cells, as in studies with oocytes. The direct injection of dsRNA duplexes may also be done. Suitable methods of introducing dsRNA are provided, for example, in U.S. 2004/0203145 and U.S. 20070265220.

Polymeric nanocapsules or microcapsules facilitate transport and release of the encapsulated or bound dsRNA into the cell. They include polymeric and monomeric materials, especially including polybutylcyanoacrylate. The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Nucleic acid moles may be formulated as a microemulsion. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system. Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

### EXAMPLES

The present methods, thus generally described, will be understood more readily by reference to the following examples, which are provided by way of illustration and are not intended to be limiting of the present methods and kits.

### Example 1: Differentially Expressed INCRNA Transcripts in Prostate Cancer Cell Lines

To identify which IncRNAs are differentially expressed in prostate cancer compared to normal prostatic epithelium, total RNA from human prostate epithelial cells and the prostate cancer cell line PC3 were screened using Ncode human microarrays. The Ncode human ncRNA microarray is designed to interrogate 12,784 IncRNAs and the expression of 25,409 mRNA target protein-coding genes. In addition, genome-wide expression analysis was performed on total RNA extracted from two prostate cancer cell lines (PC3 and LNCaP) and epithelial cells using the Agilent SurePrint G3 Human Gene Expression v2 microarray. This array measures expression of 16,472 IncRNAs and 34,127 mRNAs genes, and has an overlap of 460 lncRNAs and 8,877 mRNAs with the Ncode array. Therefore, by using these two arrays, a total of 28,796 IncRNAs and 50,659 mRNAs were examined.

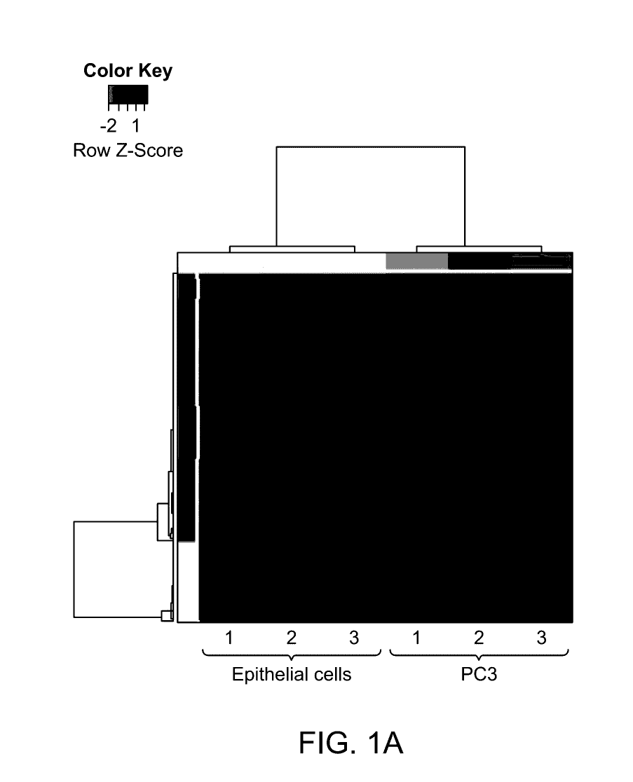

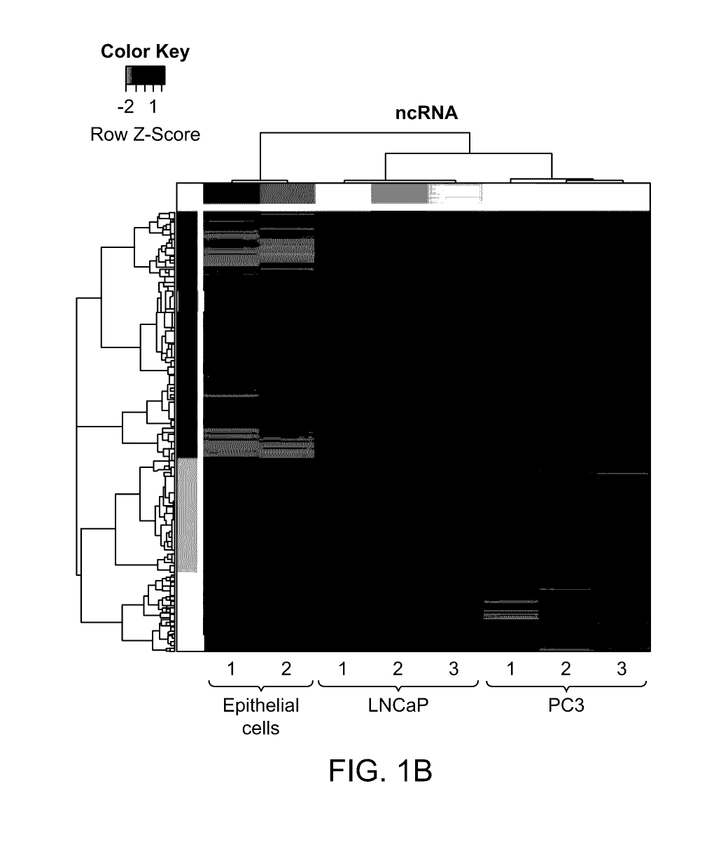

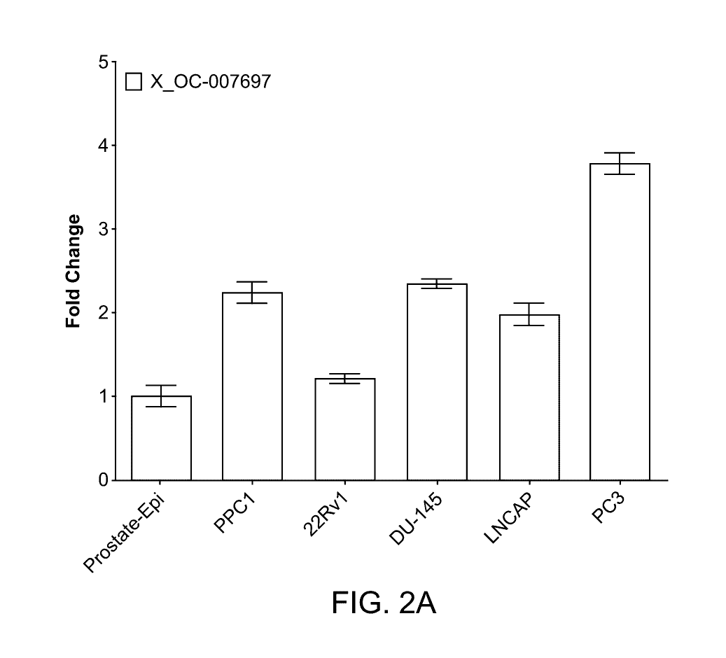

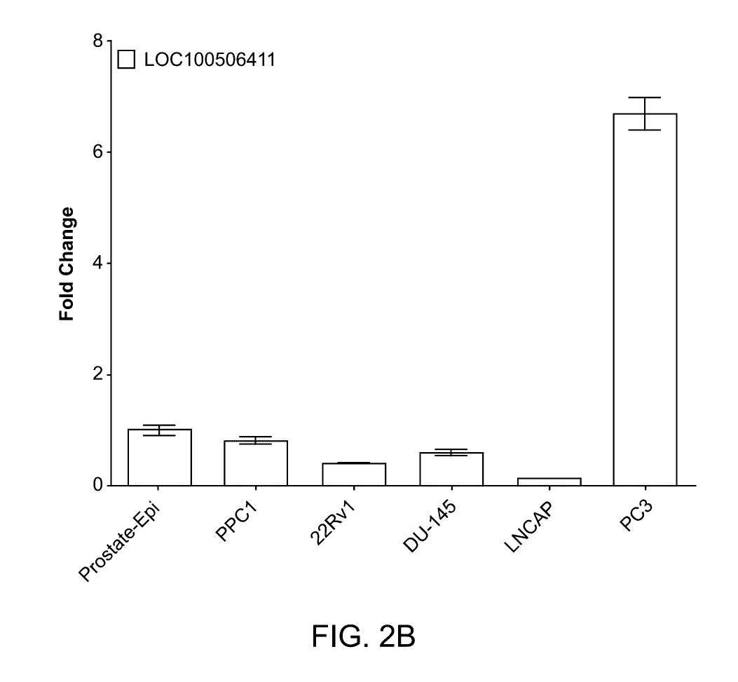

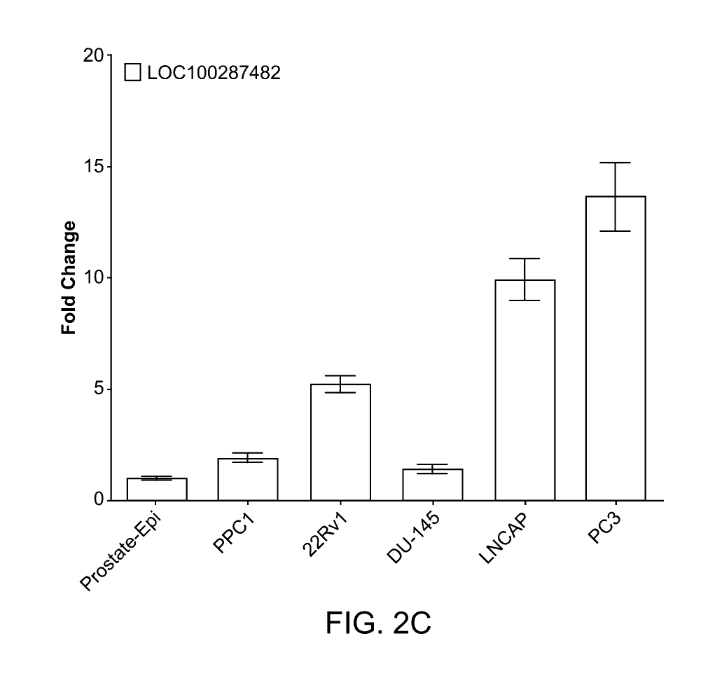

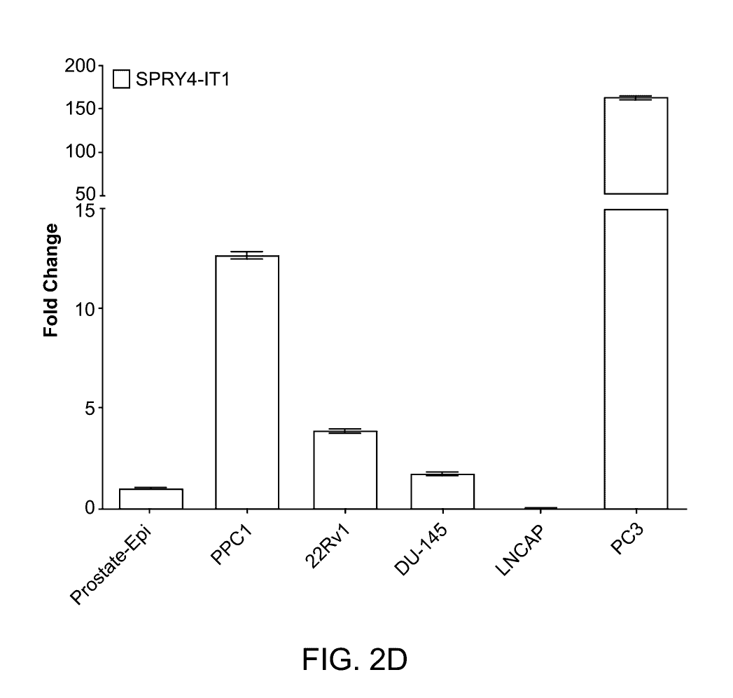

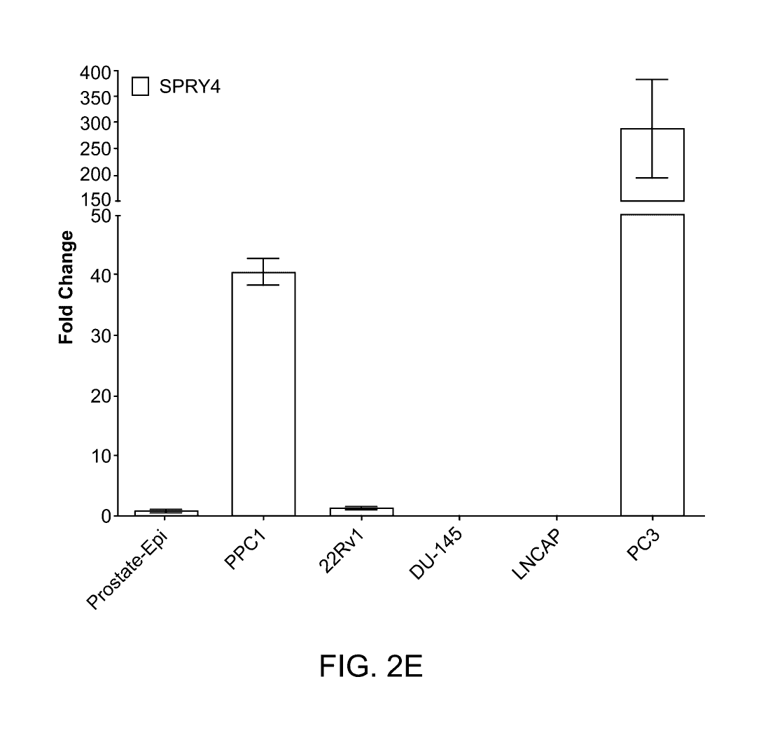

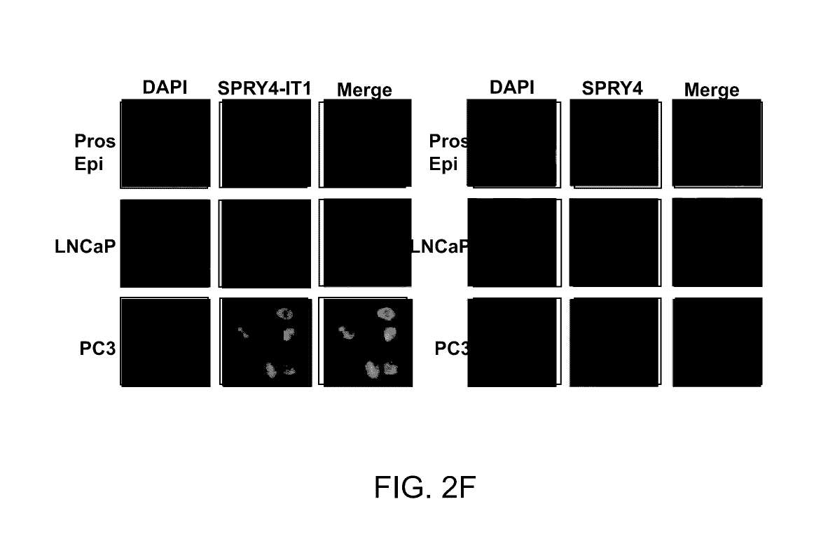

AS shown in FIGS. 1A and 1B, hierarchical clustering of differentially expressed genes using the two arrays are examined. Those genes with differential expression between PC3 and epithelial cells with P-values less than 0.015 are listed in Table 1. The expression levels of four top-ranking candidates: AK024556 (i.e., SPRY4-IT1), XLOC-007697, LOC100506411, and LOC1000287482 were further confirmed by qRT-PCR of total RNA extracted from a panel of five common prostate cancer cell lines (PPC1, 22RV1, DV-145, LNCaP, and PC3; FIG. 2A-E). Although the expression of all four IncRNAs varied between the cell lines, they were increased in the majority of the prostate cancer cell lines. More specifically, all four IncRNAs were highly upregulated in PC3 cells, which are androgen-insensitive prostate cancer cell lines and are highly metastatic compared to DU-145 and LNCaP (Pulukuri et al. 2005. J Biol Chem, 280, 36529-40). Table 1 illustrates a second group of differentially expressed prostate cancer IncRNAs candidates in PC3, LNCaP, and prostatic epithelial cells.

TABLE 1
A summary of upregulated IncRNAs detected using microarrays in prostate cancer
cell lines (epithelial cells, PC3, and LNCaP)

| | | | p. value | p. value | p. Value | |
|---|---|---|---|---|---|---|
| Gene Symbol | IgFC (PC/EP) | IgFC (LN/EP) | PC vs EP | LN vs EP | LN vs PC | Genomic Coordinates |
| AF087978 | 5.8 | | 1.16E−11 | | | chr5: 92955865-92955925 |
| uc002lic | 4.8 | | 4.64E−10 | | | chr18: 69150768-69167330 |
| EF177379 | 4.3 | | 3.43E−10 | | | chr11: 64949929-64949969 |
| BC013821 | 3.8 | | 3.08E−11 | | | chr6: 533914-533974 |
| uc001pyz | 3.7 | | 7.44E−13 | | | chr11: 123003624-123003684 |
| AK024556 | 3.7 | | 3.07E−11 | | | chr6: 141677414-141677474 |
| AB116663 | 3.8 | | 2.33E−11 | | | chr1: 76682805-76682955 |
| BC012000 | 3.5 | | 3.50E−00 | | | chr8: 29240600-29240050 |
| LOC100506303 | 2.6 | 5.1 | 0 | 0 | 0 | chr14: 19662601-19662660 |
| LOC100506922 | 3.5 | 4.9 | 0 | 0 | 0 | chr2: 128145912-128145971 |
| LOC100287482 | 3.0 | 4.8 | 0 | 0 | 0 | chr7: 129152443-129152502 |
| XLOC-I2_009441 | 3.3 | 4.7 | 0 | 0 | 0 | chr22: 16148038-16147979 |
| LOC154822 | 3.2 | 4.6 | 0.00771 | 0.00103 | | chr7: 168815312-168815371 |
| XLOC_610807 | 3.6 | 4.5 | 0 | 0 | | chr14: 38208320-38203879 |
| XLOC_002335 | 3.4 | 4.5 | 0.00036 | 7.00E−05 | | chr2: 138638416-138638357 |
| XLOC_I2_009136 | 2.1 | 4.4 | 0 | 0 | 0 | chr21: 15335073-15323443 |
| XLOC_002871 | 2.3 | 4.2 | 1.00E−05 | 0 | 3.00E−05 | chr3: 149957932-149957991 |
| ANKRD2DA9P | 2.0 | 4.0 | 0 | 0 | 0 | chr13: 19415809-19415750 |
| FLJ20444 | 4.1 | 4.0 | 0 | 0 | | chr9: 66524045-66523080 |
| XLOC_003734 | 4.0 | 3.8 | 0 | 0 | | chr4: 162591561-152591520 |
| LOC100505668 | 2.1 | 3.7 | 0 | 0 | 0 | chr1: 155017772-155017713 |
| LOC100506411 | 4.1 | 3.6 | 0 | 0 | | chr14: 71281913-71281972 |
| XLOC_007697 | 3.4 | 3.3 | 0 | 0 | | chr9: 044182789-044182730 |
| XLOC_003734 | 3.5 | 3.2 | 0 | 0 | | chr4: 152591414-152591473 |
| XLOC_I2_000735 | 4.6 | 2.8 | 0 | 1.00E−05 | 6.00E−05 | chr1: 1006425-1006366 |
| LOC100129480 | 4.5 | 2.4 | 0 | 0 | 0 | chr3: 12581659-12581600 |
| XLOC_007162 | 3.1 | 2.2 | 0 | 0 | | chr8: 95650283-95840030 |
| LOC100566602 | 3.4 | 2.1 | 0 | 0 | 0 | chr3: 60405569-60405628 |
| LOC100507025 | 5.7 | 2.1 | 0 | 0 | 0 | chr6: 26261345-26261285 |
| XLOC_010813 | 3.8 | 2.0 | 0 | 0 | 0 | chr14: 041431994-041432050 |

Interestingly, SPRY4-IT1 was previously identified as one of the highly upregulated IncRNAs in human melanoma cells [43]. qRT-PCR analysis further confirmed that SPRY4-IT1 was upregulated over 100-fold in PC3 cells compared to prostatic epithelial cells (FIG. 2D). Overexpression of SPRY4-IT1 was also seen in PPC1 cells, albeit to a lesser extent (<10 fold), but no expression was observed in LNCaP cells. When compared to the expression profile of SPRY4 (the open-reading frame in which SPRY4-IT1 is embedded), the expression patterns were similar, with PC3 cells showing the highest expression levels, followed by PPC1 cells (FIG. 2E); this suggests coordinated dysregulation of both coding and non-coding RNAs in prostate cancer cells. Although both PC3 and DU145 cells are androgen insensitive, there was almost no expression of either SPRY4 or SPRY4-IT1 in DU145 cells, a pattern mirrored by the androgen-sensitive LNCaP cells. These data indicate that the expression of these transcripts may not be associated with androgen sensitivity. Consequently, staining of PC3, LNCaP, and prostatic epithelial cells using RNA-FISH confirmed that both SPRY4 and SPRY4-IT1 could only be easily detected in PC3 cells (FIG. 2F), in line with the qRT-pCR results (FIG. 2D-E).

### Example 2: Hypermethylation of the Upstream Regulatory Region of SPRY4 Appears to Co-Regulate Expression of SPRY4 and the INCRNA SPRY4-IT in Prostate Cancer Cells

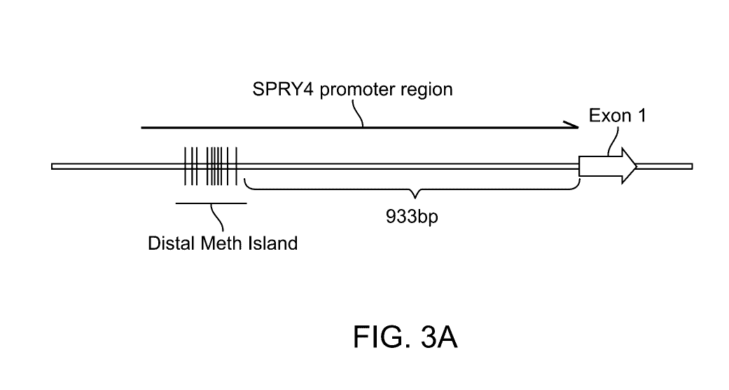

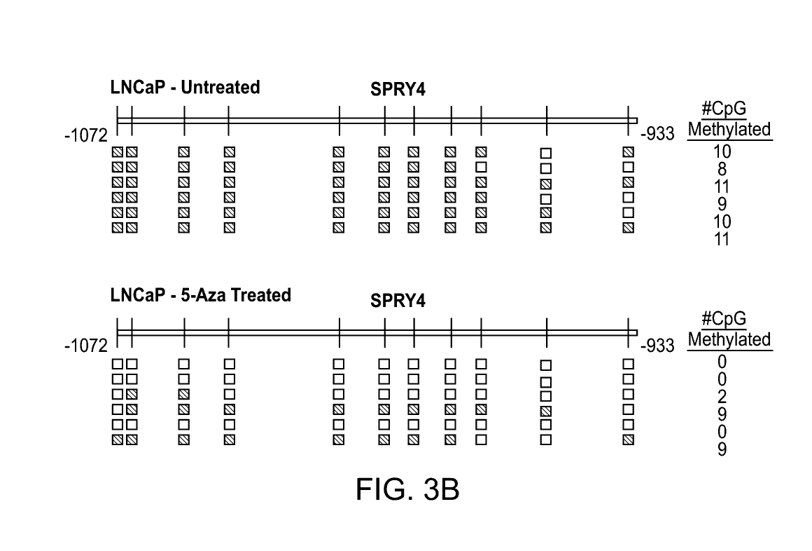

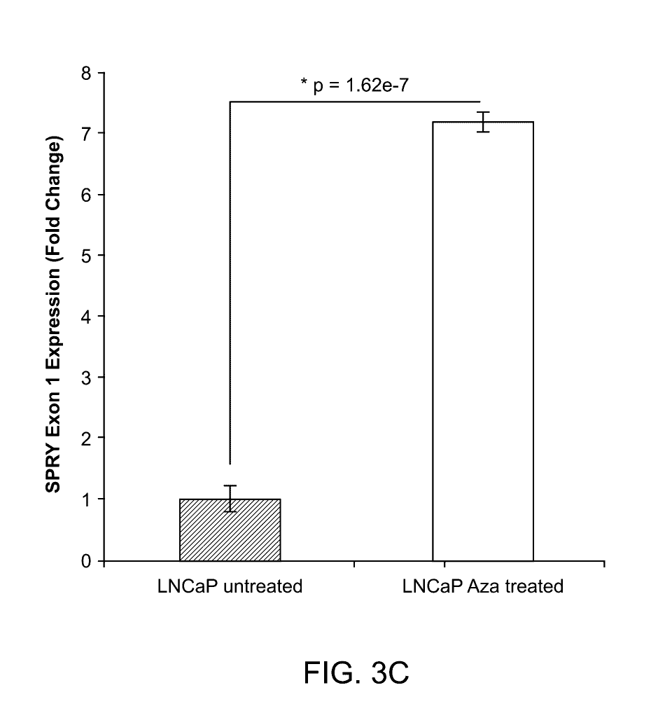

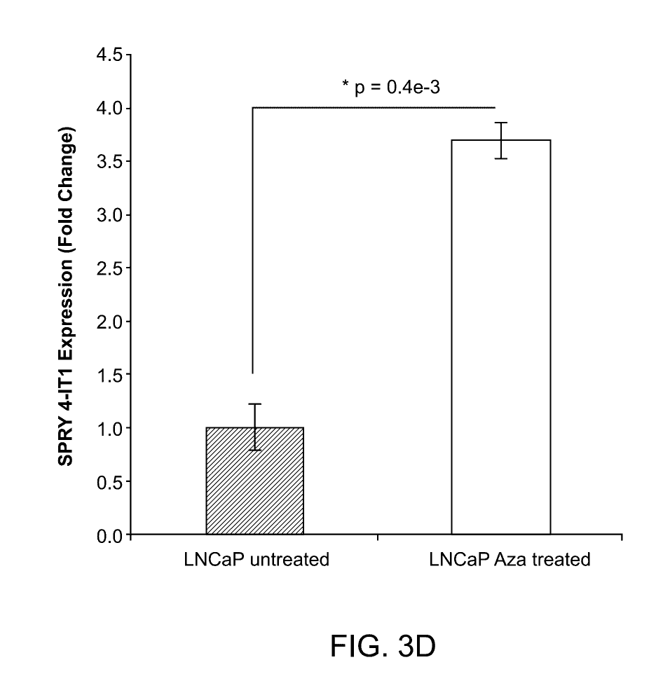

An examination of the SPRY4 gene reveals that only one CpG island exists within its genomic locus. This island is present ˜900 bp upstream of the transcriptional start site (TSS; containing 11 CpG sequences in a 139 bp region; FIG. 3A). This island has previously been shown to be frequently methylated in prostate cancer (specifically in LNCaP cells, but not in PC3 cells) [44]. Examination of this region by bisulphite sequencing confirmed methylation of this CpG island (FIG. 3B). Since inhibition of methylation with 5-aza-2′-deoxycytidine treatment of LNCaP cells has been shown to deplete methylation at this island and induced the expression of SPRY4 [44], and that SPRY4 and SPRY4-IT1 appear to be co-regulated both here and in melanoma [43], whether depletion of methylation at this CpG island induce simultaneous expression of both SPRY4 and SPRY4-IT1 was examined.

After treatment of LNCaP cells with 5-aza-2′-deoxycytidine, half of the cell samples were bisulphite sequenced. The majority of methylation at this CpG island was depleted (89% methylation vs 30% after treatment; FIG. 3B). The other half of the cell samples were used for qRT-PCR analysis of both SPRY4 and SPRY4-IT1 expression (FIG. 3C-D). SPRY4 expression increased by ˜7-fold and expression of SPRY4-IT1 also increased (by ˜3.5-4 fold), indicating that SPRY4 and SPRY4-IT1 are likely to use the same promoter and can thus be transcriptionally inhibited by the same CpG island.

### Example 3: Differentially Expressed INCRNAS in Prostate Cancer Patient Samples and Matched Normal Tissue

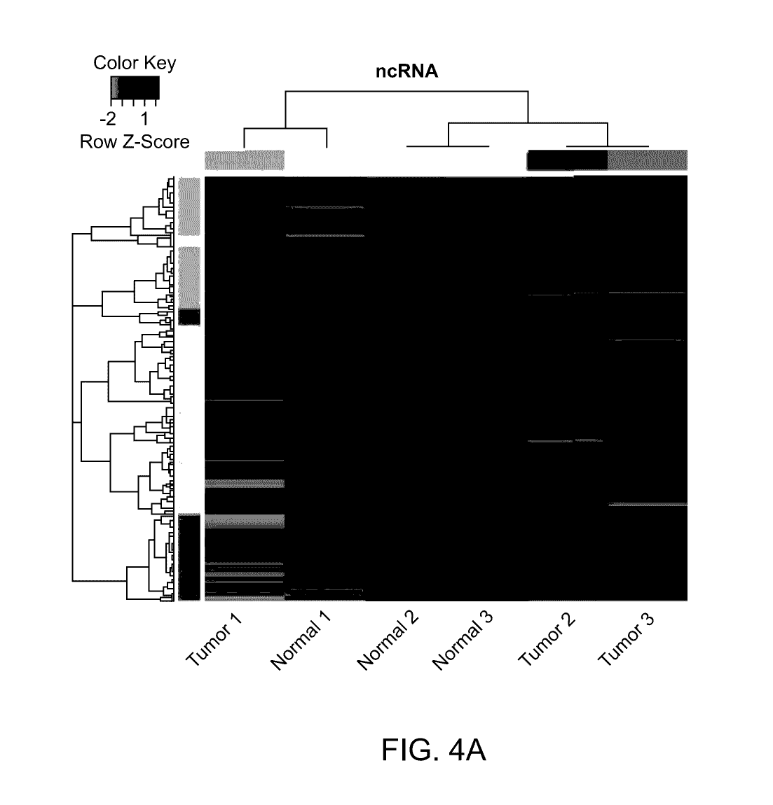

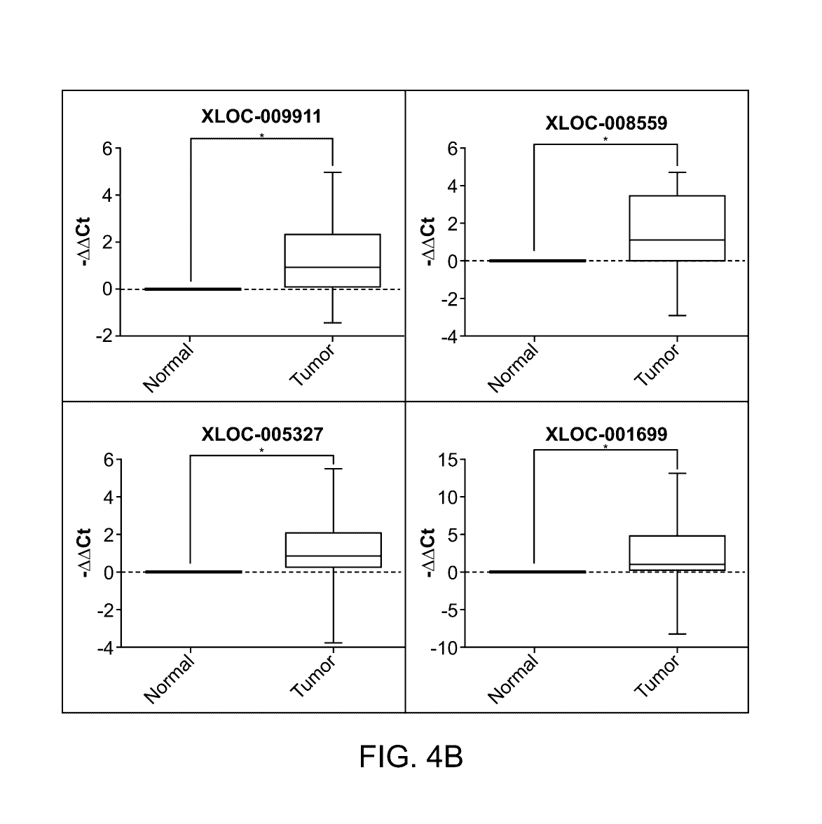

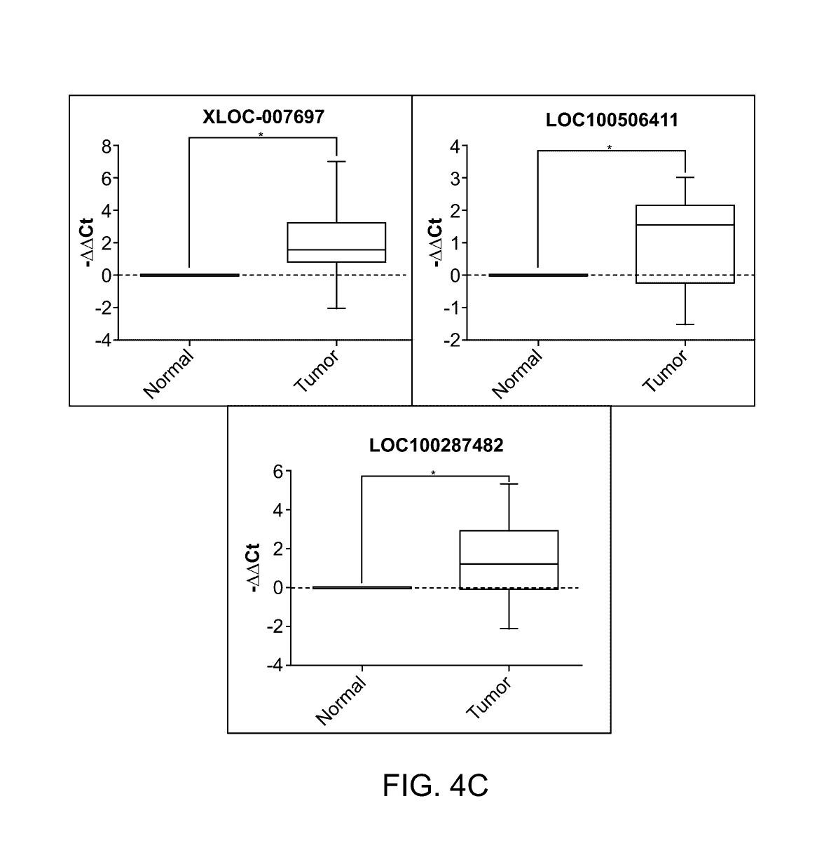

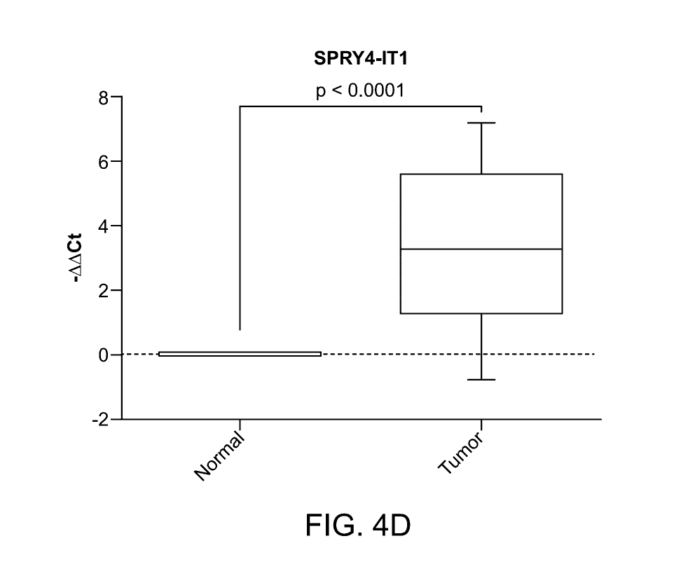

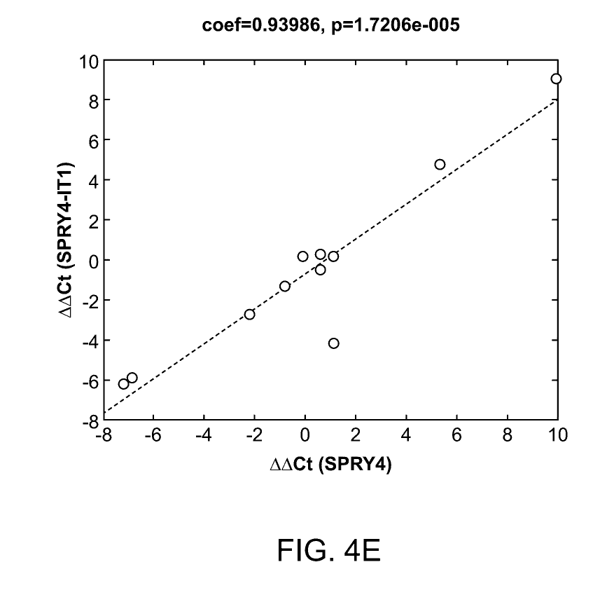

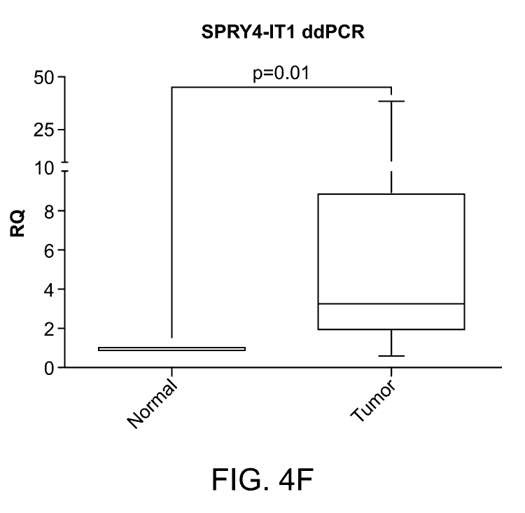

Since the global IncRNA expression profile of prostate cancer has not been fully established, IncRNA expression profiles in prostate tissue samples from patients with prostate cancer were investigated. Ten paired (tumor and adjacent normal tissue) frozen biopsy specimens were obtained and total RNA profiled using the Agilent SurePrint G3 Human Gene Expression v2 microarray. Hierarchical clustering of the differentially expressed genes is shown in FIG. 4A, and the IncRNAs upregulated in tumor tissues listed in Table 2. Several differentially expressed genes (XLOC-008559, XLOC-005327, XLOC-001699, and XLOC-009911) were further validated in an independent set of prostate cancer tissue samples. XLOC-008559 is located on chr10:92749981-92750040, while the other three are located on chr6, chr2, and chr12, respectively (Table 2), in large intergenic regions. XLOC-005327 and XLOC-009911 have two and four transcript variants, respectively. qRT-PCR primers were designed for common exons for each IncRNA, and the expression level of each IncRNA was measured in 15 paired (tumor and adjacent normal tissue) formalin-fixed, paraffin-embedded (FFPE) tissue samples by qRT-PCR. The expression of XLOC-008559, XLOC-005327, XLOC-001699, and XLOC-009911 were all significantly higher in prostate tumor tissues (FIG. 4B, P=0.03, 0.03, 0.05, and 0.01, respectively) compared to matched normal tissue. Three of the IncRNAs (XLOC-007697, LOC100506411, and LOC100287482) were further validated identified as upregulated in the cell lines (Table 1) in FFPE samples by qRT-PCR. As shown in FIG. 4C, all three IncRNAs were significantly upregulated in tumor tissues. There was no correlation between each IncRNA expression level and clinicopathological features (data not shown).

TABLE 2
A summary of upregulated IncRNAs detected using
microarrays in ten pairs of primary prostate cancer
tissue samples and adjacent normal tissues

| | IgFC | p value | |
|---|---|---|---|
| Gene Symbol | (T/N) | T vs N | Genomic Coordinates |
| XLOC_001699 | 4.3 | 0.0006 | chr2: 147600077-147607078 |
| XLOC_005327 | 4.1 | 0.0096 | chr6: 53495636-53495697 |
| LOC400956 | 3.9 | 0.0003 | chr2: 65129700-65129721 |
| XLOC_008559 | 3.7 | 0.0036 | chr10: 92749981-92750040 |
| LINC00340 | 3.0 | 0.0010 | chr6: 22110000-23111000 |
| XLOC_000465 | 3.0 | 0.0475 | chr1: 105632440-105600104 |
| RPS10 | 3.3 | 0.0007 | chr6: 33244222-33244279 |
| XLOC_012294 | 3.3 | 0.0124 | chr17: 67709660-67841626 |
| XLOC_I2_008560 | 3.3 | 0.0045 | chr20: 16465441-18485738 |
| XLOC_009911 | 3.2 | 0.0056 | chr12: 121343059-121343118 |

### Example 4: SPRY-IT1 Transcript is Upregulated in Primary Human Prostatic Adenocarcinomas Compared to Matched Normal Tissues

SPRY4-IT1 expression levels were measured by qRT-PCR in a total of 18 matched normal prostate and prostatic adenocarcinoma tissue samples, with expression values normalized to 1 in the matched normal tissue. The expression of SPRY4-IT1 was variable in both normal and cancer tissues, probably due to variability in tissue composition (i.e. epithelial and stromal composition) and variable expression per cell. However, SPRY4-IT1 was significantly upregulated in cancerous tissue (FIG. 4D), with its expression increased in 16 out of 18 cancer cases (89%) relative to paired normal tissue samples. The expression of SPRY4-IT1 was further confirmed using a droplet digital PCR (ddPCR) system, which has the advantage of being able to detect target molecules in very small quantities of sample RNA. This is particularly useful for FFPE tissue samples, since the recovery efficiency of RNA from FFPE is generally poor. Using only a third of the amount of cDNA compared to qRT-PCR, there was upregulation of SPRY4-IT1 in tumor compared to matched normal tissue (p=0.01; FIG. 4F). Although the exact relative expression levels of SPRY4-IT1 measured by ddPCR were not identical to qRT-PCR values, the overall pattern of SPRY4-IT1 expression in each patient sample remained the same (Table 3).

TABLE 3
Comparison of relative SPRY4-IT1 expression between
ddPCR and qRT-PCR in patient samples.

| | RQ | RQ |
|---|---|---|
| Patient | ddPCR | qRT-PCR |
| 1 | 35.1 | 62 |
| 2 | 3.6 | 15 |
| 3 | 1.0 | 17 |
| 4 | 2.1 | 1.5 |
| 5 | 2.8 | 7 |
| 6 | 08 | 0.9 |
| 7 | 1.5 | 2 |
| 8 | 38.6 | 45 |
| 9 | 0.6 | 0.6 |
| 10 | 15.0 | 103 |
| 11 | 3.3 | 5 |
| 12 | 6.6 | 12 |
| 13 | 41 | 7 |
| 14 | 6.8 | 23 |
| 15 | 23.5 | 151 |
| 16 | 2.1 | 2 |
| 17 | 3.0 | 3 |
| 18 | 3.2 | 128 |

Since SPRY4-IT1 and SPRY4 can both be regulated by methylation of the same promoter (FIG. 3), the expression of both SPRY4-IT1 and SPRY4 mRNA levels were compared simultaneously by qRT-PCR in 11 paired samples. The tumor tissues with high expression levels of SPRY4-IT1 were also found to highly express SPRY4, compared to matched normal tissues (FIG. 4E), further suggesting that these two RNA products are co-regulated.

### Example 5: In Situ Hybridization Confirms that SPRY4-IT1 Expression Specific to Prostate Cancer Patients

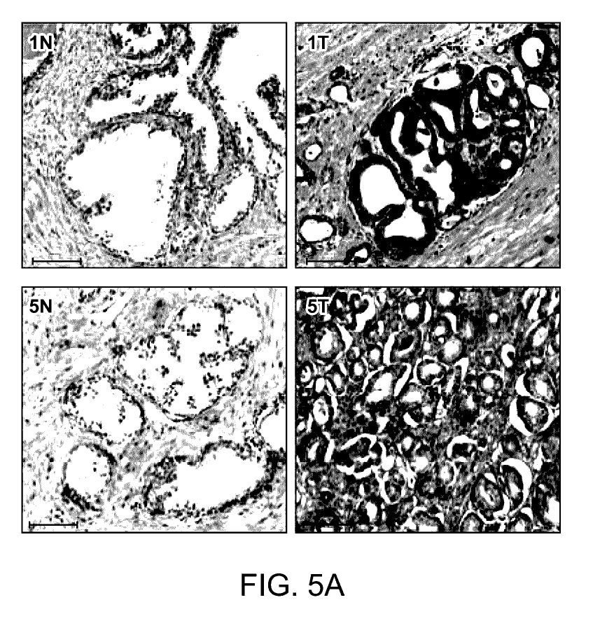

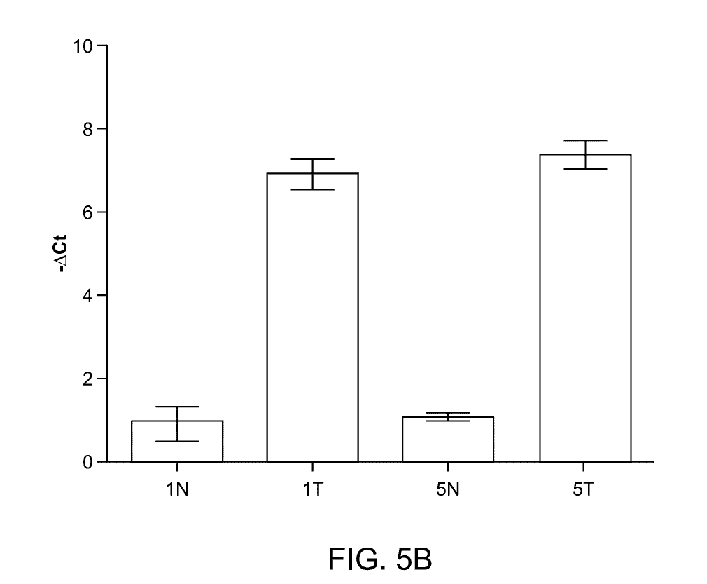

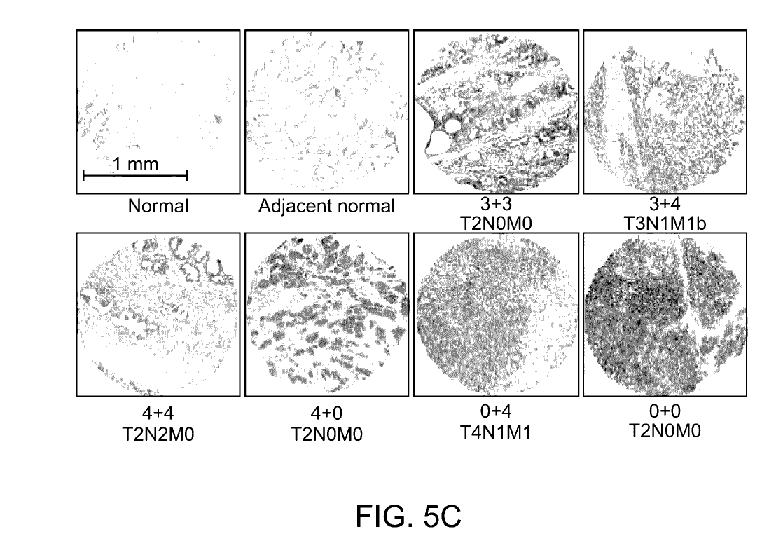

Having confirmed that SPRY4-IT1 is overexpressed in primary prostatic adenocarcinoma by both ddPCR and qRT-PCR, SPRY4-IT1 expression in situ was visualized using RNA-CISH of tissue sections. Two matched tissue samples were selected for RNA-CISH and simultaneous comparison by qRT-PCR. There was a large difference in expression (an average increase of ˜7-fold) between the tumors and matched normal tissues (FIG. 5A-B), which was confirmed by strong staining in malignant glands, but not normal prostatic glands, by RNA-CISH.

RNA-CISH was performed on a prostate cancer tissue array in order to confirm specificity of expression in prostatic adenocarcinoma and assess associations with Gleason grading. SPRY4-IT1 expression was easily detected in all adenocarcinoma samples (Gleason scores 6 (3+3), 7 (3+4), 8 (4+4), 9 (5+4 & 4+5), & 10 (5+5)). However there was little or no staining in either normal (no cancer in the patient) or normal tissue adjacent to the cancer. These data indicate that SPRY4-IT1 expression is specific to adenocarcinoma and can be detected using standard clinical staining procedures, suggesting that this biomarker may be a viable diagnostic tool.

### Example 6: Molecular Function of SPRY4-IT1 in Prostate Cancer Cells

Previous study of SPRY4-IT1 in melanoma indicated that loss resulted in several negative phenotypes in the SPRY4-IT1-expressing cell lines examined [43].

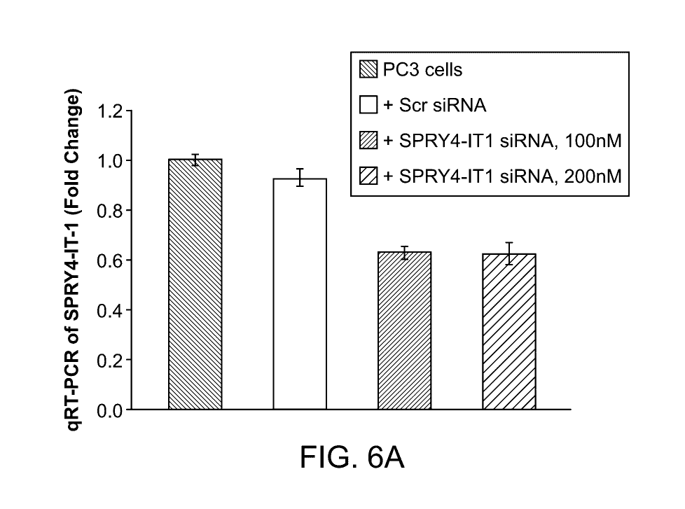

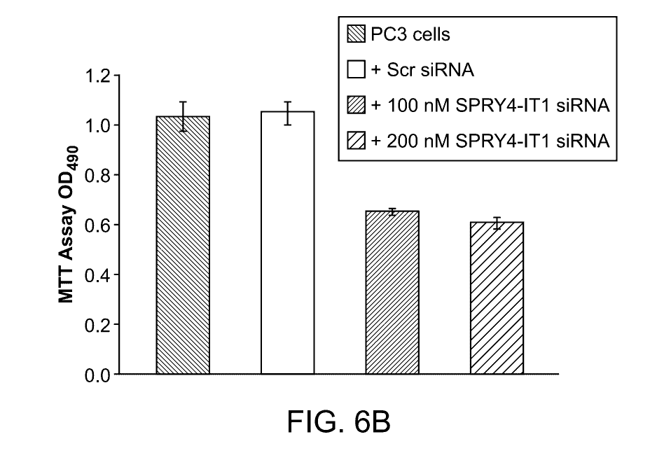

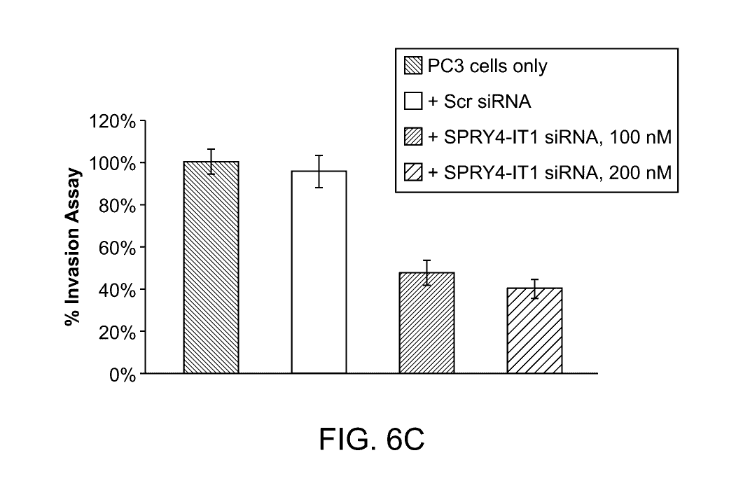

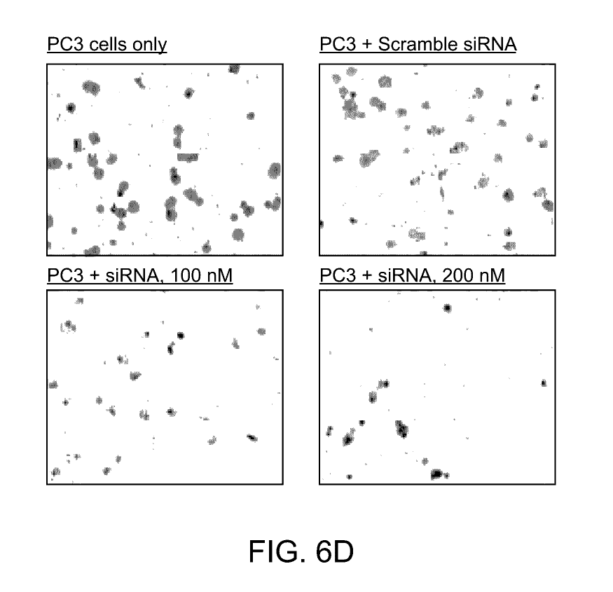

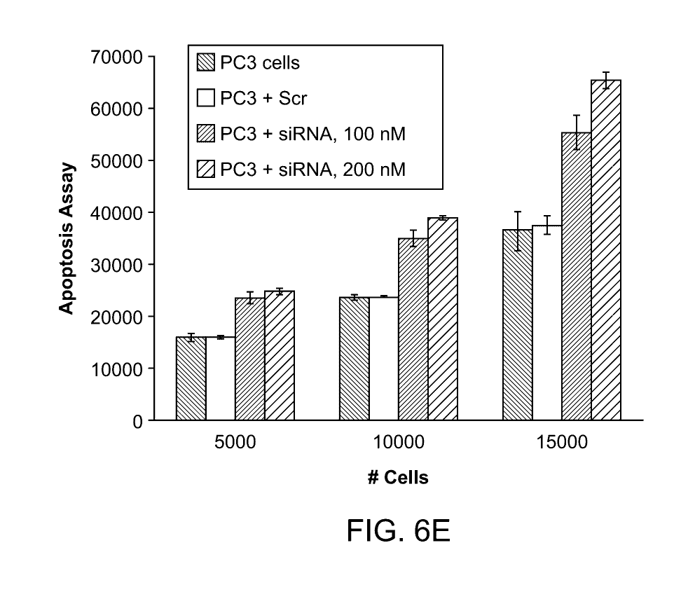

To establish whether knockdown had similar effects in prostate cancer cells, PC3 cells were transfected with siRNAs specific to SPRY4-IT1. qRT-PCR indicated that knockdown equal to ˜40% loss of SPRY4-IT1 was achieved after 48 hours at both 100 nM and 200 nM siRNA concentrations (FIG. 6A). There was a 40 and 50% loss of cell viability in cells transfected with SPRY4-IT1 siRNA compared to negative controls (FIG. 6B). Furthermore, a cell invasion screen performed using standard Boyden chambers indicated that cells transfected with SPRY4-IT1 siRNA had significant defects in invasion, with cell counts equal to only 50% of control at 100 nM, and 40% of control at 200 nM (FIG. 6C-D). Finally, apoptosis was assessed by measurement of caspase 3/7 activity in siRNA-transfected PC3 cells, which revealed a 50% increase in activity at 100 nM and an ˜60% increase at 200 nM, compared to controls (FIG. 6E). Together, these data confirm that loss of SPRY4-IT1 in prostate cancer cells results in decreased cell viability and invasion and increased apoptosis, similar to melanoma cells.

### Materials and Methods

### Cell Lines

All experiments described in this manuscript utilized at least one of the following human cell lines: prostate epithelial cells (ScienCell, HPrEpiC, Cat No 4410), PPC1, 22Rv1 (ATCC® CRL-2505™), DU-145 (ATCC® HTB-81™), LNCaP (ATCC® CRL1740™) and PC3 (ATCC® CRL-7934™) prostate cancer cell lines.

Prostate epithelial cells were grown in Prostate Epithelial Cell Medium (ScienCell, PEpiCM, Cat No 4411), whereas the prostate cancer cell lines were grown in DMEM (Invitrogen, Carlsbad, Calif.), supplemented with 10% FBS and Penicillin/Streptomycin.

### Affymetrix Arrays

The purity and integrity of the total RNA were analyzed on RNA Nano chip (Agilent Technologies) using Eukaryote Total RNA Nano series protocol. The total RNA was subjected to single round of linear IVT-amplification and labeled with Cy3-labeled CTP using One-Color Low Input Quick Amp Labeling Kit (Ambion). The resulting Cy3 dye incorporated antisence RNA (aRNA) was quantified using ND-1000 spectrophotometer (Nano Drop Technologies) and 600 ng of labeled aRNA was hybridized onto Ncode human ncRNA microarray (Life Technologies) or Agilent SurePrint G3 Human Gene Expression v2 (Agilent Technologies). After hybridization, the arrays were washed following the manufacturer's protocol using Gene Expression Wash Pack (Agilent Technologies) and scanned using the Agilent C Scanner. The intensities of the scanned fluorescence images were extracted with Agilent Feature Extrcation software version 10.7.3.1.

### Quantitative Real-Time PCR

Total RNA from all cell lines was isolated using the Trizol method (Invitrogen/Life Technologies) with all quantification and integrity analysis performed with the NanoDropND-100 spectrometer (Thermo scientific, Wilminton, Del., USA). RNA (2 ug) was then used for cDNA synthesis in a 20 uL reaction volume using a high capacity cDNA reverse transcription kit (Applied Biosystems, Foster city, CA, USA). For detection of SPRY4-IT1 and SPRY4, qRT-PCR was performed in triplicate using a Power SYBR Green PCR master mix (Applied Biosystems, Warrington, UK) in the 7500 Real-Time PCR system (Applied Biosystems, Foster city, CA, USA). A final reaction volume of 20 ul was used, containing 2 ul of cDNA template, 10 ul of 2× Power SYBR Green PCR master mix, and 0.2 uM of each primer. The reaction was subjected to denaturation at 95° C. for 10 min followed by 40 cycles of denaturation at 95° C. for 15 sec and annealing at 58° C. for 1 min. SDS1.2.3 software (Applied Biosystems, Foster city, CA, USA) was used for comparative Ct analysis with GAPDH serving as the endogenous control.

### RNA-FISH Analysis

Locked nucleic acid (LNA) modified probes for human IncRNA SPRY4-IT1 (TCCACTGGGCATATTCTAAAA), SPRY4 (GATGTTGCAACCACTGCCTGG) and a negative/scramble control (GTGTAACACGTCTATACGCCCA, miRCURY-LNA detection probe, Exiqon) containing biotin labels were used for RNA-FISH (Khaitan et al, 2011). In situ hybridization was then performed using the RiboMap ISH kit (Ventana Medical Systems, Inc.) using a Ventana machine. Cells in suspension were diluted to 10,000 cells/100 uL, pipetted on to autoclaved glass slides and allowed to adhere for 4 hours. The slides were then submerged in cell media (as above methods), then the following day removed from the media, washed with PBS and fixed in 4% paraformaldehyde/5% acetic acid. The slides were then subjected to the hydrochloride-based RiboClear reagent (Ventana Medical Systems) for 10′ at 37° C., followed by the ready-to-use protease 3 reagent. Cells were hybridized with antisense LNAriboprobe (40 nmol/L) using RiboHybe hybridization buffer (Ventana Medical Systems) for 2 hours at 58° C. after the initial denaturing prehybridization step for 4′ at 80° C. The slides were then treated to a low-stringency wash with 0.1% SSC (Ventana Medical Systems) for 4′ at 60° C. and 2 additional wash steps with 1% SSC for 4′ at 60° C. All slides were fixed in RiboFix, counterstained with 4′-6′diamidino-2-phenylindole (DAPI) using an antifade reagent (Ventana). Imaging was performed using the Nikon A1RVAAS laser point- and resonant-scanning confocal microscope equipped with a single photon Argon-ion laser at 40× with 4× zoom.

### RNA-CISH Analysis

The 5 um cut paraffin sections and a prostate tissue array (Biomax us, PR8011 tissue array) were placed on Ventana's Discovery XT platform (Ventana Medical Systems, Inc., Tucson, Ariz.) for Chromogenic in-situ Hybridization (CISH). The deparaffinization of the sections was performed by the protocol that was selected on the instrument. All subsequent pretreatment steps were performed on the Ventana platform using FISH protocol and Ventana specific products. Offline detection staining was accomplished by Alkaline Phosphatase technique using Fast Red as chromogen. The custom made LNA probe with a dual FAM label from Exiqon was used during the denaturing and hybridizing steps and was incubated for 4 hours at the probe's optimal temperature for annealing. Three separate temperature controlled stringency washes were performed to wash away probe that was loosely bond. The primary rabbit anti-fluorescein antibody at a 1:100 dilution was applied with heat for 1 hour followed by Ventana's UltraMap anti-Rabbit-Alk Phos multimer detection for 20 mins no heat. The chromogenic detection was performed offline using the components of the Ventana ChromoRed kit. Slides were dehydrated and coverslipped to complete the protocol.

### 5-Aza-2′-Deoxycytidine Treatment of LNCAP Cells and Isolation of Bisulfite Treated Genomic DNA

10⁷ LNCaP cells were plated into 2 75-cm² flasks and treated with either 10 ug/mL 5-aza-2′-deoxycytidine or left untreated. For 5 days, the cells were washed with PBS, fed fresh medium, and treated as above. After the fifth day all cells were washed with PBS, trypsinized, and centrifuged at 1200 rpm for 5′. The cell pellets were washed once with PBS, and purified using the QiaAmp DNA mini kit (QIAGEN). The samples were then quantified using the NanoDropND-100 spectrometer (Thermo scientific, Wilminton, Del., USA). 500 ng of genomic DNA was selected from each sample and treated with sodium bisulfite using the EZ DNA GOLD methylation kit (Zymo Research), eluting in 10 uL elution buffer.

PCR Amplification and Sequencing of Products Acquired from Bisulfite-Converted LNCAP Genomic DNA

50 ng of bisulfite-treated genomic DNA was used for bisulfite PCR using the following primer combination: 5′ Distal SPRY4 For (ggttttatttatttatttggttagtttt) and 5′ Distal SPRY4 Rev (taaatatcctttctctatcccaatc) to produce a 139-bp product. PCR was performed using a 2-min hot start at 95° C., followed by 40 cycles at 94° C. for 30 s, 48° C. for 35 s, and 72° C. for 30 s, ending with a 10-min extension at 72° C. using GoTaq green (Promega, Inc.). PCR products were run out on a 1% agarose gel, gel purified using the QiaQuick gel extraction kit (QIAGEN), and cloned into pCR4-TOPO (Invitrogen/Life Technologies). Six clones for each sample were sequenced using M13 forward and reverse primers (Retrogen, Inc.) and the results were aligned using VectorNTi AlignX (Invitrogen/Life Technologies).

### Cell Culture Transfection for Knock Down of SPRY4-IT1 in Prostate Cancer Cells

Knock-down of SPRY4-IT1 was performed using a 25-mer double-stranded RNA oligonucleotide complex siRNA (gctttctgattccaaggcctattaa, labeled #594, Khaitan et al, 2011) and transfected into cells using lipofectamine RNAiMax (Life Technologies) in 6-well plates using manufacturer's protocols. A total of 250,000 cells were aliqouted into each well and the RNAi duplex-lipofectamine RNAiMAX complexes were added and mixed gently by rocking the plate. In all cases, cells were incubated for 48 hours at 37° C. in a CO² incubator. Cell samples and gene expression levels were measured by quantitative real-time PCR (qRT-PCR, as above).

### Metabolic Viability by MTT Assay

The MTT (3-(4,5-dimethyl-2-yl)-2,5-diphenyl-211-tetrazolium bromide) assay was purchased from Roche. 96-well plates were used, plating 25000 cells in 100 uL DMEM per well (transfected as above). 48 hours after of transfection, 20 uL MTT solution was added and the cells were incubated at 37° C. in the dark for 4 hours. Generated formazan was measured at OD₄₉₀ nm to and compared to control cells to determine the cell viability on the Flex station (Molecular Devices; www.moleculardevices.com).

### Invasion Assays

The invasion assay was performed using BD BioCoat™ growth factor reduced insert plates (Matrigel™ Invasion Chamber 12 well plates). These plates were prepared by rehydration of the BD Matrigel™ matrix coating and its inserts with 0.5 ml of serum-free DMEM media for 2 hours at 37° C. The media was removed from the inserts and 0.75 mL DMEM w/10% FBS was added to the lower chamber of the plate, with 0.5 mL of cell suspension (5×10⁴ cells, transfected as above, in serum-free DMEM) added to each insert well. The invasion assay plates were then incubated for 48 hours at 37° C. After incubation, the non-invading cells were scrubbed from the upper surface of the insert. The cells on the bottom surface of the membrane were fixed in methanol, then stained with crystal violet, and washed in MQ H2O. The membranes were then mounted on microscopic slide for visualization and analysis. All slides were scanned (using the Scanscope digital slide scanner) and the number of cells remaining on the insert were counted using Aperio software. All data are expressed as the percent (%) invasion through the membrane versus the migration through the control membrane.

### Apoptosis (Caspase 3/7) Assays

PC3 cells were plated in 96-well plates at 5000, 10000, & 15000 cells per well in triplicate for each transfection condition (transfected as above) and allowed to culture in DMEM w/10% FBS for 48 hours before harvesting for assay. Samples were then prepared using the Caspase-Glo® 3/7 Assay kit (Promega) and analyzed by a GloMax luminometer (Promega) using conditions designed for the Caspase-Glo 3/7 Assay.

### Patients and Tissue Samples

This study included 18 pairs of formalin-fixed paraffin-embedded (FFPE) blocks of the prostate cancer and adjacent normal tissues. For the microarray experiments, 10 paired biopsy specimens were used for preparing RNA samples. These tissue samples were collected at Florida Hospital Celebration (Celebration, FL, USA) in 2008-2012. The use of tumor samples was approved by the institutional review board of the Florida hospital.

### RNA Extraction and Quantitative RT-PCR of Patient Samples

Twenty consecutive 18 um sections were cut from each patient block on a Leica 2235 microtome (Leica 2235) and placed into 2.0 ml microcentrifuge tubes. RNA was extracted with an RNeasy FFPE kit (QAIGEN). RNA yield and A260/A280 ratio were monitored with a NanoDropND-100 spectrometer (Thermo scientific, Wilminton, Del., USA). All qRT-PCR conditions performed were as in above methods. Fold changes in SPRY4-IT1 and SPRY4 expression in tumor tissue relative to the expression in normal tissue were calculated.

### Urine Collection and RNA Isolation.

Urine samples were collected (30˜50 mL) using Urine Collection and Preservation Tube (Norgen Bioteck, Thorold, ON, Canada) and stored at −80° C. till further analysis. Total RNA was isolated using the Urine (Exfoliated cell) RNA Purification Kit (Norgen Bioteck, Thorold, ON, Canada). The purified RNA was quantified using the NanoDropND-100 spectrometer (Thermo scientific, Wilminton, Del., USA) and stored at −80° C. till further analysis.

### CDNA Synthesis and Pre-Amplification

RNA (100 ng) was used for cDNA synthesis in a 50 uL reaction volume using a high capacity cDNA reverse transcription kit (Applied Biosystems, Foster city, CA, USA). 5 ng of cDNA was used for pre-amplification in a 50 ul reaction volume containing 25 ul of 2× Power SYBR Green PCR master mix and 10 nM of each primer. The reaction was subjected to denaturation at 95° C. for 10 minutes followed by 14 cycles of denaturation at 95° C. for 15 seconds and annealing/elongation at 60° C. for 4 minutes.

### Quantitative Real-Time PCR (QRT-PCR)

qRT-PCR was performed in triplicate using a Power SYBR Green PCR master mix (Applied Biosystems, Warrington, UK) in the 7500 Real-Time PCR system (Applied Biosystems, Foster city, CA, USA). A final reaction volume of 20 ul was used, containing 1.14 ul of pre-amplified cDNA template, 10 ul of 2× Power SYBR Green PCR master mix (Applied Biosystems, Foster city, CA, USA), and 0.2 uM of each primer. The reaction was subjected to denaturation at 95° C. for 10 minute followed by 40 cycles of denaturation at 95° C. for 15 seconds and annealing at 58° C. for 1 minute. SDS1.2.3 software (Applied Biosystems, Foster city, CA, USA) was used for comparative Ct analysis with GAPDH serving as the endogenous control.

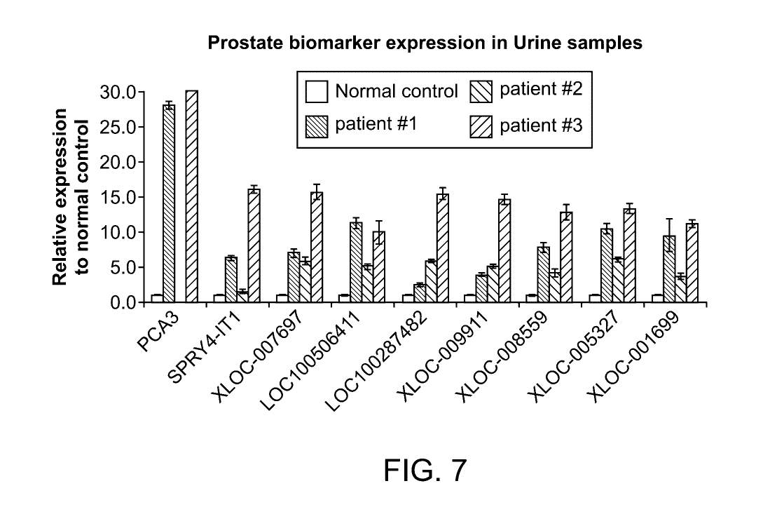

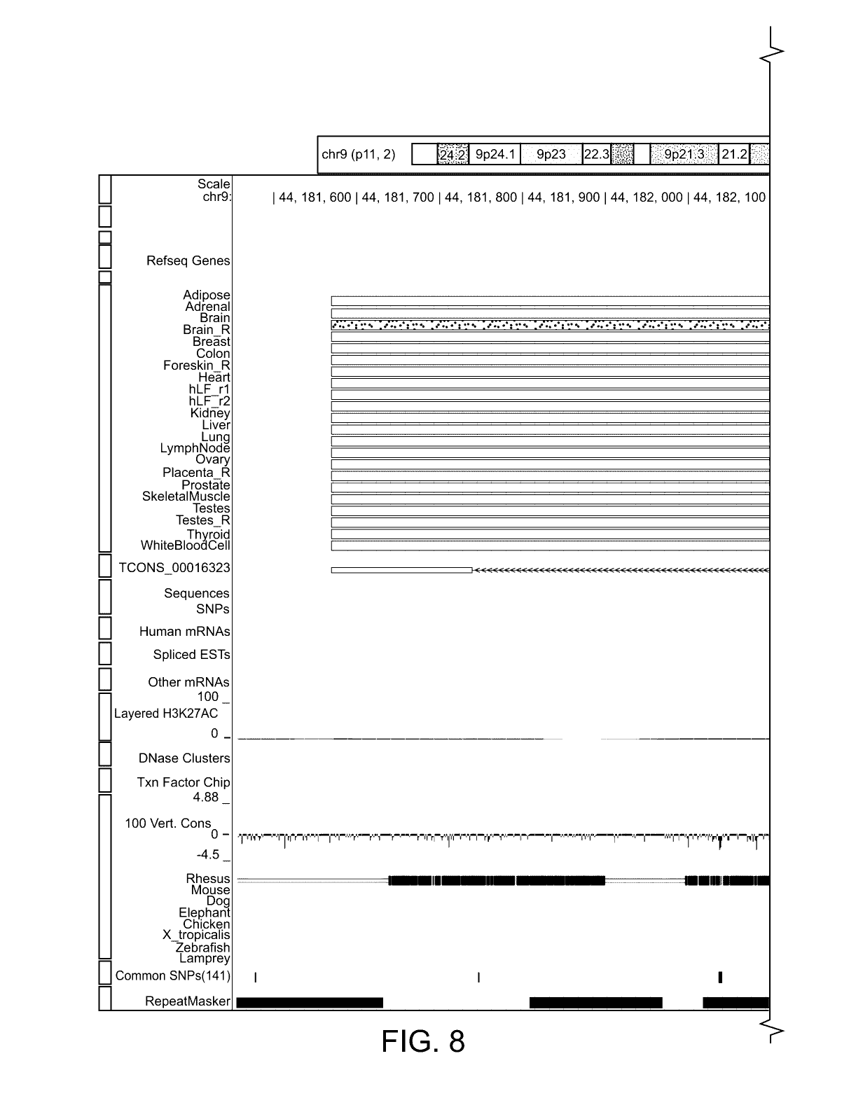

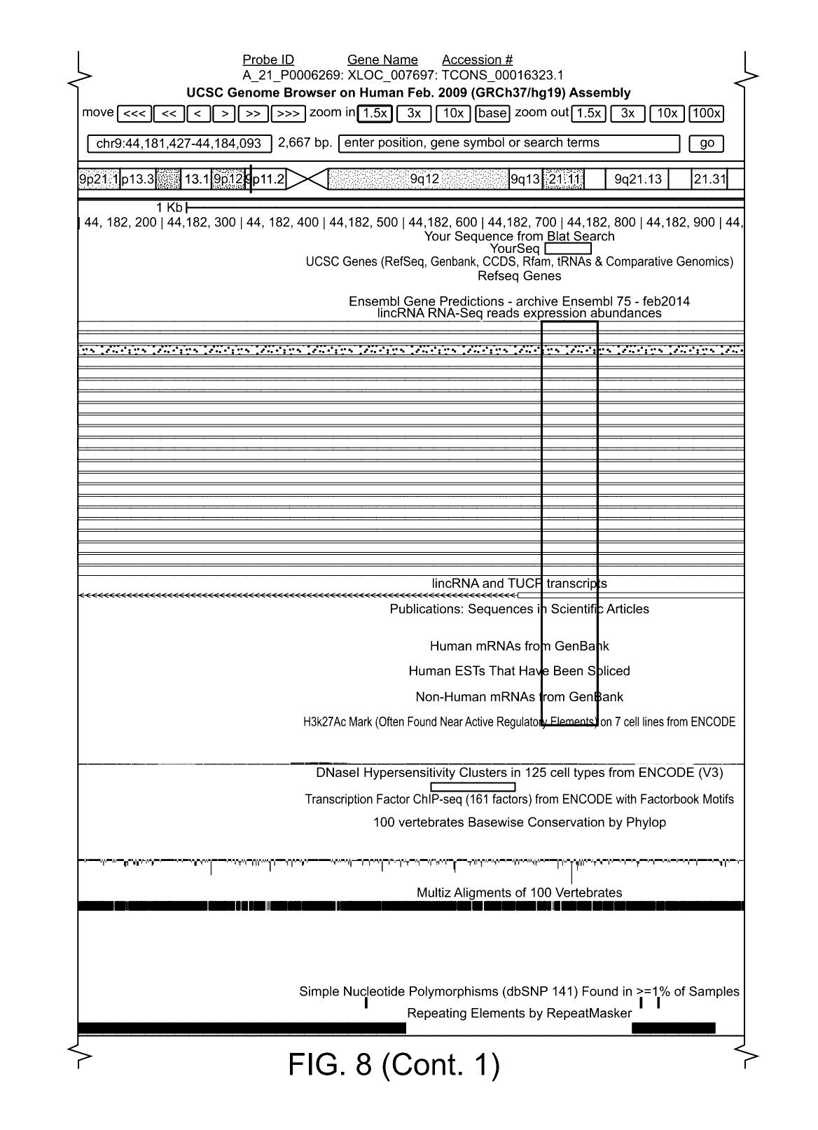

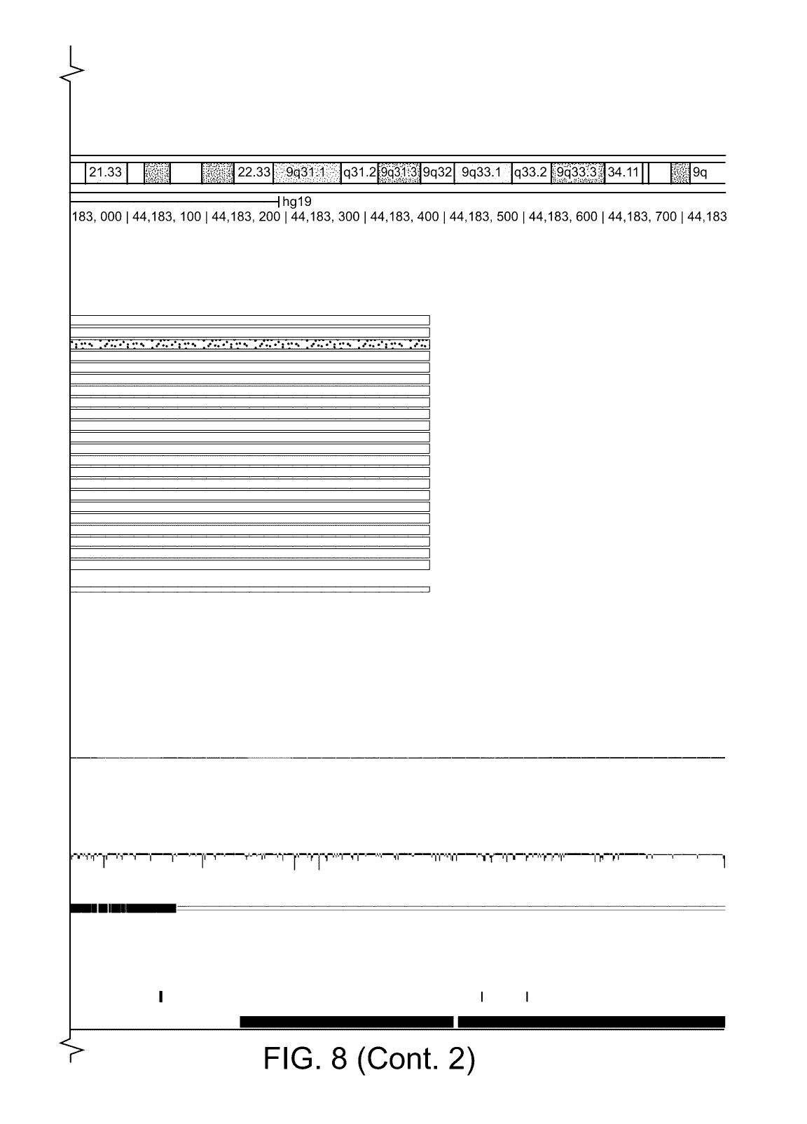

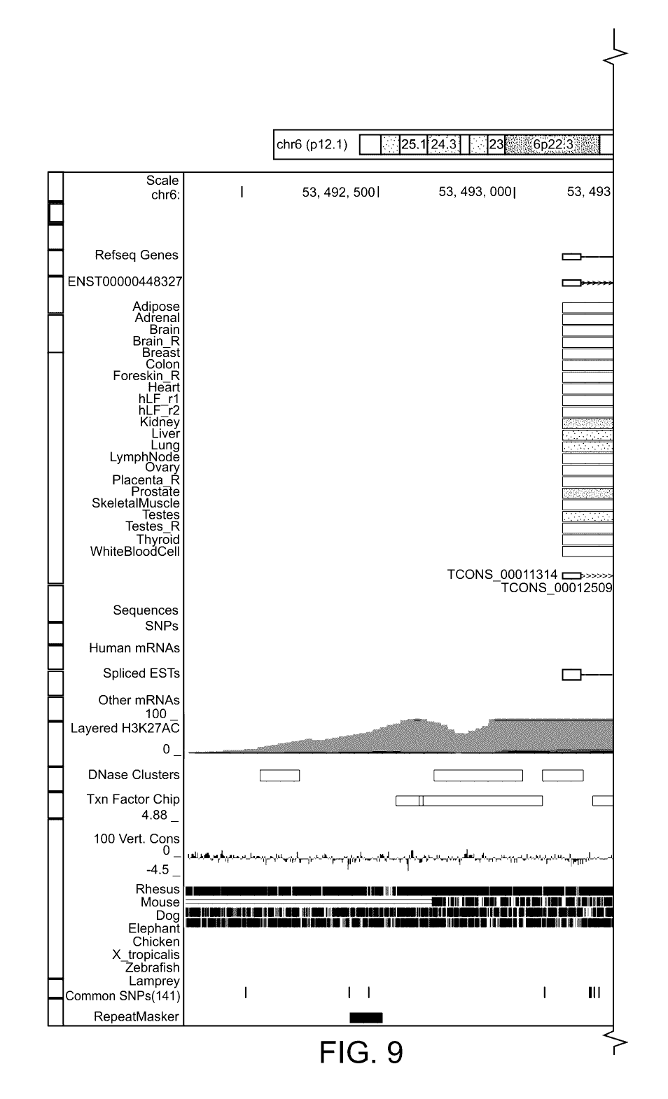

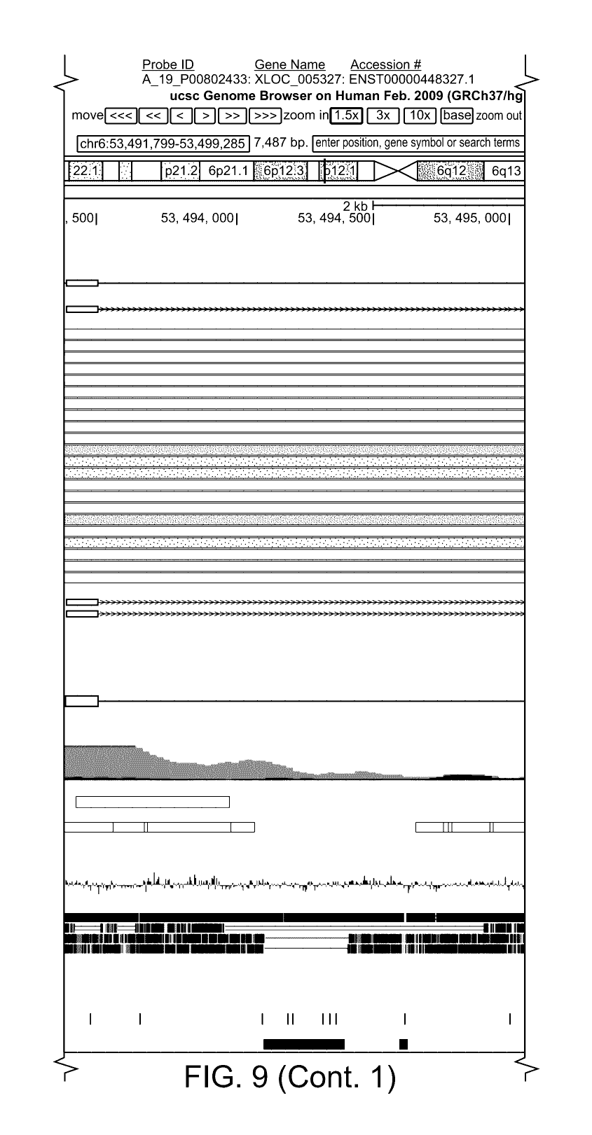

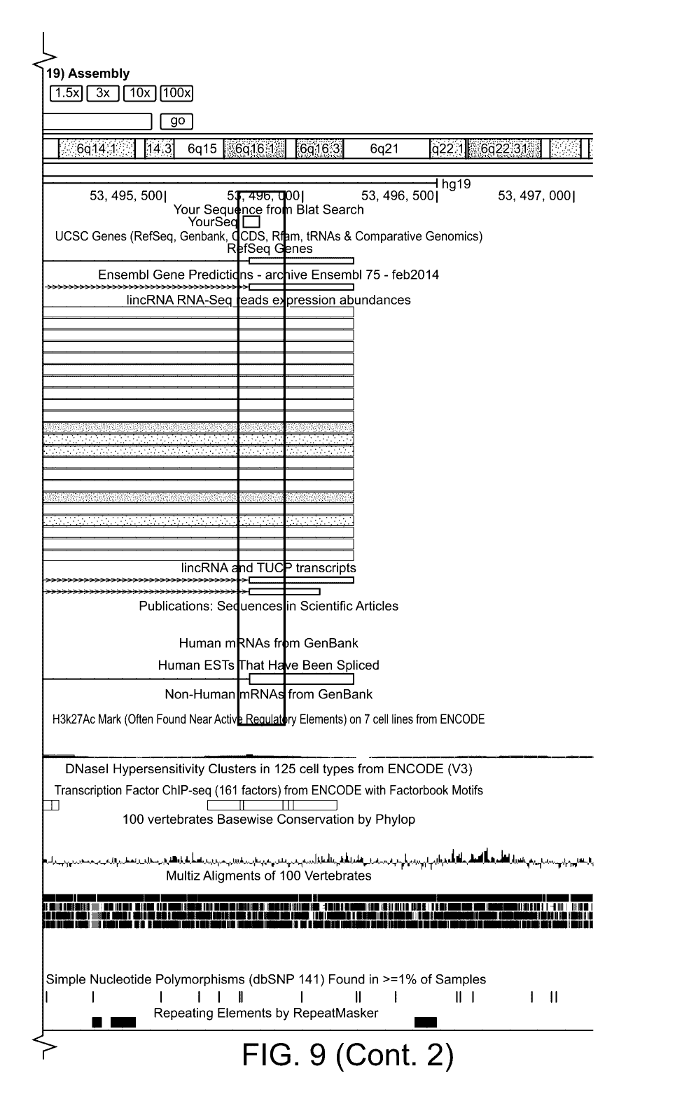

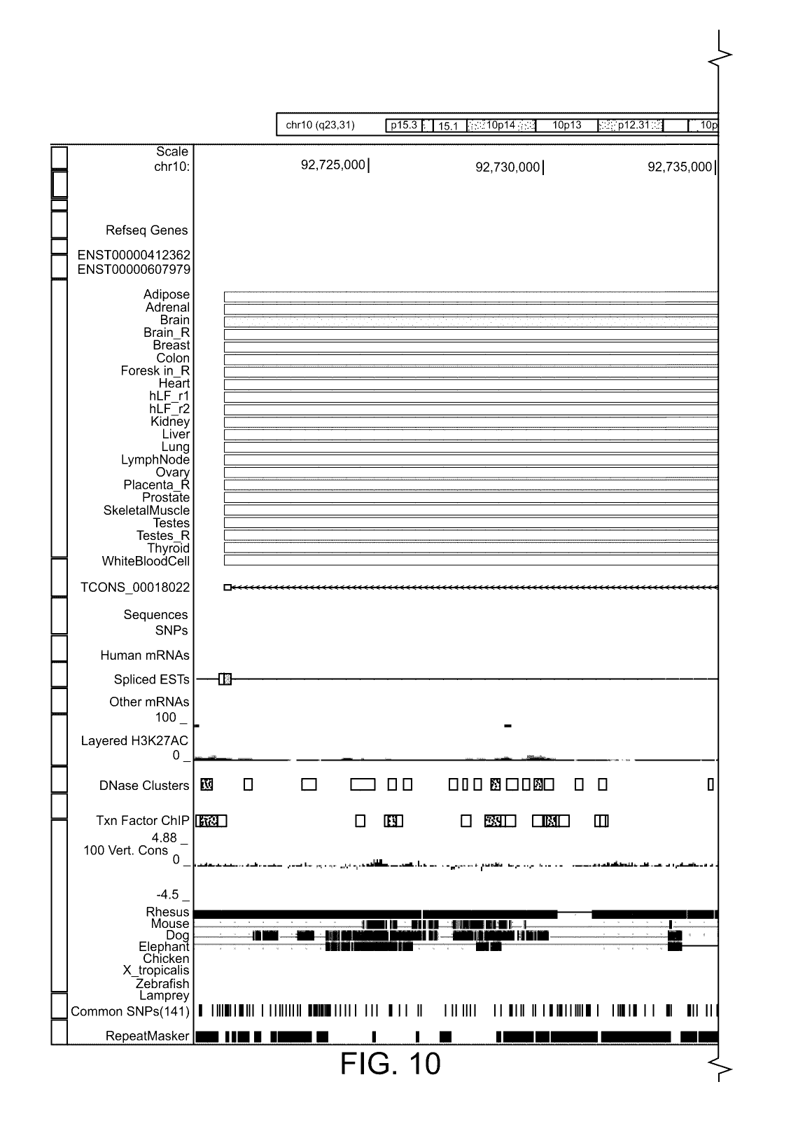

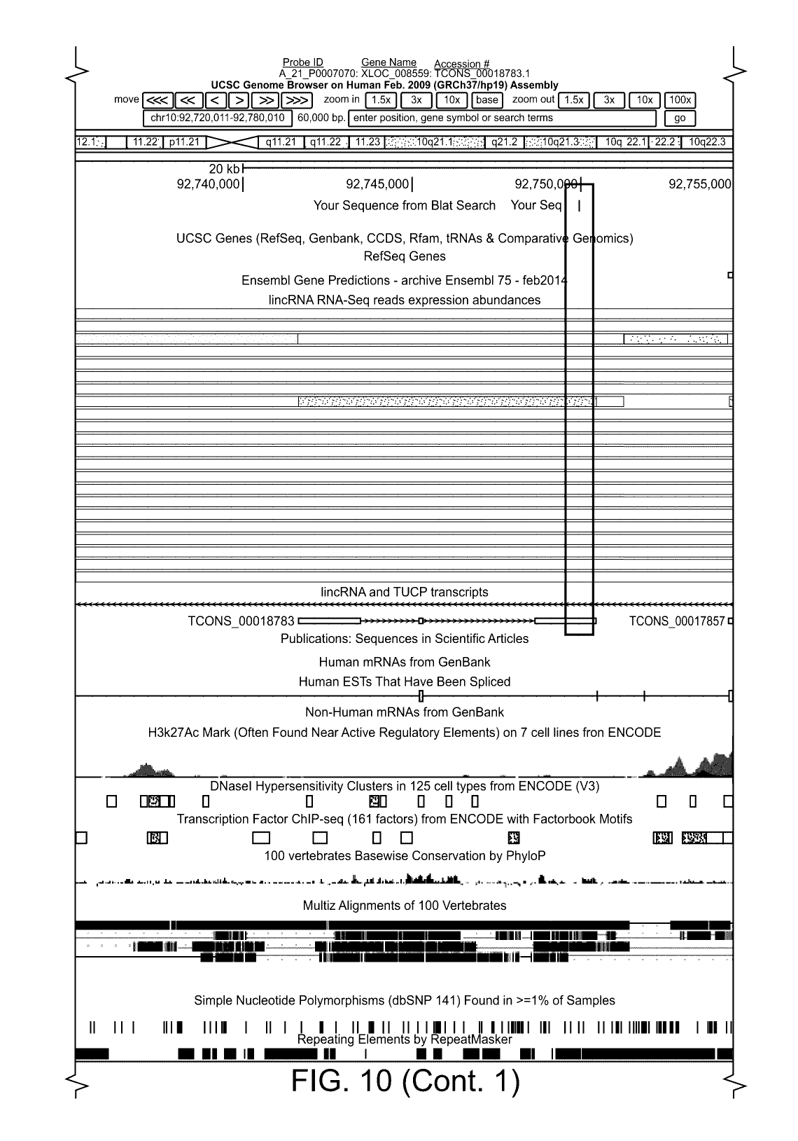

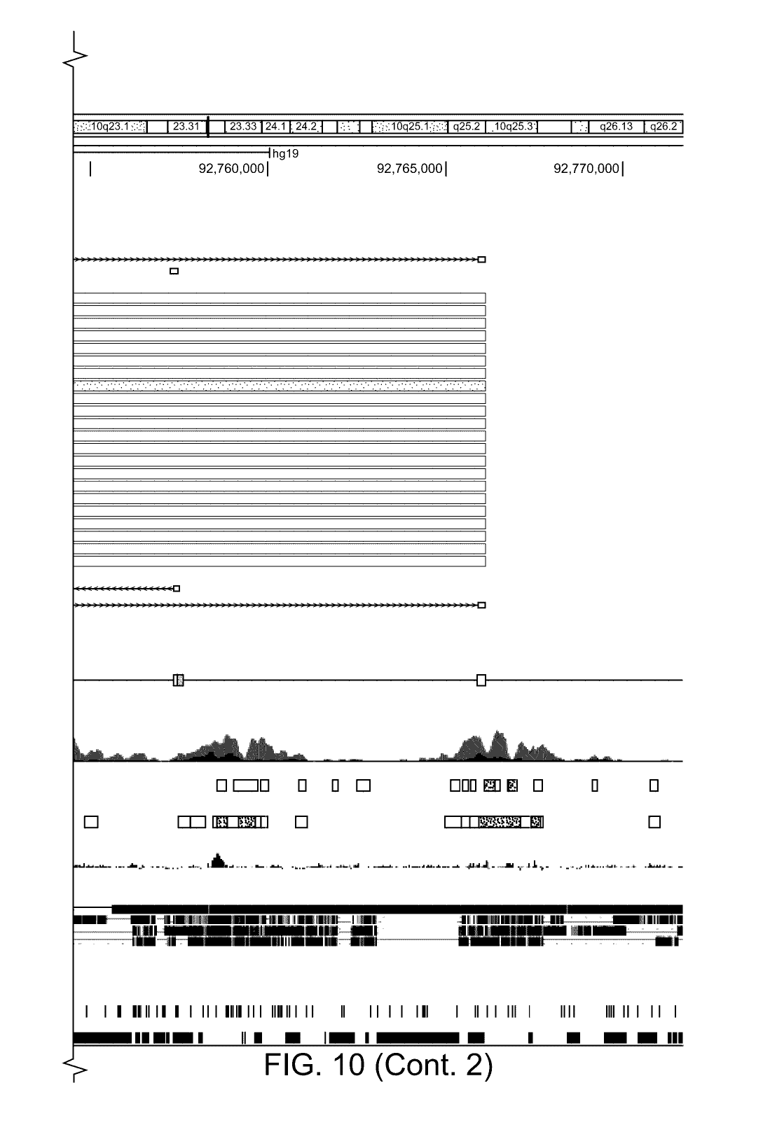

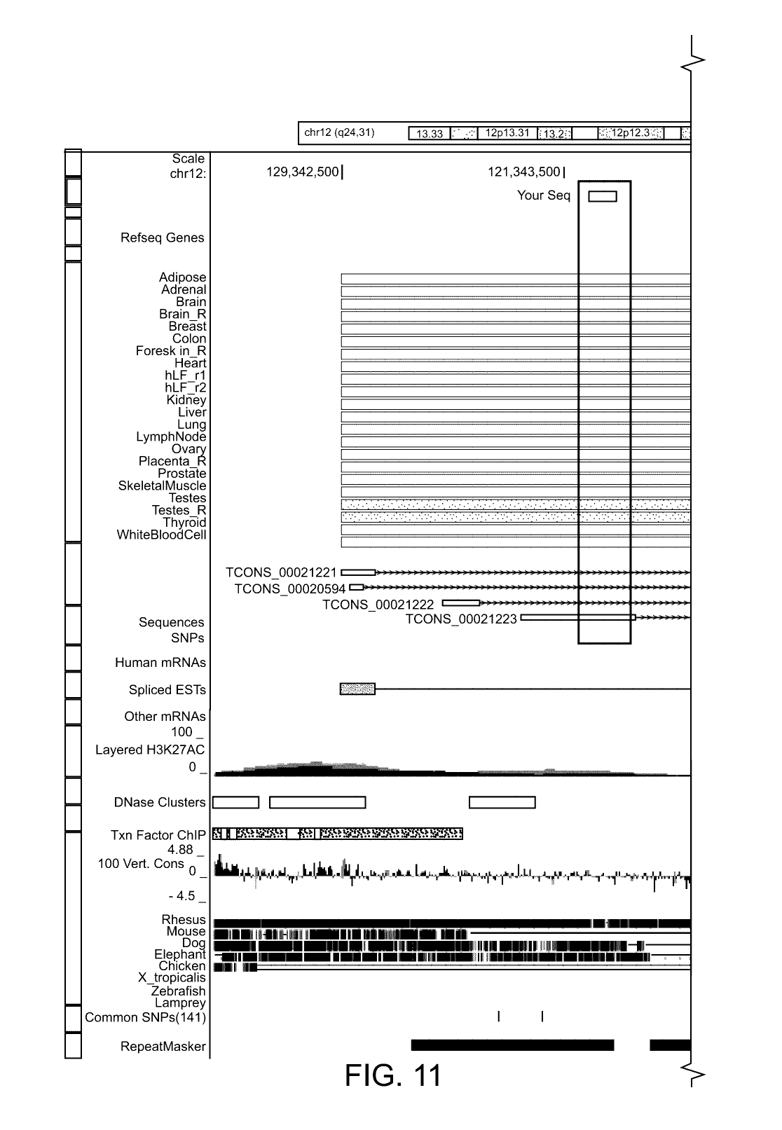

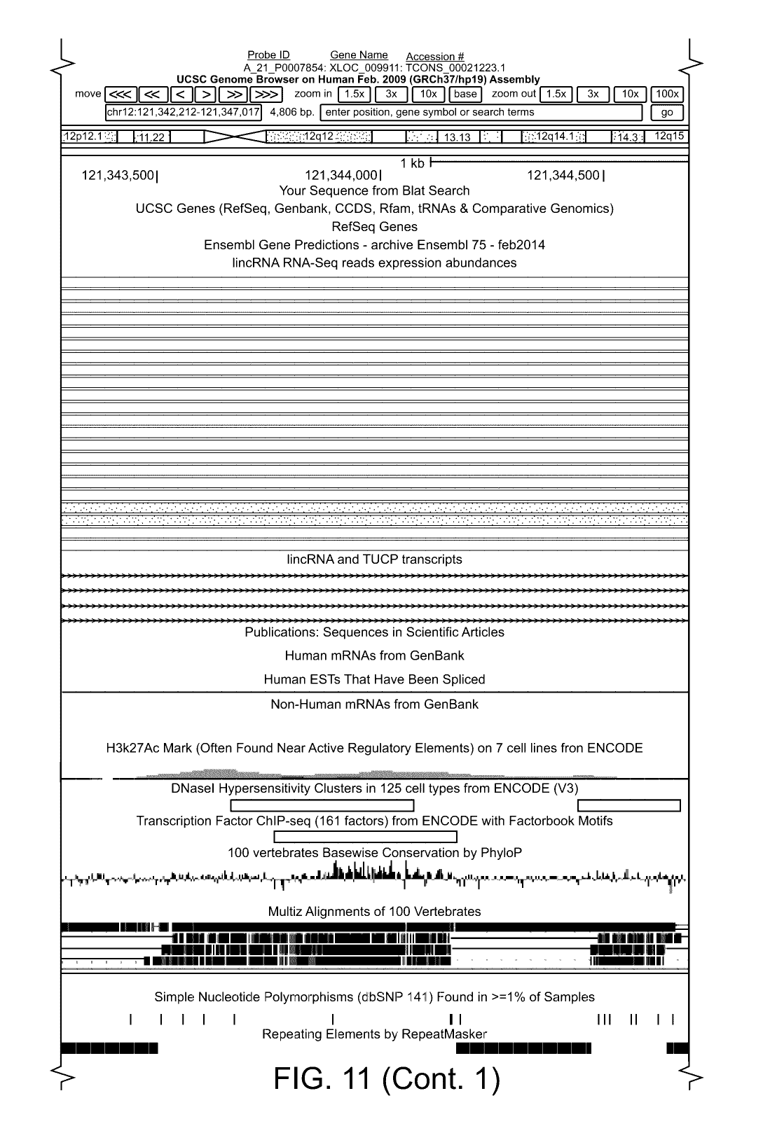

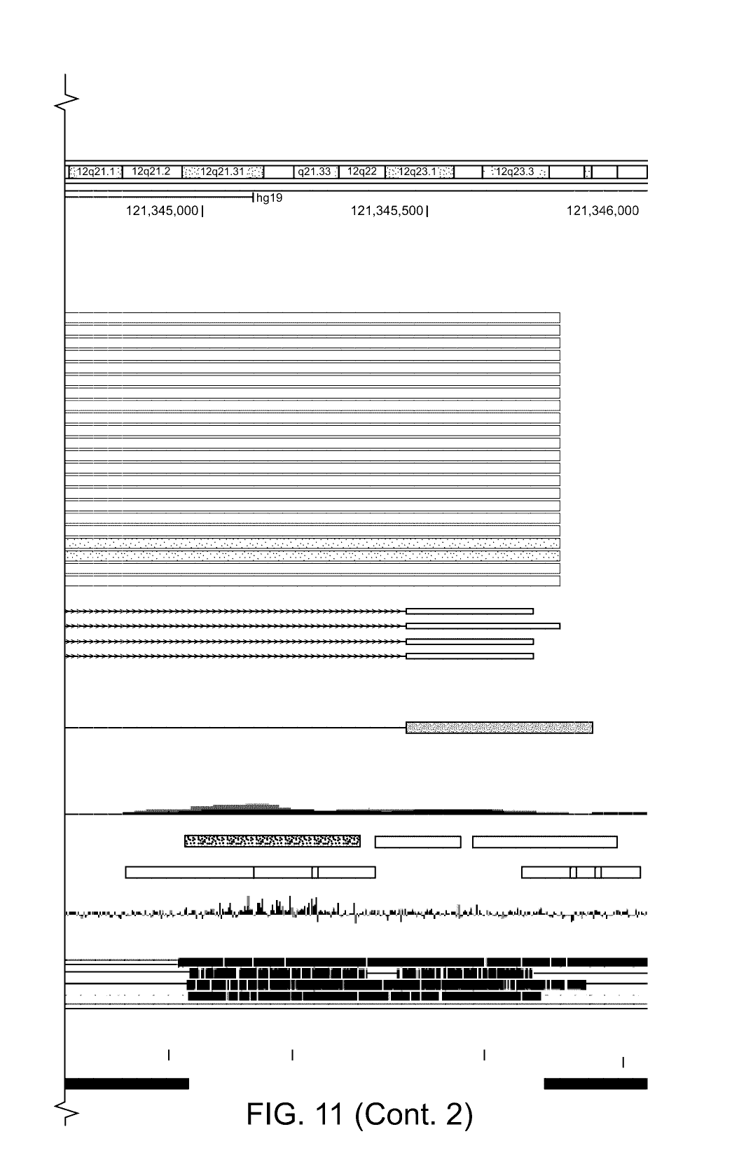

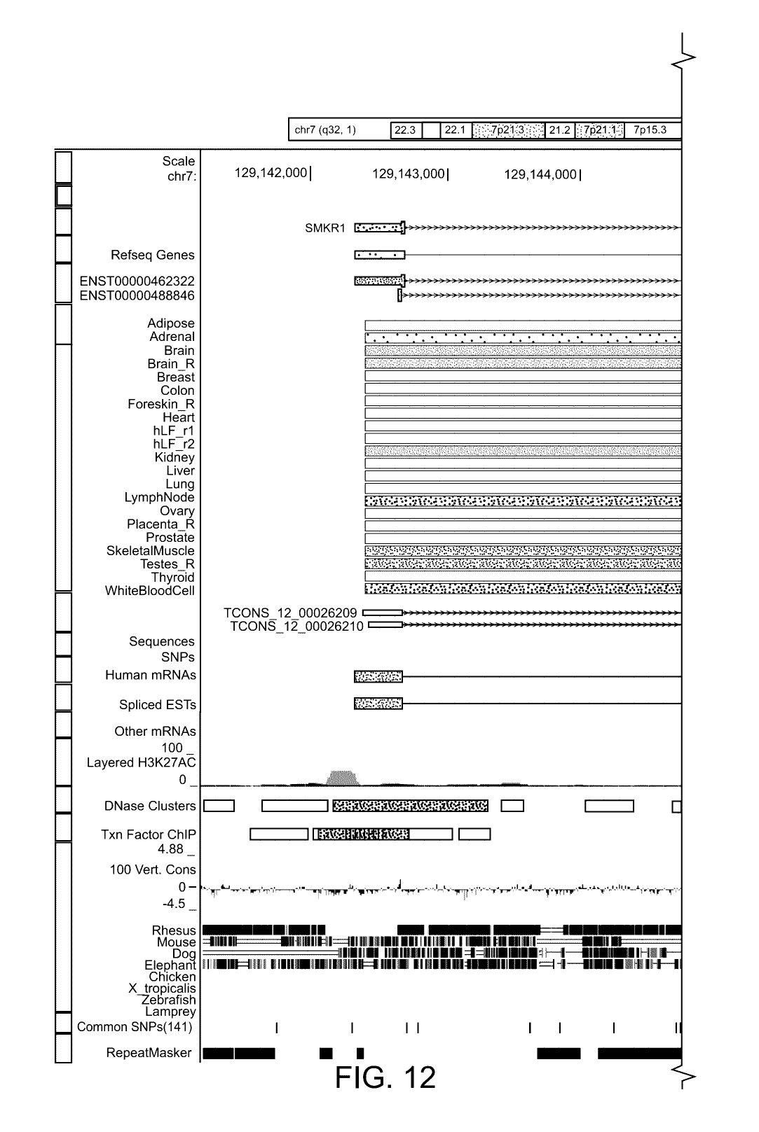

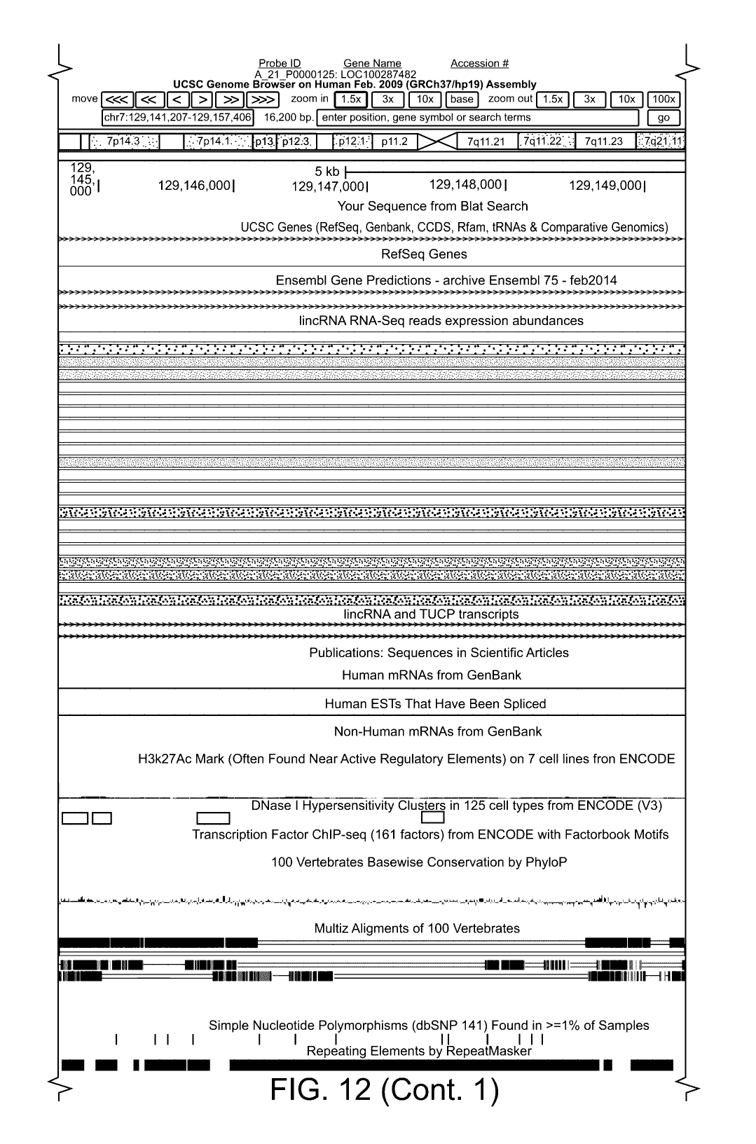

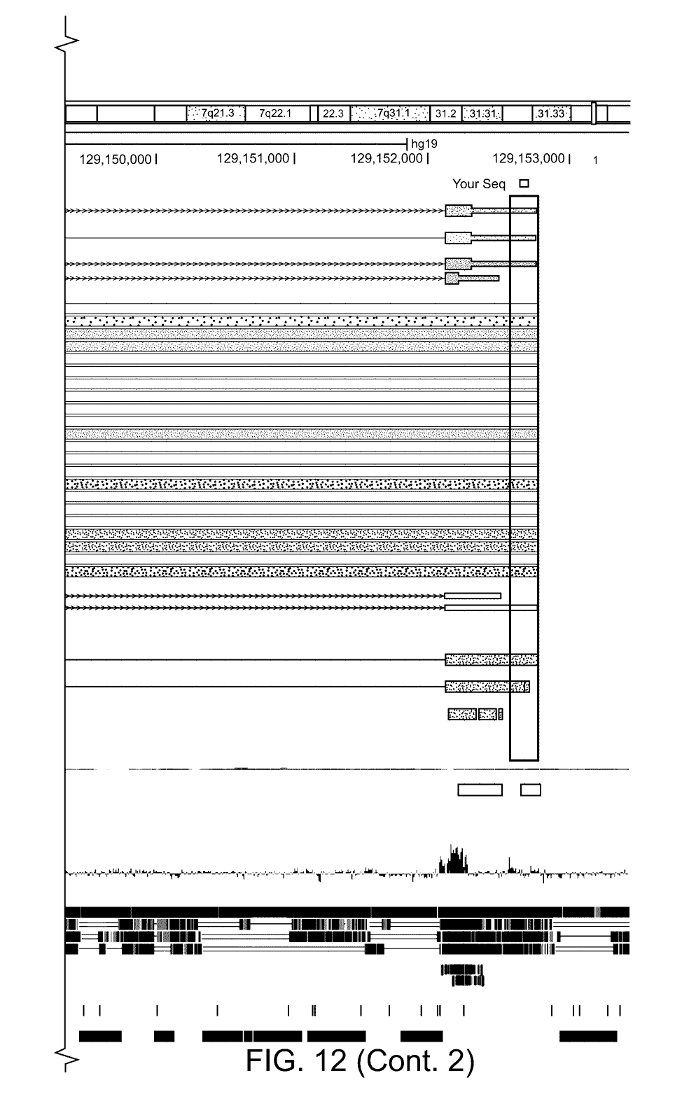

Putative prostate biomarker expression in urine samples was examined. Expression of eight lncRNAs (SPRY4-IT1, XLOC-007697, LOC100506411, LOC100287482, XLOC-009911, XLOC-008559, XLOC-005327, and XLOC-001699) and PCA3 was measured by qRT-PCR in one normal and three prostate cancer patients as shown in FIG. 7. The relative expression to normal control is presented as fold change for each gene. The expression of all eight lncRNAs and PCA3 were significantly higher in prostate cancer patients.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

### REFERENCES

1. Etzioni, R., et al., Asymptomatic incidence and duration of prostate cancer. Am J Epidemiol, 1998. 148(8): p. 775-85.
2. Potosky, A. L., E. J. Feuer, and D. L. Levin, Impact of screening on incidence and mortality of prostate cancer in the United States. Epidemiol Rev, 2001. 23(1): p. 181-6.
3. Lu-Yao, G. L., et al., An assessment of radical prostatectomy. Time trends, geographic variation, and outcomes. The Prostate Patient Outcomes Research Team. Jama, 1993. 269(20): p. 2633-6.
4. Ploussard, G. and A. de la Taille, Urine biomarkers in prostate cancer. Nat Rev Urol, 2010. 7(2): p. 101-9.
5. Elliott, R. J., et al., alpha-Melanocyte-stimulating hormone, MSH 11-13 KPV and adrenocorticotropic hormone signalling in human keratinocyte cells. J Invest Dermatol, 2004. 122(4): p. 1010-9.
6. Consortium, I. H. G. S., Finishing the euchromatic sequence of the human genome. Nature, 2004. 431(7011): p. 931-45.
7. Ravasi, T., et al., Experimental validation of the regulated expression of large numbers of non-coding RNAs from the mouse genome. Genome Res, 2006. 16(1): p. 11-9.
8. Mercer, T. R., et al., Specific expression of long noncoding RNAs in the mouse brain. Proc Natl Acad Sci USA, 2008. 105(2): p. 716-21.
9. Cesana, M., et al., A long noncoding RNA controls muscle differentiation by functioning as a competing endogenous RNA. Cell, 2011. 147(2): p. 358-69.
10. Hutchinson, J. N., et al., A screen for nuclear transcripts identifies two linked noncoding RNAs associated with SC35 splicing domains. BMC Genomics, 2007. 8: p. 39.
11. Sone, M., et al., The mRNA-like noncoding RNA Gomafu constitutes a novel nuclear domain in a subset of neurons. J Cell Sci, 2007. 120(Pt 15): p. 2498-506.
12. Clemson, C. M., et al., An architectural role for a nuclear noncoding RNA: NEAT1 RNA is essential for the structure of paraspeckles. Mol Cell, 2009. 33(6): p. 717-26.
13. Sasaki, Y. T., et al., MENepsilon/beta noncoding RNAs are essential for structural integrity of nuclear paraspeckles. Proc Natl Acad Sci USA, 2009. 106(8): p. 2525-30.
14. Sunwoo, H., et al., MEN epsilon/beta nuclear-retained non-coding RNAs are up-regulated upon muscle differentiation and are essential components of paraspeckles. Genome Res, 2009. 19(3): p. 347-59.
15. Blackshaw, S., et al., Genomic analysis of mouse retinal development. PLoS Biol, 2004. 2(9): p. E247.
16. Rinn, J. L., et al., Functional demarcation of active and silent chromatin domains in human HOX loci by noncoding RNAs. Cell, 2007. 129(7): p. 1311-23.
17. Dinger, M. E., et al., Long noncoding RNAs in mouse embryonic stem cell pluripotency and differentiation. Genome Res, 2008. 18(9): p. 1433-45.
18. Pollard, K. S., et al., An RNA gene expressed during cortical development evolved rapidly in humans. Nature, 2006. 443(7108): p. 167-72.
19. Pheasant, M. and J. S. Mattick, Raising the estimate of functional human sequences. Genome Res, 2007. 17(9): p. 1245-53.
20. Ponjavic, J., C. P. Ponting, and G. Lunter, Functionality or transcriptional noise? Evidence for selection within long noncoding RNAs. Genome Res, 2007. 17(5): p. 556-65.
21. Guttman, M., et al., Chromatin signature reveals over a thousand highly conserved large non-coding RNAs in mammals. Nature, 2009. 458(7235): p. 223-7.
22. Prasanth, K. V. and D. L. Spector, Eukaryotic regulatory RNAs: an answer to the ‘genome complexity’ conundrum. Genes Dev, 2007. 21(1): p. 11-42.
23. Peters, J. and J. E. Robson, Imprinted noncoding RNAs. Mamm Genome, 2008. 19(7-8): p. 493-502.
24. Royo, H. and J. Cavaille, Non-coding RNAs in imprinted gene clusters. Biol Cell, 2008. 100(3): p. 149-66.
25. Zhao, J., et al., Polycomb proteins targeted by a short repeat RNA to the mouse X chromosome. Science, 2008. 322(5902): p. 750-6.
26. Allen, T. A., et al., The SINE-encoded mouse B2 RNA represses mRNA transcription in response to heat shock. Nat Struct Mol Biol, 2004. 11(9): p. 816-21.
27. Espinoza, C. A., et al., B2 RNA binds directly to RNA polymerase II to repress transcript synthesis. Nat Struct Mol Biol, 2004. 11(9): p. 822-9.
28. Mariner, P. D., et al., Human Alu RNA is a modular transacting repressor of mRNA transcription during heat shock. Mol Cell, 2008. 29(4): p. 499-509.
29. Fejes, T., et al., Post-transcriptional processing generates a diversity of 5′-modified long and short RNAs. Nature, 2009. 457(7232): p. 1028-32.
30. Ishii, N., et al., Identification of a novel non-coding RNA, MIAT, that confers risk of myocardial infarction. J Hum Genet, 2006. 51(12): p. 1087-99.
31. Pasmant, E., et al., Characterization of a germ-line deletion, including the entire INK4/ARF locus, in a melanoma-neural system tumor family: identification of ANRIL, an antisense noncoding RNA whose expression coclusters with ARF. Cancer Res, 2007. 67(8): p. 3963-9.
32. Faghihi, M. A., et al., Expression of a noncoding RNA is elevated in Alzheimer's disease and drives rapid feed-forward regulation of beta-secretase. Nat Med, 2008. 14(7): p. 723-30.
33. Sonkoly, E., et al., Identification and characterization of a novel, psoriasis susceptibility-related noncoding RNA gene, PRINS. J Biol Chem, 2005. 280(25): p. 24159-67.
34. Daughters, R. S., et al., RNA gain-of-function in spinocerebellar ataxia type 8. PLoS Genet, 2009. 5(8): p. e1000600.
35. Mercer, T. R., et al., Long noncoding RNAs in neuronal-glial fate specification and oligodendrocyte lineage maturation. BMC Neurosci, 2010. 11: p. 14.
36. Guffanti, A., et al., A transcriptional sketch of a primary human breast cancer by 454 deep sequencing. BMC Genomics, 2009. 10: p. 163.
37. Woo, C. J. and R. E. Kingston, HOTAIR lifts noncoding RNAs to new levels. Cell, 2007. 129(7): p. 1257-9.
38. Pibouin, L., et al., Cloning of the mRNA of overexpression in colon carcinoma 1: a sequence overexpressed in a subset of colon carcinomas. Cancer Genet Cytogenet, 2002. 133(1): p. 55-60.
39. Fu, X., et al., Regulation of apoptosis by a prostate-specific and prostate cancer-associated noncoding gene, PCGEM1. DNA Cell Biol, 2006. 25(3): p. 135-41.
40. Calin, G. A., et al., Ultraconserved regions encoding ncRNAs are altered in human leukemias and carcinomas. Cancer Cell, 2007. 12(3): p. 215-29.
41. Lin, R., et al., A large noncoding RNA is a marker for murine hepatocellular carcinomas and a spectrum of human carcinomas. Oncogene, 2007. 26(6): p. 851-8.
42. de Kok, J. B., et al., DD3(PCA3), a very sensitive and specific marker to detect prostate tumors. Cancer Res, 2002. 62(9): p. 2695-8.
43. Khaitan, D., et al., The melanoma-upregulated long noncoding RNA SPRY4-IT1 modulates apoptosis and invasion. Cancer Res, 2011. 71(11): p. 3852-62.
44. Wang, J., et al., Sprouty4, a suppressor of tumor cell motility, is down regulated by DNA methylation in human prostate cancer. Prostate, 2006. 66(6): p. 613-24.

### LncRNA Sequences and Probe Positions (Underlined)

>SPRY4-IT1
(SEQ ID NO: 1)
GTAGAGATGGGGGTTTCATCCTGTTGGTCAGGCTGGTCTTGAACTCCTGACCTCA
AGTGATCTGCCTACCTTGGCCTCCCAAAAGGCTGAGATTACAGGCATGAGCCACT
GCGCCAGGCCTTCTTTCTTTTCTTTTTTTCTTTCTTTTTTTTTTTTGAGACATCATTT
AGCTGTGCTGAGGGGTTCTTAAATAGGCAGCTCAGAAAATTGTTTTCCTTTGTCA
GCCACATAAATTCAGCAGAGGCTCTTGGAGGGTCCCTGCTGGTGAGGGGTGAGG
CCAGCAGTGGAACTCTGATTTGGTTTTTGCTGAGCTGGTGGTTGAAAGGAATCCT
ACTACATCGGGGTTATAATAGGGAAGATACATTTTAGAATATGCCCAGTGGAGC
CATCGGATGCTGCATCGTCCCCAGAGAGCCAAGTCATCGTGGGCCAAGCTCCCAT
CCCCATGTCTGGCCTCAACTGCAGGCCCAGAATGTTGACAGCTGCCTCTTGGAGG
GTTATGGGAGCCTGTGAATGCCAACATCCCCATTTGCCTGCAGCGGCTGCTCCCA
TCCTGGCTTCCTGGTGGGACTTTTCCATGAATTGGGGAATCTGCTTTCTGATTCCA
AGGCCTATTAAAATTTCTGAGCATTGCCCATTTCTTTTGCTTTATCTGTAGGACAT
GGGCTGTTTTTAAAGAACCTCACAAATGAAAAAAAAAAAAAAAAA
>XLOC_007697
(SEQ ID NO: 2)
CAGACTTTCTTGTTTGCCTCATCCCTACCAGTGTCTTTCTCCCTACACCTAAGGTC
AATTACCAGCTGCCCTTTATCGTTGAACTTGATGCTTTCTTCTCATAGTAGAATTA
AGAGGAAAGTAAAATATTTTTTGTACCTATATCTTTATTATATTTAGACAAATCA
CAGAGTGAGAGAGTAGGGGTTTCAAGAAAAATAGGAGAGAGATAAAGGAGAGA
GAAAGAACTGCTTGTGGAAATACAGAATATCCCACATTTTCAATGTGGAAAGTGT
ATGAGGGTATGAAAGAAAATACTCAGTTTTTTTTGTCCTGTAAGAGGCAGCATTG
ACAAATGTGTACCAGAGTTTGGGTACATTTGAGCCAGTTCTTCAGAATCGTGGGG
TGGGAAATAGAACAAAATTATTTACACCTAATTCTAGGCAGATAAGTGTGCTTCA
AGGAAAGGCAAGGGCCTGGCTAGATTCTAGATGTTTTTAAACTGGAGGCCAGAG
ACAGCTTTAGGGAGTCCATATACAGGCACAAATTTATTTCTTTTATAGTCTTCTTG
CTCTTTGAAAATGGTCTTTATGCAAATACTCACTATATAACCAAAGTTTCTCTTTG
TTCCAGGCAGCAGTAGGGCTGATTGGAGCCATTGTACGTGTCGGGAACATATCA
GAACACCGAGAATAGCGTCATGTCATAAGGACTCAGAGCAGGTGGACCCTGCTG
TGATGCACAAAGAGGACCACGCAAGATATGATAAAGATCTATGTCACTGAATTT
TGGTTCAATTTTTGTATCTCAGCTTCCCGGAAATAAAAAAGAATTCTAACATTCA
TACTTTCAGTATTTTATGTGAGAGGTTTTGTTGTCAAAATCAAGTCTGAGAGCAA
TGTTTATTGGGGTCTTTAATTGGAGTCACCA
>XLOC_009911 (=lnc-HNF1A-1:4, TCONS_00021223) 541 bp
(SEQ ID NO: 3)
GAATGATGACAGAGAGCTGGCCTTGCAAAGATCCACAGGAAAAGAGTTCCTGGC
AGAGGGAACAGCAAGGGCAGAAGGCTCAGGAAACCGTCCATTTGGAGGTCTGG
AAACCGGCACAGAAATAAACACGGTAGAGCTAGACCAGAGACCAACAAAGTGA
ATCTGGAGCTTAGATGGAGAGAGAAGAGAGAGATTAATTGAGGCCCCAGGTACT
GCGGAATGCTTCCCCAGGAGTGGATGAGGCCGTCTGAAAGGAGACCTCCGAAGT
GTTTCTTGAGGAAATGTGGCTGCAGACCCTAGAAGAAGCTACACAGCACTTGCC
AGGGCTGGGATGATGTCCAGGCCATGGAAACACCGTGTACCTGGTCCCAGGAAG
ATGAAGTGTGGGCCCAGAGACTAATGGCTTGAGCATCTCAGGCTAAGGTTGCCG
AGAAGTAGACAGCACCTCTAGATCCTAGTCAACATCTCTACAGGCTTGAAGTCTC
CCCAGAGGGCAAGGTTGGAATAAATCTGAAGCCTGTGGCTTGCCTGGGAGCTGC
CC
>XLOC_008559 (=lnc-RPP30-2, linc-PCGF5-3, TCONS_00018783) 3779 bp
(SEQ ID NO: 4)
CTGCACTCCAGCCTGGGCGACAGACCAAGACTCTGTCTCAAAAAAAAAAAAAAG
TTATAGTTTAATTTTTAAGGTTAATTTATTATTGAAGAAAAATTTTTAATGAGTTT
AGTGTAGCCTAGGTGTACACTAGGTGTTTATAGAGTCTACGATAGTGTACAGTCA
TGTCCTAGGCCTTCACATTCACTCATCACTCACTAACTCACACAGAGCAACTTCT
GGTCCTGCAAACTCCATTCGTGTTGAGTGTCCTATGTAGGTGTATTACTTTATATC
TTTTGTACTATATTTTTACTGTATTTTTTCTTTGTTTAGAAATGTTTGGATACACAA
ACACTAGTGTGTTACAATTGCCTACAGTATTCATTCAGTACAGTAACATGCTGTT
GCAACCTAGAAGCAATAAGCTACACCATATAGCCTAGGTGTGCAGTAGGCTACA
CCATCTAGCTTTGTGTAAGTACATTCTTTGAAGCTTGCACGATGACAAAATTGCC
TAATGACACATTTCTCAGAACATAACTCCATCATTAAGCTACATAACTTAAACCC
CTGCTATGCAATGAAACTCAGGTAGCATATTAAAAAATAGATAACTCAAGCATT
GCATACAGAGAAGCCATTCTTGGAACACCAGACAATAAGCATTGCATTAGATCA
GAGCAGTTCTGGGCACATCTATGGTCAACAAGAAATATTCTCAAAGTCTGAACTT
TGAGCTATAGTAGACAGACAAACTAAGAATTCCTCAAAGTTAGTATTTCCAACCG
TGATGTAAGAGTCTATTCTGAGTGTTGTGACAAACTATCTCCAGATCTCGCTAGA
GTAACACAATAAAGGTTTGTTTCTCACCCATCACAGTCGGGTATGGCTGTATGAG
GGAGGCGTGAGGAAGGATCTGCTCCTCGCCCATCACAGTCGGGTATGGCTGTAT
GAGGGAGGCGTGAGGAAGGCTCTGCTCCATGCATTCATGGAAGTGGCCTTGATC
ACCAGCCTAGCACTTCACTGGCAGGGCTCAGTCAATGACATCTAGTGGCTGGGA
AGCTCGGAAATGAGCTTTCCTTTGTGCTCAGAAGTAGGACTTGGGCGAACACATA
GCAGTATCTCTGCTCCATCCACATAAACGGGCTCAGAACTTAAATGGAAAGAGA
CGCTGAAGAGGGCATCAAATATATGAGAACTGGAACAGGGAAAGGAACAAAGA
TCTGAACAGGATCAGATAGAGATATTTGCCTACAGACAAGTCCTTGGTTAAAAG
ACCGTGGAAATTGATTCTAGAACTATATATTATTTATGGCTTGTGGGACGCAGAA
ATGTGTTCTGGTTACCTGTGCAATAAACTGTACATACTTCTCATTTCAGAGTTGGA
GTCAATCACTCTCTGTTGGCCTTTTTTGCTGTCTTTACAAAGTCATGGGTTAACGA
ACCCTACTGGGTACTTCTAACATGAGGTGTCTGGGCTGGGAGAGTCTTACTGGCA
ATTGATGTCAAGATTCTTCGTCCAGAGGCACAGAGCAGAAAGGTTCTTGGTCCAC
AGACACCTTAAAACAAGGCCACCCTGGCCAGGTTTATTCCCGTCTGGCGGCCTAC
ACATTTCTTATATCCTGGAAAAACTGGTGAGCAAGCAAGTGTCGACCTCAGAGTC
TCTGACAGGGCTATTTTGAAACCACACACCATGAAAACTCTCAGGGAAGTTAAA
AAACAAACAATCATAACCAAGGCAGTTTAGCTGTTTTGAAAAGAGATGGAGCTT
CATTACTTCAAACCCAAATTTCTGCAAGCCTGACAACCACCTTACATCAAAATAA
ACGTCTACCTGCTAGCTGAAATGTTTAAAAACACAGTTACCATGTGAGGTAAGCA
GAGCTGACCTTGACTGGCATCTCTATCAGCAGCTCAGTGGGATTAAATGGCTTGC
CAATGTCACAAGAATGTGAGCTCCTTTCTTCATCTTTCTGCTCCAATGTAGCAACT
ACCAAGGGGCCACCTGACAGAACATGGCCGCTGCAGAGGAACCCTGCTACCTGC
AGTTGGTGACATGGCCTAGGTCCCAGAGGCCTCGTGGTGCCACACACACAAGAA
CAGGCACCAACAACCAGTGACATTTTGACAGTCAAATGGAACCTGTGACTGCCA
TCTGTAGATGTGCCAGCCAAGAATGTGACCCTGGGGAAAGCCCTTCACACAGGT
CTTTCCTTGGTGTATTTATATTTAGTTCCAGCGAAAAACTGCAGTTGTTTTTCTCA
GTGACAGGCATCAAACGATAACCGAAAAGAATGAGAAATAATTGTTCCCTTTCT
CCCTGTTAGGAGATTGTACTCTTTGAATTTGGGACCACAGCTCTCTGAACAGCTA
GCTCTCCCATGCCTGGCTCATGAGACATCATAAATGTTGATTGTATTAAAGACAA
TTTAGAGGGAAAGGACTTGAATTCTGGTTCTAAGCTATTAAAAATATTTCTACAT
TTTAATTTTTAAATTAAGAAAGATTTTGTACATATGGAAAGGTGCAGAATATAAA
ACAGACAACCATATGCTTACCATCCAGATTAAACAACTGTTAACGTTTTCTCGTA
TTTACTTCAGATCACTTGAAACAAAAGAAAGACAAAAAGATACGGCTAAAGCCT
TGGCCCCCTTCACTCACATCCCTCCCCTCCTCCCCTCTGCAGAGCAACTTCTGCCT
GAAGCTGGTGTGTGTCATTTCCATGCATGATCTTGTGCTTTCAGTACATATTTGTA
TATCCAAAACAATATTTACTATTGTTTTGTGTGCATTCTTAATTTACATAAATGGC
ATCATATTGTAAATTCTCTTGCAACTTGGCTTTTCTTACTCAACAGTACATTTTAG
GGACTTATTTATGTTGTGTGGATACAGTGTAGACCTAGTTCATTCATTTTAACTTA
ATTGTGAAATACCATAGTTTACTTATCCATTTCCCTATTGGGTAAAATTAGTTATT
GCTTTATTGTCGTTGTTGTTTATTGCAATGAACATGCCTGTGCATGCATCTTTGTG
CACGTGTTTGTTAGTGTAAATGCCCTGAAGTGAAATTGCTAATTAGTAGGAAATA
TACTTCTGCACCTTCCTTAGCAGAGACAAATTGTTCTCCCAAGTGGTTGTACCTAT
TTGAACTCATGCTAGATTAGAAATCCCTGTGTTCCTACATCCTTACCATCATTTGT
GAGGCTTTCAATTTTTCTTATCCAATAAGTACAAATGACATTTTATTTTTTTAATT
CACATCTCTCTAATTATTCATGAGCTTAAGCATTTTTACATGTTTACTAACCAGTT
GTGTATGTGCATGTGTGTGCATGTGAGAGAGAGAGAGAAATAGGTTTTAATCCTT
TGTTCTTTTCTTATAAATTTATAGTTGTATTTATTCTGAAGTTCTTATCTGAGTTGA
AAAGTGTTCTCACAAATGGTATCTTGCCTTTTAATTTTGTTTATGTCATGTTCTATT
ATAAATAGCTTTTTAATTTTCATGTAGTTAAATTTATATGTCTTTTCAAGGTTTGT
GGGCATTTGTCCCTTAGTTAATAAATCTGTTTCTAACTCTACATTCAAGATATTCT
CCCACATTGTTTTCTAAAAATTCTAAATTTTTTTTCCCTTCACATTTAAATTTTTGT
CCATCTGGAATTTACTTTTGCTTATGTGATGAGTAGGGATCTAATTTTATCTTTTT
CCAAGCAGAAAGTTAATTGTCAAGGATGATCCAGACTTTCCCGCTGTTTGAAATG
TCATTTCTGGTGTTTTTTTTTTTTTTTTTT
>XLOC_005327 (=linc-LRRC 1-1; ENSG00000235899.1; RP11-345L23.1;
OTTHUMG00000014881.1) 566 bp
(SEQ ID NO: 5)
CCAGGCGGCACATACATGATCCCAGACACCGAAGTAACCTCTGTCTCACTCCTCC
ACTTCCAGCAAGGGATGGAAAACAAACTGAAACTGGCTCAAGTGAATGCTCACT
GGAAGGCTTACTGGAAAACTTACTGGAAGGATGTGAGGACATGTTCGGGAATCT
ATTTGCAGAAAACATATTCAGCCCTGTCCACCACAGCCAGCTGGCTGAAGAGCTC
AAAAGGCAAGAAATCAGCAAGAGAGAGAGATGAAGCATGAGAAATGAGCAAAA
AACACCCAGCACATCATAATCTTGGACAGTTTAGCAGTACATGAAAATAGATGG
TCCTCGCCCCAAGGGACTGCAGTAACCCTGAATAAACAGGATGTCTCTCACTTTT
AGCAGTTCTTTCTGTGCTAGTATTGGGGAAATATATTTTTGGCTGCATGCAAAAT
GGTAAAAGACATCTATTAAGAAAATGAAAACAATGCTTCTGTTTTAGACGAAGC
TTTTGAAGGTTTAAGGATCACCTATTTATTGACAAAATTGTTTCCGTGGCTTAAAA
ATAAAATACAAACAAATACTA
>LOC100287482 1035 bp ENST00000462322
(SEQ ID NO: 6)
CGAGGCCCTGCCCCACGCCCGGTGATTGTGCGCGCGGCCCCGCCCCCGAGGCGC
ACGCCGGCCCAGCGCCCACAGCTGCGGCGGCCTAGGTGCCGCGTGGGGCAAGCA
GGTGCCTCGCGTCCAGGCGGCTCCGCGGCTGGCTGCCTCCCGAGCCGGCCGCGCT
CCTCCCAGCGAGGCGTGGCGGGGAGGCGTAGTGAGGCTGGGCCCGTGGCGGTTC
CCTGAGGAGGGCCGAGAAGGGGCCGGGGGTGCTAGGGGAACGGGCGCTGGGGG
CAGCGGCCCCGGTGGATGCTAAGGGCTTCGGGATCGGGAGAGTCCACCACGCCT
GCCTGCTCGGCTGAGAATCGCCATGCCAGCTAAAGGGAAAAAAGGAAAAGGCCA
GGGCAAGTCTCATGGGAAGAAACAGAAGAAACCAGAAGTGGACATTCTCAGCCC
CGCGGCCATGCTGAACCTCTACTACATCGCCCACAACGTCGCTGACTGCCTGCAT
CTGCGAGGCTTCCATTGGCCGGGTGCTCCCAAAGGAAAGAAAGGGAGAAGCAAG
TGACAGCATTTCACAACACATCTCTGTTACAGACAACAGGACCTGGGGAAGAGA
AGTCAGGATAACACAACTGTTGCCAGCAACATAGACTTTACTCCAGACGACTTGA
GATGCAAATTAAGTGTGCTTTTCTGTGATGGTGGAAGATCAGGAAATGCACCTTA
CTTCCTCTGTTATGCCAGATATGGTTAGCCACTTTGGTTTTTTAGGAGCTATAGGA
TGGGAAAAGCCTGAGTAATTCCTACACAGTGTGCTGAAATTAATAGAACTTTCAG
AAATTATTATAATTCTGGGTCAGGATTAAACTTTGCTCTCAGAAGGCAGTTCTAG
TTGCATTAATTGTTTTCTTTTGCCAAAGAGCGTTTGTCATTTAGAGAAGACACGGC
AAGAAACACTGGGTTTCCTTAGGAACATTCCTCTCTTGGGCACCATTTCCTTTTTT
TTTTTTAATGGAAAATAATAAATACTTTGTTTCTATAATTTTCTTCTCAGCA
>LOC100506411 Agilent Human SurePrint G3 Probe: A_19_P00807053
Primary Accession: ENST00000554032
(SEQ ID NO: 7)
CCCATTGGGATGTTCATTAGAACTCTGAAAACTACAGTTCTCCCCTTTATGAGGA
CTGCACCACAGCTCGCCCTCTCCTGGGTTCCGCCTGGTTGCAGAGTGAGCCCATG
GGACAGCCCTCTGAAATTATACTGCTTACAACCATGCTGAGTCTGCAAGGACTTC
GTCCAAGCCTTTCCGTCCAGGACCTCAAACAGATCCAATCACAAGAAGAGAGAT
TTCAGGAAAGAGAAAATTATTCCTATCATCGGGGTTTTTGAAGAACATGAAATGA
CTGGGAAAATAATCATGTTAAGTGGAAAAAAAAAAGAAATCTATCTGTTGTAAT
TTTCAAATAATTTTTAAATAAATTTGAAAAATTAAGAGAA
>LOC100129480 Agilent Human SurePrint G3 Probe: A_21_P0000128
Primary Accession: NM_001195279
(SEQ ID NO: 8)
ATGCACTGCGCAGAGGCTGGGAAGGCTTTAATTAAATTCAACCACTGTGAGAAA
TACATCTACAGCTTCAGTGTGCCCCAGTGCTGCCCTCTCTGCCAGCAGGACCTGG
GCTCGAGGAAGCTGGAGGACGCACCTGTTAGCATCGCTAATCCATTTACTAATGG
ACATCAAGAAAAATGTTCATTCCTCCTCAGACCAACTCAGGGGACATTTCTTAGA
GAGTATGATGGAAGGTCTGATCTTCATGTTGGAATAACTAACACAAATGGGGTTG
TGTATAATTACAGTGCACATGGTGTCCAGCGAGACGGAGAAGGGTGGGAAGAGA
GCATAAGCATCCCATTACTGCAGCCCAACATGTATGGAATGATGGAGCAATGGG
ACAAGTACCTGGAAGACTTCTCCACCTCGGGGGCCTGGCTGCCTCACAGGTATGA
AGACAACCACCATAACTGCTACTCTTACGCACTCACGTTCATTAACTGCGTTCTG
ATGGCAGAAGGTAGACAGCAACTGGACAAGGGTGAATTTACGGAGAAGTACGTG
GTCCCGCGGACAAGGCTGGCATCCAAGTTCATCACACTCTACCGGGCGATACGG
GAGCATGGCTTCTACGTCACTGACTGTCCCCAGCAGCAGGCACAACCCCCTGAGG
GCGGCGGTTTGTGCTGAGAGCTATGTAAGCGCAGCCTGGACGCTGGAGGGTAGG
GTGGTTGCTACCTTTAATCAGTACTATGGATTTCTAAATGCATTTAACTGTGGTTA
ATAAAAGCGTGTATGGGCCGGGCATGGTGGCTCACACCTGTAATCCCAGCACTTT
GGGAAGCTAAGACAGGTAGGTCACCTGAGGTTGGGAGTTTGAGACCAGCCTGAC
CAACATGGAGAAACCCCGTCCTTACTAAAAATATAAAATTAGCTGGGCATGGTG
GCGCATGCCTGTAATCCCAACTACTAGGGAGGCTGAAGCAGGAGAATCGCTTGA
ACCCGGGAGGCGGAGGTTGGGATGAGTTGAGATCGTGCCATTGCACTCCAGCCT
GGGCAACAAGAGTGAAACTCCATCTCAAAAAAATAAAAAATAAAAAAT
>XLOC_002335 Agilent Human SurePrint G3 Probe: A_21_P0002106
Primary Accession: ENST00000458351
(SEQ ID NO: 9)
TTTCTGTCTTCCTCAACCCCTCAAGATCAGCGCTTTAGCTGCAAGTAAATGCCTTC
TTGCATTGGATTCTTCCCATAAACTTCCCTGCTCATTTCTCCCGTGGATTGGGCCT
TCTATGACTGCACATATATAGTCGCTTCAGAATAGAAAGCCGCTTTCTCCCTTAG
CAAGATGCTCTTGTTTGGAGGTGCCTATGGGCTAAGGTTTGCAGAATCAGCTCCG
AGACCACCCCGACTGGGAAGTCAGATGAGATGGTCTGTCCTCTTCAGCTAATGCC
CATTGTCCTTACTGTGGAGTATCAAAAGAATAACGGACATCACTGAAGAAAATG
CACTTAACATCCTGTTATAAAACATATTTTTATTTATTTTTTTCACGTGACTACTTT
TCTCTTCACCCCCTACTTTATTCACACTTTGAGAACAGACTGAAATGCATGTATTT
GTATCCTAAGTGCTCAGATCTGATAAGGTCTGATTGCTGGAAAACAATGCATGAG
AGTTTATATTCATTTAGCAACAACACACCAGTCTTCTAAACTTATTCTAATTTAGA
CATGTAAAAAGTACAATAGCAATGCATCTGTATCTGTCAGACTAAGCTAGCTTAT
GCTACAATTGTATATAAAACAATAGCCTCAGTGACTTAAAACACAAAAGCCTCAT
TTCTCACGCATGCTACATGTGCATTGCAGTGGAGTTTGTGCATCATAATGACTCA
GGGATCCAAGCTGACTGAGGCTCTATCTCCACTTGTTTCCATGATCACAAACACA
GGAGGAGAGGGAAATGTGAAGGACATGCTGGTTTCACAAGATTTTGCTCAGGAG
ACAGATGTCAATTTCCCTCACAGTTCATTGATCAAAGCAAGTTGAAAGGAGAAG
ATAGATATGAATGGGGTAGAGAATTCTAATCCTCTCCTAAAGAGATAATGAATAT
TGCTCCCAAATATTTTCCCCAAAGCTAGGAGAAGAGGCTTCAAATTCAACAAATC
AGGCTGAAAAGCCTATACTCTTAATCCTATCAATCTATCTGTGTAATTACTATAC
ATAACTATATGTGCTATCTCGGAACACATACAAACATACACATACTCACACAAAT
ACATAAGTAGATGTATATTCCTTTTTAGCGTATTACAAAATGTAAAACCATTTCC
AGATTTCTGTCCACATCTAGATCTCCCTTTGCCCCAATATTACAAACTTGGTGTTC
ATACTTTCAATGTGCATATTTTCATAATTTCATAATAAAGTTATCAATAAAAATA
>XLOC_002871 Agilent Human SurePrint G3 Probe: A_21_P0002781
Primary Accession: ENST00000498005
(SEQ ID NO: 10)
ACCAATGTGATGAGTGTGGGGAAGGCCATAGAAAGGACCGGCGAATGCTGGCAT
TGATGTGTGTTATTTTAACATTTCTGAAATCCTGTTCTTAGTCTGCACACCTTGTC
CGAGGCTCCGATGTTATCCAGGTCACCAGGTATGCCCCTGGGCTCCTGCCGCAGC
TGATCGGGTGCTAGGTGCTGAGGATACACGTCTGGGAGAAAGCAATTGGAAGAA
ATGCAAAGCTCTTCAAAGGAGACCTATAAAGTCATCTTTGTTTTGTTCATTCTTCT
CATGTTTCTGCATTCTGGGCATTCTCCTAAATTGGGGAGAAACCAAAATGCCCAG
AAGTCAAATTCTGCAACTGTCATCATGCAAAATGTCAAATGAGAGAACCAAAGT
ATGCTGGATTCTATATTGTTAGGAAGGGATGGTTAATTTGATTGACTCTTGGGAG
CTATTTTTCTAGCATTAAGTAATTCTAGGGAACCCTTCTGTGATCATCTCTGAGTA
AATAAAGAAGTGAAATTGCAATTCAAATAA
>XLOC_003734 Agilent Human SurePrint G3 Probe: A_21_P0003853
Primary Accession: TCONS_00008904
(SEQ ID NO: 11)
GAATGGTTTTTAGGATAATTTTGCCTCAGTAAATCCTCTCTACATTCAGGCATTTA
TTAGGCCATTACTTGTTTTGGGACTACAGATTATCCTGGCAGCTCAATAACTGGA
TAAACAGGACTTTAGTGAAAGATTTTCAGAGGTTCTTTAGGGAAAAGAATGACC
AGGAGAAGGTGGGTGGAAGCCTTCAGTTCTTTGACCTCTTGCACGTAGAATCCTA
AAACTGATCATGATTTTAGCTAGGACTGACCTTTCCTAGCTTGTAGGGTCACTGT
GAATTTTGTTCATGTCTTAAAAGGTTTAAGTTAACCTAGTTCACTGTTACCTACAC
AAGTAACAAGACGGCCAATAGGACCTGTCAGCATGACTTCGACATGCATTCCAG
GCATCTTTCGGGGAGTTTAGATTTACTGTGTCATTTCAGAACCCAACAAAGGTGA
TGGAAGCTCTTAGGCCAGATTAAATTTCATGGAACGGAGGCTGCAGAAGTCTGT
GCTGCTTAGTGTGTCAGCTGACTTTTTACTGGGACAAGTCTATGAAAGGCCCACC
TGTAACAAGGCCCCTTTTTGCCCTGTGGATATTTTAAAAGAGGGAATTTGGTGTT
GACAATCTTACTTACACGACTCTTGCTAAGCTATTTGACTAAGGGTTTCAATCAG
ATGCTTCCCACCTCACAAGCAAGGGTCAGCTCTATTTGCAAATAATCCATGAATA
TGTTTGTCTAAAACCTGCTGAAGAGGCATGGCAGCCACTTCCATGCTGCTTTTGG
TAATGGGTAAAGAATATGGCCTTTCAGATAGATCTGGTGGCTTTTCCCCAATAGT
CACCATGTGGAAACTATGCAACTAAATTCAATGGAAATGAAAGATACAATATAA
AATAGCGGGTCATGGCCATAAGCTGTGTCCTGAACTAACCAACTCCAAGCTGAA
GGAGGGTGTGTACTTTCCGAAACTTCGAGGCCATCTTAGTAATTATTTTAGCAAT
AATTACTAAAATGTACATGGGGTGGGGGAGCTCAGCTAAAATATCCTTACTTTGG
TGCAATAATGATCTAGGTTCTTTTTCCTAGGCCTAGGCCTCCACCTTGAAAGACA
GGAACAGAAGTTCACTGTGATGTGTGACCCTGGACAGAGATCAAACAGCTCCTTT
CTAGACCCAGATGACCCAGAACGCAGAAGCCTAGTAGTTGGTATCACCAGTGTC
TCTTCAAAAGGGCCCCACAAAAGGCTGTCCATTAATTTGTTTCATACAGTAAGCG
AGCTTTTACTGAATACTCCCTCTGTTAGGTAGCATGCAGAGTGCTAGGGCTGGCA
CATTCCTGCCTTCCCACCAGAACCCTCCAACCTCCTCCCCAGGCAACAGAACACA
GGGTTTGGGCCTGACCAGGCAGAGCTGGTTCAAGCCAGCCTGGGGCAGAGCCAG
TTTTCCAGCACACTTCTAACTTCTAGTCAGAGCCTCAGCATTATACACCCAGCCTA
CAGGTGTGTGGATTCCTGAGACAGATGGCAATGGCATCACCTGTGGTGCCAACTC
ATACATTTTAATGAGATTTCTCCCTGAAGGGTGAACCAGTAGACCAGACTAAACG
CACACTCATGCAAGAATGTAAAATTGTATTTCACTGAGGCCCCTTTATAAGCAGA
GCCATCTTTGCGAATTTCTTGGGGTGTTAATGTAAACATATCTTTAGAATATCTCA
TCGGGTTTCAGTCAGAGCCATGCTTTGGGTTTTTCCTAGCAGCAGTGATGATATC
AACTTACAAGGTTTGGCTTTCAGGATTTCAGAAGCTGGCATTCAAGACAACAGGC
AGTTTGTCAGAGCTGAATGAGAATCAGCCTGGACAAATCAAGTGCTTTAACAAG
GGCATCTTCCTCTGGGAATAATCAGTCCTTAATACAGTTTGCACTTGACATAATA
GTTTTGGTAAATGTCTTTTTCTGGCTGCACCCCCTTTTAAGTAAGCCTTTAATTTT
AAATGGTCTGGAAAGATCTTCGATGCTTTCTGTAAGGTTTAGTCACCAAGAAGCC
AGAACTTTTGGTGAAAACAGAATTTATAAAATGAAACTGAACCTTCTCCTTTCTT
ACAAAATAAAGATCCTGTCAGACTCCAGTCTCAGACCACCTTTGCCCATTTGTAA
TTCAGACTTGCAGAGTGAGGAGAGAACTGCTTCAGCCTTACTGTCTTGTAGAGAG
ATTTGGTGAAAATCATGTTACTTTAGACCCAGTAGTTTTCAGGACCGCAACAGGA
TGCGGGGCACCTGGCTTCCCGGGTAAGGTCACATAGTCTCTTAAAATTCTGTCAC
TAATTTTTTTAAACGACTTTTTTTAAAAAGCCACCTCCTCATGGGTGTCCACTTTT
TTCTAGTTCCTCAGCTGCTTCTGGAGCAGTGTTCACAACGGGAATGTTTTTACTGT
CCTTGGTAGGCTACAGGTTCACAGCTTCAAATCAAGGCCTCCAAGGATTTTATTC
TCTTACATCACAGTTTTGACAAGTATGCTTTTAAAAAACAACATTTGCAAAACTG
GTCTTTAAGCGACGTGAGTCAGAGGTAACAAAGGCATATATATACCGAACAAAG
GTGCTCCGGTGCAGTGGAGAGAACAGTATTAGTGTCGCAAGCACAGGAGTGCAG
ACAGCCCCGCCTTCATCGTGATGCCTGCAGCACACCACGATTATCATGAGAGGTC
AAGATTTTGATTTACTAATTTATAATCTTATTTCCAAGCAAAACAAGTCAATTTCA
TGTTACAACTTTTTTCTTGTTTCTTTTTATCTTGTTTGGCCTGAGGGTTGGGGGATT
TGGGGGAGTTGTCAGCTGCACAATCTTTGAAGTGTAAGTTAATTTTTATGTGATA
TTTCAGTATATATTTTATTGATTAAA
>XLOC_003734 Agilent Human SurePrint G3 Probe: A_21_P0003854
Primary Accession: ENST00000508664
(SEQ ID NO: 12)
AAGATATTCTAGGCCCCTTGTTGCTTCAGCCATCAGTCTATAAATAACACAACAC
TAATTTTCCATCAAGTAACAGCTTAAAACAGAACACTGTCAAGATTTTGATTTAC
TAATTTATAATCTTATTTCCAAGCAAAACAAGTCAATTTCATGTTACAACTTTTTT
CTTGTTTCTTTTTATCTTGTTTGGCCTGAGGGTTGGGGGATTTGGGGGAGTTGTCA
GCTGCACAATCTTTGAAGTGTAAGTTAATTTTTATGTGATATTTCAGTATATATTT
TATTGATTAAATTTATTGGAAAACTT
>LOC154822 Agilent Human SurePrint G3 Probe: A_21_P0005276
Primary Accession: BC013024
(SEQ ID NO: 13)
ATGAGATGTTAGTTGGTACAGGGAGGGGTTTCCAGGACCCGCACGCCCTTGCGG
AGTGCCTGCTGGAGGGAGCCGGTGTGTCCAGGACACCCTTGCGGAGTGTCTGCTG
GAGGGAGCCAGTGTGTCAGTGAGATGGCTATGCCCCTGGGCTGCTGTGTCCCAG
GTTTCCTCAGTCTCTAACCCTTTGTTCTCACAGGGGATGGACTCTTGCTTCTTTTC
CCAACTCCACCAAGAGGGACCGTCCCAGGACGTCCTTCCCCGGGCATCTGGCCCT
ACAGCTGCCTGAGGTCTCCATCACCGTTGGCGCCATCAGTCTGCTGTGCAGCCAG
CTGTTGGTTTGGAGAGCCTGAAGAACTGCAGTTCACGTCTCATCTAAAGGAGCTG
AAATGATATTGCAGCTTTTTCTTTTGGTTGCGTGCAGTGAGAATCTGGGAGCTGA
ACCTGTTATCTGCATGGTCTTCAGAAATCAGGCAAACTCGGAAAATGCCAACGCC
AAAAATGCTGATGGGTGACAAAGTGTCACAGGTGTGATGCATTACAAATCTCAG
GACTTTTGTTCACTGGATTTGAAAGGTCAAGCTTCACAGGAAAATGATGAAGTCC
CAAAAGACCAGAAATATATTTCAGAAGATGCCAGTTACTACTTTAAATGTCAAAC
CAACATTTCAGAAATAACTTTCAATGATTATTTCCTGCCAAGAAGGTGAACGCTG
GAGACCTTAATGGTGGAAGATGGAGGGCGTCTTTCCTTCTGTTAAGCTGACAACT
TGGCTTCCATCTTGTGAGGACCTCACCCTACCTGGTGGCAGAGGACGTCTGACGC
CCTCAATCATTGCCATTACACTTCCCAGCCTGGTGGTCAGTCTCCTGGGGTCTGTG
TGTTAACAAACCATCGACTGGACAATCGCAGTTTTCCTTATGAAGGCTTACTTTA
AAAAGGCTCTGGATTTTCAGAAGCGAAGTCGCTTTCATCCCCGATTCAGACCCAT
CCTAGTGGAGGAAAAATCCTACCAGAAGAAGGGCTGACCATAGGAACTTGCCAT
TTCCTTGACCCCATCATATCTGAGGAAAAAACAACAGAAAAGGTCAAAACCCAC
GTGTACGCCCAACGTCCTGATTGACGACTTTGCCTGCAGCTTCTGCTTTCCTGAAA
TTCGCTGCTGCCTTTAGAACCCTTGTCTGCAGCCAGTGGGGAGTTCAGGACTTAG
GCGGAGCTGCCCCACCCTCCTGCTTGGCACCCTGCAAATACATGCCCTCCCTTCC
ATCGCTGCAGACCTCAGAGTGGGCGTCCGGTCTCCTGTGCGGGATGAGAATACA
CACCCTCCCTTCCATCGCTGCAGACCTTAGAGTGGATGTCCGGTCTCCTGTATGG
GATGAGAATACACGCCTTCCCTTCCATCGCTGCAGAGTGGACGTCTGGTCTCCTG
TGTGGGATAATACACGCCCTCCTTTCAATCGCTGCAGACCTCAGAGTGGACGTCC
GGTCTCCTGTGTGGGATAATACACGCCCTCCTTTCAATCGCTGCGGACCTCAGAG
TGGACGTCCGGTCTCCTGTATGGGATGAGATACACTCCTTCCCTTCCACTGCTGC
AGACCTCAGAGTGGACGTCCGGTCTCCTGTGTGGGATGAGATACACTCCTTCCCT
TCCACTGCTGCAGACCTCAGAGTGGACGTCCGGTCTCCTTTGTGGGATGAGAATA
CACTCCTTCCCTTCCATCACTGCAGACCTCAGAGTGGACGTCCGGTCTCCTGTGC
GGGACAAGAATACACTCCTTCCCTTCCATCACTGCAGACCTCAGAGTGGACGTCC
AGTCTCCTGTGCGGGATGAGATACACTCCTTCCCTTCCATCGCTGCAGACCTCAG
AGTGGACGTCCAGTCTCTCTGTGCGGGCCAAGTGTACACAGTTTTGTTCCGTCAC
AACTTCCACGACAGGCCAGTGTGAGGTTTTTGAGCTGGTGCTGACTGAAAACTGT
CAGCTGCCCAAGGACCTGGGAGCTCTGCTCCCCACTCCTGGTGTGCGGTCTTGCG
CCTGGCCTCCCTGCCTAGGTTACATGCAGTGGTCATCCCGGTCGCTCCCACACCC
GTGTGGGCTCTGGGATCCCCTCTTCCAGCCAGCCCAGGGGACATCTGGCTGTCTC
AGGACCCAGCCATCTGTAAAAATTAGGCAGGTCCCTTCAGTATGCTCCTGGTCAA
CAAAGAAAAACTTCAATTTTGAGAATGGCATCTGTATTCCGAAGTGTTCTCTCAG
ATGTTTGAGTTCCACTAAGTAGATTTTCTTAGTCTGCTGTATCAATGACACAGAG
AGACGTGCATTAAAACCTCAACCATGTGGATCTATTTCTTTTCAGTTAATTTTGCT
TCATGTATCTTGAAGCTCTGTTATCAGGTGCATGCACATTTGGGATTGTTATGCTT
TCCTGATGAACTGACCTTCTTTCATTATGCAAGGGGAAGAAGATGCTGCATACAG
GATGGAATATCCAGGGGAAGACGTCTAAGGAGAGATGCCCAGCTGGGAGTCCTA
TGCAAGGGGAAGAAGATGCTGCATACAGGATGGGATATCCAGGGGAAGATTTCT
AAGAAGAGATGCCCAGCTGGGAGTCCTATGCAAGGGGAAGAAGATGCTGCATAC
AGGATGGGATATCCAGGGGAAGATTTCTAAGGAGAGACACCCGGCTGGAAGTCA
AGATATGTCAGTTGTTTCCATTATAATAAAACCACTCATGTTAGATGAGCTGAAC
TTTCCCTTTTCCCCAGTTCTTACGATCAAAAAGTGGCTGTCCTAAATTTCATCACT
CAATATCCTTGCTAGAGTCTTCCTTTGTCAGCCAGGCTGGAGTGCAATGTGCAAT
GGCACAATCTTGGCTCACTGCAACCTCTGTCTCCTGGGCTCAAGCAATTCTTCTGC
CTCAGCCTCCTGAGTAGCTGGGATTACAGGTATGCACCACCATGCCCAACTAATT
TTTGTATTTCAGTAGAGACGAGGTTTCACCATGTTGGCCAGGCTGGTCTCGATCT
CCTGACCTCAGGTAATCTGCCCACCTTGGCCTCTCAAAGTGCTGGGATTACAGAC
ATGAGCCATCATGCCTGGACATAAGTGAGTTTTATATTGTATTATAAGACTATGA
TACAGTAAAACCATGAAATCCAAATTTATAATATCACACTACATAATACAACTGT
AACCTCACCGCCCTATCCTGGGATGTGTGTCATTTTTATAGCCAATTATGGCCCCC
AGCTTTAGTTTTCTTTTGCTTATTGGAGAGTGTAATTCTCCCTTATTCTTTTTGCTT
TCTACAGTCTTGTGTACATCAGTTATCTGTTTTTGTCCTTTTGCCAGTGTTCAAAG
TGTTATTTTTCGTATTTACTTAAGCTCCTGCAGGGAGATTAGAATTTCTTCCCCTA
AGAAGAAATAAGTAATAGCGGAGACCTGCTGGGCACTGGTGGCGCCAGGCTTGG
CTCTGGGGCTGCCCATCCATCCTCACAGCATGGCGACTGGAGGGTCTTGCCCTGA
GGTCCCGTGTGCGGAGCAGGGCTTGGCATTCACTCCTAGGCACTGCTGACTCAGT
CTGTCCTGGTGGTGCTGGGAGGCCGAAACCCGTCATGCATGTAAACCGCCGGGC
CCCGTCTGGCATGGTGCACCTGTGCTGGGAGTGCCTATAGAGTAGGAAAAGTATT
CCTGGACCTTTAAAAAACTTAGGCCAAAAAAGTGTTTTGGTTGAATCTTTGGCCA
AATTGGAACTGCAAACTCTGTATTATCTCCCCTTTTGTGAAATTCTATGGAAAATT
CGAGCAAATAAATATGCATTTCCCAGTGAAAAAAAAAAAAAAAAAAAAAAAAA
AAAAAAAAAAAAAAAAAAAAAAAAA
>XLOC_007162 Agilent Human SurePrint G3 Probe: A_21_P0005873
Primary Accession: TCONS_00015107
(SEQ ID NO: 14)
CGCACCTGTAATCCCAGCTGCTCGGGAGGCTGAGGCAGGAGAATCGCTTGAACC
TGGGAGGCGGGGGGTTGCAGTGAGCCGAGATCTGGCCATTGCACTCCAGCGTGG
GCAACAGAGTGAGACTCCATCTCAAAAAAAAAGGTTAATCTTTCCAACTAGATTT
TCAAGGATGAGGATTTTGTTGTTGTTGTTGTTGTTGTTCTCAAATGTATTCCCAGG
GCTTGGAACAGAGCCTGACATATACTAGGCACTCAACAAATATTTGTTGAATGAT
TGTAATGAGTAACACCCATTTTTGCAGATCTTTGTCTTCTGAGCCTAGGGCATAG
GTCATCACTGCAGGGGTGAGATTGTCAAAATGGGAGTCTACAGCGCCAGAGACC
CAAGTTGAGGAACAGCCTATAAAATAACTGGC
>XLOC_007697 Agilent Human SurePrint G3 Probe: A_21_P0006269
Primary Accession: THC2779256
(SEQ ID NO: 15)
CAGACTTTCTTGTTTGCCTCATCCCTACCAGTGTCTTTCTCCCTACACCTAAGGTC
AATTACCAGCTGCCCTTTATCGTTGAACTTGATGCTTTCTTCTCATAGTAGAATTA
AGAGGAAAGTAAAATATTTTTTGTACCTATATCTTTATTATATTTAGACAAATCA
CAGAGTGAGAGAGTAGGGGTTTCAAGAAAAATAGGAGAGAGATAAAGGAGAGA
GAAAGAACTGCTTGTGGAAATACAGAATATCCCACATTTTCAATGTGGAAAGTGT
ATGAGGGTATGAAAGAAAATACTCAGTTTTTTTTGTCCTGTAAGAGGCAGCATTG
ACAAATGTGTACCAGAGTTTGGGTACATTTGAGCCAGTTCTTCAGAATCGTGGGG
TGGGAAATAGAACAAAATTATTTACACCTAATTCTAGGCAGATAAGTGTGCTTCA
AGGAAAGGCAAGGGCCTGGCTAGATTCTAGATGTTTTTAAACTGGAGGCCAGAG
ACAGCTTTAGGGAGTCCATATACAGGCACAAATTTATTTCTTTTATAGTCTTCTTG
CTCTTTGAAAATGGTCTTTATGCAAATACTCACTATATAACCAAAGTTTCTCTTTG
TTCCAGGCAGCAGTAGGGCTGATTGGAGCCATTGTACGTGTCGGGAACATATCA
GAACACCGAGAATAGCGTCATGTCATAAGGACTCAGAGCAGGTGGACCCTGCTG
TGATGCACAAAGAGGACCACGCAAGATATGATAAAGATCTATGTCACTGAATTT
TGGTTCAATTTTTGTATCTCAGCTTCCCGGAAATAAAAAAGAATTCTAACATTCA
TACTTTCAGTATTTTATGTGAGAGGTTTTGTTGTCAAAATCAAGTCTGAGAGCAA
TGTTTATTGGGGTCTTTAATTGGAGTCACCA
>XLOC_010807 Agilent Human SurePrint G3 Probe: A_21_P0008324
Primary Accession: TCONS_00022478
(SEQ ID NO: 16)
TTACTTTACATCAACATAGCAGAACAAATTTTTGGTGTTTCTTACCAAGAAAATC
TGCATCATTTGAAAGTATCCAAAAATGGTTTAGTGCACAACCTACACAACTAAGG
CGAGTAAAATCTTCTGTAGACTTGAGGAAGGAGAAGATCATAGCTCCTTTGGAA
ATCAAGAATGATATGCAAAGCAGTATAAAAGAGGTTATGTTTCAGAAAGCAAAG
GAATTGAAACGTCAGCTCCAGCTCACTAAGCAAAATAAAACTGAGGAGCCCAAC
TATGTGAAAGAAAGTATAGATGACATCTTTGATAACATGTGCGAAAAACACAGT
TTGAGAAATCTCTCTTTGACTCTCATTGAAGCGTCTAAAAAAGCTGGCATTAGTT
ACATTGTTTATCCCAAGAAAAAGAAGATGAGATGGAAGAAAAGATTGAAACAAC
AAAAACTTATATTCGTGCATGAAGAGTTATCCAAGCCTCCAAAATCTCTTGAAAG
GTCTTGTTTAAGTGATTTTCTTATAGTTTAAGAAATATATTGTGGTTTTGACCTTA
ATTTTATAATCTCACCCCATGAAGTTATTATTTT
>XLOC_010813 Agilent Human SurePrint G3 Probe: A_21_P0008331
Primary Accession: THC2542080
(SEQ ID NO: 17)
GGGAACTCCCTGACCCCTTGCGCTTTCTGAGTGAGGCAGTGCCTCGCCCTGCTTC
GGCTCGCACACGGTGCGCGCACCCACTGACCTGCGCCCACTGTCTGGCACTCCCT
AGTGAGATGAACCCGGTACCTCAGATGGAAATGCAGAAATCACCCGTCTTCTGC
GTCGCTCACGGTGGGAGCTGTAGACTGGAGCTGTTCCTATTCGGCCATCTTGGCT
CCTCCGCATTTGTTTTTATGGTGGGTTTTGTATTGTTTTTATAGAGCTGCCCTCAC
ATGCTTCAGCAACATTAGATGTTCTGGAGACTGGAAAGTCCAAGATCATGGTGCC
TGAGGATTCAGTGTCTGGTGGACCCTATTTAATGTGGGAGAGGAATACATAAAA
AGGTGGATATCATGAGGTGAAAATCATTGGAGACCATCTTGGAAGCTGTCTAAC
GCAACAAGAATTATTTTATTTATGATTTATTTGAAGTCTTTTTTTATTTATAATCTG
TTTTACTTGGAATGATTGGTTATCAGACTCAGCACGTTTTCAAATCTGTATAACAG
ATGCTATCTTGTTTGTCATTAGGTAAGTTCACCTGAAACCCTCAGGCAAGCCTTTC
AGAACTAATGCCTGTTGTAATTCCCTTATTTCTTATGTGATTTAAATTGTGAAAAG
CCCATATTTCTTTTGAAAAATCATTGCTTGTCTTTTGTCTCTGATGTTAGCATGTTT
TCAGGCAATGTTTTTGAAGTTAAATATATTTGTTGTTTAGAGATGACTTTTCTCTG
CCCTTTTATTTTACATACAAGGGTACTAGTATCCAGAGAGGTCAAGTGGCTAAGG
ATGCAAAATTTGTAATAAAATTTAGGTTTTCTGAATCTT
>XLOC_12_000735 Agilent Human SurePrint G3 Probe: A_21_P0010596
Primary Accession: TCONS_12_00000977 (Probe is in reverse
compliment orientation)
(SEQ ID NO: 18)
TTAAAAGGTACAATTCACAAGGTTGGAGGGGTAGCTGGAAGTTTCTGTGGTTACC
TTGCACTGGGGGGCTGCCCTGCCTCCACTCTCTCCCCACAGTCCGAGGGCAAGAT
GAGCACCCCCACCCAATGGCAGGACCAGCCCTGCGGGGAAATGTCAGCATGAGT
GGAAGCACGGCAAGGCCCCTTCCTTCTTGGCAAGGGGCTTCCCTGGCAGGCAGTT
CACAGGGTGTGTGGGTGGGGGGGATGCTGACCAGCTGCTCTCCTGGACCCTTCCT
GTACGAGCCTGTTTTTTTTTGTTTTGTTTTGAGACAGGGTCTCCCTCTGTCGCCCA
GGCTGGATGCAGTGGTGCAATCTTGGCTCACTGCCACCTCCACCTCCCCGGTTCA
AGCAGTTCTCCTGCCTCAGCCTCCCCAGTAGCTAAGAGGCACCCACCACGATGCC
CGGTTAATTTTTGTATTTTTAGTAGAGATGGGGTTTCACCATGTTGGCCAGGCTGG
TGTCAAAATCCCGACCTCAAGTGGTCTTTCTGCCTCAGCCCTCCAGAGTGCTGAG
ATGACAGGCGTGAGCCACCGCGCCCGGTGAGACTGTGGTTCTTGGAGGCTTTGG
GGATCCTCTTGTCCACCCCGTCAGGACCCAGCCTGGAGAATGAGGGGTGGACAA
GCTAAATGGAGCCTGGTCTTGGTGGGGCCCCGGTGGAGTCCTCAGAGATGCCAG
GCTCCTTTCGCGTCCTCGGGGACCGACTTCCAGTGGCTGCTGTGCCCTTGGGCCC
CCCAGTGGGGGACGCCCCATGGAGCTGGGCGAGGGCGGCTGACCTGGGCAGAGG
CTGCTGGCCCTAATTATCAGTCAGAGGCCCGAGGGGGGAGGCGGCTGTGCTGGT
GGCCGGGGGCCGGGGGGGCAGGGGCAGGCAGCGCAGGTTCCCGGTCTTGAGCGC
GCACTGCACCGGCCAGAGTGCCACACAGAAGAGCATCAGCAGCAGGGCAGAGA
CCAGTGCCATGCGCCTCCAGTCCCTGCAGCGCGCCCAGCAGCGGGCCAGGCGGC
CCCGGCGGGGGGCAGGGTCCCGGGCGGGCGCGGGCGGCTCGGCAGGCTTGCTCA
AACCCACGTCCACGCATACGAAACCGGGCTCGCGGCCAGGTGTGGTGGGCAGTG
GCTGGCAGCACAGCTTGGTGCCCTCCAGCCACACAGGCTCCTCACGCCGCAAATG
CGCCGGCATCCGGGCCTGCAGCTGGCGGCTGGTGCACAGCGCGGGGGCTCCGGC
GGGCGGCACGGCCGTGGGCTGCCTGCAGAAGGGGCAAGGTACAGCCTCACCACC
GGGGCGGCCCACAGGCTGAGCAGCCGCCAGCCGGGCCAGGCACTCCAGGCAGA
AGACGTGGGTGCAGGAGAGCTCCTTGGGTGTCTTGAAGATGTTGTCATAGCCTGA
GAAACAGATGGAGCACTCCAGGGGGGAGGCCACCCTCTCCGAGCCAGGGGTGCC
AGGGGACCTGGGGCTGCCGGCCGAGCTGGGGGACCTGGGCATCGAGGCTATGGA
GCTGCTCCGGCGAGGGGGTGGCACAGCCGTGTGCCACACCTGCTGGCCTGACGA
CATGTCTCTGAGCTGTGGGACAGGGACTGTGGTAAGCAATCACCGGCCGCCCCTT
TCTGGTGGTGTTTTATCTCTCCCTCCCCTCTCTCGCCCCAGAGATCCCAGGGAAGG
ACTCTGTTTCCTGCGCGCCACTCCAGAAAGTTCCTCCGGTGCCCCTGGAGGTCAT
TCTGCCCCACGTGCAATCCTGTCCTCTCCACCCCATCACATGGCTGCACCGGGGT
GAGCCTCCCACAGGGCCCCAGGCCTGCTCCGGGAATGCAGGCCGTGTGTAGGGG
GGTCTCACTGACCGCTCGGCAGACACCTCCTGTTGGCCCTGCCCCACCTGGCTGG
CCCTGCTGCCCGGGCAGAAATAATGGTGAGGATGACAATAGCCACAGTCGTCAC
TGTTTATGTCGGAGCTCTGCAAGGCTGGGCCCACATCACGGGACTCACACAACGC
CACAGTGTGGAAAAGGCCGCCCAGAGCATGGGTGACTCGGCCAGGGCCACCCCA
AGGGAGCTGGCGGGCCCTGGACCCTGGCAGATACGGCTCTCAGGCAGGCCAGGG
ACTCCAAGTCAAGTGAAGTGAGTTTGAACTCAGATCCCAGGATGGGTGCCTGGCT
TGGGCGGTGCAGGCCTGATTTGTAGGCAGCTATGTGAGGGTGGGGTGTGGGGGT
CTCTGGGTCTGGGGACCGGGCTGAGCCCCGGGGGCTTTGGGACGACAGGGAGGG
CCCAGGCAGGGGCAGGGGTCAGTGCCCGAGGAAGGTGCACGTCAGGCACGACCT
GCGGCCTGCGGGGCCGGCTTGTCTAGCTGCTGAGGGTCTGATGTGCACAGTGTGG
GGGTGGGACTTGGATAAGCCCAGCCATTCCCTCTGGGCCAGCCCACTGCCTCATG
GTCAGGTGATGGTCAGGGCACCCTCAGCCGCCCACTGAGTGGGTGTTTCTTCTCC
CTGACCCAATCCCACTTCATGGCAGGGACCCTGGGGGACGGACACTGGGGGATG
CTGCTCTGCCCCTGGGCATGGCTCAGGTGGGCATCTCAGCTGACCTGGGACCCTG
CTCCACCTCCCGCCCCTCCCCTGCACCCAGGATCCGCTGCAGGGAGCCACAGGGG
TCCCACCTGGAGGGAAGTGGGCAAGGGTGACAGTGAGACTCAAGGGCCTGGCCG
TGCGTCCCCGTGGGGCCCAGGAGGCTGCCCCAGAAGTGACTCCTGGCACTGCCCC
GCCCCACCCCTGACTTGCCAGTGAGTCCCAGACAGGCTGGCGGGATGACACAGG
TCACTGTGACCACCTGAGTCACACGCCGTCACTGTGAGGCCGTGAGTGCCCCAGG
CACCGGGACCTGGGGACTGTGCTCTGCGGCCTGTGTACCCCACAGAACCGGTTCC
TTGGCACGAGGCCCCACCCCTCCACGATGGTGCCCCACCCTGAGCCTGTGCAGGT
AAGGGGTGAACACGGGCTGAGCTGGCCTTACCTGGTGGCCGGGGGTCAGCGGGC
CTGGGCGTGGTCCTCCTCGCCGGCCACGGTTGGGCTCCAAGGCCCTGGGCTGCCC
TGCCGTGGCAGTGTCTGCTTCCTCTTCTCCGGGCCCGGCCCGGCCTGTGCTTCACC
CAGCAGGTATCCCTCCCCGGGGCCGGCCACCAGCAGCTGTCCCGGTGGCACTGGT
CTGGCAGGTGTGGCTTCTGCTCTGTCCAAGACAGGCGGGGACACAAGGAATGCG
TGCGCCGTCACCCGCACAGAGCTCTGGTCTGAGGCAG
>LOC100506922 Agilent Human SurePrint G3 Probe: A_21_P0011848
Primary Accession: XR_109888
(SEQ ID NO: 19)
GCGGCCGCGGCACCCTCGTCAGGCGCCGCCGCTGAGGGCAGGCAGCCCGGCAGC
CACTACACACGGACCCGTGACGTCGGGCGTAGCGCGGCGCACGTCACGGCCGCT
CGCTCGTGCGCGCGCACCCCTCCGCCCGGCGGTAGCGGAACCCGCCGCGGGCGC
GCGCCCGGCCCAGGGGAGTGGGTCGGCGCCTGCGCAGAGGCCCGCCACGCCCAC
ACACAGGCCACCGCCCCCACCGGCCGGACGGCGCGGGGATTCCCAGTCCTGGCT
CCGCCCCGGCCTCGGCCCCGCCCCCGCCCCTGCCCCGGGGCAGCCTGTGCTGTTC
CGTGTGCGCGGCGCATACGCACCTGGGTTGTCTCGAGCCTGCGGTAGTGGCCAGA
TCCCAGACATCCGAGTAGATCCCGTGAAAAGGTCTCCCACGTGGGCTGTGGACA
GGGCCCAAGGGTAGCAGAGCTAGCAGAGGCAGTGACGGACTGTGTGGCAGGTCA
TTTGCAAGGAGAAAAGCCGTCTGCCTCTTAATTTGTGGCTCAAGTTTCAGAATTT
TTTTCCTGAGGGACTTTAGAAATTACTTCAGGCTTGCCACCTAACCTTAAACCAC
CCCCTTGGAGACTGGCTAAGTGTTATTTGTGTTTTCTGTTTAGTTCTTATCACCAT
CGATACTTGGTTATGACTGGTTGTGTACATTGGTTAGCCCAGCAAGTATTACTTCT
CCAGCTTAACAGATGTGGAAACTTAAGCCCAGAGACATGAGTTGACACCCCACC
CCCAAAGCTAGAGTCTAAAACCCTTTCTTTCGCTCCTCATCTCCCACAGGATAAA
ATGCAAATTAATCAGACTAGTGGTGAGGCCCTCCGTGGTGTGACTAACCTGCATC
CCGACGTTTTCACCCTACTTTGATCCAGAAAGCACCTTTCCGCCCCATCTCTTCTC
CTTTCCTTAAATACCCCTTACAACTTCCTGTACCATTCTTCCCTGTTCAGCTTCTTC
TTGGTTTCTTCGTACATTCTGGATCCACCCCTTTCATGCATATTCCAGACCACATT
TCCACTGGAGCAGTTGAAATGAGAGAGATGGGAATGGGACTCACCCGAACCAGA
GGAATTTTTATTACAGACCCATTAACAGAGGTGTCAAAGTCACAGGAACAAGGA
TGTGCACCTCAGAAACACAGAGGTCAGTGGAAAATCAGTTTGCTTCTATTTGTTT
AAAAAATGGGGGACTTATGCATAAATCTAAGACCTTCTTGAATCTAACATTCTAA
GACCTGTATGCCACAGAAAGGAGGGTCTCAGAACGCCGGAGGATAGTATTTAAA
TCTTAAATATCTATATTGTTCTCCACAGTTACTGGGTCACCACATAGCAGGCATTC
AATAAAAACGTGTTTGTTTACTAAGTAA
>ANKRD20A9P Agilent Human SurePrint G3 Probe: A_21_P0012182
Primary Accession: NR_027995
(SEQ ID NO: 20)
AGGACAATAATACCCCACTTTTATTCGCTATAATTTGCAAGAAAGAGAAAATGGT
GGAATTTTTATTGAAAAACAAAGCAAGTACACATGCCGTTGATAGCCTGAGATG
GTACAGTAGTTCTTTTTAAATGAAACCTGAGTATTCTAGAGTGGTAACAGTCACT
CAAGTCAGAAATACTAATAAGAAGATTAATGTAATTATTGGCATGTAGTGAAAA
ATGTCACCATGAATAATCAGATAGATCAGCAAATATTTAGACTGAGTAACATAA
AGAACAGTATATAGTAGGATTCATCTTCTCCTATAATACAGAGTGTTTGTTATTTA
TAATTGGATGTTTTTGGTACTGTAATCTTTTATTAGCTAAAGGGTTTTGTATTAGC
TTTATTAAGTTTTTTTTGAGATGCAGTCTGGCTCTGTTGCCAGGCTGGAGTACAGT
GGTGTGATCTTGGCTCACTGCAACCTCCACCTCTCAGGCTCAAGCGATTCTCCTG
CCTCAGCCTTCCAAGTAGCTGGGACTACAGGCGCGCACCGCCATGCCCAGCTAAT
TTTTGTATTTTTAGTAGAGATGGGATTTCACCATGTTGGCCAGGATGGTCTCGATC
TCTTGACCTTGTGATCTGCTCTCCTTGGCTCCTCAAAGTGCTGGGATTACAGGCAT
GAGCCCCTGCACCTGGCCAGTTTTATTAATTTTTAAAGTGTGGACTTTTAGTTTAT
GACTACTAGTATTATCATCATCATCATTATTGTTGTTGTTGTTTTCAGTCTGCAGA
TAGCTCTTATCTGACCCCTAGCTGATATAAATTACAATATATCAGACTAGGAAAG
CAATGGGGAAATCTTCATCTAAATCTTTACCTGCTTTAGATAAGTGACCTCAGCA
CAGTTTCTTGGCCATCAAAGGACTATGAGTTAGCAACTTGTATTATGTCATACCC
CAGTGGGACAGGAGGCTTCCTTATTGTCCTTTTCTTTTAGACTTGGTGACAATTTA
TAAAGATGAACACCTGAGCACCCTAGATGCTTATAGACCCAAGCTAGTACATGC
AAAATGTTATTATGTCTACACTGACAGGTGGATATTAAATTGGTAAAGTGTATCA
AACTAGCTGTTTAAAAAAGTCTTTATTAAAGTTCTTGAGTGGAGTGATTTCCTTGT
TATTTTAGAACAGCCCTTATGCTTGCTGTGCACTATGACTCACCGGGTATTGTCAA
CATCCTTCTTAAACAAAATATTAATGTCTTTACTCAAGACGTGTGGACAAGATGC
AGAAGATTACGCTATTTCTCGCTGTTTGACAAAGTAAGTGTTTATGTTAAAAGGC
CAGTTAATATTGAATTGAAGTTTAAAATAATTGCAACTATTCCATCTTATACATTA
GGTGAGAGTTCCTAGTTTTGTTCAGATGGTTTGAAATAGCAATGAGTTAGTCTAC
CTTTTAGCCAGAAATCAAGCAGAAGTCTAGATTAGTTAGAAGTAGAGTGCAAGA
TGTTTTCAGGATTTTTAAGACCTTTATCCCTAGGGATCTCAATGTTGTTCATTTTA
TTCTAAGTATAATCCCCATGCATGGGATAAAAAGAGCCACATTTTTTACTTCTTTT
CCTTTCTTTTCTTTTTTTTTTTTTTTCCTTGAAACAAGGTCTCTGTTGCCCTGGCTG
GTCTTGAACTCCTGAGCTCAAGTAATCCACCTGCCTCGGCCTCTCACAGTGCTAG
CCACCGTGCCTGGCCTGACTTTTCTAATTAGTTATTGGGTCTTGAAATGTCCAATT
TAGCAGAAAATCTTGTATTTTCCCGTGGGGCTATCTCCTGTGTCTTCCTTCTTTGA
ATTTTCCAAGAAGCTAAGGGGTTTCCTAAGTCCAAGGAAGGCAATCTTTCTTTAC
AAGTCAGAAGAAGGGGGAAAAAGGCCATTCTGATCTTTCTGTTGTTTCCATGGTC
TCACTTCCTGTATTGTTGCCATTGTAACCAGTCCTGCAATCTGATAATGATTGACC
TTTGCCACCAGGATGCCTTCACTCATTCAGACCCCTCAGTTTTTGTGGTGATTCAC
ACATAGAGTTCAAAGCTACGGTGTTTATTAATTTATGTACTTATGCTCAGTCGTTG
TTCCCAGCACCCTGATCTGGCAGCTAGGCCTCCTAGCTTTACCCACACAAATGTC
GAGCAAGTTGATCCTCACCCTACAGTAAAAACCTTATTTGGAGCCCACATCTTAG
CTAGACTTAGCCTAGGCATTCATGGTAAGTTATCCTTTGAGACCCGTGTTTGTCTG
TTCTTTAACCAATATTAGTTGGGATTGCTCTCAACAGTCAGGGATGTTAAAATCA
TGTTGCAGGAAGAGATTAGGGTTCCCTTTCCCTTTTGCTATCAGATCTGTACCTTG
AGGCCTTTTTACATCCTGTGGAGCAGCTTTTGTTAGATAGCAGAAGGTTCCATGT
TATCTTTCCACCGAGTAGTGGGAACCAACTTGCAATTGGCCCCTCAATTAATGTG
TCTCTATGATAATGAAAATCTCCTGGGCTAATCACAACTCTTCCAGGAGTTTTAA
ATGTATTTTAAAATTCTACCTCACAGGAAGCCATTCAATAAAATTCTCTGAATCT
AAAGTCAGTGAGTTAGATTTAACAGAGCTAAGCCTCATCCATAACTCATGAGTAT
CCATTTATCAAACAGGGCTTTGTACTTGTTTCAGCAGCACATATTTTAAAATTGG
ATCAATACAGAGCAGGTAAGCATGGCTGCTGCCTAGGGATGGCACACAAATTCA
GAAAGCATTCCATATTTTGCATAGTCCCGGGAAGGTCATTTGACTATTTGTTGAG
TAGCTCCAAGGAAGCAGTGTGAGTGAAACCAAAACAGAAGACACCCAATATTGA
AATTGTGATTATCACTATAAAACTATTGATGTAAGGTGATCTCTGAAATGAGAAC
AGAGCTGAGTAATAAGGGGATGTTACTTGTTGCTAGTACATGTCTTGGAAATGAC
AAAATGTCAACTTGCATTTCCTTCATGGAAGTGAAAAACAATAAAAGCAGGGTTT
TGTCTCATCTGTTAGTTGGAGAGGACCATGGAGATCCAGCGTCCTAGCACAGATC
TGCTGGCTCAGAGTTTGAGGAGGTAGAGAAGGAGAGGTAGTTGTCCAAGCCCAG
GTTTTGACACCTATTAGTTTTCTGCCTTTGGTGTGATTGATGAGCTCAGTGATGGG
AGACAATTAGGTAATCTATTTTAATCAGATTAGTTATGAATTAGGTAAAATGCCC
TGAATTACAAGCCACAAAGAATACAACTTAATAACCAAAATTAGCACTTAATAA
CATTTTCTGAAAACTGCAACAATTGAATATTAGAACTTATAGAAAAACACACACC
AAGCAATAAAGTTCAAGAATAAATCATTCCATTGCTTTACTATTTCCTGAACATT
TAAACATGTAATCTTATTACATCTTCCTAACAACCTACTGAAGTAAGGTAGCAAA
ATCCTTATTTTTTAGAAGAAACCAGGGAGCCTAAGAGAAGCAACTTGTCTGAAA
ACAAAATATCTATTACAGAGTGAGGATTTATTCTGAGTGCAGGACATGTTACATG
ATGTCCAGCTAACTAGAGTTCATTTACTGAGCTATGCTTCCTCCATTTATGAGTAC
TTCACTTTTTTTCTTCTTTAATTATAAGCTTAATAAGCTTGTAAGGTTTAAAAATTT
GAAGTATATGGGATATTAAAATTCTGATATTAGGTCTGATATTGCCTGAAATGGT
TTTAGAATTTAATATGTTTGGTAAATATTTTTTATTTCAGTATTAAAATAGCAATT
TTATTTATTACTTTTGTATACGTAGAATTCAACAACAAATTTTGGAACATAAAAA
GATGATACTTAAAAATGACAAACCAGGTAAGACTTCTGATAGTGAATTTCTTATT
TCTCTTGGTGTTCCTACTCTTGATTAGAAAGTAAAAAGTAAGATGTAAGATTAAG
GTAGTGTTAATAAAAAAAGACCAGTTTAAAAATATATGTAAATTAAATGTGCAT
ATATGTATATACATATGTAAATTAATTTTTAAAATTTAACTTCTTTAGTTTGAAAT
TCAGATTTATTTAAGAAGGTAGTTGTAGCTAACTTATAATCTCAAACATGATTGT
CTGAAAAAATTCCTTTATTTAATTATGATCCCTAAAATCCTATGTAATATTTTTGC
GTAAATAAGAAAAAAGACTTTTAAGTTAGTATGTTGTATGTTTCCTCTATAGTCA
CATTATAACAAATTGGACTTGTTATACAAATGGATCTTCTATTTCATTTTTATAAT
AAATTGTTTATATTTAGTAAACAAATAATTACAGTTGACCCATGAATAATGTGGG
GGTGAGGTATTCTGATCCCTGTGCAGTTGAAAATCTGAGTATAACTTTTGATTCCT
TCACCTTAGCTACTAATAGCCCACCATTGACTGGGAGCCTTCCTGATAACATAAA
CAGTTGGTGGACACCTATTTTGTTTGTGCTGCATTATTATATACTGTGTTCTTGCA
ATAAAGTAAGCTAGAGAAATGAAGCGGTTAGAAAGAAAATCATCAGGAATGAT
ATATTGACTTTTCATAAAGCATTAAGTAGATCCTGACAAAGGTCTTCAAGATCTT
CAGGTTGATTAGGCTGAGGAGGAAGAGGAGAGGTGGATCTTGCTGTCTCTGGGT
TGCAGAGGCAGAAGAACATCTGCATATAAGTGAGTCGCTGCAGTTGAAACCCTT
GCTGTTGAAGGGTGAACTGTATTACATATTGATTTGTGTCACTAAGAAAGTAACT
ATCTTTAGAACCAGGAACTCAGCAATTCCTTTCTGGTACCATAAATAAATGGCAA
TAAGAACTGTAAAACTGAACCAGCATGCACCCATACAAATAAGAGATTATTTTTT
GAGGATAGCTACTGAGCACAGAAGACAGAAAAGCAATTCCTTCATGAGAAGCAC
AAGTTATATTACATATTCTTACACAAGCAAAATGGTTTTATCTGTCATAGTTTATA
CACATACACATACACACATGCACATATACACATACATATGTGCGCACGCATGTGC
ACACAGACACCAAGTTAAAAGTCCTGCTGATTCTTAATGACCAAATCCAACTGTT
CGCGGGGAGTGGTGGATAACACATTCTACAGTTTGGATGCAATTCTTTTGACTTT
TTGACTTGTTCTGTAATGAACTGCCTTTAATGGGTGAATCATGTTTTTAGTTTTAT
AAGAAACAAAGAAAAAGATTAGAAGCAAGTAAACAGAAACTCTATGATCAGTA
GTAGACTATTATAGTATATTCAATAGTCATATGTTTTTCTCCAGTTATACAATTTA
CTTGAATGATGCACAATTAATCAATTATTATTATTATAGGAGATGGGGTCTCTCT
ATGTTGCCTAGGCTAGAATAAAGTGTCTATTCATTGGTGCAAACATAGCTCACTG
TAGCCTTGAACTCCTGGGCTCAAGCAGTCCTCCTACCTCATCTTCCTGAGTAGCTG
GGACTACAGTTTTGTATGGTTATATCTGGCCTGATACACAATTGTTTATTTATTTA
TTTTTGATACAGTGTTTCCCTCTGTTGCTCTGCACTGGAGTGCAGTGGTGCCATCT
TGGCTCACTGCAACTTCTGCTTCCTGGCCTTAAATGATCCTTTCACCTTTCACCTT
AGCCTCCCAAGTAGCTGGGACCCCAGGCATGCACCACCACACTTGGCTAATTTTC
TTTTTAAGGTTTTTTTTGTTTTTTGTTTTTTTTTGTTTAATAGATGAGGTCTCACTAT
ATTGCCAGGCTGGTCTGGAACTTCTGGGTTCAAGTGATCCTCCTGCCTCAGCCTC
CCAAAATGCTGGGATTTACAAGTGTGAGCCACTGCACCTGACCTGCACAATTATT
ATAAAAAGGAATTAAGCCCAGTTGAGTTGCAGAAAATTGACCACCTTTTCATTAT
TTTTTCTAGAAACATTCATATTGTACAACATATTGTCAATCACCCAGATTCTCTGT
TTTTTATTCAGTTAAAATAGGATTGCTGCTTATTCCACATTATTTTCTGATATTATT
GTGTCATTTATTCCTTTTATGGCTTTATTCCTGTGGATAGATATAGAAATACAAGA
ATCTCCAAGTCAAATATCAAGGGAAAAAAAGAAAAGAAAAACAGTTTAGGGAA
AATTATTCTGTGAAATAGCCATCTGATTACAGTTACATATATCATATCAACTTAAT
ACAAATCTTACACAATGTATTTGTGTCAAGGTTTCCCAAGACCACCCCAGGTTTG
ATGGTTCACTAGAAGGACTCACAGGACTCAGCAAATAGTCATACTCAGATCTTTA
ATTGATTACAAGAAAGGGGACAAGCAAAATTAGTAGAGGAAAAAGGTGCTTGTG
GTCAATTCTGGAGGAAACCAGGCTCAAGCCTCCAGGAGTTCTCTCCTGTGGAGTG
CCCGGGATCTGCTTAATTCTCCCAGGCCCACATTTTGACAACATATGTGCAGTGA
TGTCTACCAGTACCAGAGTCTCATTAGAGACGAAGTATCCAAGTTTTTCTGTGGA
AATTACTCTGCCTTACATGTACCCAAATTCCAGACTCGTACAAGGAAAGCAGATG
TTCAGAGTAACCACACTGTTTCTATAAACACTTTAGACACAGTGAGCCACTCTTC
TCAGGGAATGGTGGAAACCCTCCCAATTCCAATTTCCTTAACACCAGCCAAGGGC
CAGCCTTGCATGCAGGCCTTTCTAAGGATGGCAGTCTCTTGCCTGCTATATGAAA
TCTTTTCTGCACAACGCTTATAGCCCCAATTTAATTTTTGGGTTTGTTTTAAAATTT
CATTTTAATAAGACATAATATTATAAGATAAGGTAACTTGGTACTAATTTCTGTT
GTATGATCCATCTTAAGTTGCAATGCTGGTTACTTTTTGACTTTTGGTGACTAACA
GGTATTTGTATATAAGTTACCATAGCAATGTTAGGTAATTATAATCTGTCCTTTTT
ATCTCATTAAGCTTTCAGTAGAATTGTTAAATTAAATAAGCATAATAATTTTTGA
GTTAAAATTAGAATAAAAATTGTATTTTATTTTGATTACATGAATAATCTAGTTTT
CATATTGTGCTAAATCCCTGTTTAGAATTATGAAATAAGATATTCAATCATTTTTA
ACAATATTTTCTTACCTAAGCATGCAATTAAATTTATTTATTTTATATATTTCATA
TACTTCAATTTGAGAAATATAATGACCACATGCTGTCACTTTGGTCTTCAATGATC
TCCAATTTCTAGGGTCACTGTCTCTTGCTTAAATATATCATCATAACAGGTTCAGT
GAATATCTTTATTTTTTATTTATTTATTAATTTTTTTGCGACAGAGTTTTGCTCTGT
TGCCCAGGCTGCAGTGCAATGACAGAATCTTGGCTCACTGACACCTCCACCTCCC
GGGTTCAAGCAATTCTCCTGCCTCAGCCTCCCAAGTACCTGGGACTACAGGCATG
CACCACCATGCCCGGCTAATCTTTTGTATTTAGTAGAGACGGGGTTTCATCATGTT
AGTCAGGCTGGTCTGGAACTCCTGACCTCAGGTGATCCACCCACCTTGGCCTCCC
AAAGTGCTAGGATTACAGGCATGAGCCACTGCGCCCGGCCATATTTTTTTTATTA
TTTTAATTATTCTGGAGATCCTGGGATGCATAAACAGTGAATATCTTTTTTTTTTT
TTTTCTTTGAGATGGAGTTTCACTGTCTCCCAGGCTGGAGTGCAGTGGTGCGATTT
TGGTTAACCACAATCTCCGCCTTCTAGGCTCAAGTGATTCTCCTGCCTCAGCCTCC
CAAGTAGCTGAAATTACAGGTGCCTGCCACCTTGCCCAGCTAATTTTTGTATTTA
GTAGAGGTGAGGTTTTGCCATGTTGGCCAGGCTGGTCTTGAACTGCTGACCTCAG
GTGATCCACCCGCCTATGCCTCCCAAAGTGCTGAGGTTACAGTCATGAGCCACTG
AGCCCTGCTGTGAATATCTTTTTTAAATCAATAACTTTATTTCTTAGAGCAGTTTT
AGGTTCACAGCAAAATTGAGAGGAAGGTACAGAGATTTCTTGTATATTCCATGCC
TCCAACACATGCATAGCCTCCCCCATTATTAGTATTTTCCACCAGAGTGTGGTAC
ATTTGTTACAACTGATGAACTTACATTGACACATTATAATCACTCAAAGTTCATA
GTTTACATCAGGCTTCACTCTTGATGCTGTACATTCTGTGAATTTGGACAAATGTA
TAATGACATGACATGTATCTATTACTGTTATATTATTGACAGAACAGTTTCACTGC
CCTAAAAATTCTCTATGCTATGCCTGTTCATCTCTCCCTTTCTCCCTAGCAACTTG
TGGCAGCCATTGATCTTTACTCTGTCTTCATAGTTTTACTTTTTTCAGAAGAGTCA
TATAGTTGGAATAATACTGTGGATATCTTTTTGAATAGTTAAAAAAATCAAAGCT
CCATGGCAATTGAAGGTAGTCATTTAAGATGTTCTTTGTCCTTTTGTTTTTCTTTTG
CTTTTTTATCATTGTAAAGAATGATATATGCTGATGAGGTATGCTTTACATACTTA
GAAAACATGATTTGTATAGATATTTGGCACATAATGGAAAGGGTTGAGGAAAAG
GACACCACGCCGTACCACACAGCACAACCTGGAGCATCTTGCTCTGTGAGGTGG
GTCCAGATACACTGTCTAGCAATGGAAGGGGGCAAGCGCAAGGGGTTGTACTTT
ATAAAACTGGAATCACAAAGTCTTTCATACTTACCTTCGGTTGGAAATAAGACCA
GGCAGTGAATGCTATAGGTAAATACATATGTTCCTCACTGATCCTCTTCCTTTGA
GGGATGAGGTTGACAACAGCCTGTGTTATGATGACATGACTCACCTACAACTAG
ATTCTGTTATGAGGGATGGCAAGGGAGTTTTGCTTTATGTGAGGTGAAAAAGAAT
TTTTTTCTCCTACTAGGGAGAACAGCAAGCATTGGCACATTCTGGTAGTAAAAGG
GCATTGATAGTTTTCTTTCTATATATTTTTCACATCAGATAATACTGCCCAGCAGC
CTGCCACACCTCCCCAGTGTTTCTTAAGCTTCTCTCTGAATGTGGATAAGCTCTTA
AAGGAGTGATCTTTCCAGTGGTTCTTTCTGTGGGAGGTAAAATGGCAGGTGAACA
TGGGCCTTGTTATATGTAGGGCAGAGCAAATAGCTACAACTAAGGAAACCACCC
AGCACCTTCCCCAGAAGAGTATTAGCCAGAGTAACACAGTGGTCTCTCTCGAGCT
CTTCTCCACTGGCAGCTGCAAAGTTTTTGCAAGGATTCCTGTTTCTGGTCTGATTA
CTATGTTTTGCTGGCTTCTGGTGATAGGGTGTTTTATCCTAAACTGAACAGTTTGA
ACTGAAGAGCTAGAGAGGCTGTGTTGTGTTATAACAAAATAAGTGCAGTAGTTC
CCCCTTAATTGTGGGAGATACATTCCAAGACCCCCAGTGGATGCAAGAAACCAT
GAATAGTACTGAATCACAAACTGTTTTTTCTATACATACATATCTATGATAAAGT
TTAATTTATAAATTAAATCTGATGTAATCTGAAGATAGGGTGTGAGAATTGAATC
GTGCCATCAGCAGGAATGATTGCTTGCTTTTTGGTGGGGAAAAACTCTCCACACA
TTTGGTCACAGAAGCCTTCTTTGTTGATGATTGTTGCTGTGGCGTGAGAGCAGAG
AAAAACATGTCAAGTATGTCTTTCCGCACATACAGTGGATAAGGGGTACTACTGT
ATCCTCTAACTGCTCCTCATATTTTGGTCCAGAAATCATGCTCTTTGACACTGTTG
ACTCATCACACCTGTTCTGCTAACAATACCATTTTTACTCAATCTCATAGGGTTTG
GCTAGGATGACTTGTATACTGCAGTTCACTTGTAGATACCAAATTTTAATAAATT
TATTCTTCTTTACATCTAATAAATACAAAGGGAAGAGTTCTTACTGCATTAATTAC
CTACCAATAGGTACAATTAATGTTAATTCTAATAAGGTCCCAGGCATGCTCCCAA
AGGAATTCTTTGTAACAAAGCATCAGTCTTATGCTTTTAAAAAACAAACCAAAAC
AAAACCACCACCACCAACAACAACAAAAACAGGATCTAAAGCATACACACAAGT
GTGCACAACTTTTTTTATGAAGGTAGTGTCTTACTATGTTTCCCAAGCTGTTCTCA
AACTTCTAGATTCCTCAAGTGATCCTCCTGCCTCATCCTCCCGAGTAGTTTGGATT
GCAGGCATGCATCACTGTGCATTCTTATGCTTTTAATATTCTGTACATTTATTATT
GATTTAAAATGCATTCTACCTTTTTCTTTAATAGATGTTGGAAGTTCTGATGAATC
TGCAGTCAGGTAGGATTTTATAGATTTAAAGAATTATGTTAACTAAGAAAACATA
GATGGAAGAAACTAGTATCTGTTGAGTGTTATATTCTGGGCTAGACATCCTAATA
TGTTCTATGCATTTATCATCTCATAAAGCCATCACAACATCTGTGTTCCTATAACC
TACTGTTTATTAAATAAACAACTATGGATTAGAGCAGTTGATTAATTGCCTTATA
ATCTCATAGTTAACAAAGTAGCTGGCCTACAGTTGGACCGTCAGCCTGCCTGGCT
TCCAAATCCCTTCTCTTGCTCCTCAGCATAGATTGATAGACATCCATGCAGCACTT
GGATCAAGGTATAGGTCTGAATCAGATTAATCAGATTCATTAATTTAATTAATGT
CTAAATTAATGAGAGTTTAAATACCTTAAATACCTTAAACTCTCATTTAAGGTTA
TTGTTAGAATGTGGTTAGTGGAAGAGATTGTCCAGATAAATTTGACAATTTCAGT
GGTAACCAGTATCTTATTTTTACCATCAAAGGCTTTAGGGCAAATCTTACTTAGCT
TTGGCCATAGGACTGTAAGTTTTACAAAAGCAAGTTTAGGCAAGTCTTAGAGAG
AAATCATTTGACTTCCCAGTTTGGTTTTCCATTTAGGCAAGTATTTCTGCTACTTC
CATAATACTTTTGTTAGTCTTGTTTCTTTTTCCATGACTTTTCTATAATCTTGTCTT
GATTTTTTAGAACTTTCTTCTCTGCTTTTCTTGCTGTTTCTTTTGTTCTATTATTTTT
TAAAATTCTGCTGGGTATGTATTCCCAGTTTTCCTATAGACAGAATCAAGAGGAC
ATAGAATTACAGAATTTTAAGGAATCTTAGAATTAATTAAAATACTTTCTAGTAT
TTTTACCTGTATTGAACATTCTGGTCAAGTGATTCTCAGAGAATGTGAGGCTCAA
AGAGATTAGGATGCTTTTTTTTTAGACATAGGAATTGGCAGAAATGAGATTTGAA
CTCATTTTGAGGCCCAGTACTCTTCCTTCTTTTTATATCCTATTTGCATGTGCTTTA
ATAATACAAATGGGAGTGAGTCTGGTGCACCCAGTGGATAGTATGAGAATGGAA
TTAGCTGGTGAACCCAATGGAAGTAGATAAAAATGGAATGAGCAGGGGAAGGCC
AAGTTTGAAGAGAAACAACACTGGATTGGATAGGAGTATGGACTCTTCAATAAG
AGATCAAAATATTGGGGTTTATGAGAAGTTTGATAAAGAGTTCAAGGGAGCTCT
AAAAAGTTCGCTCCTTTTGTTTAAATCAAGGACTGACAAACTTGAAGAATTTTAC
TGAAAGATGCTAAAACATTTTGAGACACTGGGAGGAGTGTCTACAGCAGATAGA
AATGTAGTGTCATCTACTTCCGTCCTGACTTTCAGAGGGGTGGCTTAGAGCCCCT
GGAGTACGAAGGGGCTGGAGATTGCTGGACTACATAGATGTGTGGCCCAGGACA
GGTGGCCTCTTCACCTCTGCCTCTGTTCCCGATTCACTGATGTCCTTCCCATGTCC
ATGTAGGCTGGGTCAGGGGCATGATTGGCTGGCAAATCAGTCATGGAGTTCAGTT
GGGTAGTTGGTAGTGTGTCTATGCTGGGTGCAGGTGATGGAGACTCCAGTTAGCT
TGTTTTTCAGGAGCAGGGATATAGAGGGCTCCTACTCCTGGTCATTTGAGGCCAT
CCTTTCAGGAATCTGTGCTTTCATAGGCTGAAGATTTGAAGATTGGAGACTTCTG
TGGAGCCCTGCAGAAGTGGAATCTGGAAGTGGGAGCCCATAGGAAGACAGATAC
TTAGATAGTACTTAGGGAAATAGAGGTACAACTACCAGGACTCTGTTTTTCTGGC
AGTCTCTCTCCTTGGGTGTCTGAGTGCCTATGAAAATTTTTAAGGGCTTGCTAGTT
TATGTGGACCTGAATAAAGTAGGACCTATAGAGTGAAAATAATGGGATTTTATA
ATTGCTAATATTTTAATCTTTCTGGGAAAAGTATTCTCAATAAGAACATACACTTT
TGTTATTTGATTTTTGTACATGTAGCTTTCATACCTTTCAAATATTGCATGGGATT
TCCTGTACCGATTTAGGGCAAAGGAAAGCAATAGGACATTCCTAAGTGGGTTCC
ATGTTGAGGAATCAAGACTGCCAATTTGAAGTGATGCAGATTAGTCTTTTAACCA
GAGATAGATTATGGAAAAGAGACAGTGGATCTTTCTACCTTGTTTTAGGTCATCA
GTTTTCTTCCAGTTTAGGTAACAAAATTTATGTCATCCATTAATTGAGTTTTAAGT
TCAGCTTCAGGACAGATAATTTGTGAGGGCAAATTATTGTCAGGCTCTGCCAATA
TATTGACTGTCACTATTTGTTATGAAGCTGAAGGTTAGTTTTCATTGAACATTTTA
TAGATTTAGACAGGTGGAGGCAGAAATAGGTAACTAAAATCTATTTTTAGAACA
GAGGACCTATTTTAATTATATCAAGAATCATAATTTAATATATAGATCACTGACC
TTTCCCCAGATTATTCTTTCCTTTTTTGAGGGGGAAGCTGGATATAAACTGGCAGT
TAAAAAATTGTAAAGAAATCAACTTGCTCATTTTCATTGTGTATTTTTGCTCCCAA
GCATTTTCCATGAACTGTGTGTGGATTCATTGCCTGCATTAGATGACGAAGTCTT
GAGTGTTGCTGCTAAGGTAAATTGGTCTCTTGTAAAATTAATTTTCTCACTCTGAA
TGTAGTTTTGCAGAGTATTTACTTTTCAAACTTAGCAGTGGTTTATCTGTCATTGT
TTTATGGTGGTAACGGAAAGTGGGTCAGAGAAAAACATATATATGGCTAGTTGA
TTGAAAAAATTTGTTTAACTTTGGTAACTAACAAAGATTGATAAGTACCGTGACA
GGGTAGGAGCTGAAAAAAAATGAACTGGAAAATAAGTAGTGACAGGAAAATCA
CATTAGGAAATGCTTTCTCCAATAGAGGAAATATGAAATTTGATTAAGCTTTATT
TGGATAAATACTAATACTTTGAGTTTTAAATCCTGTGAGTGTGACTTTCATAATAT
TTATGCCTGTATAACTCTTCAGTGGATCAAATTATTTGCAGTAATCATGGAATCCT
CCTGGTAACTTTTAGTTGCAAAAAGGTTCAGCACATAGCATATAGCTTTTCTTCTT
GGAAACTTATTATTTTGGTATCATATAGTTTTTAGGAGAGATTGTTTCTCTACTTA
TATTATTGGTTCTATAGTGAGACTAAAATAATATTAAAAATTGTAGAAAAATAGC
TGAGTGTGGTGGTGTACACCTGTAGTCCCTGCTACTTGGGAGTTTGATGCAGGAA
GATTGCTTGAGCCCAGGAGTTTGAGAACAGCCTGGGCAACATAACAAGACTGTA
TCTGATTTAAAAATATAAATTGTGGAAACATAGAAATTTAAATTTATGTTCTCAA
AATTTGTATTGCGAAGGGATTTTTGTGTGTTTTATGAGTTGTCCATGAAGAGTTTA
TATAAAACACTTCATCTAATTGAATAACATGTATTTTGCTGCAAATAACCAGTTC
TAGAAGCAGAGACTCTTAATACCAATATGGTAAGACTTTATCATCATAATTTTGT
CATTGTAGTTTATTTAAAATATTTAGTTGGCCAGACGTGGTGGCTCACACCTGTA
ATCCCAGCACTTTTGGAGGCCGAGGTGGGTAGATCACCTGAGGTCAGGAGTTCA
AGATCAGCCTGGCCAACATGGTGAAACCCTGTCTTTAAAAAAAAAAAAAAAAAA
AGCACAAAAATTAACCAGGCGTGATGGTGCATGCCTGTAATCCCAGCTGCTCAG
GAGGCCACGGTGGGAGAATCGCTTGAACCTGGGAGGCGGAGGTTGCAGTGAGCC
AAGATCGCACCATTGCACTCCAGCCTGGGTGACAGAGCAAGACTACATCTTAAA
AAATAAAATAACCACTCAAAGTCCTTATATCCTATTCTGAAATTTTGAATGTCAG
AAGGTTTTCTATTTAGTTGTTTAAATAATCATTGGAAGCTCCTGCATACTATAGGC
TACTGGAGGTCAGTAAACATATTTGTGTGTATCCTGGAGTACCTAGAATATAGTC
TTCCATGTAAGAAGCATTTTACTTGTTGTTTTTTGAGATGGGGTTTCACTCTGTCA
CCCAGGCTGGAGGGCACTGGTGAGATCTTGGCTCACTCCAATCTCCATTTCCTGG
GCTCAGGAGATCCTCACACTTCAGCCATCCAAGTAGTTGAAACAGTAGAGCTATG
TCACCATAGACCTGTGTCACCATGCTCGGCCGAGTTTTGTAGAGACAGGGTTTTG
CCTTGTTGCCCAGGCTGGTCTTTAACTGTTGGGCTCAAGTGTTCTGCCCGCCTCAG
CCTCTCAAAGTGCTGGGGTTACAGGCATGAGACATTCAGCCTTAATAGTTGTTTA
ATCTGAATAAATAGACAAATGAATTTTTATATAATGGAATGTTATAAGTAATATA
ATATACCTAATGTATCTAACAATTAAATATTGTATTTAAAATATTGCTTACATTTG
TATTATTTTTTAATATTTAAGGGAGTATAAGTTTTGATGTGTTATGTTGAGAAATT
ATGCCATAATTAAAAAGGAAATAAATTAGAAATAGGTCATCAGTAGCAAAGAGG
GTTACAATATATTTTCTAGTATCATTGAACTGGAATCTTAACATTGAGATTTTAGA
TTAACATTTCTTAAGCTTTTTATTAGTCCCAACTCAGGTTCTATTAAATATACCTT
TTCAAGCCATACATTACCCTTTATTATTATTATTATATTTTAAGTTCTCGGGTACA
TGTGCACAACGTGCAGGTTTGTTACATATGTATACATGTGCCATGTTGGTGTGCT
GCACCCATTAACTCGTCATTTACATTAGACATATCTCCTAATGCTATCCCTTCCCC
CTCCCCCCACCCCACAACAGGCCCTGGTGTGTGATGTTCCCCTTCCTGTGTCTAAG
TGTTCTCATTGTTCATTTCCCACCTATGAGTGAGAACACGTGGTGTTTGGTTTTCT
GTCCTTGTGACAGTTTGCTCAGAATGATGGTTTCCAGCTTCATCCATGTCCCTACA
AAGGACATGAACTCATCCTTTTTTATGGCTGCATAGTATTCCACGATGTATATGT
GCCACATTTTCTTAATCCAGTCTATCATTGTTGGACATTTGGGTTGGTTCCAAGTC
TTTGCTATTGTGAATAGTGCTGCAATAAACATACGTGTGCATGTGTCTTTATAGC
AGCATGATTTATAGTCCTTTGGGTATATACCCAATAATGGGATGACTGGGTCAAA
TGGCATTTCTAGTTCTAGATCCTTGAGGAATCACCACACTGTCTTCCACAATGGTT
GAACTAGTTTACAGTCCTACCAACAGTGTAAAAGTGTTCCTATTTCTCCACATCCT
CTCCAGCACCTGTTGTTTCCTGACTTTTTAATGATCGCCATTCTAACTGGTGTGAG
ATGGTATCTCATTGTGGTTTTGATTTGCATTTCTCTGATGGCCAGTGATGATGAGC
ATTTTTTCATGTGTCTGCCATACATTACTCTAGAATTCTGGTGACCAATTCTTTTTC
TGGGTGGAACGTTGATGGAAAGTTCCAGTTTTCTCTCTCTGTTATAATAATGTTCT
TTCAGGTAGTGGTAGATGACCATATTTAGCTAATTGAATGTCTTATAGTAATAAA
CTCTATCACAGAAGTACTTACAAAAAACTAATTGTAGCATAAATATTAATTAGTA
TTATCAGGGATATGAAAGACCAAAAGGCTCTGTTATAGATCTATTTCCCCATGTA
CTTTATTGTACTTCATGTTGTTTCTTTTCTTTCTTGGCTTAAGCTCATATTTCATTG
ACCAATTAGGCTTCTTTTTTGTTTGTATCTCTCTTCATTCTTACATTTTAAATTGAT
ATTTTTGGGGAGTCAGGGTCTTGCTCTGTTGCCCAGGCTGCAGTGTAGTGGCATG
ATCTTGGCACCCTACAGTCTCCACCTCTCAGGCTCAAGTGATCCTCCCACATCAG
CTTCCCAAGCAGCTGGGACTACAGGCACACACCATCATGCCTGACTCCTTTTGGT
ATTTTTTGTGTAGAGATGTGTTCTCATTATGTTGCCCAGGCAGGTCTCAAACTCCT
GAACTCAAGCAATCCACCCACCTTGGCCTTGCAAAGGGCTGAGATTACAGGTGT
GAGCCACCATGCCTGGGCAACATTGAGACTGATTTAAAGAAATTGATTAGGGCT
GGGTGTGGTGGTGCACACTGCTTATCTCAACACTTTGGGAGGCAGAAGTCGAAG
ATTTACTCGAGCCTAGGAGTTTGAGACCAGCCTGGGCAGTATAATGAGGCCTTAT
TTCTACAAAGATAACAATAGAAACATTAGCATGGCATGATGGTATGCACCTGTA
GTTCCAGCTATTCAGGAAGTTGAGGTGGGAAGATTGCTTGAGGTCAGGAGTTTGA
GACCACAGTGAGCCATAATCAGGCCCCTGCATTCTAGCCCTTGGTTGACAGAGTG
AGACCCAGTTTCATAAAAAGAGATTGATAAGAAGCTCTTGATGCAACTCATAATT
TTAAAATGGAAACTAATTCTTGATATTACCTTAGCAGTGTGTCCCCGAGAAAGTG
TCAGAGCCTTTATGTGGACCTTCCCATGGAAAAGGAAAACAGAATAGTCAATGG
AAAAGGAGAAGGTGAGAACTGTATTTTATTTAAAAAGTCATTTGTTGGAGGCTG
GGTGCAGTGGCTCACACCTGTAATCCCAGCACTTTGGGAGGCCGAGGTGGGCGG
ATCACAAGGTCAGGAGATCGGGATAATCCTGGCTAACATGGTGAAACCCCATCT
CTACTAAAAATACAAAAAATTAGCCAGGTGTGGTGGCGGGCCCCTGTAGTCCCA
GCTACTTGGGAGGCTGAGGCAGGAGGATGGTGTGAACCTGGGAGGCGGAGCTTG
CAGTAAACGGAGATCACTCCACTGCACTCCAGCCTGGATGTCAGAGTGAGACTCT
GTCTCAAAAAAAAAAAAAAAAAGTCATTTGATGGAATGTTTCTTTGAAAATATG
AGCACTAATAGAGTCTAATAGCTAAAGAAAATGTCCTATTAACTGTATAATAAGT
AAAGGAGAAGTGAAATGGTGATAAGTTGTGTCTCTAACCAAGGGTCAGCAGTTG
ATTCTATTGGGAGTACCACTAAAGGAGCTGAGTTGTGGGTTCCATTTTAAGATAC
TCTAAGACCTGAGGCAAGTCAGGAGAGAGGGAAGAGGAAATGAATAAAAGAGA
AAGAAAGATGAGGAGGGCAGAGTATACATGGAATAAATAAAAACACATATGCG
GATGTATGTAATAGAGGGTAGTAAAGTCTAATTGATCTGTAGAAGAAGGAAGAA
CAGGGTGTTAGGAATAGGAAGGAAGATAAAGTGAGCTTCCCGTACCAACATATG
TCAGAGAATTAGAGTAACATTTTCCTACTCTTGCTGTCATCCTCACTACTGGGGA
GGCATTAAGGATTGAGGTATTTTACCACACAGACCTGTGTTTTATCTACCATAGA
TGAACATCACCATAAATGGTCAGCCATGTATGGCTATAATTTGGTTTTAAAGAAA
ATGTTGTAACCTCATAGGATAGTATCATATAGGCCAAATTAACATAATTGAAAAT
AATAGTGTTGGGTGATGTATGGAGAAGAAATTAATTAGAGAAGGTATTACCTGA
TTAAAAGTTCATTAGAAACATTATGGCTTATAATGTAGTATTAAATTCAGAGACA
TAATAGGGAAGAAATTGAGTCTAGGCCAAAAAGGGCAATTAGGGTAAACTAATA
TGGAAGCACATAAAGTGTAAAACAGGGCATTCAGATAGTCATGAATTAGTTGAG
GAACTTCTGGAAACTGCACATTCTGATTTAGCAGGTATAGGAGTCTGCATTTCTC
ATGAGTACTCAGGTGATGTTGTTGCTGGTCCTTGGACACAGCTCTGAATAGCAAG
GGAATAGCCTTCCTTTAGAGAAATCTGGAAAAAGAACCACTGGAGAGCAATTTG
AATAATAGCAGAATCCAGGGAAAGCATTAATTTCCTTTTATTTCTGAGCACGATT
CTAGCCACAGGGGAAGGAAAATGAGATGAAAAAAGAGAGATTACAGGTGTATA
CTACTGCTGAATACAGATGAAAAAAGTGGTCACAATTATCCATAAAAAGCAGTT
AGGAAGGGAAGCATCAGGATGACAGATCTAAAAATCACTTTTTCAAAGGAAGAG
GGATTGTGAAAGGACACAGAGGGAGGAAAGAAAGACATTTGCTGGGGTCTTGGG
AGTTAAAGCCAAGTAAACTTGAGACAACTCACTTCCAGTTGCTTCAGCATATGCC
CAGTCTCACAAAAGAGGTTATTGCTGTGGAGAGTACTGGAGGCAGGAGGGAGTG
CTAGAATTGGGGTAAACCACAGCAGCTCATTTCACTTCATAATTGTCAGGCCTCA
GAGAGAGAAGTTTCATTGACATGAGTGAATAAGATGTGATTAAGTTGCATATAG
ATGCTTTGGCTAATTTTTTTTGAGACAGCCAGTTCTTTGATATGATAGCTGCTTTA
TAAAAGTCCTTTACAGTGTAAGATGATATACCAAACTTAGTTAATTTTAGAAGCA
ATTGTATTATAAAATTCATTCTGTGAATACCAAAATACTCATTTTCAATAAGTACT
GCACTGATTTTGAAATATAAATGTGTATTCGTATCCAGCAAGTCTGTGGTAATTC
AGTGTTTTCTTTTTTGATAAATATTTTGATATTGGAAGCTTATTCGACATGGTTTA
TTTGATGTGTTTTATGGACCACCTCGCACAAGTGGATCAAGGAGCTCTAACTCAA
GGCCAAATGAGGGGATAGGAGAAATGTAGGTGCTGCAGTAGCCCATGTGATCAT
GGGAAAAATGAGTATTTTGATTAGCTGTTATTTCATAAGTGTGCGTCCTAGCTGA
TCAATGTAGAACACTTTCTTTGATGAGAGGTGAATCACACATTCACCTGAACTGT
CATCCCAACTGTGTATTTCCTCAGCGACAGGACAAGGGGAATTTATCTGTGGTGT
GCTGGCAGCAATGCCTCTGATGTGTTGAGTTAAAATACTCTGTACATTCACCATC
AGCTTTGACATCGATTCCCTCAGGTTTGATTTGCTCCTCTGTTTAGTGGTCCCTTTT
CTCCTCATCAGCCCACGTGTTCACAGTGATATCCATGCTTTTCTATTTTAGGTATA
GACATTTGAAACATAATCTCACTACTGAAATGTAAGCTGTGCATTTTAGGAATCC
TGTATTCCTATTTTCCTCATTAGGTTTCTGTCATGTTGCTGTCCTAGGCAATGAAA
AGAAGAAGCCAAGAAGAACCCTCAAAACCTTAAGTAATTATTTTCATAGCCAGG
CATGAGAATTCAGCTCGATAGTAACACTGCATGAATGGTTGGCCCTGTCATACTT
ACATATAATTGATGACATATCCCTTTTGCTTTGTAGGGCCTCCTGCAAAACATCCT
TCCTTGAAGGTAATTAATTATGTATATTTTTTGAATCACTAACTCCACATTGTATA
AAATATATATGATTTATGAATCATTTTCTTTTAAAACCCATTCAGCCTAGCACTGA
AATGGAAGATCCTGCTGTGAAAGGAGCAGTACAAAGAAAGAATGTACAGACATT
GAGAGCAGGTACATATTCAATACAAATGGAATGCCGGAAATAAGTACATTCAAT
GATTGGAAGTACTCACATTATTCTTACCCCTAATTCTGTTTGTTCAAAACTGAATG
GAAGGCATTGACGTAAATGTTATTGTTGGTATCCATATTTGAATAACAATAAATT
TAGAAGCATAAAAAAGATTTTAAAAATGTAAGCTTTAACTCAGATGTTTCTCTTT
TAATGTTTTGAATAGCATGAAGTTTTCAGTATAAAATTTTTATACTTGTCAGGGAT
TAAAAGCACTGAATTTTGAGACTCTAAGATATTTCCATTGATTTATGTGCTAGTTG
GCGCTCTGATCTTTACGTAGAGGAAAGCTTTTCTTATTAACGTGTCAGTTTCTGTT
TTAACTTTAGAGGCTTGCTGCTAGTGTTATTACACTGATGATCTGAAGCCAATCA
GATGTTCTAGTGAGCAAGACTGTGTGTGGATGTGGATGTATAGGTGTGTGTATGT
GTGTGTTTGTGGCATCTTTGACTACTAAAAATGAGGAAAGTAATTATTCATTTAT
AACTGGTAGACACAGTCTTTTAAAACGGTGATTTTGAGCCTTTTTGGTGTTAAGG
TTTTTAAAACGTGATTGCATAGCGGCTACCAACATCATAAGTTGGTTGTTTTTCAT
TTCAATGCCCTTTTGAAATCTTTAACTATATTGTGATGCTCAGAAATAATATGCAG
AATTTTTTATTTGTGTCCCAAAATGGTATGTGAGTGGTTATACACTTTACATACCT
TTCTGCCACTTTCTTTGGTGTATTTTGTATTATGTTTTCCAGATGTATCCACATTGA
TATGATTATCTCTGGTTTAATTCATTTTATACTTTTCATTGTATTCCCTTATACCAC
TTTACCACATTTAGTTAGACTCTCCTGTTGCTGATAAATGAAGAAAGAAAGAAAA
ATAAAAATAATGTCAGATTAAGAGGGCTTTTCTTTAGTCAGTTTGTATAAATTAA
TATTTACTACATGAGAGTTTAAAGTTGAAAAGTTCAGAATACAAGCATGCACCAC
CATATTTTATAAATGTCCTTAGAACTGTGACTCATGAGCCTTTAGCCTATGAAGTT
AGGACAATTCATTTCTCTGAAGAAGTTTGCTGTGCTATTCTCAGAAAAGAAAACT
GAAAATAGCAAATGATATTGTCTTATTTGACCTCTTGGACATCCTTGAATGAAAC
TGAAACTCTAGGGATACTCGGATCAAAATTCAGAACTAATGTTTTGAACAATATA
GTTTGTGAATGTCCAGTGATCATGAGCCCTTGATGGGGAAATGACCTTTCAAGTT
TCACTTTTGCATTTTTTGCTCTTTTCCTTGACTTGTCTTAAAAGCTTAAATTCAACC
GTTTTATTTTTACAGAAACCAGGAATATAACTTTTAAAATATATGTCTGTCCTGTC
TCACGGTGGTGTGTACTCTTCAGATCTTTTGTGAACATAGACTTATATGGGAACA
ATTATGTTTTTTGTTTGTTCGTTTGTGTTTTTGAGACAGAGTCTTGCTCTGTCACCA
AGGCTGGAGTGCAGTGGCTCAGTCTTGGCTCATTACCACCTCTGCCTCTCAGGTT
CAAGCAATTCTCCTGCCTCAGCCCCTTGAATAGCCGATACTACTTGCACGTGCTA
CCATACCCTGCTAATTTTTCTATTTTTAGTAGAGATGGGGTTTCACCATGTTGGCC
AGGCTGCTCTCAAACTCCTGACCTCAGGTGATCTGCCCACCTCGGCTTGCCAATG
TGCTGGGATTACAGGCGGGAGCCACTGTGCCAGCTACAAATAAGATTTTTAAGG
CTATTATATTTTATACAATTCTTTGGTTTATGTGAATTCTGAAGGCTTTCATGCAT
TGAGGGAAGATTATATCAGTTTAATGAAAGCAGTTTTTAATTTAATGTATATTCA
TTAAAATTTTTTTTGAAGTTTTTGTCTCTAGTACATAGAAATACACAATAATGTCA
TGGGTATTTGACCTTTATGTGTTTATGCACAAACTTAGTTATTCAAATATTTTCTT
ATCCCTAAAGAATCTTAATTACTAATAAACAAATTTCTCATTGAAAACAACATAT
ATAATAGAGATCGTTGAGTGATTGAAAGTAAATTGTAGTAAATAACAGAAGCTT
AGAACAAGTTAAGTAAACTTGTCTGAGTTAATAGCAATTACAGGACTTTTAAAAT
ATGTTAGACCATGAGGGAGTGGTGTGTTTGTGGGGTAGAAGACAACATGGTACT
GCTTCAGTGAAGAAAGAACTTTTACAACTTATTACAATTTGTATTACTATTTACAT
TCTAATAAATAAAAACTTTATTTTCAGATATTTTAGATTATGTTTCTACTAGTTGA
ACCATCAATAGTAAGACTTTTCAAAGATTTGGGAAGTTGTGAGTTGACGATAAAT
ATCTGTATCGCCATCCGTGATCAAAAATCAGACAGCAACTACAGACTTTGGACAC
GCGAACTTCATAGTTAAAGAAAGGATTAATTTTGGAGCTGTGTTTCTATCAGGGA
ATTATACTCTTCATTGCCTGCATGAATCGCAGTTATTAGAGTAGAAAGAGAGCAA
AGAAGGGAAAGAAGCATAGAAAATTTTATTCTAGATTACCTCGGTTGGCTTCATG
CTACCATAGTTCTGACTTTTAAAAAGTCATTTTGTGATCAAAGGTACTTTGTGTTT
ACTCCCCTTATGCAGGCTACAACCAAACAGAATGGTTCTTAGCAAGGCATTTGTA
TTCTTCCCTTAAGGAAAGCAACATATAAATAAAGAGAATGAGGAGAAAGAGTGA
TTTCATTGAGGTTGTTATTTAACATAAATTTGAGTGTGGGTACCATGATTATATTT
AGAATTTTGGGACTGGATGGGAAAACCAGCTAGACATCTACAGATTCCCTACTCA
AACACAATGTGCCTTTGTTTTATTTTTATATCTCTAATTTTGCAATTATTCAGTAC
AACTGTATGCAGTGTCACTAAAAATACCTTCCCAAACCAAATATTAAATAATGCC
TATGGCTTTCTGTTTTATAGTGTTGATTTTCCCAATATTAATGGGAACCATTGAGC
ATTTGCCTTGTGGTGTCTCCTCAGCTGTATTCACACATTCCATCACCTTGTCTTAA
TGGATAATCATACACTAGGAGTACGGTTTTCAGAAGAGCTGTGTCATTTAAAGAT
AACACAGGAGCATCAAATTTAATTCTGCTAGAACACCTGGTCTACTGATTAACTG
CAGCTAATGTGGGGTCTACTTCACATACAAGTTAAATTCAGTGCCCTTAATCAGT
CATATGATCAGGTCAACAGTAATAAATTATGCAATATTTTCCCCCACCCCTATAG
TTTTAATTTCTTTTTCCCCTTATGTCTAGAATTAACATTTTGTTTTATAAAACATGA
TGATAATCTTCTAGAGTAGTGATGACAAGCTATAAATCCAAAGTTTGTTACTTAT
GCAGATGACTTGTTTGCTTCTATTTTCTCATGAGCTTGGTAGATCCAGGAAACAG
AACTTTTAAAACAAAATCCCCATATGTGGCTGGGCGCGGTGGCTCGTGCCTGTAA
TCCCAGCACTTTGGGAGGCTGAGGCGGGTGGATAACCTGAGATTGGGAGTTTGA
GACCAGCCTGACCAACATGGAAAAACACATCTCTACTAAAAACACAAAATTAGC
TGTTCATGGTGGCAAATACCTGTAATTCCAGCTACTTGGGAGGCTGAGGCAAGGA
GAATCGCTTGAACCTGGGAAGCAAAGGTTGTGGTGAGCTGAGATCATACCACTG
TACTTCAGCCTGGGCAGGAAGAGTGAAACTCCATCTCAAAAAACAACAACAATG
ACAACAACAACAACCAACACAAAACCCAAATGCATTTCCTTGGCACAGTAAAAC
TGAAACAGAAAAAGTGTAAAGTAAATACAAGTAACTGAAACAGTTTATGTATAT
TATCTTACTTCTCATTTGATAAAATTTGTAAAGTAATGAGCAGAGGGTATTTCTCC
AGGGACCTGGATATATACATTTATTCATTCAATAAAAATTCATTCTTATAATGGC
CACTGATACTTGTATCCTAATCATTTCTGAAAACATCTCCTCAGGCCTGCATCATC
TTTGCAACATTGCCATATTTTATCTTTGTTCATTTATTTATATGCCTCAGAATTTTA
TGCTCCTCACAGTATTTAGAGTTAATTATCTCTAATGTAAATAGATCCATGAACC
ACTCCTGAATACCTAATGTCCAAGCATCTTAAAGCTTTATATAAGGATTTCAGAA
ACTGACTTCTGGGTTGGGCACGGTGGCTCATGTCTGTGATCCCAGCACTTTGGGA
GGCTGAGGCAGGTGGATCATTTGAGGTCAGGAGTTCAAGACCAGCCTGGCCAAC
AAGGTGAAACCCCATCTCTAATAAAACACAAAAAATAGCAGGTGGTGGTGGCAT
GCACCTGTAATCTCAGCTACTCCGGAGGCTGAGGCAGAAGAATTACTTGAACCC
AGGAGACCGGGTTGCAGTGAGCCAAGATCATGCTACTGTGCTCCAGTCTGGGAG
ACAGAGTAAGACCTTGTCCCAAAAAAAGAAAAGAAAAGGAAACTGATTTCTGCC
CAAATCTCTATCCGTAGCCCTTTCCCCATCTGCCTTTTTCTCTGGAATTACTCAGC
TGCTGGTAATGGCCCCCTCACCATTCCTCTTTTGCAGAGAAATACATACTCTCTTG
GAGGCTTCTCTCCCTCTCTTGTTGCTGCCTGGCATGTGCTAACCCTTTCCTGCCCT
CTGCCTCACTTAATCTGGCGAACCTCACTCTCTAATTCTCAGCTCATGCATGATCT
TTAGGAAAGCCATCCCTGACAGCTTTTATGTTCCTTCCTTATACCCCAGTGCCTAA
CACTTAGCAGGAACTCAATAAGTAATTATTTAGCAAAATTAAGACTGTTTATACA
AAGATGATTCAAAAGATTGTCCTCTACAGTCTAGCAGCAAAGGGGATTGACATG
TAAAGACATGATGTGCAGTTCAGGTGGTAAAGTGACACTAGAAAAATTGACAAA
GTACTAAGGGACCGCAATGAAACAGACACCTGTGTGTGTGGAGAAAGATAGCTA
GAATCAAGGAAGACTTCACACAGCATTCTGAGCCTTTTTTTTTCTCTTTTTCTGTT
GTTGGAGACAAGTTCTTACTCTATCACCCAGGGTGGAGTGCAATGGCGTGATTGA
AACTCACTGCAACCTCAAACTCCTGGGCTCGAGGGATCTTCTCACCTGAACTTCT
TGAGTGGCTGGGACTACAGGCACATATCACCATACCTGCCTAATTTTTTGTAGAG
TCAAGGTTATCTATGGTTTCCAGGCTGGTCTTACAGTCTTGGCCTCGAGTAATTCT
CCTATTTTGGCCTTCCAAAGTGCTGGGATTACAGATGTGAGCTATTATGTCCAGC
CTACTTTCTGAGTCTTAAAAGATGAAAATAAATTTTTCAGAATAGCAGGGGGAAA
ACATTTGCGATGTAAAAAATGGTGTGCACACTAATTAAGATATAAACAATAATTT
TGCAAATTAGTAACTGCCAACTCAATTAGTGTCTTGTTAAAAAGATACTGTTATG
TACATTGTATATTTTGACTGTATTTCAAAATTTTGTTTTGTTTCCAACAGTTTTGTT
GATTTATGTTGGGTGGAACAATTTGTGAGTGACCCTGAGATTTTGTATGGCTTGA
ACCTGGTGATATCTAGTGTCTCCCCAAATGGTTTGTTGAAGTTTTGGATAATTAG
AAGTATTTCTTAAAGAAGTAAATATTTCAGTAAACATTAAGCTTCATTTAAACCC
TCAAAATATAAAATACGAAGAAATGTTATTTTCTATTTATTTTTATAAAGATTATA
GTCTTTATCTAACTGTTCTTAGTTCATTTGAACTAAACCAATGAATTTGTCAACAG
AACAAGCCTTACCAGTGGCTTCAGAGGAAGAGCAACAAAGACGTGAAAGAAGT
GAAAAGAAGCAACCACAGGTATATGAAAATTTAAGTTTCTTGTTTAATATTAGGT
TTTTTTTTGCTTTAGTAACAAAGCATAGTCCAAATGACATGACCTTTTAGACTATA
CCTTTAGAATCCAATAGATCATAATTTTATATTTAATTTTTAAAACATTTTAACCA
GTTATGAAACTTAAGATATTCTTACTATCTCTAGTAACTTATTCATTATTCTAGTA
ATTCTTACTATCTCTAGTAACTCATAGCTGTCTTTACCCTTGGAATTGAGGCAAGA
ATTTTTCACAATTATCTTGCTCTTTTATTTGTATAACCTTACTCATAATACAGAAG
GTAACATGAAATATTGGGTCATATTACTAAGGAATAGAAATTATGAACAATTTAA
TAACGATGGCCGCTGAGTTAAACTAGTGTTAAAAGAGTCATCATTGCCAATGGTT
CAAATGTTGCAGTTTTATATTGCTGGTCATCAGTGCCGAGGTTAAAGATTTATTCT
GTTTTGTGGTCACCAGTTGACTTCTGTGTCTGTGTTCAGGGAGTGAATGGGGTCA
TAAAAACCAACCCAGTTGCCTTTTAAGAGAATCCTACCTTGCAGAATGGGACCTT
TGGTATCAGGGTACAAACAATAACTTTATTTCAACATAAATACATAGTAAATATT
ACTAAAATTAAAAAAATCCAAACACTATCACTACTGGAACTTAAAATATATTAG
AAGTGGATATCATAGTATATCATTTGAATTAGAATTTAAAATTTTGCTTCTCTTTC
TTATTGGTGTTCAGTTTGGCTCTTAATAATTTAGTGTTTGCCTAGTCTCTAGTTAA
TCTTCAGAAATATACATGCACTGTAGGGGCTCACTCTTTCTGGTATGCTGAGGTA
AAGTCTTTGTAAGAGAGGAAGCTTTTATAATACTACCTATCATCTTTGAATTCATT
TCTGGTAGACTTTACACATAATGCATTAAGTTTAGTCCAAACAAACACTGAGAGT
TCAGCTTGCCGGTTTATGTTTCTGTCCTATGTTAAGCCAAGGCAAATTATTTTTCA
CTTTTTAGTTACAATCCCATAATTGAAGAGTGGCAACACGCAGATTAAGTTTCAC
AGTTAAATTTTAATTATTTTCTAATATTTGTTTATACTTGATTAAAGCTAATTTTA
GAACATGCACTCTGACAGAAAAGACATCTGAGAAACAAAACAAGCAAATTTGTT
TTCCATTTTGCACCTGCCAAAAAAAAAAAAAAAAAAAAAAAGCCTCAAGAACCA
GAACTGGGTAAGAATTGTGATAAAGGGAATCTATCTGTATATTCACGACTTTCTT
TAAAATTCATTACAAACAAGTTCAAGCTGAATATTGGTAAAGGTTTTGGAAACTC
CAAAATTACTGCTTGCCCTGAGGAAGAGCTTCTACATAGTAACTCTAAAGAGGG
ATGAACGAAAAAGGAGTGCCCTCTGATCTGATGAATCAGGTCCCTGATTGTGAG
GAGAAAAATGCATCTGGAGGGTCTAACTCTGTGGCATTCCAAGCAGCACCTGAA
TAGAGGAATCCCATGTCAAATGTCTTTTTATTCCATTCACACTCCAGGTCCCTGAA
ATACACTTACCAGTCATCTTCTAAGCTTCATTTAAATGAAAATAAATCAGACTAT
GAAAATGATAACAAACCAGACACATAGCTTGTTTCTAACACAGATGATGAAAAT
TTTTGTAATGATACAGAAACTGAAACATTAAGGAACCCAGTAATTATGATTGAAA
TGAAAGATGATTAAGAGTTTCACATGCAAATGGCAAAAAATACAAACCCAAATA
CCACTAATTGGAAATTAGACATTAGGCATTGGCCTCAGTCTAGAGATCCAGAAA
GTCTTTTTGATTTGTGGTTTACCCACCCCAAAGAAATGAAGCATATGATTCAGAT
AGAAAGCCACAGTATTTCTGCTGCTACAGATACTTATAAAAACAGAAAACCAAT
ACAGTGCTTACTCCAGAAGCCACTATATGACAATCCCAGTGCTAATAACTACAAA
AGCATGAATCTTGAATTATAAAGTGTGGGTTTATTCTTTGCCACATAGTGAGAGA
ACATCAAAAATATAGCTAGAAGACACAGCAAGATATTCCAAGGTCACCAACATG
GCACATGTATACATATGTAACAAACCTGCACGTTGTGCACATGTACCAGAACTTA
AAAGTATAATAAATAGTAAAAAAGAATGAGGTAGCATGTTACAAGTAGAGTTCC
TGGCTTTGGAAAAAGAGAAAGTCCAACTTCAAAAAGACAGAGGTTCACTTGCTG
CTGCTTTTTTCTCTTTGTCAATTATTTGATTTAGTCAGATTTTCTATTCAAGAAAAT
CTCATGTGTACAGTTACAGTGGGGTTATCTAAATGTGTAATTGTGTGTCAAAGTA
GATTAGTTTTGCTATCTAAATAATGGTTCTGGAGAATGTTCTCATAATGTTTGTTC
ATTAATCAACCTAAGTCTTCCTATCAGTCTTCCAAGTGGCGTATGAGCTGGGAAA
CTAATTCAGCCATATACCATGTGACCTTTATGAACCAGATCAACATAAAGAAATT
GCTAAAGAAATAAGCTCTAGATTCTAGATTCTTTTTTCTGTATTCATTTAGAGATG
AATTACATTTATTTAATGATAGAATGGTAATACAATGGGAGGGAAGCAATGACT
GAGATGAGCCACAAAAACACGTCTAGCCTTGAGAGTTGCAACGAATATTCCCAG
CCAAATGAGTCTGTTTAATATGTTTTCATGCATGCAAGTTTATCTGCTTAGCTCAA
ACTGCTTGAACTTATAGTCCCATCATGGTTATTTCCAATATTTTGAAAACAAATAT
ATACTTCCACATATTTTTAAAAATCACCACTCTCCAATATTTCTGTTGAATCAGAC
CTTACATTATGTTGTTTAATAAAGTATGGTAAGTTTTGGCATGTATGATTTTTATC
ATGTAAGAAGCATAATTTCTTAGTCAAAAATTTAGCCTTTGACTCTTTAGTAGAA
AGCTGAGTTCTGTACATTGTGTTCTAAAGATAGACAAAAATCTAGAGATTTTCTT
CTTTCAAAGTAAAAGCAGATGAGGCCTTTTTCCACCCTCTGAGGCATTAAATTGC
TTTGCTCAAGTTAGACTTTTAATATATTTAATTTGATAAATTTATCTGGTAATATA
TGTAATTCAGCAATATGGAATTGTATCATGTTATATGGTGCCATGAAATGCTAGG
GAATGCCACCTCAAGAGCTCTGGATGAAACATTTAATATGTCTTGGTTGGTTTGA
CTCCCATTATCAGTAGATAATGGGGCTAAAGTAGGTAACTGTATCCTATGTTTTC
CACCTATAAACTTTTGTGGTAATAGAATGTGAAATCTGGGAAGCATGTCGTTTTC
CAGAATTCTGCACTAGAAACTCAGCAGTTTCACTCTGCTTCTTGTGTTGTGGCAA
ACTTTGGTTCCCATAGTTCAGGGAGCACCTTTACTTTTTTGATATCCCAGGATTCA
AAAAAAAAAAAAAAAAAGAGAGATAAAAGGCACTGGGGAAAAGAATAGCTTAG
TGCAGAAAAGGGAAATCTTCTTTACTGTTCCTGAAGCCCTACAAAGTCACATCCT
CTAAATCTGGCTATTTCATGTAAAATCCAGGTGGTAAAGACAGAAGACATATGTT
ATGCCTGTGTCTTTTTATTTCTCTGTTTCTGCCAGTCAGATAGCATAAACATTTAT
GTCAGATAGCAAAGAGTGGATGGGAATAAAAGCACAAAATGGAGAAGAGGACT
TTTTAAAATTTTGGAAAATTCTTCCATTCACTCAAACAGAAATGAGCAGACTTGA
CAAAAATTTCATTGATAAAATGGTGAGTACCTTATAATTATAATAATTATGTATA
ATGATAAAATTAAAGTAAGCACAAAATACCTTTATCATTAAAATGGTGATAGTTA
ACCTGAATCAAGTGAAAAAATCAGGGAAAAAGTTCTTTTTATGAATAAAATAAT
AATTATTATTCATATTACTTTTATTAAAGGTCAAAGAAGGAAATAATACATACAA
AAGTGAAAAAATACAACTATCAGAAAATATATGTCATAGTACATCTTCTGCTGCT
GCTGACAGATTAACCCAACAAAGAAAGATTAGGAAAACAGCCTCAGCAATTTCC
CAAGAAACTGAAGGAAGAGCATGATAGGTAAGTAAGCCTATTGCAGTGTGTTTG
TTTTGTTTTGTTTTGTTTGGGTTTTTTTTTTTTTTTGAGATGGAGTTTCTCTCTTGTT
GCCCAAGCTGGAGTGCAATGGTGTGTTCTTCCCTCACTGCAACCTCTGCCTACTG
GGTTCAAGTGATTCTCCTGACTCAGCCTCCCTAGTAGCTGAGATTACAGGGATGT
GCCACCATGCCCGGCTAATTTTTTGTATTTTTAGTAGAAATGAGGTTTCACCATGT
TAACCAGGCTGGTCTGGAACTCCTGACCTCAGGTGTTCTGCCCATCTCAGCCTCC
CAAAGTGCTGGGTTTACAGGAGTGAGCCACTGTGCCTGGCCACCTATAGCAGTAT
TTCACAGGAGATAATTGTCATTGTGCTATAAACTAATTCAAAATTGGACTAATAT
TCCTTATGATTAACAAGTTTTATATTTTTACCAGGGATATTTAGCCCTGTCTGGTA
ATCAGAAAAATGTAAATTAACATAAAATAAGATATATTTTGTAAAGTCATGC TGA
TATTTAAAAAGTAATTACTCATGTTGGCAAATGTGAGGAAAAAGGCATTCTCATA
CACTGTTGGTATATGAAATTGGTAAATTATTTGTGAAGGGTAACTTAGTGCTGTG
TATCAAAATTTCAAATAACCTGACATCCCTTTAACTCAACAACTCCACTTCTGGG
ACTAGATTTCCCAGGAAAACATAACTTGTGTAAACATACACAAACTTATTAAGGG
CATTAATTATATATTACACATAATGAACAATAGGCTAATTAATATATAAAATATA
TGTAATAAGAAGGTGAATTGAAAGTATTAAGAAAGAATTATAAAAAGTGTGAGG
TAACAGATGTTAGACTCTTTAGCCTAGTTTTGGATGACAGTCATTTGCAGATATA
GTTTTTGTGAGAGACATCTTACTCTGTAAATCATTTGGAGAGACACCCGCAATAT
TTCGTAAAGATGAAAATTTATTTCTAGTGAACTTATACACTTGTCAGTAAATAGT
AACTTTAAAATTTTAGTTGATTGTAAATGACCTTTTCTAATTAGGGAGTAATTATG
ACTGTGTGATTTGAAAAGGTAATTTTGAACTTGTAACTTTACTGAATTATCTCCAG
TATCCTTTTTTATAATATATACTAGAGTCACTAGTAATAAAACCTTTAGCAGAAT
ATTCTTTCCTTACTACTTCTCAAGTATATGCATTCTTTTGAAGATGTTGAAGTGAG
AATTTAAATATCTGAGAACTGCAAAGGAAAAATAATCCAGAACATAGAAATTTT
ATTAGGATAATAAACAACATCTGCAGAGGTAGATAACAGGATGAACTCTTTATTT
TTTAACAAAATGAATTTCAAGATAAATGTCTTTATCTGCAGATGCACCTTACAAC
AAGAAAATGAAGAAAAAACAAATGTTAATATGCTGTACAAAAAAAATAGAGAA
GAATTAGAAAGGAAAGAGAAACAATATAACAAAGAAGTTGAAGCAAAACAACT
TGAACCAACTGTTCAATCACTAGAGATGAAACCGAAGACTGCAAGAAATACTCC
AAATCAGGTAAATCAATCTTTGGTAAAAATTCTATATTTTAAACTTTATTTTATCA
GTGTTACTTACAATATCCACTTGATTTAATATATATTATTTAGGTAAAAAACAAA
CCAGAAATGTTATCTCATTTTTAAAAATGAGTGATGACACTTACAGGTACAATTA
TTAATATATATTATAAATCTTGGCATCCACATAGGATATTATTTTATTACAAAGA
GCTTTTGAAAACAATAATATGCCATAATATATACTTAGTGATAACTTATTGATAA
AGATTTTGTTCCCAGTAAAATTGTTCTTGTACTTTCCCCTATTTCATATTGATTACT
GTACCTAATATTATAAAGAAGAAACAGAAATTATTGCAATCACAAATAATCTCAT
GATATTCTTAGAAGAGGTCTATAAATTTTATCTTATTTACCACTGGTGTTTTGAAA
TAAAAGTTTTCTTTCGTATGGATATATTTACACCACAGAAGTAACTGTGATCTGTT
GGAGAACTAGAAGTAGAGTCAGAAGTCCTGGGGAAAATCCTGTAGCTTGCTTAT
ATTTTTAACCTTTCTTTCTCAAAATTATGGTAACTAGATGAGTTCATCAATGAATG
TATATAGGAGTGACTAGTATAATGTGTAGATTTATGTTAGTAAATGTAATTCTTA
TAACTGACTATAAAAGTGTTAAAAGAGTCAAATTGGAATAGAATGTTATCAGTG
AAACAGAACTGTAATAACTCTGGGAAATTTCATCTGTCCAAATACGTGTGAACTA
AGGTTCTTACTATAGGGTGGTGTATAGGTTAGATATCAAAGTGTAAATGCAATTT
TTTGACATATTTTAATTTAGTCAAATTTGTTAATGCTTTAATTTATACTTTTGAGTT
TGTTGTAATTCAGGGAAAGGCTTTTCCAATTCTGAAATTCTTAAAAATTCTCTGGT
GTGCATGTGTGTGTGTGTGTTTACTTTTATAAATTCATTGACTTTAAATAAATTTC
TGAACTTTTTGGAATTTATGCTCTATAAGGTTCAAAATTTTGCTTCAACTTTTTCT
CCAGTTGGATATCCACTTACAGTAACCTTTTTAGTGCATGGATGTGCAGGTTATTC
TTTAACTTCAGAGGTAATCATGATATGTTATTTTATTGAGTACTAGCTAAAACTTT
CTGTTGTTTTATTTAGGATTTTCATAATCATGAAGAAATGAAAGATCTGATGGAT
GAAAATTGCATTTTGAAGACAGATATTGCTATACTCAGACAGGAAATATGCATA
ATGAAAAATGACAACCTGGAAAAAGAAAATAAATATCTTAAGGACATTAAAATT
GCTAAAGAAACAAATGCTGCCCTTGAAAAGTGTATAAAACTCAATGAGGAAATG
ATAACAAAAACAGCATTCCGGTATCAACAAGAGCTTAATGATCTCAAAGCTGAG
AATACAAGGCTCAATTCTGAACTGTTGAAGGAAAAAGAAAGCAAGAAAAAACTG
GAAGCTGAAATTGAATCTTATCAGTCTAGACTGGCTGCTGCTATAAGTAAACACA
GTGAAAATGTGAAAACAGAAAGAAACCTGAAACTTGCTTTAGAGAGAACACAAG
ATGTTTCTGAACAAGTAAAAATGAGTTCTGATATTTCCGAAGTAGAAGATAAGA
ATTAGTTTCTTACTGAACAACTTTCTAAAATGCAAATTAAATTCAATACCTTAAA
AGATAAGTTCCGGCCGCCGCCGCCACTGCAGCCTGCTGGGCTGGAGGAAGCAGA
GCTGGTGCTGTCCCGGCTCTCTTGCGGGGAAGCAACTGAGGGGGCGCCTTGGGGT
GGGTGCTCCTGGTGAGAGGAGTCCACTCCATGCATGTGGGCGGAGGCCATCCCC
CGAGAGCCGCCGACATGAAGAAAGACGTGCGGATCCTGCTGGTAGGAGAACCTA
GAGTTGGGAAGACGTCACTGATTATGTCTGGTCAGTGAAGAATTTCCAGAAGAG
GTTCCTCCCCGGGCAGAAGAAATCACCATTCCAGCTGATGTCACCCCAGAGAGA
GTTCCAACACACATTATAGATTACTCAGAAGCAGAACAGAGTGATGAACAACTT
CATCAAGAAATATCTCAGGCTAATGTCGTCTGTATAGTGTATGCCGTTAACAACA
AGCATTCTATTGATAAGGTAACAAGTCGATGGATTCCTCTCATAAATGAAAGAAC
AGACAAAGACAGCAGGCTGGAGTGCAGTGGTGGGATCTCTGCTTGCTACAACCT
TCACCTCCCAGCCGCCTGCCTTGGCCTCCCAAAGTGCTAAGATTACAGCCTCTGC
CCACCCGCCACCCTGTCTAGGAAGTGAGCAGCGTCTCTGCCTGGCCGCCCATAGT
CTGGGATGTGAGGAGCCCCTCTGCCCGGCCGACCCGTCTGGGAAGTGAGGAGTG
CCTCTGCCTGGCCGCCACCCCGTCTGGGAAGTGAGGAGCATCTCTGTCTGGCCGC
CCATTGTTTGGGATGTGAGGAGCGCCTCTGCCCTGCTGCCCCAAATGGGAAGTTA
GGAGCGCCTCTGCCCAGCTGCCCCAAATGGGAAGTGAGGAGTGCCTCTGCCTGG
CTGCCCTGTCTGGGAAGTGAGGAGCGCCTCTGCCTGGCTGCCCCAAATGGGAAGT
GAGGAGCGCCTCTGCCCGGCCGCCCCATCTGGGAAGTGAGGAGCGCCTCTGCCC
GGCCGCCCCGTCTGGGATGTGGGGAGCGCCTCTGCCTGGCCGCCCTGTCTGGGAA
GTGAGGAGCGCCTCTGCCCGGCTGCCCTGTCTTGGAAGTGGGGAGCGCCTCTGCC
CAGCCGCCCCGTCTGGGAAGTGAGGAATGCTTCTGCCCGGCCGCCACCTGGTCTA
GGAAGTGAGGAGCGCCTCTGCCCAGCTGCCCTGTCTGGGATGTGAGGAGCATCT
GCCCAGCTGCCCTGGCTGGGAATTGAGGAGCACCTCTGCCCAGCCGCCCTGTCTG
GGAGGTGAGGAGTGTCTCTGCCCGGCCGCCCCGTCTGGGAGGTGAGGAGCGTCT
CTGCCCGGCTGCCCCGTCTGGGAAGTGAGGAGCACCTCTGCCCGGCCGCCCCATC
TGGGAGGAAGTGAGGAGCGCCTCTGCCCGGCCGCCCCATCTGGGAGGTGAGGAG
CATCTCTGCCCGGCTGCCCTGTCTGGGAATTGAGGAGCGCCTCTGCCCGGCTGCC
CATTGTCTGGGAAGTGAGGAGCACCTCTGCCCGGCTGCCCGGTCTGGGATGTGAG
GAGCGCCTCTGCCCAGCTGCCACCCTGTCTGGGAAGTGGGGAGTGCCTCTGCCCG
GCCGCCACCCCGTCTGGGAGGTGAGGAGTGCCTCTGCCTGGCCTCCCCATCTGGG
AAGTGAGGAGCGCCTCTGCCCGGCAGCTGCCCCGTCTGGGAAGTGAGGAGCGTC
TCTGCCCGGCCGCCCCGTCTGGGAAGTGGGGAGTGCATCTGCCCGGCCGCTCCGT
CTGGGAGGTGAGGAGTGCCTCTGCCCGCCCGCCCCATCTGGGATGGGGGGAGCG
CCTCTGCCCGGCCGCCCATCATCTGGGAAGTGGGGAGCGCCTCTGCCCGGCCGCC
CCATCTGGGAAATGGGAAGCGCCTCTGCCCGGCCACCCCATTTGGGAAGTGAGG
AGTGCCTCTGCCTGGCCGCCCTGTCTGGGAAGTGAGGAGCGCCTCTACCCCGCCA
CCCCATCTGGGAGGTGTACTCAACAGCTCCGAAGAGACAGCGACCATCGAGAAC
GGGCCATGATGACGATGGCGGTTTTGTTGAAAAGAAAAGGGGGAAATGTGGGGA
AAAGAAAGAGAGATCAGATTGTTACTGTGTCTGTGTAGAAAGAAGTAGACATAG
CAGACTCCATTTTGTTCTGTACTTAGAAAAATTCTTCTGCCTTGGGATGCTGTTAA
TCTATAACCTTACCCCCAACCCCGAGCTCTCTGAAACACGTGCTGTGTCAACTCA
GGGTTAAATGGATTAAGGGCGGTGCAAGATGTGCTTTGTTAAACAGATGCTTGA
AAGCAGCATGCTCCTTAAGAGTCATCACCACTCCCTAATCTCAAGTACCCAGGGA
CACAAACACTGCCTAGGAAAACCAGAGACCTTTGTTGACGTGTTTATCTGCTGAC
CTTCTCTCCACTATTATCCTATGACCCTGCCACATCCCCCTCTCCGAGAAACACCC
AAGAATGATCAATAAACATTAAAAAAAAAAGGTACAAGAAAAAAAAAGATAAG
TTCTGTAAGACAAGAGATACTCTCAGAAAAAAGTCATTGGCTTTAGAAACTGTAC
AAAATGACCTAAGCCAAACACAGCAGCAAATAAAGGAAATGAAAGAGATGTAT
CAAAGTGCAGAAGCTAAAGTCAGTAAATCCACTGGAAAGTGGAACTGTGTAGAA
GAGAGGATATGTCAACTCCAACGTGAAAATCCATGGCTTGAACAGCAACTAGTT
GATGTTCATCAGAAAGAGGATCATAAAGAGATAGTAATTAATATCCAAAGAGGC
TTTATTGAGAGTAGAAAGACCTCATGCTAGAAGAGAAAAATAAGAAGCTAATGA
ATGAATATGATCATTTAAAAGAAAGTCTCTTTCAATATGAGAGACAGAAAGCAG
AAACAGTAGTAAGTATCAAGGAAAATAAATATTTTCAAACTTCTAGAAAGAAAA
TTTAAACATTTGGTTCTGGATACATGTTGAACCTAGTTGAATATAAAAATCAGTA
GATAAAAAGTGTGTTTACTATACTGTATAATTCCATTTACATGAAGCATCCAGAA
AAGAGAAATGTATAGGTACAAAAAGTAGATTAATGTTTGCAAAGGGCTGGGGCT
GGAAGGTGGTAGTGACTGCTAATGGGCGTGAGGGATCTTGCAGTGATGGAAATG
CTCTAAAGTTGGATTGTAGAGATGGCTGCACAGCTCAGAAAATGTACTGAAAAT
CTTTAACTTTATGTTAAAACAGATACATCTATAGTATGTAAATTATATTTTAACAA
AGCTTTTTGATTTAAAAAAAAAAGAAAAATGTGTTTATTACATCAGCTTAGAAAC
ATACCTTGTTTCCATAGAGGTGAGAGATGATTTACTTTGAGAGAAGACATTGTGT
CACCTATGACATTTTATTAGGCACAGAGTCATATTTTAAGGTAGATAGTCCTGTA
GTGCTGAAATAATAATTTTAATGTCTTTATGTTGCCACATGTTAAGACCATGATG
AAGGTATAAATGGAAATGTTTACACCTGAAATGAGTGTTTTCAAATTAAAATTTA
ATTGATTTGCTTCAACACTTAATTGTAGATTTCCCAGATGAAGTGTATTGCTGTGT
CTTGTAATATCTTGCTTTAAGTAGTTTTTTATATATTTTAGTTGGTATAGCTTTATT
ATTATTCATATTAATTTAACTTAAATCTGAAAATATGTCAGTCTCAAATTACATAT
TTTTATGACCATGTAATGTTTTAAAGGCACCTACTTGTTATAAAATTATAATTTAG
GGTAAATGTAAATTTTAGCAAAACTATATTTGATTTAGTCTTCCCACTGGTATTCA
TAATTTACTTTGAATATTTTTATTAATAATTAGCTCATAATTTTTA
>XLOC_12_009136 Agilent Human SurePrint G3 Probe: A_21_P0012220
Primary Accession: ENST00000429521
(SEQ ID NO: 21)
GGACTATTTAATAATAAGGAAAATAAGTGCATTTGAAGCCAATCTCTCTTAATTC
AAAGCTCATTTCCATAGTGACCCATTTGGATCAGGAGTGCCTGACATTCGCATCT
GGGATCCTGACACCATTGATAGAAAACAGCCCTCATGCTTGCTGTGCACTATGAC
TCACCGGGTATTGTCAACATCCTTCTTAAGCAAAATATTAATGTCTTTACTCAAG
ACATGTATGGACAAGATGCAGAAGATTACGCTATTTCTTGCCGTTTGACAAAAAT
TCAACAACAAATTTTGGAACATAAAAAGATGATACTTAAAAATGACAAACCAGC
AACTCGTGGCAGCCATTGATGTTTACTCTGTCTTCATAGTTTTACTTTTTTCAGAA
GAGTCACATAGTTGGAATAATACTGTGGATATATTTTTGAATATTAAGAAAATTA
AAGCTCCATGGCAATTGAAGGACCTCCTGCAAAACATCCTTCCTTGAAGCCTAGC
ACTGAAATGGAAGATCCTGCTGTGAAAGGAGCAGTACAAAGAAAGAATGTACAG
ACATTGAGAGCAGAAAAAGCCTTACCAGTGGCTTCAGAGGAAGAGCAACAAAG
GCGTGAAAGAAGTGAAAAGAAGCAACCACAGCTAATTTTAGAACATGCACTCTG
ACAGAAAAGACATCTGAGAAACAAAACAAGCAAATTTGTTTTCCTTTTTGCACCT
GCCAAAAAAAAAAAAAGAAAAGCCTCAAGAACCAGAACTGG
>XLOC_12_009441 Agilent Human SurePrint G3 Probe: A_21_P0012326
Primary Accession: ENST00000447898
(SEQ ID NO: 22)
AGAGCGAGCTTCGGAGAAGCAGTGGTGGGTTCCATGTGATGGTGGAGTAGGAGG
CAGGTCTCCGCGTCTCGCTGTATTGCCCAGGCTGGAGTGCAGTGGCATGATCTCA
GCTCACTGCAAGCTCTGCTTCCTGGGTTCACGCCATTCTCCTGCCTCAGCCTCCTG
AGTAGCTGGGATTACAGGCACCCGCCACCACGCCCAGGAAAGAAAAAAGAAGA
AAACAAACCTCCATACGAGAATGGGTCTAAAGGAACTTCCCAAACCTCCATGAT
TTTGCAGGAAACAAGATAAAGGTGGTTTCCACAAGAAAAATGGCACAATGTTTC
TCAGAAGACAATTACATAAGAATCAGCATACTTCAAATTCACAGCAAATAATCA
GACAATTGATGAAAATACTTACCCAAACACTAATTGTAGACTATGCCTTCTGAAT
ATGTTTGTCATAAACTTGGAGTAAGGAATCCTCACAGGCACTGGACAATTCAAAA
AACGTAAAGTTGTTTGTTAGAATACTGGTGCTTTTGGATAGAAACCCTCATCCAT
ATCCTGGTAAGGCTTGAAGTTGCACAGGAGTTTTCATTTGTCAAAACCCAGAAAA
CCATAAGCTTTAGATTTGTGAATTTTATATTGTATTATATGTGACCTTTCTTTTTAA
AAAATGAGCTGTAAGCAGTCTCCCAGACAGTAGCTCAGCCTCCAGAACTCTCTTT
CTGCATAGTTGAAGACCCCTCTTCACACAAGATGGTAGCAACAAATCATAGGTGC
AATTGCACCAAATTCACAGAAGATCAATTGAAAATCCTCATCAATACCTTCACTC
AAAAACCTTACCCAGGTTATGCTACCAAACAAAAACTTGCTTTAGCAATCAATGC
AGAAGAGTCCAGAATCCAGATTTGGTTTCAGAATCAAAGAGCTAGGCATGGATT
CCAGAAAACACCAGAACCTGACTTTAGATTTAAGCCACAGCCATGGACAAGATT
AACCTGGTGTGGAGTTTCAAAATAGAGAAGCCAGATGGTGTTGTACCACCTATA
GCACCTTTCAATTACACACAGTCATCCATGCATTTATGAAAAACCCATACCCTGG
GATTGATTCCAGAGAACAACTTGCTGAAGAAATTGGTGCTTCAGAGTCAAGAGT
CCAAATTTGGTTCCAAAATCAAAGATCTAGATTTCATCTCCAGAGAAAAAGAGA
ACCTGTTATGTCCTTAGAATGAGAAGACCAGAGAAGACCAGGGGCAAGGTTTCT
GAGGGACTTCAAGGTACAGAAGATACACAAAGTGGCACCAGCCTCACTAGCACT
CTCATTTCTCAAGAGCCAGAACATGGTGAATACAGTCAAGTTCAGTGTATTTGAT
AATATCAATTTGGGCCCCAAATCTCTCTCACAGTCTTCCTGGGAGTCTATTCTTCT
TCCAAAAGTGCAAGCTAAGCCTTCTGAAGATGGTAAAGAACTTGGCCGGGTGTG
GTGGCTCATGCCTGTAATCCCAGCACTTTAGGAGGCTGAGGCTGGAAGATGGCTT
GAGCCTAGGAGTTTGAAACCAGTCTGAGCAACATAGTAAGACCCTGTCTCTATTC
TAAAAAACAAAATAAGTAAAAAGGACTGTAGGAGGCCAAGACAGGTACAGGAG
GCACCACACTACCCTGTTGACACAGCCTGGATCCAGAGTTCAGCAGACCTTGAGA
CAATGAAAACAAACTTAGTAATAATCATTTTTCAATCATTGCAGTAATTATTGAT
TTGGACAAAAATCAATTGATGTCAAAACCTTAAAGTGACGTTTCTCTGCCTATGG
AGTGGTCATTCTTTTATTCCTTTAGTTTCATAATAAATTTTCTTTTACTTAAAAAA
ACTTATAGTTTGATGAAGAGTGAGATATATACCTCATCTCAAAGAATCTTCACAC
ACGCACTTATTAATTACAAAAGGAAAATCAGTAATTTTGCAGTGGAGACATATG
GCCAACTCCACCTTACCCAAGTGGCTGAAAGTCACTGCACCAGTAATGGCACAA
ACCAATGTGAGATGATTCCTGATATGATACACTAAAAAGGGCACTGTCTCTTCTG
CATGTTGCAGACAAAAAGTGGGTAAGCTGACACTGAAACTAATAATTAGGCAAT
GTCAAGCAAATACAAATTCAGGTTGACAGTCTGCAAAGTAACATCCATGTACTCT
TCAACAATGGATCGACCCTAGCTACTCAGGAGGCTGAGGTGGAATAATTGTTTGA
GGCCAGGAGTTCCAGATCAGCCCGGGCAACATCATGCGACCCCATCTCTAAAAA
CATCTTTTTAAAAATGAGCCAGGTGTGGTAGCATGCACCCGTAGTCTCAGCTACT
CAGGAGCCTGAGGCAGGAGGAAGGTTTCAACATAGGAGATCGAGGCTGCTGTGA
GCTATGATCGTGCTACTGCACTCCAGCCTGGGTGACACAGCAAGTTCCTGTTTCC
AAACAACAACAAGAAAACAAAACAAAACAAAACAAAAAATAGATAGAATAGTG
ACAATAAAAATGGAGAAACAGTAGGCTGACTCAGGAAATGCTTAGAAAGTACAG
CCATACCTCAAAGATATTGTAGATTTGATTCGAGACCACCACAATAAAGCAGATA
TTGCTACAAAGTGAGTCACACAAATTGTTTTGTTTCCTTGTGAATATGAAGTTATA
TTGGCTGGGTGTGATGGCTCATGCCTATAATCCCAGTACTTTAGGAGACGGAGGC
GGGAGGGTCACTTGAGCCCAGGAATTGTGAGATCAACCTGGGCATATAGGGAGA
TCCTGTCTCTATTTAAAAAAAGAAGCTATGTTTACACTACACTATAGTCTATTTAA
AGTGTGAAATGGCGTTATGTCCTTAATTTTAAAACTCTTGATGCTGGCTGGGTTC
GGTGGCTCATACCTGTAATCCCATCACTTTGGGAGGCCAAGACAGGTTGATTACT
TGAATTCAGGAGTTCAAGACCAGCCTGGACAACATGGCAAAACACGTCTTTAAA
AAAAGAAAAGAAAAAAGAAAAACAGAAAGAAAAAGAAGAAAAACTACTTGCTG
CCCTTACTTGAAGCTCAATTATTTAAAACAAAGAAAAAATATAAAAATCTTTTAT
TGCTGAAAATGCTAATGATCACCTGAGCCTTCAGGGAGTCTTAGTCTTTTTGCTG
GTGAAGGGTCTTGCCTTGATGTTGTTGGCTGCTGCCTGATAAGGGCGATGGTTGC
TGAATATTGAAGTGGTTGTAACAATTTCTTAAAAGAAAACAATGAAATTTGCCAC
ATTAACTGACTCTTCCTTCCACGAAAGATTTCAGTGTACCATGCGATACTGTTTGA
TAAGCATTTTACCCATAGTAGAACTTCTTTCAAAATTGGAGTCAGTCCTCTCACA
CCCTGCCACTGTTTTACTATGTTTATCAATATTCTAAATCCTTTGTTGTAGGCTAA
ACAATATTCACAGCATTTTCACCAGGAGTAAATTTCATCTCACAAAACCACTTTC
CAGGCTCTTTCTGGACTGTAGAGTTCTTTCCAGGCTACCTTGTGGCAGTTTAAGA
GTCTGGCATCATTTTCCGCTGGGACCTAAGGATCGAGGAGGTGCTTGTGACTAGA
CTGCCAATGGACCCATCACAAAGTTTAACCCAACCTTGATCCCCGAGTCTTCACA
AATGCTCACTGAAGAAAATTCCTGGAACAATTCAGGGTCCTTTCATAACCTCTAC
TCTGAGGTGTTAATAAAAAACCTTAGTAACTTAAAAAAAATGAGCTGTACACAA
ATACTGAACAATAATGCTACATATGTTAAGTATGTAAGAAAAATATATACTTTGA
CATAAATAAGAAACGGTGAGTTGATAATTGGATAGAATGGTGGATAGAGTGATA
GATATGTAGTAAAGCAAATATAACAAAATGATAATTGTACAATCTAAGTGGTTG
GACTATAAATATGCACTTCCCACAACATTTTTATATGTTTAAACAGTTTTATAATA
CCATATTAGGGAAACTGTTTGTCTCAAGGAAATAGAGATTGTGATATGTTCTAGT
ACAATGAAGTGTAATCATGTAAAATAAAAGCTTTTACTTCTGGCAATTAAAGTTA
ATCATGTTAGAACACTGTCTAGGAATGGTTGG
>LOC100287482 Agilent Human SurePrint G3 Probe: A_21_P0013271
Primary Accession: NM_001195243
(SEQ ID NO: 23)
CGAGGCCCTGCCCCACGCCCGGTGATTGTGCGCGCGGCCCCGCCCCCGAGGCGC
ACGCCGGCCCAGCGCCCACAGCTGCGGCGGCCTAGGTGCCGCGTGGGGCAAGCA
GGTGCCTCGCGTCCAGGCGGCTCCGCGGCTGGCTGCCTCCCGAGCCGGCCGCGCT
CCTCCCAGCGAGGCGTGGCGGGGAGGCGTAGTGAGGCTGGGCCCGTGGCGGTTC
CCTGAGGAGGGCCGAGAAGGGGCCGGGGGTGCTAGGGGAACGGGCGCTGGGGG
CAGCGGCCCCGGTGGATGCTAAGGGCTTCGGGATCGGGAGAGTCCACCACGCCT
GCCTGCTCGGCTGAGAATCGCCATGCCAGCTAAAGGGAAAAAAGGAAAAGGCCA
GGGCAAGTCTCATGGGAAGAAACAGAAGAAACCAGAAGTGGACATTCTCAGCCC
CGCGGCCATGCTGAACCTCTACTACATCGCCCACAACGTCGCTGACTGCCTGCAT
CTGCGAGGCTTCCATTGGCCGGGTGCTCCCAAAGGAAAGAAAGGGAGAAGCAAG
TGACAGCATTTCACAACACATCTCTGTTACAGACAACAGGACCTGGGGAAGAGA
AGTCAGGATAACACAACTGTTGCCAGCAACATAGACTTTACTCCAGACGACTTGA
GATGCAAATTAAGTGTGCTTTTCTGTGATGGTGGAAGATCAGGAAATGCACCTTA
CTTCCTCTGTTATGCCAGATATGGTTAGCCACTTTGGTTTTTTAGGAGCTATAGGA
TGGGAAAAGCCTGAGTAATTCCTACACAGTGTGCTGAAATTAATAGAACTTTCAG
AAATTATTATAATTCTGGGTCAGGATTAAACTTTGCTCTCAGAAGGCAGTTCTAG
TTGCATTAATTGTTTTCTTTTGCCAAAGAGCGTTTGTCATTTAGAGAAGACACGGC
AAGAAACACTGGGTTTCCTTAGGAACATTCCTCTCTTGGGCACCATTTCCTTTTTT
TTTTTTAATGGAAAATAATAAATACTTTGTTTCTATAATTTTCTTCTCAGCAAAAA
AAAAAAAAAAAAAAAAAAAAAAAAAAAAA
>FLJ20444 Agilent Human SurePrint G3 Probe: A_21_P0013726
Primary Accession: XR_132891
(SEQ ID NO: 24)
TCTTCCGTGCAGGCAGGCTCTCCTGGGGACCTCAGAGATTCTCTCCAGCGGCAGC
GGAAAACGGACAATGGGTGGATTCGGGTCCAGATTCTGGTAGGAGGGAGTTTGG
GATCGAGATCTGGAAAAAAGCACTAGACTGGAAGAGGACGCGATGGAGTCGGA
GCCGCTGGCGGGGACAAAAACCAGAGGCCGGGGAAGGCGCCGGTGGGAGGCAA
GGCACGGATGGACTTTACCTGCGCACGCGTCGCAGCCATCTCCGCGCACAGTGGT
GGCCACCGCGACTGGTGCTGAAGTGTTGGCGCGTGCCGGGCGCTCCGCTGGGAC
CCGGGTTGCTGGCCCTGAGTCTCAGCTTTCTCATCTGTACGGTTGGGACAAGTAC
AGTAACCCTCGCCCGTCAAGACGGGCCAGGGCTGTGGCGAGGGTCCACGCCTTA
GAGCAGGTACCTATCTTGTGCAGGGCCCTGAGATGGGGTCTGACTCAGTTCCTGC
GGGGAACTTCACCAGTGACCCAGTCAGTGCCCTTCAGTTAAAGACCACCAGGAG
CACACTTGCAGGAGTAGGGCTGATTGGAGCCATTGTACAGTGTCGGGAACATAC
CAGGACACTGAGAATAGTGTCATGTCATAAGGACCCAGAGCAGATGGACCCTGC
TGTGATGCACAAAGAGGACCACGCAAGATATGATAAAGATCTACATCACTGAAT
TTTGGTTCCATTTTTGTATCTCAGCTTCCAGGAAATAAAAAAGAATTCTAACATTC
ATACTTTCAGTATTTTATGTGAGAGGTTTTGTTGTCAAAATCAAGTCTGAGAGCA
ATGTTTGTTGGGGCCTTTAATTGGAGTCACCAAGCGATAAAGGGGACATTGTCCT
CAACAATAACCCTATAATAAACACGTTTTGGACAATAAATATATGACAATTTCTT
AAAAGCAATTTCTTGGGCAATCAAGACAGTATGGCTTGAGTATGGAGTTATACG
ATGGTTTGGATTAATCCAGTATTAAATCTTTGGTTATTACAGAAA
>LOC100505666 Agilent Human SurePrint G3 Probe: A_21_P0014077
Primary Accession: NR_040772
(SEQ ID NO: 25)
GCCCGCGCTGCTCAGCGCTACCGCTTCCCCGCAACTGTGCGGAGTGGGAGCCGGT
GCCCGGTCCGACCGGCTTGGGCGGCGCGCCTTCACCCGGCGCCAGGTCCGGACC
CCTCCCTAGTAGCTTCGCGGCCTCCCTGCCTCCTGTGCGCGGCCTGGCTCGGAGA
GGTCGGGCGGGCAGGCTTTCCCGACTGCAGGCGAGGCAGTGCGCGGCTCACCCC
AGTCCCCGACCCACGTGAAGCGTACAGGGCATTTTATTAACCGGGAAGGACGGT
GCGGAAGAGCGAGCAGGACGCCTCTTCACCCCGCGTAGGCAGTGTCGTCGTTGC
TGTCACTAAAGGCGGAGGAAGAGAGCTCTTCGCGGGGCGTGCAGACCGGGCACC
GCTGCCGCATGTCGTCCCAGCACGACCAGCAGTACACGGCCTCGCAGTCCAGCGT
CCGGCACACGTAGGACTCGGGCGTCTCGGGTGCCTGGCACACCACGCAGCGCCG
GCACAGCCAGCGGCGCAGGAGCGGGCAGCCGCGGTGCAGGATATCCGCCAGCG
GGTGGCGCTGTTGGGAGGTGAGAAAACTGATGCTTGGAGATGTGATCACTGCCC
AGGGTCACCCAATGATAACATGCATGCATATGGAACTTGCTGCATGCCAGCACC
ATGAGTCCGCTCCCCATGCTGTCCTCACCACATTGCTCATTTCTGAGGCCTGGATG
GTGGGCTTGCAAGGGAAGATGACGGTTTTCTCCTCAGCTTTGCGGAGTGGCAGCA
GAGTCCGTTTGCCCTGGAAAACAAATGTCCACACAGTTAGGAAGCCCAAGGGCC
CTCTGCCCTTTCCTCTCTGCCTTCCTGGAGCATGAACCCACACAGGGCACACAGC
AGCAAGGCATCCCCGGGCAGTGCCGTGCCCACTCACCAGCTTCTTCCTGCGGTCA
TCGATCTGGCAGAAGTTCTCCTCATCTATCCCCAAACATGGGCTTCCTTGAGGCA
CAGTCATTCAACCAACCAGCCAGCATTCATTGAGCACCATCTATGTCCTGGGCAC
TGCTAGGGGATGGTGATAACAGGGAGAAGACTCTGTCCCTGCCTTCCAATTGTGT
AGAGGAAGACATCCCCCTACATGATGGGTGAGACATAGCAGAAGTGAGTAGGGG
ATGAGGTGGGGGCTCAGAGGAGGGCATGGTCAGCCTGTCTGGGAGGGAGTTGCA
TGTGTGCATCTGAGGTAGGGACAGGCATGCATCTTACAGGATGAATATCGAGCA
GAGTTACAGAGAGGGGGAAACTCCTTGAGGTTTCAGGAATCACCTAATCCACTG
TGACTCACAAATTCCTGCCTCTTGGCTTTGCCTGCAGCATATCTCCTGGAAGTGTG
CTGGGGCAAAACTCATCCCAGACCACCATCTCCATCCTCCCCCAATACACCCTGG
CTCTCCCTGGCTACCCTTGAGCACGGTGCACGTGTGCATGGGTGCATGCCTGCAT
ATATAGCTATCCCCCATGTATTTCCCAAAGCCCTACATAATGCTTCAGTTTGCTAA
GGAAAAAATGTTAATTACTGCAAATGTGTTTAAAACTGTAAAAGTACATTAAAC
AAACTCTGTAAAGTGTGAAAAAAAAAAAAAAAAAA
>LOC100507025 Agilent Human SurePrint G3 Probe: A_21_P0014172
Primary Accession: ENST00000289352
(SEQ ID NO: 26)
AGCGTTCGTAAGGTTCTCAAAGACTACAGAAGTTGGAAACTTCGCGGAGAGACT
GCAAGTTACCCTTTCCAAAATGGCGGGAAGGGCTAAAAACAAAGAAAGCTCGCA
CCCAGACGGCGGGCCTTAAACCAAGGCGAATCCGTGAGCGCAACACATCTGCTT
CTGTGGCTCCTGATGGATCTGAGAAGATGGACGTGGAGGATGAAAATCTGTCTG
ATTATTTTGAACTGATGTTTGTTGCTATGGAGATGCTGCCTATATGTTGATGTTGC
AGACGTTAAGTCACTAGCCCACAGCCTTGTATTCCATACTCAGAGACCCTGCTAC
TTACTTGACATCTCAACTTGAAAGTCCAATTAATATGCACTTCAAACTTTAATAG
GCTTCAAACAGAATTTCTTTCATTATCTCTGCAAAACAGCTTCTCTCATCATCTTG
AAATTAGTGAATGGCATTTTACTGTTTTAGTTGGAGTCATTTCTGTGGTTTTCTTT
CACATCCTACATAACAATCCATCAGTAAGTTCTATGAGCTCTTCTTTGAAAACAA
ACAGAATCCAACTGTTTCATTCCCACTTCTGCTCTGGTCAAGCCACTGCCAACAC
TCACCTTTATTATTGTAGCACCCTCATTGCCTAGTTCTGTCCCACAGATTTCCAAT
AAAAGGTGAATAAAATCAGGTCACTCTTCT
>LOC100506303 Agilent Human SurePrint G3 Probe: A_21_P0014553
Primary Accession: XR_110283
(SEQ ID NO: 27)
GGCACCCGCCACCACGCCCAGGAAACTCCAAACTGTCCAAGGAGATAGTTCTGT
TGTGATTACTTCATTGAGAAATTTAACTTATGAGCCGTTGAAAGGAATGCAAGTT
GCTGCAAAATCCGAATGAAGAGTGCAAAACGACTAAGCTACAATGTTTTGTCATT
ATTCACTCTGATGTGAAAAAGGCAGTGAATTTAATAGAAAATAACTTCGTAGAG
CAAAATCTCAGGTGTGTTTTTTTAGTGCCGCAGTCTTGGATGATGGGTTCCTAGA
AGCTCTCAACATCTCTTCTTAATTGGAGAAAGTGTTAAGCCCCAAAGTAGCTGGA
GCAGTACATCTTCAATTTTTGACAAGAAAGCAGGAACTTGATTACTTTGAGTGCT
ATTCATTAGTTTCTGCTTTCATTGAGAATGCAACAAAAGCCAACTAGGCTGCTGC
TAACTCCTTGCTGGACTTCTTCTGCCACTGTCACAGGAACTGTAATCTCACTGGAC
AATTAACTAGGGAGTCTTTCATCTTGAGTGACTGCTGCACAAATGATCTTCAAAG
CATTTTAGCCACCAGAGGAATTCTCTTGAAATACCCAAAATCCATCAGTATCTTG
AATCATGCTGGATTTTGAAGAATTCTTAACAAGCCATGTAAAGGGGGCTCTCTGG
CCTTGAAATAGTGATGTTTTTTATACAGAAAGGAGAATGCAGAATGGTCAGACTA
CCATGCACTGTTAAATTTGATTTCAAGAAATTACAGGAAAACTTTCCAAAGTTCC
ATCTCACAGAAATTATTTTTACAAAGAATTCCAAGATAAGTTTAGTTTTATGGAA
GACTTTTATGTGGTTTTTACTCACTCTTCATCTCAGACATCAACAGATGATTACAT
CACTTATTTAGCTAGTAAATTTATTAATATAAAAACTCAGAGACATTCCAATATC
CACATTGCTTACACCATTAGGCATAGATTCAGTGTCAGCTATGACAATTGAAAAT
AAGCTGTTTTGTGATTTAAAGGTTTAAATTTCTCTAACCAAACTGCTTGATCCAGA
TGCAGGACTGCAAATGTTAATATTTGTTCTGGAAGAACAATCAAATAAGACTTAA
GAGGAAAAGGAATGGCCACAATCCACCTGAAATTTTTTTTTAAAAAGTGTGCAG
CCTACTAAATCAGAATGAAAATAGAAGTACAAGATTATAAACAAAATGCAATCA
AACTTTTCTTAAGCTTACCTAAAGTTATTTCATCTGAAAATTTCAAGCAACTTTGT
TCAACATTAAATTGACAATCTAAACTAACAAGTCTTTTGAATTTATGCATGGTAG
TAAACATTCTCTCTATTAACTGTATTACCTAAGGCTAAACCTAAAATTTTTAAGCA
AAATTAGAAAAATAGTCTTCACTCATCAAAAAATAAAGTTTGTTACATTTAGTAT
TTTCCCAATAAAATTGGTCGTTCTTGGTTTTTTATTTGGAGAGTCTGTGCAAAATG
TCACTAAAAATAAATTAGCACTAGAAATTATTTCTAAATACCAAAAAAAAAAAA
ATGAAGAATGGTT
>LOC100506802 Agilent Human SurePrint G3 Probe: A_21_P0014847
Primary Accession: XR_132718
(SEQ ID NO: 28)
AATCTGCAACGGTGGGCTGCAGTGGAGAGAGGGGCGTGGACTGCCACTGCTGCC
CCTCGCCCTAGGTCACCCCCAGCTTTATCAAATGTCAGAGCACCAGGAATCCTCC
ATCATCAATGAGGACACAGAGCTGGGTGATGCCTACGTGTTGAGATCCTGGTCCC
TCCACACACGCTCTACCAGCTGCTGCGTGATGCCCGTGTCCAAGATCAGGTTGTG
CAGAAGGAAGTTGTTGCCTGGAACAGGAGGGGAGGGGTGGGGGTGGGGGCATC
TTCTTGCAGCTCCTTGCCCACCCTCACCCCCACCCTTAAGGCTCCACCAGGAGCCT
CCTCCATGACCTGGCCCTGGCCCAGGCCCAGCCCTTAGCTTGTGCCTGCTTATTTC
CACACCTGCCCGGCCTCTGGGTTCCTCTGGGCTGGCCCCATGCTGCCTGGGCACT
GCCCAGAGCCAGCTGCCCTGCCAGGCACTCACACTGCTTGGAGTCTGGAGTCACT
TTCTCCATGAGCTCAATAAAGTTTTTCAGGAACTCGG
>AB116553 NCode human ncRNA array Probe: IVGNh00466 Primary
Accession: AB116553
(SEQ ID NO: 29)
CCCAACCCTTTGGTGGAGCCTGAAAAAAATCTGGGCAGAATGTAGGACTTCTTTA
TTTTGTTTAAAGGGGTAACACAGAGTGCCCTTATGAAGGAGTTGGAGATCCTGCA
AGGAAGAGAAGGAGTGAAGGAGAGATCAAGAGAGAGAAACAATGAGGAACATT
TCATTTGACCCAACATCCTTTAGGAGCATAAATGTTGACACTAAGTTATCCCTTTT
GTGCTAAAATGGACAGTATTGGCAAAATGATACCACAACTTCTTATTCTCTGGCT
CTATATTGCTTTGGAAACACTTAAACATCAAATGGAGTTAAATACATATTTGAAA
TTTAGGTTAGGAAATATTGGTGAGGAGGCCTCAAAAAGGGGGAAACATCTTTTG
TCTGGGAGGATATTTTCCATTTTGTGGATTTCCCTGATCTTTTTCTACCACCCTGA
GGGGTGGTGGGAATTATCATTTTGCTACATTTTAGAGGTCATCCAGGATTTTTGA
AACTTTACATTCTTTACGGTTAAGCAAGATGTACAGCTCAGTCAAAGACACTAAA
TTCTTCTTAGAAAAATAGTGCTAAGGAGTATAGCAGATGACCTATATGTGTGTTG
GCTGGGAGAATATCATCTTAAAGTGAGAGTGATGTTGTGGAGACAGTTGAAATG
TCAGTGCTAGAGCCTCTGTGGTGTGAATGGGCACGTTAGGTTGTTGCATTAGAAA
GTGACTGTTTCTGACAGAAATTTGTAGCTTTGTGCAAACTCACCCACCATCTACCT
CAATAAAATATAGAGAAAAGAAAAATAGAGCGGTTTGAGTTCTATGAGGTATGC
AGGCCCAGAGAGACATAAGTATGTTCCTTTAGTCTTGCTTCCTGTGTGCCACACT
GCCCCTCCACAACCATAGCTGGGGGCAATTGTTTAAAGTCATTTTGTTCCCGACT
AGCTGCCTTGCACATTATCTTCATTTTCCTGGAATTTGATACAGAGAGCAATTTAT
AGCCAATTGATAGCTTATGCTGTTTCAATGTAAATTCGTGGTAAATAACTTAGGA
ACTGCCTCTTCTTTTTCTTTGAAAACCTACTTATAACTGTTGCTAATAAGAATGTG
TATTGTTCAGGACAACTTGTCTCCATACAGTTGGGTTGTAACCCTCATGCTTGGCC
CAAATAAACTCTCTACTTATATCAAAAAAAAAAAAAAAAAAAA
>AF087978 NCode human ncRNA array Probe: IVGNh01580 Primary
Accession: AF087978
(SEQ ID NO: 30)
AAAGCATGGGAAAAAGAGACTCTTTTAGGATCAGATCTGTGAGCACGTTGGCGA
GGAAAAACAAAACAAACAAAAAAAAGAACCTTGTGTCTGTCTGGTGAAAAAAA
GAAAAACAAATTGGAAGAGAGGACCATGAGAATTTTAATAAAACAGAAGGAAA
CTAATGGACCTTCCAGGATTTATTGTGGACGGATGTGGATATATTCTGTACAGGA
ACAACACATATGGAAGTGGACTGAAGCCTATGTAGAAACACACACACACTGAAC
ATTGTTATTCATTTTGTAAAATACTAGTCTTTATTTTCATTTTTTGTAAAATTTAAA
CATCGTATGCGCATAAAGAAAAAGGAAACAAGAATTAGGGGAAAATAACATTTT
CCAAATAATTATAAAAAATTGTCCTGTGTCTATGTATCTATATCTGTTTTGTATTT
TTTTCTGGTTCCAAACCAGATTTCCTGTGATTCTATACTAATAATTTTTGATATAA
CCCTTTGCTTCTTATAATGAGTGCGATATATGTTGTCGAGGCTGTTCTTCAAGAAT
TAAAATTGAAGTGAAAATTTAAACAAAAATAAAAGAATTTAGCAAAAAAAAAA
>AK024556 NCode human ncRNA array Probe: IVGNh04604 Primary
Accession: AK024556
(SEQ ID NO: 31)
GTAGAGATGGGGGTTTCATCCTGTTGGTCAGGCTGGTCTTGAACTCCTGACCTCA
AGTGATCTGCCTACCTTGGCCTCCCAAAAGGCTGAGATTACAGGCATGAGCCACT
GCGCCAGGCCTTCTTTCTTTTCTTTTTTTCTTTCTTTTTTTTTTTTGAGACATCATTT
AGCTGTGCTGAGGGGTTCTTAAATAGGCAGCTCAGAAAATTGTTTTCCTTTGTCA
GCCACATAAATTCAGCAGAGGCTCTTGGAGGGTCCCTGCTGGTGAGGGGTGAGG
CCAGCAGTGGAACTCTGATTTGGTTTTTGCTGAGCTGGTGGTTGAAAGGAATCCT
ACTACATCGGGGTTATAATAGGGAAGATACATTTTAGAATATGCCCAGTGGAGC
CATCGGATGCTGCATCGTCCCCAGAGAGCCAAGTCATCGTGGGCCAAGCTCCCAT
CCCCATGTCTGGCCTCAACTGCAGGCCCAGAATGTTGACAGCTGCCTCTTGGAGG
GTTATGGGAGCCTGTGAATGCCAACATCCCCATTTGCCTGCAGCGGCTGCTCCCA
TCCTGGCTTCCTGGTGGGACTTTTCCATGAATTGGGGAATCTGCTTTCTGATTCCA
AGGCCTATTAAAATTTCTGAGCATTGCCCATTTCTTTTGCTTTATCTGTAGGACAT
GGGCTGTTTTTAAAGAACCTCACAAATGAAAAAAAAAAAAAAAAA
>BC012900 NCode human ncRNA array Probe: IVGNh15798 Primary
Accession: BC012900
(SEQ ID NO: 32)
GTGGAACAGTCTTGTTATGGAGTGCCAGCTTAGAGGTTGTTGCAAACTTGTCTAG
AAGTGAGAGCATGGTTTTTTTTAGCCCTTTGAGAGTCTACATCTAATGAACATTCT
TGCTCACCCATAAATAACGTCAAGCCTCAATGTCACCGTCACGTTGGGATACTCT
TTCTCATCTGGCATCCTAGACAGGACAAGGTTGGTTACCTTTCCTTCCATGAACC
ATGAACCTGTGACGGCATCATTCATCCTGACTTCACCAAGCTCCGCCTGTGGGTG
AGGCCAGAGCTCCCACTGGCAATTTTTAGAAGAGCCAGAGGCTCCCTGCTTCCTC
TAGAAATAACAGTTCAGGGTGAAGCATGGAGGGTTTCAGTTCCCAGACAATGGA
ACCATTTAGAGACAACACAGTTGGACATTTCCACTTTTTCCTTGATTCCTGGAAGT
CCAGTGGGTTCTGCAGCTGAAAAAGCCCTGGGTCCCAGCAGCAGAGAGACAGGA
CAGAGGGGATGCTTGGGCGGGGAGGGACGGTAACCTGCAGAACAGATTCCATTT
TTATAGAACGAGTACACGTTTGCTAAAACAGTCCTGCTTTCCCAGACTGGATTCC
CACCACAGGGACAGTCGGAACTCAGGACTAGCTCCAGCGACATCTTTCCTCCGA
ATTCAAGCCTTCTATCACAATGTCAAAACAGCTATTTATAAAGCCATTTTCATTGT
ACTTGATAACAGCACGAGTCCCAAAACTTTTAGAAATAAAATAGGACATTGGCTT
GATTGAAAAGAGGGACTTTTTAAAAATTGTTCTTTCGTCAGAAGCCTTTTGGATG
ACTTACAATAGCTCTGATGAAGATACCACCCCAGCGTCAGTCCAATAGGTCAGTG
AGTTTCAACAGGCATCCATCCCTCCCATGAAGGGATTCTGGTGATGGGAAGTTTC
TGTAATGACAGGAAAGCATTGACCCTCATTGATTGTCAACTTTGGTATTAGCCAT
GAAAGACAGGATGCTCATTGGGTGTTCTGTAGAGTGAGGAATGCTGCCTATTCCC
TCCCAGAACGTCTGACCCAGGGGTGTGTGTTGAGGAGCCCTGGGGGAAATGGAC
CAAGTTTTCCCACAGAGCAGTATTAGGCTGAAGAGCAGGTGACTGGTAGGCCCC
AGCTCCCATCATTCCCTCCCAAAGCCATTTTGTTCAGTTGCTCATCCACGCTGGAT
TCCAGAGAGTTTTCCAATTTGGGAAGCCATGAGAAAGGTTTTTAAATCTTGGGAA
GATGGAGAGAGGGACATAGGATAGTTGACTCCAACATGACAGGAAGAGGCTGG
AGATTGGGAATTGGCCATCAACCAAGCCTGTAGTAGTAAAGCCATGGTCCCGCA
TTGGAATTACTTGGGGAACTTATACAGTTCTGATACCCAGGCTCTCCTAGACCAG
TTCAACCAATTCTAGGTGGGGGACTCAGGCATCAGTGTGTTTCGTAGCTCCCCGG
GTGTTTTCCCTGTGCAGCCGAGCTTGGGAAACTGCCATGCTTTTTGGATGTCAAG
GCGCTGTTGGAGGCTGGGTGTGACAGCACAGAGCCAGGTTGTCTTGTGGAAACC
ACAGCCACGGGTTTGCCACTGGCTCAGCATGGCCTCACTGCCAGTCCCAGCCTGG
CTGAGGGACAAGATGGTTTCTCTTGGGAGTTCCTGAGTGGAGCACCCTTCCAGGC
TTTTTGAAAGCCAGCTGATCTGTGGAGCCTTGTTAAGGGACTCAATACGGTGTTT
GGATATTGATGTTTTTCCTTGAGACTGTCTTGTCCATCAATAAAGATGGAGGATG
TCTCCTCTTTGAACCCCGCTTCCCCACCAGTACTCTCTCTCCCTTAGAGTTTATGA
GTTATTCAAGGAGGAGACTTCTTAAAGACAGCAACGCAATTCTTGTAACTTGTGT
AAATAGCCCCATCTTTCAGAGTGATACCATTTCTACATTTGATAATGCCTGTATTC
CTGTAGGATGTATATAGTTTAGGGGATTTTTTTTTTGTTTGGTTTTGTTTTTTAGAA
GTCAATATGTCTGGTTTTATTTATTGCTTGAAAAAGATCATTTGAAAAAAATAAA
TACATTTTCAACCACAAAAAAAAAAAAAAA
>BC013821 NCode human ncRNA array Probe: IVGNh15835 Primary
Accession: BC013821
(SEQ ID NO: 33)
GGGCTCTGTCCTTAGGGAGGAGCTGCGGAATCCCTGCAGCTGTGCCCCCAGGCCC
TGCCTTGCACACTTCCTGCAGCCAGGGCGCCCCTGGGGAGGTCAGGGCAGGCCG
GGGAGGCTGAGGCCCACCTGCCATAGTGGGCAGGTGCGGGAGCCAGGGCGGCA
GTGGCCTCGGGGCTGGGTGGGGCGCCTGGCCTCTGGTCTCTGGAGCAGTCAGGG
GCTCTGCAGACGCTGAGAGGCCTGCTCATAGTGGACTGGGAGATGCTGGAGCAG
CCTCAGAGCCATGGCCGGCCCACGGCGGGAGACGGCCCTGCTGCTGCCCCTCTGC
CTGTGCGTGTGCACCTGTGGGCACCTGCGTGTGCTGGGGCAGGCAGGGCTGTATT
GGGACCAGGTCCTGTAACAGCCTGCCTGCTTACCGTCTGCTCCCATCCCTGGGGA
AAGCAAGGGAGCTCGGGGTCCTAGGACCTGACCTCAGCGCTCACCCCCACCAGC
ACCACAGTCACCAGGACTCTGTGACTCAGTTTACCCCACGAGAGCCCCTGGGATT
CCCAGGGCATCAGAAGGCCCATCAGCCTCCCGTGAACTGCTGGGGTGGGCCTGG
CCTTGGGACGCGGGTGCAGGGGCCTCTCCTCACTGCCCCCATGGCACCCACAGCC
AGTGCCCGAGCCTGCTGCAGCCCCGACCCGGCAGAGCAAGCGGCTCTGCTACCT
CAGCCACGTAGCTGATGGCATCCTTCAGGTTCAGCTCGTGGAAGACATTCAGGAT
CCGGTCTCGAGACTTCTGGGCCGACCGTCTCATGAGGACCCTGCTGAGGAACTTC
CTGTCGAAGTGGGACCACCTGTAGGGACAGACCTTGGGTGTGAGCCTCAGGTGA
CAGGCGCCCTAGAGCCCGCCGGACGCGTGGCCCGGCCCCTTCTCTCCTGAATTTT
GTTTGCTATAGTGACCCTGTAGGCGCGTTTAAAATGAGGGAAGCAGCCCCTGCCA
CACGCCCAGGCCGTCCGCCGTTCTCCCGCCTGTCCTGTTGGATGGAGGCCGTTAG
ACGCATATGAAACTGCATGCCGCCTCCTCCAGAGGGTGGCTCAGGACACGGTGG
GTGTCAGGCCTGGTCAGGCAAGGGGGCTTTGGCCACATGGGGGGCACCTTCAGG
TGCACAGGAGGAAGGGCAGGGGCGGACAGACACCCTGAGCCCTTAGACTTGTGG
GAGCCAAGCTGACCAGAGTGAGGTTTTTTTTAGCCTAACGGAATTAGAGTATTCG
CTGGTTATCCGGATCAGAAGGGACGGTGGCCTGGCCGGACTTAGAGGAAACTCT
GGGGCACAAGGAGGTGATGCCTGTCACTTGGACATGGGTGCAGCCGCCAGAGCC
GCCCTCCAGGGCACAGGGTGGGCCCGGGTGAGCTTGTGTGCTCACACCTGGGCA
GGCCCCGCGGCAGCAATGGCAGCTCTCCTGTACAGGCTGAGTTTCAGCCACACCA
AGAAGTCAAAGCTAACCGAGGCTGTGCCTTCCGAGACCCCCGGGATGGCCCCTG
GGAGGCCAAGGAGTCGGGGACTGGGTACCCGGAGCAGAGTCACTGTGGCCACGG
AGAACCGCAGCTGAGCTTTATGAAGCCACGTGGCCACACCTCCCGGTGCCTCCAC
CCCAAGCAAACACAGATCGCTCAGAAAATGGGAACCCAGGGCAAATTGTATGTG
CTCCTTACTGGGTTTATTATAAGTGTCACATGTTTTTTATAATAAAACATAGGTGA
TTTCACCTTAAAAAAAAAAAAAAA
>EF177379 NCode human ncRNA array Probe: IVGNh23506 Primary
Accession: EF177379
(SEQ ID NO: 34)
GGAGTTAGCGACAGGGAGGGATGCGCGCCTGGGTGTAGTTGTGGGGGAGGAAGT
GGCTAGCTCAGGGCTTCAGGGGACAGACAGGGAGAGATGACTGAGTTAGATGAG
ACGAGGGGGCGGGCTGGGGGTGCGAGAAGGAAGCTTGGCAAGGAGACTAGGTC
TAGGGGGACCACAGTGGGGCAGGCTGCATGGAAAATATCCGCAGGGTCCCCCAG
GCAGAACAGCCACGCTCCAGGCCAGGCTGTCCCTACTGCCTGGTGGAGGGGGAA
CTTGACCTCTGGGAGGGCGCCGCTCTTGCATAGCTGAGCGAGCCCGGGTGCGCTG
GTCTGTGTGGAAGGAGGAAGGCAGGGAGAGGTAGAAGGGGTGGAGGAGTCAGG
AGGAATAGGCCGCAGCAGCCCTGGAAATGATCAGGAAGGCAGGCAGTGGGTGC
AGGGCTGCAGGAGGGCCGGGAGGGCTAATCTTCAACTTGTCCATGCCAGCAGCC
CCTTTTTTTCCAGACCAAGGGCTGTGAACCCGCCTGGGGATGAGGCCTGGTCTTG
TGGAACTGAACTTAGCTCGACGGGGCTGACCGCTCTGGCCCAGGGTGGTATGTA
ATTTTCGCTCGGCCTGGGACGGGGCCCAGGCCGGGCCCAGCCTGGTGGAGCGTC
CAGGTCTGGGTGCGAAGCCAGGCCCCTGGGCGGAGGTGAGGGGTGGTCTGAGGA
GTGATGTGGAGTTAAGGCGCCATCCTCACCGGTGACTGGTGCGGCACCTAGCATG
TTTGACAGGCGGGGACTGCGAGGCACGCTGCTCGGGTGTTGGGGACAACATTGA
CCAACGCTTTATTTTCCAGGTGGCAGTGCTCCTTTTGGACTTTTCTCTAGGTTTGG
CGCTAAACTCTTCTTGTGAGCTCACTCCACCCCTTCTTCCTCCCTTTAACTTATCC
ATTCACTTAAAACATTACCTGGTCATCTGGTAAGCCCGGGACAGTAAGCCGAGTG
GCTGTTGGAGTCGGTATTGTTGGTAATGGTGGAGGAAGAGAGGCCTTCCCGCTGA
GGCTGGGGTGGGGCGGATCGGTGTTGCTTGCCTGCAGAGAGGGTGGGGAGTGAA
TGTGCACCCTTGGGTGGGCCTGCAGCCATCCAGCTGAAAGTTACAAAAATGCTTC
ATGGACCGTGGTTTGTTACTATAGTGTTCCTCATGGCGAGCAGATGGAACCGGGA
GACATGGAGTCCCTGGCCAGTGTGAGTCCTAGCATTGCAGGAGGGGAGACCCTG
GAGGAGAGAGCCCGCCTCAATTGATGCCTGCAGATTGAATTTCCAGAGGCTTAG
GAGGAGGAAGTTCTCCAATGTTCTGTTTCCAGGCCTTGCTCAGGAAGCCCTGTAT
TCAGGAGGCTACCATTTAAAGTTTGCAGATGAGCTTATGGGGGGCAATCTTAAAA
AGTCCACAGCAGATGCATCCGGCTCGAGGGGCCATCAGCTTTGAATAAATGCTTG
TTCCAGAGCCCATGAATGCCAGCAGGCACCCCTCCTTTCCTGGGGTAAAGGTTTT
CAGATGCTGCATCTTCTAAATTGAGCCTCCGGTCATACTAGTTTTGTGCTTGGAAC
CTTGCTTCAAGAAGATCCCTAAGCTGTAGAACATTTTAACGTTGATGCCACAACG
CAGATTGATGCCTTGTAGATGGAGCTTGCAGATGGAGCCCCGTGACCTCTCACCT
ACCCACCTGTTTGCCTGCCTTCTTGTGCGTTTCTCGGAGAAGTTCTTAGCCTGATG
AAATAACTTGGGGCGTTGAAGAGCTGTTTAATTTTAAATGCCTTAGACTGGGGAT
ATATTAGAGGAAGCAGATTGTCAAATTAAGGGTGTCATTGTGTTGTGCTAAACGC
TGGGAGGGTACAAGTTGGTCATTCCTAAATCTGTGTGTGAGAAATGGCAGGTCTA
GTTTGGGCATTGTGATTGCATTGCAGATTACTAGGAGAAGGGAATGGTGGGTAC
ACCGGTAGTGCTCTTTTGTTCTTGCTTCGTTTTTTTAAACTTGAACTTTACTTCGTT
AGATTTCATAATACTTTCTTGGCATTCTAGTAAGAGGACCCTGAGGTGGGAGTTG
TGGGGGACGGGGAGAAGGGGACAGCTTGGCACCGGTCCCGTGGGCGTTGCAGTG
TGGGGGATGGGGGTATGCAGCTTGGCACTGGTACTGGGAGGGATGAGGGTGAAG
AAGGGGAGAGGGTTGGTTAGAGATACAGTGTGGGTGGTGGGGGTGGTAGGAAAT
GCAGGTTGAAGGGAATTCTCTGGGGCTTTGGGGAATTTAGTGCGTGGGTGAGCC
AAGAAAATACTAATTAATAATAGTAAGTTGTTAGTGTTGGTTAAGTTGTTGCTTG
GAAGTGAGAAGTTGCTTAGAAACTTTCCAAAGTGCTTAGAACTTTAAGTGCAAAC
AGACAAACTAACAAACAAAAATTGTTTTGCTTTGCTACAAGGTGGGGAAGACTG
AAGAAGTGTTAACTGAAAACAGGTGACACAGAGTCACCAGTTTTCCGAGAACCA
AAGGGAGGGGTGTGTGATGCCATCTCACAGGCAGGGGAAATGTCTTTACCAGCT
TCCTCCTGGTGGCCAAGACAGCCTGTTTCAGAGGGTTGTTTTGTTTGGGGTGTGG
GTGTTATCAAGTGAATTAGTCACTTGAAAGATGGGCGTCAGACTTGCATACGCAG
CAGATCAGCATCCTTCGCTGCCCCTTAGCAACTTAGGTGGTTGATTTGAAACTGT
GAAGGTGTGATTTTTTCAGGAGCTGGAAGTCTTAGAAAAGCCTTGTAAATGCCTA
TATTGTGGGCTTTTAACGTATTTAAGGGACCACTTAAGACGAGATTAGATGGGCT
CTTCTGGATTTGTTCCTCATTTGTCACAGGTGTCTTGTGATTGAAAATCATGAGCG
AAGTGAAATTGCATTGAATTTCAAGGGAATTTAGTATGTAAATCGTGCCTTAGAA
ACACATCTGTTGTCTTTTCTGTGTTTGGTCGATATTAATAATGGCAAAATTTTTGC
CTATCTAGTATCTTCAAATTGTAGTCTTTGTAACAACCAAATAACCTTTTGTGGTC
ACTGTAAAATTAATATTTGGTAGACAGAATCCATGTACCTTTGCTAAGGTTAGAA
TGAATAATTTATTGTATTTTTAATTTGAATGTTTGTGCTTTTTAAATGAGCCAAGA
CTAGAGGGGAAACTATCACCTAAAATCAGTTTGGAAAACAAGACCTAAAAAGGG
AAGGGGATGGGGATTGTGGGGAGAGAGTGGGCGAGGTGCCTTTACTACATGTGT
GATCTGAAAACCCTGCTTGGTTCTGAGCTGCGTCTATTGAATTGGTAAAGTAATA
CCAATGGCTTTTTATCATTTCCTTCTTCCCTTTAAGTTTCACTTGAAATTTTAAAAA
TCATGGTTATTTTTATCGTTGGGATCTTTCTGTCTTCTGGGTTCCATTTTTTAAATG
TTTAAAAATATGTTGACATGGTAGTTCAGTTCTTAACCAATGACTTGGGGATGAT
GCAAACAATTACTGTCGTTGGGATTTAGAGTGTATTAGTCACGCATGTATGGGGA
AGTAGTCTCGGGTATGCTGTTGTGAAATTGAAACTGTAAAAGTAGATGGTTGAAA
GTACTGGTATGTTGCTCTGTATGGTAAGAACTAATTCTGTTACGTCATGTACATA
ATTACTAATCACTTTTCTTCCCCTTTACAGCACAAATAAAGTTTGAGTTCTAAACT
CA
>uc001pyz NCode human ncRNA array Probe: IVGNh27660 Primary
Accession: uc001pyz
(SEQ ID NO: 35)
GAACAGCTATAGGATCTAAAGTTCCATTACAGCTTACTGTGAAAGAATTGACAA
GACTGGCCTCAGACAAGCTAATCATGGTGCGACTCTCTCCCTTCCTCATCCACCT
CTTTGGGGACAAGAGGATTACATCTCAGGCCAGCAAGATCAGCTGCTTGAAGCT
CTGTGTAAGAGCACTGCACTGACGGTTTGGAGACCTGAGCCTGGGTCCTGACTTT
TCCATTGACTAAGCTCTGTGGCCTTGGGCAAGTCACTCCCCCTCTCTGAGCTTCAG
TATCCTCCTGTCACAGGAGGGAGTTGGGCTAGATCATCTTTAAGGTAGGTTCTAG
CTTTGACATCATCTTGGGGGTTAGGCCAGAGGCTGGGAAGACTGGGTGGACTTTC
TCAATTGCTCTGCCAGGAGGGAACAAGCCCAGAGGCTGAAGCTTCCCAGTATTTA
GAGGTGTGGTAGGGCAGTGTCTGCATTCCCAGGAGACCCAGGGTGATTAAAATT
TATTCTTTAGGTGGCTAGGAGGGCTGGGGAGGCCCAGTGGAAGAGAGAGAGAGA
GAGAGAGAGAGAGAGAGAGAGAGAGAGAGAGATCGAGCTTGATGTATTGCTCA
GTATTCACTTAGAAGGGTTTCTTTCTCTTTGGCCTAGTTTGTGAAGGGATCTTCCT
TTGGACTTTCTCTAAGTTGGGAGAAGAACATTCTTTTCAATGGAGCTCATCTTCTA
TCTCTAGGGTCTGTTCAGCCTTTCATCTATCCATCCTTCCTCTTTATTGGTAGAAG
AAACAGTGGAGAGTAGCCACTTCTGGTTCTAGCACTTCTCTTTTGTTAAGATAGG
GTTTGGATTTAGTATGAAGCTTTGGCTAAAACCCTTGGGTTTGCCTTAGAACACT
GACACTAAGAACCTGGAATGACATGGGGAGGACAAAGAGAGCTCAAGAGGAAT
GCTTTGTGAGAAGTGGATTCTCTCCGTGTCCCTGCCCCCCACCCAAACTTGAACT
ATACCTATTACATTTCCAGGCAGTATCCCTAAGATGAGATCCTGGAGAAAGGACT
AGGGGAAGTATCTTTCTGGATGCTTGTGGTCCCAGAAGGGTACTTTCTGTGTCAT
ACCATGCCACTTCTTTAAGCTCTTCAGGGCAGCCAAAGCCAGCCCTTTTCTCCTAC
TGCCCCCAGGAGAAATAGCACTCTTCTCCCTTCCCCCAGATGGCAGGGCTCTGGC
CTCCCTACACCTCATACCCTGCCTGCCTCCTCCAGGAGGAATCTCCGGGGCCCCT
TCCTGACTCTCCCCACCTTCGCCACTTGTCTCTAGGCTATGGGACAATCATCCCAT
TCACCACTTGACATCCTTGACATCCTTGACTTTCATTCCCCCAACCTCCAGCAGGT
TGGCCCCAATCCTCTTCACCTCTGTGTTTTCTTCTAGAAGATGCATTTTGGGTCTG
AGAGGAGCATTTTCCTGGAAGGCCATCTTTTAAGGCCCCTGCTTGCTGTCATAGT
GCAGAGCAGAAACTTGCACACTATTTAGAGAGCTCCCTTCCCACCTCTCTGCCCA
GCCTTGTTACCTCACTTCTGCTCTGGCCATGGCTGTGAAGGGCCCAGCCAGCTCC
CTGTTTTGATGTTCTGTGCAACAGCTCCGGGGTCTTGTGACTGGAGATCCTCAAC
AGGCCCTGGAGCCAGGACTGGAGTCTTGGCAGCTGATGAGCAGCACCTTGCCGG
CCAGGAGGAGCTGATGCTGACGATCTCCCCAACATCTGAAGGCTTAAAGAACAT
TGTCGTTCTTCAGCCCTCCTTGCTTCTCTCAATACAATAAGACATTGCAGAAGCA
AAAGGGTGGCCTCTGCTCCAGGCAAGGCAGCTGGCTCTGTCTGGGGCATCGGCCT
GGGGCTTGGGTGCCACGTGCTGAGATTGCATAGTCAAAACAGCCATTTTTGCCAA
CAATAGCTTGTGGCTCCCCACATTTTCCTACCCTGCACTCAAGGGCCAGACCACT
CTCTGCATGGACCAGACCATCTTCCCAAACCCATGGTGCTTTTTCCCCAACTCAA
CCTAGACTCCAAGGTGGGGAGGGATGGGTCAGAGGCCATAGTGGCCCCTGGATA
ATCCTGACGTGGGGTGGAGTGGGGTGAGGCAGAGGGAGCAGCCCCAACACCTGC
ACTGGGCCATCTATGGGAAAGAACACGGGTCGAGTGCAGTCGAGTTGTCTGGCC
ATCTGTATTTGGATCTATAACTGTACTTTGCCTGGCGCTGTGCGCAAGGTCAGAA
AACTTACTGCTAGTACCTAGAAACACACAAGGCTGCCCAGCCAAATCTTAATGTA
AAGTAGCTAGAGCCATGGAAGTACAGTATGAATTAAAAAGAAAAAAGTATTGAA
CTACA
>uc002llc NCode human ncRNA array Probe: IVGNh31353 Primary
Accession: uc002llc
(SEQ ID NO: 36)
GCTGACTCTCTTTTCGGACTCAGCCCGCCTGCACCCAGGTGAAATAAACAGCCAT
GTTGCTCACACAAAGCCTGTTTGGTGGTCTCTTCACAGGGACACGGATGAAATTT
GGTGCCGTGACTCGGATCGGGGAACCTCCCTTAGGAGATCAATCCCCTGTACTCC
TTTTCTTTGCCCTGTGAGAAAGATCCACCTATGACCTCAGGTCCTCAGACCGACC
AGCCCAAGGAACATCTCACCAATTTTAAATCAGACCTTGAAGATTTGTTGTTCAA
GGAGAAACTGAAGAGCAAGAAGGAAAGTGAGAGCCAGCAATACCAGCAGAGCC
AGATCTGAGCTGGGAGAAGGGGAGAAAGTTTGTGAAGAGGAGATCGGTGACCTG
GGCTCCTTATGTGCCTGAAAGAGTTTGAGTTTCCTGTTAACTCCAAATCAACAGT
ATTTTCAACAAGAAATGTGCAATTGAAATCAAGTGCTGTTTAAGTGCAGCTAGGA
TTTCCACAGGAAGACACTTGCAGTGAACAGAGTTATGGAGCAGCAAAAACACAG
ATCTATTTGGAAAAAGAGAAAACATATGCGTTGTATTTTGCTTCAATTATAAAAT
ACCATCCTCTCAAAGGTGGTTCTAAATTACAAAGGACTTTGATTTCTAGGTAGAT
TCTGGGTAGAGACTTCCTTTCATATTGAGGCATTAATGACACCTTTTAACCTGGG
AAGCAATATGACTGGAGTTGTACTTTGAGAAGATTAATCAGGTTTGGTTGCAGAA
TGAAAGAGAAGATGAAGTCAAGAGATTGGTTTAGAGGCTCTAGCAGAAGCTTAG
TCATATTTCAAAATGATCAAATATCAAGAAAAATTCTGAGCTGCATAACTTGTAT
AAAGTAATTTTCAGTGATTTTTTTCATGGTTATGATAAAAGAACTGGATTAGCAG
AAACTTTTACCCTGAATCAAGATTTAATTTTTCTTTGAGCTCATCTTAAGGATATC
GGAACATAGGGAGCAAACGATGGTGTGGCTGCCTCAGTGCTTGATTTTTAACGGT
TTTGAAGAGAATAGTTACATTTCTTCTCCTAGTAAGAACTAATAAATACATTAAC
AGAAATGAATTCCCTATCCCTTTGTACACTGGTCTATTTCTTCAAAACATTAAATA
CTATTGATAAGAT
>LOC100506411 Agilent Human SurePrint G3 Probe: A_19_P00807053
Primary Accession: ENST00000554032
(SEQ ID NO: 37)
CCCATTGGGATGTTCATTAGAACTCTGAAAACTACAGTTCTCCCCTTTATGAGGA
CTGCACCACAGCTCGCCCTCTCCTGGGTTCCGCCTGGTTGCAGAGTGAGCCCATG
GGACAGCCCTCTGAAATTATACTGCTTACAACCATGCTGAGTCTGCAAGGACTTC
GTCCAAGCCTTTCCGTCCAGGACCTCAAACAGATCCAATCACAAGAAGAGAGAT
TTCAGGAAAGAGAAAATTATTCCTATCATCGGGGTTTTTGAAGAACATGAAATGA
CTGGGAAAATAATCATGTTAAGTGGAAAAAAAAAAGAAATCTATCTGTTGTAAT
TTTCAAATAATTTTTAAATAAATTTGAAAAATTAAGAGAA
>LOC100129480 Agilent Human SurePrint G3 Probe: A_21_P0000128
Primary Accession: NM_001195279
(SEQ ID NO: 38)
ATGCACTGCGCAGAGGCTGGGAAGGCTTTAATTAAATTCAACCACTGTGAGAAA
TACATCTACAGCTTCAGTGTGCCCCAGTGCTGCCCTCTCTGCCAGCAGGACCTGG
GCTCGAGGAAGCTGGAGGACGCACCTGTTAGCATCGCTAATCCATTTACTAATGG
ACATCAAGAAAAATGTTCATTCCTCCTCAGACCAACTCAGGGGACATTTCTTAGA
GAGTATGATGGAAGGTCTGATCTTCATGTTGGAATAACTAACACAAATGGGGTTG
TGTATAATTACAGTGCACATGGTGTCCAGCGAGACGGAGAAGGGTGGGAAGAGA
GCATAAGCATCCCATTACTGCAGCCCAACATGTATGGAATGATGGAGCAATGGG
ACAAGTACCTGGAAGACTTCTCCACCTCGGGGGCCTGGCTGCCTCACAGGTATGA
AGACAACCACCATAACTGCTACTCTTACGCACTCACGTTCATTAACTGCGTTCTG
ATGGCAGAAGGTAGACAGCAACTGGACAAGGGTGAATTTACGGAGAAGTACGTG
GTCCCGCGGACAAGGCTGGCATCCAAGTTCATCACACTCTACCGGGCGATACGG
GAGCATGGCTTCTACGTCACTGACTGTCCCCAGCAGCAGGCACAACCCCCTGAGG
GCGGCGGTTTGTGCTGAGAGCTATGTAAGCGCAGCCTGGACGCTGGAGGGTAGG
GTGGTTGCTACCTTTAATCAGTACTATGGATTTCTAAATGCATTTAACTGTGGTTA
ATAAAAGCGTGTATGGGCCGGGCATGGTGGCTCACACCTGTAATCCCAGCACTTT
GGGAAGCTAAGACAGGTAGGTCACCTGAGGTTGGGAGTTTGAGACCAGCCTGAC
CAACATGGAGAAACCCCGTCCTTACTAAAAATATAAAATTAGCTGGGCATGGTG
GCGCATGCCTGTAATCCCAACTACTAGGGAGGCTGAAGCAGGAGAATCGCTTGA
ACCCGGGAGGCGGAGGTTGGGATGAGTTGAGATCGTGCCATTGCACTCCAGCCT
GGGCAACAAGAGTGAAACTCCATCTCAAAAAAATAAAAAATAAAAAAT
>XLOC_002335 Agilent Human SurePrint G3 Probe: A_21_P0002106
Primary Accession: ENST00000458351
(SEQ ID NO: 39)
TTTCTGTCTTCCTCAACCCCTCAAGATCAGCGCTTTAGCTGCAAGTAAATGCCTTC
TTGCATTGGATTCTTCCCATAAACTTCCCTGCTCATTTCTCCCGTGGATTGGGCCT
TCTATGACTGCACATATATAGTCGCTTCAGAATAGAAAGCCGCTTTCTCCCTTAG
CAAGATGCTCTTGTTTGGAGGTGCCTATGGGCTAAGGTTTGCAGAATCAGCTCCG
AGACCACCCCGACTGGGAAGTCAGATGAGATGGTCTGTCCTCTTCAGCTAATGCC
CATTGTCCTTACTGTGGAGTATCAAAAGAATAACGGACATCACTGAAGAAAATG
CACTTAACATCCTGTTATAAAACATATTTTTATTTATTTTTTTCACGTGACTACTTT
TCTCTTCACCCCCTACTTTATTCACACTTTGAGAACAGACTGAAATGCATGTATTT
GTATCCTAAGTGCTCAGATCTGATAAGGTCTGATTGCTGGAAAACAATGCATGAG
AGTTTATATTCATTTAGCAACAACACACCAGTCTTCTAAACTTATTCTAATTTAGA
CATGTAAAAAGTACAATAGCAATGCATCTGTATCTGTCAGACTAAGCTAGCTTAT
GCTACAATTGTATATAAAACAATAGCCTCAGTGACTTAAAACACAAAAGCCTCAT
TTCTCACGCATGCTACATGTGCATTGCAGTGGAGTTTGTGCATCATAATGACTCA
GGGATCCAAGCTGACTGAGGCTCTATCTCCACTTGTTTCCATGATCACAAACACA
GGAGGAGAGGGAAATGTGAAGGACATGCTGGTTTCACAAGATTTTGCTCAGGAG
ACAGATGTCAATTTCCCTCACAGTTCATTGATCAAAGCAAGTTGAAAGGAGAAG
ATAGATATGAATGGGGTAGAGAATTCTAATCCTCTCCTAAAGAGATAATGAATAT
TGCTCCCAAATATTTTCCCCAAAGCTAGGAGAAGAGGCTTCAAATTCAACAAATC
AGGCTGAAAAGCCTATACTCTTAATCCTATCAATCTATCTGTGTAATTACTATAC
ATAACTATATGTGCTATCTCGGAACACATACAAACATACACATACTCACACAAAT
ACATAAGTAGATGTATATTCCTTTTTAGCGTATTACAAAATGTAAAACCATTTCC
AGATTTCTGTCCACATCTAGATCTCCCTTTGCCCCAATATTACAAACTTGGTGTTC
ATACTTTCAATGTGCATATTTTCATAATTTCATAATAAAGTTATCAATAAAAATA
>XLOC_002871 Agilent Human SurePrint G3 Probe: A_21_P0002781
Primary Accession: ENST00000498005
(SEQ ID NO: 40)
ACCAATGTGATGAGTGTGGGGAAGGCCATAGAAAGGACCGGCGAATGCTGGCAT
TGATGTGTGTTATTTTAACATTTCTGAAATCCTGTTCTTAGTCTGCACACCTTGTC
CGAGGCTCCGATGTTATCCAGGTCACCAGGTATGCCCCTGGGCTCCTGCCGCAGC
TGATCGGGTGCTAGGTGCTGAGGATACACGTCTGGGAGAAAGCAATTGGAAGAA
ATGCAAAGCTCTTCAAAGGAGACCTATAAAGTCATCTTTGTTTTGTTCATTCTTCT
CATGTTTCTGCATTCTGGGCATTCTCCTAAATTGGGGAGAAACCAAAATGCCCAG
AAGTCAAATTCTGCAACTGTCATCATGCAAAATGTCAAATGAGAGAACCAAAGT
ATGCTGGATTCTATATTGTTAGGAAGGGATGGTTAATTTGATTGACTCTTGGGAG
CTATTTTTCTAGCATTAAGTAATTCTAGGGAACCCTTCTGTGATCATCTCTGAGTA
AATAAAGAAGTGAAATTGCAATTCAAATAA
>XLOC_003734 Agilent Human SurePrint G3 Probe: A_21_P0003853
Primary Accession: TCONS_00008904
(SEQ ID NO: 41)
GAATGGTTTTTAGGATAATTTTGCCTCAGTAAATCCTCTCTACATTCAGGCATTTA
TTAGGCCATTACTTGTTTTGGGACTACAGATTATCCTGGCAGCTCAATAACTGGA
TAAACAGGACTTTAGTGAAAGATTTTCAGAGGTTCTTTAGGGAAAAGAATGACC
AGGAGAAGGTGGGTGGAAGCCTTCAGTTCTTTGACCTCTTGCACGTAGAATCCTA
AAACTGATCATGATTTTAGCTAGGACTGACCTTTCCTAGCTTGTAGGGTCACTGT
GAATTTTGTTCATGTCTTAAAAGGTTTAAGTTAACCTAGTTCACTGTTACCTACAC
AAGTAACAAGACGGCCAATAGGACCTGTCAGCATGACTTCGACATGCATTCCAG
GCATCTTTCGGGGAGTTTAGATTTACTGTGTCATTTCAGAACCCAACAAAGGTGA
TGGAAGCTCTTAGGCCAGATTAAATTTCATGGAACGGAGGCTGCAGAAGTCTGT
GCTGCTTAGTGTGTCAGCTGACTTTTTACTGGGACAAGTCTATGAAAGGCCCACC
TGTAACAAGGCCCCTTTTTGCCCTGTGGATATTTTAAAAGAGGGAATTTGGTGTT
GACAATCTTACTTACACGACTCTTGCTAAGCTATTTGACTAAGGGTTTCAATCAG
ATGCTTCCCACCTCACAAGCAAGGGTCAGCTCTATTTGCAAATAATCCATGAATA
TGTTTGTCTAAAACCTGCTGAAGAGGCATGGCAGCCACTTCCATGCTGCTTTTGG
TAATGGGTAAAGAATATGGCCTTTCAGATAGATCTGGTGGCTTTTCCCCAATAGT
CACCATGTGGAAACTATGCAACTAAATTCAATGGAAATGAAAGATACAATATAA
AATAGCGGGTCATGGCCATAAGCTGTGTCCTGAACTAACCAACTCCAAGCTGAA
GGAGGGTGTGTACTTTCCGAAACTTCGAGGCCATCTTAGTAATTATTTTAGCAAT
AATTACTAAAATGTACATGGGGTGGGGGAGCTCAGCTAAAATATCCTTACTTTGG
TGCAATAATGATCTAGGTTCTTTTTCCTAGGCCTAGGCCTCCACCTTGAAAGACA
GGAACAGAAGTTCACTGTGATGTGTGACCCTGGACAGAGATCAAACAGCTCCTTT
CTAGACCCAGATGACCCAGAACGCAGAAGCCTAGTAGTTGGTATCACCAGTGTC
TCTTCAAAAGGGCCCCACAAAAGGCTGTCCATTAATTTGTTTCATACAGTAAGCG
AGCTTTTACTGAATACTCCCTCTGTTAGGTAGCATGCAGAGTGCTAGGGCTGGCA
CATTCCTGCCTTCCCACCAGAACCCTCCAACCTCCTCCCCAGGCAACAGAACACA
GGGTTTGGGCCTGACCAGGCAGAGCTGGTTCAAGCCAGCCTGGGGCAGAGCCAG
TTTTCCAGCACACTTCTAACTTCTAGTCAGAGCCTCAGCATTATACACCCAGCCTA
CAGGTGTGTGGATTCCTGAGACAGATGGCAATGGCATCACCTGTGGTGCCAACTC
ATACATTTTAATGAGATTTCTCCCTGAAGGGTGAACCAGTAGACCAGACTAAACG
CACACTCATGCAAGAATGTAAAATTGTATTTCACTGAGGCCCCTTTATAAGCAGA
GCCATCTTTGCGAATTTCTTGGGGTGTTAATGTAAACATATCTTTAGAATATCTCA
TCGGGTTTCAGTCAGAGCCATGCTTTGGGTTTTTCCTAGCAGCAGTGATGATATC
AACTTACAAGGTTTGGCTTTCAGGATTTCAGAAGCTGGCATTCAAGACAACAGGC
AGTTTGTCAGAGCTGAATGAGAATCAGCCTGGACAAATCAAGTGCTTTAACAAG
GGCATCTTCCTCTGGGAATAATCAGTCCTTAATACAGTTTGCACTTGACATAATA
GTTTTGGTAAATGTCTTTTTCTGGCTGCACCCCCTTTTAAGTAAGCCTTTAATTTT
AAATGGTCTGGAAAGATCTTCGATGCTTTCTGTAAGGTTTAGTCACCAAGAAGCC
AGAACTTTTGGTGAAAACAGAATTTATAAAATGAAACTGAACCTTCTCCTTTCTT
ACAAAATAAAGATCCTGTCAGACTCCAGTCTCAGACCACCTTTGCCCATTTGTAA
TTCAGACTTGCAGAGTGAGGAGAGAACTGCTTCAGCCTTACTGTCTTGTAGAGAG
ATTTGGTGAAAATCATGTTACTTTAGACCCAGTAGTTTTCAGGACCGCAACAGGA
TGCGGGGCACCTGGCTTCCCGGGTAAGGTCACATAGTCTCTTAAAATTCTGTCAC
TAATTTTTTTAAACGACTTTTTTTAAAAAGCCACCTCCTCATGGGTGTCCACTTTT
TTCTAGTTCCTCAGCTGCTTCTGGAGCAGTGTTCACAACGGGAATGTTTTTACTGT
CCTTGGTAGGCTACAGGTTCACAGCTTCAAATCAAGGCCTCCAAGGATTTTATTC
TCTTACATCACAGTTTTGACAAGTATGCTTTTAAAAAACAACATTTGCAAAACTG
GTCTTTAAGCGACGTGAGTCAGAGGTAACAAAGGCATATATATACCGAACAAAG
GTGCTCCGGTGCAGTGGAGAGAACAGTATTAGTGTCGCAAGCACAGGAGTGCAG
ACAGCCCCGCCTTCATCGTGATGCCTGCAGCACACCACGATTATCATGAGAGGTC
AAGATTTTGATTTACTAATTTATAATCTTATTTCCAAGCAAAACAAGTCAATTTCA
TGTTACAACTTTTTTCTTGTTTCTTTTTATCTTGTTTGGCCTGAGGGTTGGGGGATT
TGGGGGAGTTGTCAGCTGCACAATCTTTGAAGTGTAAGTTAATTTTTATGTGATA
TTTCAGTATATATTTTATTGATTAAA
>XLOC_003734 Agilent Human SurePrint G3 Probe: A_21_P0003854
Primary Accession: ENST00000508664
(SEQ ID NO: 42)
AAGATATTCTAGGCCCCTTGTTGCTTCAGCCATCAGTCTATAAATAACACAACAC
TAATTTTCCATCAAGTAACAGCTTAAAACAGAACACTGTCAAGATTTTGATTTAC
TAATTTATAATCTTATTTCCAAGCAAAACAAGTCAATTTCATGTTACAACTTTTTT
CTTGTTTCTTTTTATCTTGTTTGGCCTGAGGGTTGGGGGATTTGGGGGAGTTGTCA
GCTGCACAATCTTTGAAGTGTAAGTTAATTTTTATGTGATATTTCAGTATATATTT
TATTGATTAAATTTATTGGAAAACTT
>LOC154822 Agilent Human SurePrint G3 Probe: A_21_P0005276
Primary Accession: BC013024
(SEQ ID NO: 43)
ATGAGATGTTAGTTGGTACAGGGAGGGGTTTCCAGGACCCGCACGCCCTTGCGG
AGTGCCTGCTGGAGGGAGCCGGTGTGTCCAGGACACCCTTGCGGAGTGTCTGCTG
GAGGGAGCCAGTGTGTCAGTGAGATGGCTATGCCCCTGGGCTGCTGTGTCCCAG
GTTTCCTCAGTCTCTAACCCTTTGTTCTCACAGGGGATGGACTCTTGCTTCTTTTC
CCAACTCCACCAAGAGGGACCGTCCCAGGACGTCCTTCCCCGGGCATCTGGCCCT
ACAGCTGCCTGAGGTCTCCATCACCGTTGGCGCCATCAGTCTGCTGTGCAGCCAG
CTGTTGGTTTGGAGAGCCTGAAGAACTGCAGTTCACGTCTCATCTAAAGGAGCTG
AAATGATATTGCAGCTTTTTCTTTTGGTTGCGTGCAGTGAGAATCTGGGAGCTGA
ACCTGTTATCTGCATGGTCTTCAGAAATCAGGCAAACTCGGAAAATGCCAACGCC
AAAAATGCTGATGGGTGACAAAGTGTCACAGGTGTGATGCATTACAAATCTCAG
GACTTTTGTTCACTGGATTTGAAAGGTCAAGCTTCACAGGAAAATGATGAAGTCC
CAAAAGACCAGAAATATATTTCAGAAGATGCCAGTTACTACTTTAAATGTCAAAC
CAACATTTCAGAAATAACTTTCAATGATTATTTCCTGCCAAGAAGGTGAACGCTG
GAGACCTTAATGGTGGAAGATGGAGGGCGTCTTTCCTTCTGTTAAGCTGACAACT
TGGCTTCCATCTTGTGAGGACCTCACCCTACCTGGTGGCAGAGGACGTCTGACGC
CCTCAATCATTGCCATTACACTTCCCAGCCTGGTGGTCAGTCTCCTGGGGTCTGTG
TGTTAACAAACCATCGACTGGACAATCGCAGTTTTCCTTATGAAGGCTTACTTTA
AAAAGGCTCTGGATTTTCAGAAGCGAAGTCGCTTTCATCCCCGATTCAGACCCAT
CCTAGTGGAGGAAAAATCCTACCAGAAGAAGGGCTGACCATAGGAACTTGCCAT
TTCCTTGACCCCATCATATCTGAGGAAAAAACAACAGAAAAGGTCAAAACCCAC
GTGTACGCCCAACGTCCTGATTGACGACTTTGCCTGCAGCTTCTGCTTTCCTGAAA
TTCGCTGCTGCCTTTAGAACCCTTGTCTGCAGCCAGTGGGGAGTTCAGGACTTAG
GCGGAGCTGCCCCACCCTCCTGCTTGGCACCCTGCAAATACATGCCCTCCCTTCC
ATCGCTGCAGACCTCAGAGTGGGCGTCCGGTCTCCTGTGCGGGATGAGAATACA
CACCCTCCCTTCCATCGCTGCAGACCTTAGAGTGGATGTCCGGTCTCCTGTATGG
GATGAGAATACACGCCTTCCCTTCCATCGCTGCAGAGTGGACGTCTGGTCTCCTG
TGTGGGATAATACACGCCCTCCTTTCAATCGCTGCAGACCTCAGAGTGGACGTCC
GGTCTCCTGTGTGGGATAATACACGCCCTCCTTTCAATCGCTGCGGACCTCAGAG
TGGACGTCCGGTCTCCTGTATGGGATGAGATACACTCCTTCCCTTCCACTGCTGC
AGACCTCAGAGTGGACGTCCGGTCTCCTGTGTGGGATGAGATACACTCCTTCCCT
TCCACTGCTGCAGACCTCAGAGTGGACGTCCGGTCTCCTTTGTGGGATGAGAATA
CACTCCTTCCCTTCCATCACTGCAGACCTCAGAGTGGACGTCCGGTCTCCTGTGC
GGGACAAGAATACACTCCTTCCCTTCCATCACTGCAGACCTCAGAGTGGACGTCC
AGTCTCCTGTGCGGGATGAGATACACTCCTTCCCTTCCATCGCTGCAGACCTCAG
AGTGGACGTCCAGTCTCTCTGTGCGGGCCAAGTGTACACAGTTTTGTTCCGTCAC
AACTTCCACGACAGGCCAGTGTGAGGTTTTTGAGCTGGTGCTGACTGAAAACTGT
CAGCTGCCCAAGGACCTGGGAGCTCTGCTCCCCACTCCTGGTGTGCGGTCTTGCG
CCTGGCCTCCCTGCCTAGGTTACATGCAGTGGTCATCCCGGTCGCTCCCACACCC
GTGTGGGCTCTGGGATCCCCTCTTCCAGCCAGCCCAGGGGACATCTGGCTGTCTC
AGGACCCAGCCATCTGTAAAAATTAGGCAGGTCCCTTCAGTATGCTCCTGGTCAA
CAAAGAAAAACTTCAATTTTGAGAATGGCATCTGTATTCCGAAGTGTTCTCTCAG
ATGTTTGAGTTCCACTAAGTAGATTTTCTTAGTCTGCTGTATCAATGACACAGAG
AGACGTGCATTAAAACCTCAACCATGTGGATCTATTTCTTTTCAGTTAATTTTGCT
TCATGTATCTTGAAGCTCTGTTATCAGGTGCATGCACATTTGGGATTGTTATGCTT
TCCTGATGAACTGACCTTCTTTCATTATGCAAGGGGAAGAAGATGCTGCATACAG
GATGGAATATCCAGGGGAAGACGTCTAAGGAGAGATGCCCAGCTGGGAGTCCTA
TGCAAGGGGAAGAAGATGCTGCATACAGGATGGGATATCCAGGGGAAGATTTCT
AAGAAGAGATGCCCAGCTGGGAGTCCTATGCAAGGGGAAGAAGATGCTGCATAC
AGGATGGGATATCCAGGGGAAGATTTCTAAGGAGAGACACCCGGCTGGAAGTCA
AGATATGTCAGTTGTTTCCATTATAATAAAACCACTCATGTTAGATGAGCTGAAC
TTTCCCTTTTCCCCAGTTCTTACGATCAAAAAGTGGCTGTCCTAAATTTCATCACT
CAATATCCTTGCTAGAGTCTTCCTTTGTCAGCCAGGCTGGAGTGCAATGTGCAAT
GGCACAATCTTGGCTCACTGCAACCTCTGTCTCCTGGGCTCAAGCAATTCTTCTGC
CTCAGCCTCCTGAGTAGCTGGGATTACAGGTATGCACCACCATGCCCAACTAATT
TTTGTATTTCAGTAGAGACGAGGTTTCACCATGTTGGCCAGGCTGGTCTCGATCT
CCTGACCTCAGGTAATCTGCCCACCTTGGCCTCTCAAAGTGCTGGGATTACAGAC
ATGAGCCATCATGCCTGGACATAAGTGAGTTTTATATTGTATTATAAGACTATGA
TACAGTAAAACCATGAAATCCAAATTTATAATATCACACTACATAATACAACTGT
AACCTCACCGCCCTATCCTGGGATGTGTGTCATTTTTATAGCCAATTATGGCCCCC
AGCTTTAGTTTTCTTTTGCTTATTGGAGAGTGTAATTCTCCCTTATTCTTTTTGCTT
TCTACAGTCTTGTGTACATCAGTTATCTGTTTTTGTCCTTTTGCCAGTGTTCAAAG
TGTTATTTTTCGTATTTACTTAAGCTCCTGCAGGGAGATTAGAATTTCTTCCCCTA
AGAAGAAATAAGTAATAGCGGAGACCTGCTGGGCACTGGTGGCGCCAGGCTTGG
CTCTGGGGCTGCCCATCCATCCTCACAGCATGGCGACTGGAGGGTCTTGCCCTGA
GGTCCCGTGTGCGGAGCAGGGCTTGGCATTCACTCCTAGGCACTGCTGACTCAGT
CTGTCCTGGTGGTGCTGGGAGGCCGAAACCCGTCATGCATGTAAACCGCCGGGC
CCCGTCTGGCATGGTGCACCTGTGCTGGGAGTGCCTATAGAGTAGGAAAAGTATT
CCTGGACCTTTAAAAAACTTAGGCCAAAAAAGTGTTTTGGTTGAATCTTTGGCCA
AATTGGAACTGCAAACTCTGTATTATCTCCCCTTTTGTGAAATTCTATGGAAAATT
CGAGCAAATAAATATGCATTTCCCAGTGAAAAAAAAAAAAAAAAAAAAAAAAA
AAAAAAAAAAAAAAAAAAAAAAAAA
>XLOC_007162 Agilent Human SurePrint G3 Probe: A_21_P0005873
Primary Accession: TCONS_00015107
(SEQ ID NO: 44)
CGCACCTGTAATCCCAGCTGCTCGGGAGGCTGAGGCAGGAGAATCGCTTGAACC
TGGGAGGCGGGGGGTTGCAGTGAGCCGAGATCTGGCCATTGCACTCCAGCGTGG
GCAACAGAGTGAGACTCCATCTCAAAAAAAAAGGTTAATCTTTCCAACTAGATTT
TCAAGGATGAGGATTTTGTTGTTGTTGTTGTTGTTGTTCTCAAATGTATTCCCAGG
GCTTGGAACAGAGCCTGACATATACTAGGCACTCAACAAATATTTGTTGAATGAT
TGTAATGAGTAACACCCATTTTTGCAGATCTTTGTCTTCTGAGCCTAGGGCATAG
GTCATCACTGCAGGGGTGAGATTGTCAAAATGGGAGTCTACAGCGCCAGAGACC
CAAGTTGAGGAACAGCCTATAAAATAACTGGC
>XLOC_007697 Agilent Human SurePrint G3 Probe: A_21_P0006269
Primary Accession: THC2779256
(SEQ ID NO: 45)
CAGACTTTCTTGTTTGCCTCATCCCTACCAGTGTCTTTCTCCCTACACCTAAGGTC
AATTACCAGCTGCCCTTTATCGTTGAACTTGATGCTTTCTTCTCATAGTAGAATTA
AGAGGAAAGTAAAATATTTTTTGTACCTATATCTTTATTATATTTAGACAAATCA
CAGAGTGAGAGAGTAGGGGTTTCAAGAAAAATAGGAGAGAGATAAAGGAGAGA
GAAAGAACTGCTTGTGGAAATACAGAATATCCCACATTTTCAATGTGGAAAGTGT
ATGAGGGTATGAAAGAAAATACTCAGTTTTTTTTGTCCTGTAAGAGGCAGCATTG
ACAAATGTGTACCAGAGTTTGGGTACATTTGAGCCAGTTCTTCAGAATCGTGGGG
TGGGAAATAGAACAAAATTATTTACACCTAATTCTAGGCAGATAAGTGTGCTTCA
AGGAAAGGCAAGGGCCTGGCTAGATTCTAGATGTTTTTAAACTGGAGGCCAGAG
ACAGCTTTAGGGAGTCCATATACAGGCACAAATTTATTTCTTTTATAGTCTTCTTG
CTCTTTGAAAATGGTCTTTATGCAAATACTCACTATATAACCAAAGTTTCTCTTTG
TTCCAGGCAGCAGTAGGGCTGATTGGAGCCATTGTACGTGTCGGGAACATATCA
GAACACCGAGAATAGCGTCATGTCATAAGGACTCAGAGCAGGTGGACCCTGCTG
TGATGCACAAAGAGGACCACGCAAGATATGATAAAGATCTATGTCACTGAATTT
TGGTTCAATTTTTGTATCTCAGCTTCCCGGAAATAAAAAAGAATTCTAACATTCA
TACTTTCAGTATTTTATGTGAGAGGTTTTGTTGTCAAAATCAAGTCTGAGAGCAA
TGTTTATTGGGGTCTTTAATTGGAGTCACCA
>XLOC_010807 Agilent Human SurePrint G3 Probe: A_21_P0008324
Primary Accession: TCONS_00022478
(SEQ ID NO: 46)
TTACTTTACATCAACATAGCAGAACAAATTTTTGGTGTTTCTTACCAAGAAAATC
TGCATCATTTGAAAGTATCCAAAAATGGTTTAGTGCACAACCTACACAACTAAGG
CGAGTAAAATCTTCTGTAGACTTGAGGAAGGAGAAGATCATAGCTCCTTTGGAA
ATCAAGAATGATATGCAAAGCAGTATAAAAGAGGTTATGTTTCAGAAAGCAAAG
GAATTGAAACGTCAGCTCCAGCTCACTAAGCAAAATAAAACTGAGGAGCCCAAC
TATGTGAAAGAAAGTATAGATGACATCTTTGATAACATGTGCGAAAAACACAGT
TTGAGAAATCTCTCTTTGACTCTCATTGAAGCGTCTAAAAAAGCTGGCATTAGTT
ACATTGTTTATCCCAAGAAAAAGAAGATGAGATGGAAGAAAAGATTGAAACAAC
AAAAACTTATATTCGTGCATGAAGAGTTATCCAAGCCTCCAAAATCTCTTGAAAG
GTCTTGTTTAAGTGATTTTCTTATAGTTTAAGAAATATATTGTGGTTTTGACCTTA
ATTTTATAATCTCACCCCATGAAGTTATTATTTT
>XLOC_010813 Agilent Human SurePrint G3 Probe: A_21_P0008331
Primary Accession: THC2542080
(SEQ ID NO: 47)
CGTTTTTTTAAGCCCGCCGGAAAAGCGCAGTATTCGGGTGGGAGTGACCCGATTT
TCCAGGTGCCGTCCGTCACCCCTTTCTTTGACTCGGAAAGGGAACTCCCTGACCC
CTTGCGCTTTCTGAGTGAGGCAGTGCCTCGCCCTGCTTCGGCTCGCACACGGTGC
GCGCACCCACTGACCTGCGCCCACTGTCTGGCACTCCCTAGTGAGATGAACCCGG
TACCTCAGATGGAAATGCAGAAATCACCCGTCTTCTGCGTCGCTCACGGTGGGAG
CTGTAGACTGGAGCTGTTCCTATTCGGCCATCTTGGCTCCTCCGCATTTGTTTTTA
TGGTGGTTTTGTATTGTTTTTATAGAGCTGCCCTCACATGCTTCAGCAACATTAGA
TGGTA
>XLOC_12_000735 Agilent Human SurePrint G3 Probe: A_21_P0010596
Primary Accession: TCONS_12_00000977 (Note: probe is in reverse
compliment orientation)
(SEQ ID NO: 48)
TTAAAAGGTACAATTCACAAGGTTGGAGGGGTAGCTGGAAGTTTCTGTGGTTACC
TTGCACTGGGGGGCTGCCCTGCCTCCACTCTCTCCCCACAGTCCGAGGGCAAGAT
GAGCACCCCCACCCAATGGCAGGACCAGCCCTGCGGGGAAATGTCAGCATGAGT
GGAAGCACGGCAAGGCCCCTTCCTTCTTGGCAAGGGGCTTCCCTGGCAGGCAGTT
CACAGGGTGTGTGGGTGGGGGGGATGCTGACCAGCTGCTCTCCTGGACCCTTCCT
GTACGAGCCTGTTTTTTTTTGTTTTGTTTTGAGACAGGGTCTCCCTCTGTCGCCCA
GGCTGGATGCAGTGGTGCAATCTTGGCTCACTGCCACCTCCACCTCCCCGGTTCA
AGCAGTTCTCCTGCCTCAGCCTCCCCAGTAGCTAAGAGGCACCCACCACGATGCC
CGGTTAATTTTTGTATTTTTAGTAGAGATGGGGTTTCACCATGTTGGCCAGGCTGG
TGTCAAAATCCCGACCTCAAGTGGTCTTTCTGCCTCAGCCCTCCAGAGTGCTGAG
ATGACAGGCGTGAGCCACCGCGCCCGGTGAGACTGTGGTTCTTGGAGGCTTTGG
GGATCCTCTTGTCCACCCCGTCAGGACCCAGCCTGGAGAATGAGGGGTGGACAA
GCTAAATGGAGCCTGGTCTTGGTGGGGCCCCGGTGGAGTCCTCAGAGATGCCAG
GCTCCTTTCGCGTCCTCGGGGACCGACTTCCAGTGGCTGCTGTGCCCTTGGGCCC
CCCAGTGGGGGACGCCCCATGGAGCTGGGCGAGGGCGGCTGACCTGGGCAGAGG
CTGCTGGCCCTAATTATCAGTCAGAGGCCCGAGGGGGGAGGCGGCTGTGCTGGT
GGCCGGGGGCCGGGGGGGCAGGGGCAGGCAGCGCAGGTTCCCGGTCTTGAGCGC
GCACTGCACCGGCCAGAGTGCCACACAGAAGAGCATCAGCAGCAGGGCAGAGA
CCAGTGCCATGCGCCTCCAGTCCCTGCAGCGCGCCCAGCAGCGGGCCAGGCGGC
CCCGGCGGGGGGCAGGGTCCCGGGCGGGCGCGGGCGGCTCGGCAGGCTTGCTCA
AACCCACGTCCACGCATACGAAACCGGGCTCGCGGCCAGGTGTGGTGGGCAGTG
GCTGGCAGCACAGCTTGGTGCCCTCCAGCCACACAGGCTCCTCACGCCGCAAATG
CGCCGGCATCCGGGCCTGCAGCTGGCGGCTGGTGCACAGCGCGGGGGCTCCGGC
GGGCGGCACGGCCGTGGGCTGCCTGCAGAAGGGGCAAGGTACAGCCTCACCACC
GGGGCGGCCCACAGGCTGAGCAGCCGCCAGCCGGGCCAGGCACTCCAGGCAGA
AGACGTGGGTGCAGGAGAGCTCCTTGGGTGTCTTGAAGATGTTGTCATAGCCTGA
GAAACAGATGGAGCACTCCAGGGGGGAGGCCACCCTCTCCGAGCCAGGGGTGCC
AGGGGACCTGGGGCTGCCGGCCGAGCTGGGGGACCTGGGCATCGAGGCTATGGA
GCTGCTCCGGCGAGGGGGTGGCACAGCCGTGTGCCACACCTGCTGGCCTGACGA
CATGTCTCTGAGCTGTGGGACAGGGACTGTGGTAAGCAATCACCGGCCGCCCCTT
TCTGGTGGTGTTTTATCTCTCCCTCCCCTCTCTCGCCCCAGAGATCCCAGGGAAGG
ACTCTGTTTCCTGCGCGCCACTCCAGAAAGTTCCTCCGGTGCCCCTGGAGGTCAT
TCTGCCCCACGTGCAATCCTGTCCTCTCCACCCCATCACATGGCTGCACCGGGGT
GAGCCTCCCACAGGGCCCCAGGCCTGCTCCGGGAATGCAGGCCGTGTGTAGGGG
GGTCTCACTGACCGCTCGGCAGACACCTCCTGTTGGCCCTGCCCCACCTGGCTGG
CCCTGCTGCCCGGGCAGAAATAATGGTGAGGATGACAATAGCCACAGTCGTCAC
TGTTTATGTCGGAGCTCTGCAAGGCTGGGCCCACATCACGGGACTCACACAACGC
CACAGTGTGGAAAAGGCCGCCCAGAGCATGGGTGACTCGGCCAGGGCCACCCCA
AGGGAGCTGGCGGGCCCTGGACCCTGGCAGATACGGCTCTCAGGCAGGCCAGGG
ACTCCAAGTCAAGTGAAGTGAGTTTGAACTCAGATCCCAGGATGGGTGCCTGGCT
TGGGCGGTGCAGGCCTGATTTGTAGGCAGCTATGTGAGGGTGGGGTGTGGGGGT
CTCTGGGTCTGGGGACCGGGCTGAGCCCCGGGGGCTTTGGGACGACAGGGAGGG
CCCAGGCAGGGGCAGGGGTCAGTGCCCGAGGAAGGTGCACGTCAGGCACGACCT
GCGGCCTGCGGGGCCGGCTTGTCTAGCTGCTGAGGGTCTGATGTGCACAGTGTGG
GGGTGGGACTTGGATAAGCCCAGCCATTCCCTCTGGGCCAGCCCACTGCCTCATG
GTCAGGTGATGGTCAGGGCACCCTCAGCCGCCCACTGAGTGGGTGTTTCTTCTCC
CTGACCCAATCCCACTTCATGGCAGGGACCCTGGGGGACGGACACTGGGGGATG
CTGCTCTGCCCCTGGGCATGGCTCAGGTGGGCATCTCAGCTGACCTGGGACCCTG
CTCCACCTCCCGCCCCTCCCCTGCACCCAGGATCCGCTGCAGGGAGCCACAGGGG
TCCCACCTGGAGGGAAGTGGGCAAGGGTGACAGTGAGACTCAAGGGCCTGGCCG
TGCGTCCCCGTGGGGCCCAGGAGGCTGCCCCAGAAGTGACTCCTGGCACTGCCCC
GCCCCACCCCTGACTTGCCAGTGAGTCCCAGACAGGCTGGCGGGATGACACAGG
TCACTGTGACCACCTGAGTCACACGCCGTCACTGTGAGGCCGTGAGTGCCCCAGG
CACCGGGACCTGGGGACTGTGCTCTGCGGCCTGTGTACCCCACAGAACCGGTTCC
TTGGCACGAGGCCCCACCCCTCCACGATGGTGCCCCACCCTGAGCCTGTGCAGGT
AAGGGGTGAACACGGGCTGAGCTGGCCTTACCTGGTGGCCGGGGGTCAGCGGGC
CTGGGCGTGGTCCTCCTCGCCGGCCACGGTTGGGCTCCAAGGCCCTGGGCTGCCC
TGCCGTGGCAGTGTCTGCTTCCTCTTCTCCGGGCCCGGCCCGGCCTGTGCTTCACC
CAGCAGGTATCCCTCCCCGGGGCCGGCCACCAGCAGCTGTCCCGGTGGCACTGGT
CTGGCAGGTGTGGCTTCTGCTCTGTCCAAGACAGGCGGGGACACAAGGAATGCG
TGCGCCGTCACCCGCACAGAGCTCTGGTCTGAGGCAG
>LOC100506922 Agilent Human SurePrint G3 Probe: A_21_P0011848
Primary Accession: XR_109888
(SEQ ID NO: 49)
GCGGCCGCGGCACCCTCGTCAGGCGCCGCCGCTGAGGGCAGGCAGCCCGGCAGC
CACTACACACGGACCCGTGACGTCGGGCGTAGCGCGGCGCACGTCACGGCCGCT
CGCTCGTGCGCGCGCACCCCTCCGCCCGGCGGTAGCGGAACCCGCCGCGGGCGC
GCGCCCGGCCCAGGGGAGTGGGTCGGCGCCTGCGCAGAGGCCCGCCACGCCCAC
ACACAGGCCACCGCCCCCACCGGCCGGACGGCGCGGGGATTCCCAGTCCTGGCT
CCGCCCCGGCCTCGGCCCCGCCCCCGCCCCTGCCCCGGGGCAGCCTGTGCTGTTC
CGTGTGCGCGGCGCATACGCACCTGGGTTGTCTCGAGCCTGCGGTAGTGGCCAGA
TCCCAGACATCCGAGTAGATCCCGTGAAAAGGTCTCCCACGTGGGCTGTGGACA
GGGCCCAAGGGTAGCAGAGCTAGCAGAGGCAGTGACGGACTGTGTGGCAGGTCA
TTTGCAAGGAGAAAAGCCGTCTGCCTCTTAATTTGTGGCTCAAGTTTCAGAATTT
TTTTCCTGAGGGACTTTAGAAATTACTTCAGGCTTGCCACCTAACCTTAAACCAC
CCCCTTGGAGACTGGCTAAGTGTTATTTGTGTTTTCTGTTTAGTTCTTATCACCAT
CGATACTTGGTTATGACTGGTTGTGTACATTGGTTAGCCCAGCAAGTATTACTTCT
CCAGCTTAACAGATGTGGAAACTTAAGCCCAGAGACATGAGTTGACACCCCACC
CCCAAAGCTAGAGTCTAAAACCCTTTCTTTCGCTCCTCATCTCCCACAGGATAAA
ATGCAAATTAATCAGACTAGTGGTGAGGCCCTCCGTGGTGTGACTAACCTGCATC
CCGACGTTTTCACCCTACTTTGATCCAGAAAGCACCTTTCCGCCCCATCTCTTCTC
CTTTCCTTAAATACCCCTTACAACTTCCTGTACCATTCTTCCCTGTTCAGCTTCTTC
TTGGTTTCTTCGTACATTCTGGATCCACCCCTTTCATGCATATTCCAGACCACATT
TCCACTGGAGCAGTTGAAATGAGAGAGATGGGAATGGGACTCACCCGAACCAGA
GGAATTTTTATTACAGACCCATTAACAGAGGTGTCAAAGTCACAGGAACAAGGA
TGTGCACCTCAGAAACACAGAGGTCAGTGGAAAATCAGTTTGCTTCTATTTGTTT
AAAAAATGGGGGACTTATGCATAAATCTAAGACCTTCTTGAATCTAACATTCTAA
GACCTGTATGCCACAGAAAGGAGGGTCTCAGAACGCCGGAGGATAGTATTTAAA
TCTTAAATATCTATATTGTTCTCCACAGTTACTGGGTCACCACATAGCAGGCATTC
AATAAAAACGTGTTTGTTTACTAAGTAA
>ANKRD20A9P → Agilent mis-annotated. The ncRNA corresponding to
A_21_P0012182 is XLOC_12_009136 in chr21. XLOC_12_009136 Agilent
Human SurePrint G3 Probe: A_21_P0012182 Primary Accession:
TCONS_12_00017143
(SEQ ID NO: 50)
GCCATACATCACTCTTTAGAATTCTGGTGACAAATTCTTTTTCTGGGTGGAACATT
GATGGAAAGTTCCAGTTTTCTCTCTCTGTTATAATAATGTTCTTTCAGGTAGTGGT
AGTTGACCATATTTAGCTAATTGAATGTCTTATAGTAATAAACTCTATCACAGAA
GTACTTACAAAAAACTAATTGTAGCATAAATATTAATTAGTATTATCAGGGATAT
GAAAGACCAAAAAGCTCTGTTATAGATCTATTTCCCCATGTACTTTATTGTACTTC
ATGTTGTTTCTTTTCTTTCTTGGCTTAAGCTCATATTTCGTTGACCAATTAGGCTTC
TTTTTTGTTTGTATCTCTCTTCATTCTCACATTTTAAATTGATATTTTTGGGGAGTC
AGGGTCTTGCTCTGTTGCTCAGGCTGCAATGTAGTGGCATGATCTTGGCATGCTA
CAGTCTCCACCTCTCAGGCTCAAGTGATCCTCCCACATCAGCTTCCCAAGCAGCT
GGGACTACAGGCACACACCATCATGCCTGACTCATTTTGGTATTTTTTGTGTAGA
GATGTGTTCTCATTATGTTGCCCAGGCAGGTCTCAAACTCCTGAACTCAAGCAAT
CCACCCACCTTGGCCTTGCAAAAGGCTGAGATTACAGGTGTGAGCCACTATGCCT
GGGCAACATTGAAACTGATTTAAATAAATTGATTAGGGCTGGGTGTTGTGGTGCA
CACTGCTTATCTCAACACTTCGGGAGGCAGAAGTCGAAGATTTACTAGAGCCTAG
GAGCTTGAGACCAGCCTGGGCAGTATAATGAGGCCGTGTTTCTACAAAGATAAC
AATAGAAACATTAGCATGGCATGATGGTATGCACCTGTAGTTCCAGCTATTCAGG
AAGTTGAGGTGGGAAGATTGCTTGAGGTCAGGAGTTTGAGACCACAGTGAGCCA
TAATCAGGCCCCTGCATTCTAGCCCTGGGTTGACAGAGTGAGAACCAGTTTCATA
AAAAGAGATTGACAAGAAACTCTTGATGCAACTCATTATAATTTTAAAATGGAA
ACTAATTCTTGATACTACCTTAGCAGTGTGTCCCCAAGAAAGTGTCAGAGCCTTT
ACGTGGACCTTCCCATGGAAAAGGAAACAGAATAGTCAATGGAAAAGGAGAAG
GACCTCCTGCAAAACATCCTTCCTTGAAGCCTAGCACTGAAATGGAAGATCCTGC
TGTGAAAGGAGCAGTACAAAGAAAGAATGTACAGACATTGAGAGCAGAAAAAG
CCTTACCAGTGGCTTCAGAGGAAGAGCAACAAAGGCGTGAAAGAAGTGAAAAG
AAGCAACCACAGGTCAAAGAAGGAAATAATACATACAAAAGTGAAAAAATACA
ACTATCAGAAAATATATGTCATAGTACATCTTCTTCTGCTGCTGACAGATTAACC
CAACAAAGAAAGATTGGGAAAACATAACCTCAGCAATTTCCCAAGAAACTGAAG
>XLOC_12_009136 Agilent Human SurePrint G3 Probe: A_21_P0012220
Primary Accession: ENST00000429521
(SEQ ID NO: 51)
GGACTATTTAATAATAAGGAAAATAAGTGCATTTGAAGCCAATCTCTCTTAATTC
AAAGCTCATTTCCATAGTGACCCATTTGGATCAGGAGTGCCTGACATTCGCATCT
GGGATCCTGACACCATTGATAGAAAACAGCCCTCATGCTTGCTGTGCACTATGAC
TCACCGGGTATTGTCAACATCCTTCTTAAGCAAAATATTAATGTCTTTACTCAAG
ACATGTATGGACAAGATGCAGAAGATTACGCTATTTCTTGCCGTTTGACAAAAAT
TCAACAACAAATTTTGGAACATAAAAAGATGATACTTAAAAATGACAAACCAGC
AACTCGTGGCAGCCATTGATGTTTACTCTGTCTTCATAGTTTTACTTTTTTCAGAA
GAGTCACATAGTTGGAATAATACTGTGGATATATTTTTGAATATTAAGAAAATTA
AAGCTCCATGGCAATTGAAGGACCTCCTGCAAAACATCCTTCCTTGAAGCCTAGC
ACTGAAATGGAAGATCCTGCTGTGAAAGGAGCAGTACAAAGAAAGAATGTACAG
ACATTGAGAGCAGAAAAAGCCTTACCAGTGGCTTCAGAGGAAGAGCAACAAAG
GCGTGAAAGAAGTGAAAAGAAGCAACCACAGCTAATTTTAGAACATGCACTCTG
ACAGAAAAGACATCTGAGAAACAAAACAAGCAAATTTGTTTTCCTTTTTGCACCT
GCCAAAAAAAAAAAAAGAAAAGCCTCAAGAACCAGAACTGG
>XLOC_12_009441 Agilent Human SurePrint G3 Probe: A_21_P0012326
Primary Accession: ENST00000447898
(SEQ ID NO: 52)
AGAGCGAGCTTCGGAGAAGCAGTGGTGGGTTCCATGTGATGGTGGAGTAGGAGG
CAGGTCTCCGCGTCTCGCTGTATTGCCCAGGCTGGAGTGCAGTGGCATGATCTCA
GCTCACTGCAAGCTCTGCTTCCTGGGTTCACGCCATTCTCCTGCCTCAGCCTCCTG
AGTAGCTGGGATTACAGGCACCCGCCACCACGCCCAGGAAAGAAAAAAGAAGA
AAACAAACCTCCATACGAGAATGGGTCTAAAGGAACTTCCCAAACCTCCATGAT
TTTGCAGGAAACAAGATAAAGGTGGTTTCCACAAGAAAAATGGCACAATGTTTC
TCAGAAGACAATTACATAAGAATCAGCATACTTCAAATTCACAGCAAATAATCA
GACAATTGATGAAAATACTTACCCAAACACTAATTGTAGACTATGCCTTCTGAAT
ATGTTTGTCATAAACTTGGAGTAAGGAATCCTCACAGGCACTGGACAATTCAAAA
AACGTAAAGTTGTTTGTTAGAATACTGGTGCTTTTGGATAGAAACCCTCATCCAT
ATCCTGGTAAGGCTTGAAGTTGCACAGGAGTTTTCATTTGTCAAAACCCAGAAAA
CCATAAGCTTTAGATTTGTGAATTTTATATTGTATTATATGTGACCTTTCTTTTTAA
AAAATGAGCTGTAAGCAGTCTCCCAGACAGTAGCTCAGCCTCCAGAACTCTCTTT
CTGCATAGTTGAAGACCCCTCTTCACACAAGATGGTAGCAACAAATCATAGGTGC
AATTGCACCAAATTCACAGAAGATCAATTGAAAATCCTCATCAATACCTTCACTC
AAAAACCTTACCCAGGTTATGCTACCAAACAAAAACTTGCTTTAGCAATCAATGC
AGAAGAGTCCAGAATCCAGATTTGGTTTCAGAATCAAAGAGCTAGGCATGGATT
CCAGAAAACACCAGAACCTGACTTTAGATTTAAGCCACAGCCATGGACAAGATT
AACCTGGTGTGGAGTTTCAAAATAGAGAAGCCAGATGGTGTTGTACCACCTATA
GCACCTTTCAATTACACACAGTCATCCATGCATTTATGAAAAACCCATACCCTGG
GATTGATTCCAGAGAACAACTTGCTGAAGAAATTGGTGCTTCAGAGTCAAGAGT
CCAAATTTGGTTCCAAAATCAAAGATCTAGATTTCATCTCCAGAGAAAAAGAGA
ACCTGTTATGTCCTTAGAATGAGAAGACCAGAGAAGACCAGGGGCAAGGTTTCT
GAGGGACTTCAAGGTACAGAAGATACACAAAGTGGCACCAGCCTCACTAGCACT
CTCATTTCTCAAGAGCCAGAACATGGTGAATACAGTCAAGTTCAGTGTATTTGAT
AATATCAATTTGGGCCCCAAATCTCTCTCACAGTCTTCCTGGGAGTCTATTCTTCT
TCCAAAAGTGCAAGCTAAGCCTTCTGAAGATGGTAAAGAACTTGGCCGGGTGTG
GTGGCTCATGCCTGTAATCCCAGCACTTTAGGAGGCTGAGGCTGGAAGATGGCTT
GAGCCTAGGAGTTTGAAACCAGTCTGAGCAACATAGTAAGACCCTGTCTCTATTC
TAAAAAACAAAATAAGTAAAAAGGACTGTAGGAGGCCAAGACAGGTACAGGAG
GCACCACACTACCCTGTTGACACAGCCTGGATCCAGAGTTCAGCAGACCTTGAGA
CAATGAAAACAAACTTAGTAATAATCATTTTTCAATCATTGCAGTAATTATTGAT
TTGGACAAAAATCAATTGATGTCAAAACCTTAAAGTGACGTTTCTCTGCCTATGG
AGTGGTCATTCTTTTATTCCTTTAGTTTCATAATAAATTTTCTTTTACTTAAAAAA
ACTTATAGTTTGATGAAGAGTGAGATATATACCTCATCTCAAAGAATCTTCACAC
ACGCACTTATTAATTACAAAAGGAAAATCAGTAATTTTGCAGTGGAGACATATG
GCCAACTCCACCTTACCCAAGTGGCTGAAAGTCACTGCACCAGTAATGGCACAA
ACCAATGTGAGATGATTCCTGATATGATACACTAAAAAGGGCACTGTCTCTTCTG
CATGTTGCAGACAAAAAGTGGGTAAGCTGACACTGAAACTAATAATTAGGCAAT
GTCAAGCAAATACAAATTCAGGTTGACAGTCTGCAAAGTAACATCCATGTACTCT
TCAACAATGGATCGACCCTAGCTACTCAGGAGGCTGAGGTGGAATAATTGTTTGA
GGCCAGGAGTTCCAGATCAGCCCGGGCAACATCATGCGACCCCATCTCTAAAAA
CATCTTTTTAAAAATGAGCCAGGTGTGGTAGCATGCACCCGTAGTCTCAGCTACT
CAGGAGCCTGAGGCAGGAGGAAGGTTTCAACATAGGAGATCGAGGCTGCTGTGA
GCTATGATCGTGCTACTGCACTCCAGCCTGGGTGACACAGCAAGTTCCTGTTTCC
AAACAACAACAAGAAAACAAAACAAAACAAAACAAAAAATAGATAGAATAGTG
ACAATAAAAATGGAGAAACAGTAGGCTGACTCAGGAAATGCTTAGAAAGTACAG
CCATACCTCAAAGATATTGTAGATTTGATTCGAGACCACCACAATAAAGCAGATA
TTGCTACAAAGTGAGTCACACAAATTGTTTTGTTTCCTTGTGAATATGAAGTTATA
TTGGCTGGGTGTGATGGCTCATGCCTATAATCCCAGTACTTTAGGAGACGGAGGC
GGGAGGGTCACTTGAGCCCAGGAATTGTGAGATCAACCTGGGCATATAGGGAGA
TCCTGTCTCTATTTAAAAAAAGAAGCTATGTTTACACTACACTATAGTCTATTTAA
AGTGTGAAATGGCGTTATGTCCTTAATTTTAAAACTCTTGATGCTGGCTGGGTTC
GGTGGCTCATACCTGTAATCCCATCACTTTGGGAGGCCAAGACAGGTTGATTACT
TGAATTCAGGAGTTCAAGACCAGCCTGGACAACATGGCAAAACACGTCTTTAAA
AAAAGAAAAGAAAAAAGAAAAACAGAAAGAAAAAGAAGAAAAACTACTTGCTG
CCCTTACTTGAAGCTCAATTATTTAAAACAAAGAAAAAATATAAAAATCTTTTAT
TGCTGAAAATGCTAATGATCACCTGAGCCTTCAGGGAGTCTTAGTCTTTTTGCTG
GTGAAGGGTCTTGCCTTGATGTTGTTGGCTGCTGCCTGATAAGGGCGATGGTTGC
TGAATATTGAAGTGGTTGTAACAATTTCTTAAAAGAAAACAATGAAATTTGCCAC
ATTAACTGACTCTTCCTTCCACGAAAGATTTCAGTGTACCATGCGATACTGTTTGA
TAAGCATTTTACCCATAGTAGAACTTCTTTCAAAATTGGAGTCAGTCCTCTCACA
CCCTGCCACTGTTTTACTATGTTTATCAATATTCTAAATCCTTTGTTGTAGGCTAA
ACAATATTCACAGCATTTTCACCAGGAGTAAATTTCATCTCACAAAACCACTTTC
CAGGCTCTTTCTGGACTGTAGAGTTCTTTCCAGGCTACCTTGTGGCAGTTTAAGA
GTCTGGCATCATTTTCCGCTGGGACCTAAGGATCGAGGAGGTGCTTGTGACTAGA
CTGCCAATGGACCCATCACAAAGTTTAACCCAACCTTGATCCCCGAGTCTTCACA
AATGCTCACTGAAGAAAATTCCTGGAACAATTCAGGGTCCTTTCATAACCTCTAC
TCTGAGGTGTTAATAAAAAACCTTAGTAACTTAAAAAAAATGAGCTGTACACAA
ATACTGAACAATAATGCTACATATGTTAAGTATGTAAGAAAAATATATACTTTGA
CATAAATAAGAAACGGTGAGTTGATAATTGGATAGAATGGTGGATAGAGTGATA
GATATGTAGTAAAGCAAATATAACAAAATGATAATTGTACAATCTAAGTGGTTG
GACTATAAATATGCACTTCCCACAACATTTTTATATGTTTAAACAGTTTTATAATA
CCATATTAGGGAAACTGTTTGTCTCAAGGAAATAGAGATTGTGATATGTTCTAGT
ACAATGAAGTGTAATCATGTAAAATAAAAGCTTTTACTTCTGGCAATTAAAGTTA
ATCATGTTAGAACACTGTCTAGGAATGGTTGG
>LOC100287482 Agilent Human SurePrint G3 Probe: A_21_P0013271
Primary Accession: NM_001195243
(SEQ ID NO: 53)
CGAGGCCCTGCCCCACGCCCGGTGATTGTGCGCGCGGCCCCGCCCCCGAGGCGC
ACGCCGGCCCAGCGCCCACAGCTGCGGCGGCCTAGGTGCCGCGTGGGGCAAGCA
GGTGCCTCGCGTCCAGGCGGCTCCGCGGCTGGCTGCCTCCCGAGCCGGCCGCGCT
CCTCCCAGCGAGGCGTGGCGGGGAGGCGTAGTGAGGCTGGGCCCGTGGCGGTTC
CCTGAGGAGGGCCGAGAAGGGGCCGGGGGTGCTAGGGGAACGGGCGCTGGGGG
CAGCGGCCCCGGTGGATGCTAAGGGCTTCGGGATCGGGAGAGTCCACCACGCCT
GCCTGCTCGGCTGAGAATCGCCATGCCAGCTAAAGGGAAAAAAGGAAAAGGCCA
GGGCAAGTCTCATGGGAAGAAACAGAAGAAACCAGAAGTGGACATTCTCAGCCC
CGCGGCCATGCTGAACCTCTACTACATCGCCCACAACGTCGCTGACTGCCTGCAT
CTGCGAGGCTTCCATTGGCCGGGTGCTCCCAAAGGAAAGAAAGGGAGAAGCAAG
TGACAGCATTTCACAACACATCTCTGTTACAGACAACAGGACCTGGGGAAGAGA
AGTCAGGATAACACAACTGTTGCCAGCAACATAGACTTTACTCCAGACGACTTGA
GATGCAAATTAAGTGTGCTTTTCTGTGATGGTGGAAGATCAGGAAATGCACCTTA
CTTCCTCTGTTATGCCAGATATGGTTAGCCACTTTGGTTTTTTAGGAGCTATAGGA
TGGGAAAAGCCTGAGTAATTCCTACACAGTGTGCTGAAATTAATAGAACTTTCAG
AAATTATTATAATTCTGGGTCAGGATTAAACTTTGCTCTCAGAAGGCAGTTCTAG
TTGCATTAATTGTTTTCTTTTGCCAAAGAGCGTTTGTCATTTAGAGAAGACACGGC
AAGAAACACTGGGTTTCCTTAGGAACATTCCTCTCTTGGGCACCATTTCCTTTTTT
TTTTTTAATGGAAAATAATAAATACTTTGTTTCTATAATTTTCTTCTCAGCAAAAA
AAAAAAAAAAAAAAAAAAAAAAAAAAAAA
>FLJ20444 Agilent Human SurePrint G3 Probe: A_21_P0013726 Primary
Accession: XR_132891
(SEQ ID NO: 54)
TCTTCCGTGCAGGCAGGCTCTCCTGGGGACCTCAGAGATTCTCTCCAGCGGCAGC
GGAAAACGGACAATGGGTGGATTCGGGTCCAGATTCTGGTAGGAGGGAGTTTGG
GATCGAGATCTGGAAAAAAGCACTAGACTGGAAGAGGACGCGATGGAGTCGGA
GCCGCTGGCGGGGACAAAAACCAGAGGCCGGGGAAGGCGCCGGTGGGAGGCAA
GGCACGGATGGACTTTACCTGCGCACGCGTCGCAGCCATCTCCGCGCACAGTGGT
GGCCACCGCGACTGGTGCTGAAGTGTTGGCGCGTGCCGGGCGCTCCGCTGGGAC
CCGGGTTGCTGGCCCTGAGTCTCAGCTTTCTCATCTGTACGGTTGGGACAAGTAC
AGTAACCCTCGCCCGTCAAGACGGGCCAGGGCTGTGGCGAGGGTCCACGCCTTA
GAGCAGGTACCTATCTTGTGCAGGGCCCTGAGATGGGGTCTGACTCAGTTCCTGC
GGGGAACTTCACCAGTGACCCAGTCAGTGCCCTTCAGTTAAAGACCACCAGGAG
CACACTTGCAGGAGTAGGGCTGATTGGAGCCATTGTACAGTGTCGGGAACATAC
CAGGACACTGAGAATAGTGTCATGTCATAAGGACCCAGAGCAGATGGACCCTGC
TGTGATGCACAAAGAGGACCACGCAAGATATGATAAAGATCTACATCACTGAAT
TTTGGTTCCATTTTTGTATCTCAGCTTCCAGGAAATAAAAAAGAATTCTAACATTC
ATACTTTCAGTATTTTATGTGAGAGGTTTTGTTGTCAAAATCAAGTCTGAGAGCA
ATGTTTGTTGGGGCCTTTAATTGGAGTCACCAAGCGATAAAGGGGACATTGTCCT
CAACAATAACCCTATAATAAACACGTTTTGGACAATAAATATATGACAATTTCTT
AAAAGCAATTTCTTGGGCAATCAAGACAGTATGGCTTGAGTATGGAGTTATACG
ATGGTTTGGATTAATCCAGTATTAAATCTTTGGTTATTACAGAAA
>LOC100505666 Agilent Human SurePrint G3 Probe: A_21_P0014077
Primary Accession: NR_040772
(SEQ ID NO: 55)
GCCCGCGCTGCTCAGCGCTACCGCTTCCCCGCAACTGTGCGGAGTGGGAGCCGGT
GCCCGGTCCGACCGGCTTGGGCGGCGCGCCTTCACCCGGCGCCAGGTCCGGACC
CCTCCCTAGTAGCTTCGCGGCCTCCCTGCCTCCTGTGCGCGGCCTGGCTCGGAGA
GGTCGGGCGGGCAGGCTTTCCCGACTGCAGGCGAGGCAGTGCGCGGCTCACCCC
AGTCCCCGACCCACGTGAAGCGTACAGGGCATTTTATTAACCGGGAAGGACGGT
GCGGAAGAGCGAGCAGGACGCCTCTTCACCCCGCGTAGGCAGTGTCGTCGTTGC
TGTCACTAAAGGCGGAGGAAGAGAGCTCTTCGCGGGGCGTGCAGACCGGGCACC
GCTGCCGCATGTCGTCCCAGCACGACCAGCAGTACACGGCCTCGCAGTCCAGCGT
CCGGCACACGTAGGACTCGGGCGTCTCGGGTGCCTGGCACACCACGCAGCGCCG
GCACAGCCAGCGGCGCAGGAGCGGGCAGCCGCGGTGCAGGATATCCGCCAGCG
GGTGGCGCTGTTGGGAGGTGAGAAAACTGATGCTTGGAGATGTGATCACTGCCC
AGGGTCACCCAATGATAACATGCATGCATATGGAACTTGCTGCATGCCAGCACC
ATGAGTCCGCTCCCCATGCTGTCCTCACCACATTGCTCATTTCTGAGGCCTGGATG
GTGGGCTTGCAAGGGAAGATGACGGTTTTCTCCTCAGCTTTGCGGAGTGGCAGCA
GAGTCCGTTTGCCCTGGAAAACAAATGTCCACACAGTTAGGAAGCCCAAGGGCC
CTCTGCCCTTTCCTCTCTGCCTTCCTGGAGCATGAACCCACACAGGGCACACAGC
AGCAAGGCATCCCCGGGCAGTGCCGTGCCCACTCACCAGCTTCTTCCTGCGGTCA
TCGATCTGGCAGAAGTTCTCCTCATCTATCCCCAAACATGGGCTTCCTTGAGGCA
CAGTCATTCAACCAACCAGCCAGCATTCATTGAGCACCATCTATGTCCTGGGCAC
TGCTAGGGGATGGTGATAACAGGGAGAAGACTCTGTCCCTGCCTTCCAATTGTGT
AGAGGAAGACATCCCCCTACATGATGGGTGAGACATAGCAGAAGTGAGTAGGGG
ATGAGGTGGGGGCTCAGAGGAGGGCATGGTCAGCCTGTCTGGGAGGGAGTTGCA
TGTGTGCATCTGAGGTAGGGACAGGCATGCATCTTACAGGATGAATATCGAGCA
GAGTTACAGAGAGGGGGAAACTCCTTGAGGTTTCAGGAATCACCTAATCCACTG
TGACTCACAAATTCCTGCCTCTTGGCTTTGCCTGCAGCATATCTCCTGGAAGTGTG
CTGGGGCAAAACTCATCCCAGACCACCATCTCCATCCTCCCCCAATACACCCTGG
CTCTCCCTGGCTACCCTTGAGCACGGTGCACGTGTGCATGGGTGCATGCCTGCAT
ATATAGCTATCCCCCATGTATTTCCCAAAGCCCTACATAATGCTTCAGTTTGCTAA
GGAAAAAATGTTAATTACTGCAAATGTGTTTAAAACTGTAAAAGTACATTAAAC
AAACTCTGTAAAGTGTGAAAAAAAAAAAAAAAAAA
>LOC100507025 Agilent Human SurePrint G3 Probe: A_21_P0014172
Primary Accession: ENST00000289352
(SEQ ID NO: 56)
AGCGTTCGTAAGGTTCTCAAAGACTACAGAAGTTGGAAACTTCGCGGAGAGACT
GCAAGTTACCCTTTCCAAAATGGCGGGAAGGGCTAAAAACAAAGAAAGCTCGCA
CCCAGACGGCGGGCCTTAAACCAAGGCGAATCCGTGAGCGCAACACATCTGCTT
CTGTGGCTCCTGATGGATCTGAGAAGATGGACGTGGAGGATGAAAATCTGTCTG
ATTATTTTGAACTGATGTTTGTTGCTATGGAGATGCTGCCTATATGTTGATGTTGC
AGACGTTAAGTCACTAGCCCACAGCCTTGTATTCCATACTCAGAGACCCTGCTAC
TTACTTGACATCTCAACTTGAAAGTCCAATTAATATGCACTTCAAACTTTAATAG
GCTTCAAACAGAATTTCTTTCATTATCTCTGCAAAACAGCTTCTCTCATCATCTTG
AAATTAGTGAATGGCATTTTACTGTTTTAGTTGGAGTCATTTCTGTGGTTTTCTTT
CACATCCTACATAACAATCCATCAGTAAGTTCTATGAGCTCTTCTTTGAAAACAA
ACAGAATCCAACTGTTTCATTCCCACTTCTGCTCTGGTCAAGCCACTGCCAACAC
TCACCTTTATTATTGTAGCACCCTCATTGCCTAGTTCTGTCCCACAGATTTCCAAT
AAAAGGTGAATAAAATCAGGTCACTCTTCT
>LOC100506303 Agilent Human SurePrint G3 Probe: A_21_P0014553
Primary Accession: XR_110283
(SEQ ID NO: 57)
GGCACCCGCCACCACGCCCAGGAAACTCCAAACTGTCCAAGGAGATAGTTCTGT
TGTGATTACTTCATTGAGAAATTTAACTTATGAGCCGTTGAAAGGAATGCAAGTT
GCTGCAAAATCCGAATGAAGAGTGCAAAACGACTAAGCTACAATGTTTTGTCATT
ATTCACTCTGATGTGAAAAAGGCAGTGAATTTAATAGAAAATAACTTCGTAGAG
CAAAATCTCAGGTGTGTTTTTTTAGTGCCGCAGTCTTGGATGATGGGTTCCTAGA
AGCTCTCAACATCTCTTCTTAATTGGAGAAAGTGTTAAGCCCCAAAGTAGCTGGA
GCAGTACATCTTCAATTTTTGACAAGAAAGCAGGAACTTGATTACTTTGAGTGCT
ATTCATTAGTTTCTGCTTTCATTGAGAATGCAACAAAAGCCAACTAGGCTGCTGC
TAACTCCTTGCTGGACTTCTTCTGCCACTGTCACAGGAACTGTAATCTCACTGGAC
AATTAACTAGGGAGTCTTTCATCTTGAGTGACTGCTGCACAAATGATCTTCAAAG
CATTTTAGCCACCAGAGGAATTCTCTTGAAATACCCAAAATCCATCAGTATCTTG
AATCATGCTGGATTTTGAAGAATTCTTAACAAGCCATGTAAAGGGGGCTCTCTGG
CCTTGAAATAGTGATGTTTTTTATACAGAAAGGAGAATGCAGAATGGTCAGACTA
CCATGCACTGTTAAATTTGATTTCAAGAAATTACAGGAAAACTTTCCAAAGTTCC
ATCTCACAGAAATTATTTTTACAAAGAATTCCAAGATAAGTTTAGTTTTATGGAA
GACTTTTATGTGGTTTTTACTCACTCTTCATCTCAGACATCAACAGATGATTACAT
CACTTATTTAGCTAGTAAATTTATTAATATAAAAACTCAGAGACATTCCAATATC
CACATTGCTTACACCATTAGGCATAGATTCAGTGTCAGCTATGACAATTGAAAAT
AAGCTGTTTTGTGATTTAAAGGTTTAAATTTCTCTAACCAAACTGCTTGATCCAGA
TGCAGGACTGCAAATGTTAATATTTGTTCTGGAAGAACAATCAAATAAGACTTAA
GAGGAAAAGGAATGGCCACAATCCACCTGAAATTTTTTTTTAAAAAGTGTGCAG
CCTACTAAATCAGAATGAAAATAGAAGTACAAGATTATAAACAAAATGCAATCA
AACTTTTCTTAAGCTTACCTAAAGTTATTTCATCTGAAAATTTCAAGCAACTTTGT
TCAACATTAAATTGACAATCTAAACTAACAAGTCTTTTGAATTTATGCATGGTAG
TAAACATTCTCTCTATTAACTGTATTACCTAAGGCTAAACCTAAAATTTTTAAGCA
AAATTAGAAAAATAGTCTTCACTCATCAAAAAATAAAGTTTGTTACATTTAGTAT
TTTCCCAATAAAATTGGTCGTTCTTGGTTTTTTATTTGGAGAGTCTGTGCAAAATG
TCACTAAAAATAAATTAGCACTAGAAATTATTTCTAAATACCAAAAAAAAAAAA
ATGAAGAATGGTT
>LOC100506802 Agilent Human SurePrint G3 Probe: A_21_P0014847
Primary Accession: XR_132718
(SEQ ID NO: 58)
AATCTGCAACGGTGGGCTGCAGTGGAGAGAGGGGCGTGGACTGCCACTGCTGCC
CCTCGCCCTAGGTCACCCCCAGCTTTATCAAATGTCAGAGCACCAGGAATCCTCC
ATCATCAATGAGGACACAGAGCTGGGTGATGCCTACGTGTTGAGATCCTGGTCCC
TCCACACACGCTCTACCAGCTGCTGCGTGATGCCCGTGTCCAAGATCAGGTTGTG
CAGAAGGAAGTTGTTGCCTGGAACAGGAGGGGAGGGGTGGGGGTGGGGGCATC
TTCTTGCAGCTCCTTGCCCACCCTCACCCCCACCCTTAAGGCTCCACCAGGAGCCT
CCTCCATGACCTGGCCCTGGCCCAGGCCCAGCCCTTAGCTTGTGCCTGCTTATTTC
CACACCTGCCCGGCCTCTGGGTTCCTCTGGGCTGGCCCCATGCTGCCTGGGCACT
GCCCAGAGCCAGCTGCCCTGCCAGGCACTCACACTGCTTGGAGTCTGGAGTCACT
TTCTCCATGAGCTCAATAAAGTTTTTCAGGAACTCGG
>AB116553 NCode human ncRNA array Probe: IVGNh00466 Primary
Accession: AB116553
(SEQ ID NO: 59)
CCCAACCCTTTGGTGGAGCCTGAAAAAAATCTGGGCAGAATGTAGGACTTCTTTA
TTTTGTTTAAAGGGGTAACACAGAGTGCCCTTATGAAGGAGTTGGAGATCCTGCA
AGGAAGAGAAGGAGTGAAGGAGAGATCAAGAGAGAGAAACAATGAGGAACATT
TCATTTGACCCAACATCCTTTAGGAGCATAAATGTTGACACTAAGTTATCCCTTTT
GTGCTAAAATGGACAGTATTGGCAAAATGATACCACAACTTCTTATTCTCTGGCT
CTATATTGCTTTGGAAACACTTAAACATCAAATGGAGTTAAATACATATTTGAAA
TTTAGGTTAGGAAATATTGGTGAGGAGGCCTCAAAAAGGGGGAAACATCTTTTG
TCTGGGAGGATATTTTCCATTTTGTGGATTTCCCTGATCTTTTTCTACCACCCTGA
GGGGTGGTGGGAATTATCATTTTGCTACATTTTAGAGGTCATCCAGGATTTTTGA
AACTTTACATTCTTTACGGTTAAGCAAGATGTACAGCTCAGTCAAAGACACTAAA
TTCTTCTTAGAAAAATAGTGCTAAGGAGTATAGCAGATGACCTATATGTGTGTTG
GCTGGGAGAATATCATCTTAAAGTGAGAGTGATGTTGTGGAGACAGTTGAAATG
TCAGTGCTAGAGCCTCTGTGGTGTGAATGGGCACGTTAGGTTGTTGCATTAGAAA
GTGACTGTTTCTGACAGAAATTTGTAGCTTTGTGCAAACTCACCCACCATCTACCT
CAATAAAATATAGAGAAAAGAAAAATAGAGCGGTTTGAGTTCTATGAGGTATGC
AGGCCCAGAGAGACATAAGTATGTTCCTTTAGTCTTGCTTCCTGTGTGCCACACT
GCCCCTCCACAACCATAGCTGGGGGCAATTGTTTAAAGTCATTTTGTTCCCGACT
AGCTGCCTTGCACATTATCTTCATTTTCCTGGAATTTGATACAGAGAGCAATTTAT
AGCCAATTGATAGCTTATGCTGTTTCAATGTAAATTCGTGGTAAATAACTTAGGA
ACTGCCTCTTCTTTTTCTTTGAAAACCTACTTATAACTGTTGCTAATAAGAATGTG
TATTGTTCAGGACAACTTGTCTCCATACAGTTGGGTTGTAACCCTCATGCTTGGCC
CAAATAAACTCTCTACTTATATCAAAAAAAAAAAAAAAAAAAA
>AF087978 NCode human ncRNA array Probe: IVGNh01580 Primary
Accession: AF087978
(SEQ ID NO: 60)
AAAGCATGGGAAAAAGAGACTCTTTTAGGATCAGATCTGTGAGCACGTTGGCGA
GGAAAAACAAAACAAACAAAAAAAAGAACCTTGTGTCTGTCTGGTGAAAAAAA
GAAAAACAAATTGGAAGAGAGGACCATGAGAATTTTAATAAAACAGAAGGAAA
CTAATGGACCTTCCAGGATTTATTGTGGACGGATGTGGATATATTCTGTACAGGA
ACAACACATATGGAAGTGGACTGAAGCCTATGTAGAAACACACACACACTGAAC
ATTGTTATTCATTTTGTAAAATACTAGTCTTTATTTTCATTTTTTGTAAAATTTAAA
CATCGTATGCGCATAAAGAAAAAGGAAACAAGAATTAGGGGAAAATAACATTTT
CCAAATAATTATAAAAAATTGTCCTGTGTCTATGTATCTATATCTGTTTTGTATTT
TTTTCTGGTTCCAAACCAGATTTCCTGTGATTCTATACTAATAATTTTTGATATAA
CCCTTTGCTTCTTATAATGAGTGCGATATATGTTGTCGAGGCTGTTCTTCAAGAAT
TAAAATTGAAGTGAAAATTTAAACAAAAATAAAAGAATTTAGCAAAAAAAAAA
>AK024556 NCode human ncRNA array Probe: IVGNh04604 Primary
Accession: AK024556
(SEQ ID NO: 61)
GTAGAGATGGGGGTTTCATCCTGTTGGTCAGGCTGGTCTTGAACTCCTGACCTCA
AGTGATCTGCCTACCTTGGCCTCCCAAAAGGCTGAGATTACAGGCATGAGCCACT
GCGCCAGGCCTTCTTTCTTTTCTTTTTTTCTTTCTTTTTTTTTTTTGAGACATCATTT
AGCTGTGCTGAGGGGTTCTTAAATAGGCAGCTCAGAAAATTGTTTTCCTTTGTCA
GCCACATAAATTCAGCAGAGGCTCTTGGAGGGTCCCTGCTGGTGAGGGGTGAGG
CCAGCAGTGGAACTCTGATTTGGTTTTTGCTGAGCTGGTGGTTGAAAGGAATCCT
ACTACATCGGGGTTATAATAGGGAAGATACATTTTAGAATATGCCCAGTGGAGC
CATCGGATGCTGCATCGTCCCCAGAGAGCCAAGTCATCGTGGGCCAAGCTCCCAT
CCCCATGTCTGGCCTCAACTGCAGGCCCAGAATGTTGACAGCTGCCTCTTGGAGG
GTTATGGGAGCCTGTGAATGCCAACATCCCCATTTGCCTGCAGCGGCTGCTCCCA
TCCTGGCTTCCTGGTGGGACTTTTCCATGAATTGGGGAATCTGCTTTCTGATTCCA
AGGCCTATTAAAATTTCTGAGCATTGCCCATTTCTTTTGCTTTATCTGTAGGACAT
GGGCTGTTTTTAAAGAACCTCACAAATGAAAAAAAAAAAAAAAAA
>BC012900 NCode human ncRNA array Probe: IVGNh15798 Primary
Accession: BC012900
(SEQ ID NO: 62)
GTGGAACAGTCTTGTTATGGAGTGCCAGCTTAGAGGTTGTTGCAAACTTGTCTAG
AAGTGAGAGCATGGTTTTTTTTAGCCCTTTGAGAGTCTACATCTAATGAACATTCT
TGCTCACCCATAAATAACGTCAAGCCTCAATGTCACCGTCACGTTGGGATACTCT
TTCTCATCTGGCATCCTAGACAGGACAAGGTTGGTTACCTTTCCTTCCATGAACC
ATGAACCTGTGACGGCATCATTCATCCTGACTTCACCAAGCTCCGCCTGTGGGTG
AGGCCAGAGCTCCCACTGGCAATTTTTAGAAGAGCCAGAGGCTCCCTGCTTCCTC
TAGAAATAACAGTTCAGGGTGAAGCATGGAGGGTTTCAGTTCCCAGACAATGGA
ACCATTTAGAGACAACACAGTTGGACATTTCCACTTTTTCCTTGATTCCTGGAAGT
CCAGTGGGTTCTGCAGCTGAAAAAGCCCTGGGTCCCAGCAGCAGAGAGACAGGA
CAGAGGGGATGCTTGGGCGGGGAGGGACGGTAACCTGCAGAACAGATTCCATTT
TTATAGAACGAGTACACGTTTGCTAAAACAGTCCTGCTTTCCCAGACTGGATTCC
CACCACAGGGACAGTCGGAACTCAGGACTAGCTCCAGCGACATCTTTCCTCCGA
ATTCAAGCCTTCTATCACAATGTCAAAACAGCTATTTATAAAGCCATTTTCATTGT
ACTTGATAACAGCACGAGTCCCAAAACTTTTAGAAATAAAATAGGACATTGGCTT
GATTGAAAAGAGGGACTTTTTAAAAATTGTTCTTTCGTCAGAAGCCTTTTGGATG
ACTTACAATAGCTCTGATGAAGATACCACCCCAGCGTCAGTCCAATAGGTCAGTG
AGTTTCAACAGGCATCCATCCCTCCCATGAAGGGATTCTGGTGATGGGAAGTTTC
TGTAATGACAGGAAAGCATTGACCCTCATTGATTGTCAACTTTGGTATTAGCCAT
GAAAGACAGGATGCTCATTGGGTGTTCTGTAGAGTGAGGAATGCTGCCTATTCCC
TCCCAGAACGTCTGACCCAGGGGTGTGTGTTGAGGAGCCCTGGGGGAAATGGAC
CAAGTTTTCCCACAGAGCAGTATTAGGCTGAAGAGCAGGTGACTGGTAGGCCCC
AGCTCCCATCATTCCCTCCCAAAGCCATTTTGTTCAGTTGCTCATCCACGCTGGAT
TCCAGAGAGTTTTCCAATTTGGGAAGCCATGAGAAAGGTTTTTAAATCTTGGGAA
GATGGAGAGAGGGACATAGGATAGTTGACTCCAACATGACAGGAAGAGGCTGG
AGATTGGGAATTGGCCATCAACCAAGCCTGTAGTAGTAAAGCCATGGTCCCGCA
TTGGAATTACTTGGGGAACTTATACAGTTCTGATACCCAGGCTCTCCTAGACCAG
TTCAACCAATTCTAGGTGGGGGACTCAGGCATCAGTGTGTTTCGTAGCTCCCCGG
GTGTTTTCCCTGTGCAGCCGAGCTTGGGAAACTGCCATGCTTTTTGGATGTCAAG
GCGCTGTTGGAGGCTGGGTGTGACAGCACAGAGCCAGGTTGTCTTGTGGAAACC
ACAGCCACGGGTTTGCCACTGGCTCAGCATGGCCTCACTGCCAGTCCCAGCCTGG
CTGAGGGACAAGATGGTTTCTCTTGGGAGTTCCTGAGTGGAGCACCCTTCCAGGC
TTTTTGAAAGCCAGCTGATCTGTGGAGCCTTGTTAAGGGACTCAATACGGTGTTT
GGATATTGATGTTTTTCCTTGAGACTGTCTTGTCCATCAATAAAGATGGAGGATG
TCTCCTCTTTGAACCCCGCTTCCCCACCAGTACTCTCTCTCCCTTAGAGTTTATGA
GTTATTCAAGGAGGAGACTTCTTAAAGACAGCAACGCAATTCTTGTAACTTGTGT
AAATAGCCCCATCTTTCAGAGTGATACCATTTCTACATTTGATAATGCCTGTATTC
CTGTAGGATGTATATAGTTTAGGGGATTTTTTTTTTGTTTGGTTTTGTTTTTTAGAA
GTCAATATGTCTGGTTTTATTTATTGCTTGAAAAAGATCATTTGAAAAAAATAAA
TACATTTTCAACCACAAAAAAAAAAAAAAA
>BC013821 NCode human ncRNA array Probe: IVGNh15835 Primary
Accession: BC013821
(SEQ ID NO: 63)
GGGCTCTGTCCTTAGGGAGGAGCTGCGGAATCCCTGCAGCTGTGCCCCCAGGCCC
TGCCTTGCACACTTCCTGCAGCCAGGGCGCCCCTGGGGAGGTCAGGGCAGGCCG
GGGAGGCTGAGGCCCACCTGCCATAGTGGGCAGGTGCGGGAGCCAGGGCGGCA
GTGGCCTCGGGGCTGGGTGGGGCGCCTGGCCTCTGGTCTCTGGAGCAGTCAGGG
GCTCTGCAGACGCTGAGAGGCCTGCTCATAGTGGACTGGGAGATGCTGGAGCAG
CCTCAGAGCCATGGCCGGCCCACGGCGGGAGACGGCCCTGCTGCTGCCCCTCTGC
CTGTGCGTGTGCACCTGTGGGCACCTGCGTGTGCTGGGGCAGGCAGGGCTGTATT
GGGACCAGGTCCTGTAACAGCCTGCCTGCTTACCGTCTGCTCCCATCCCTGGGGA
AAGCAAGGGAGCTCGGGGTCCTAGGACCTGACCTCAGCGCTCACCCCCACCAGC
ACCACAGTCACCAGGACTCTGTGACTCAGTTTACCCCACGAGAGCCCCTGGGATT
CCCAGGGCATCAGAAGGCCCATCAGCCTCCCGTGAACTGCTGGGGTGGGCCTGG
CCTTGGGACGCGGGTGCAGGGGCCTCTCCTCACTGCCCCCATGGCACCCACAGCC
AGTGCCCGAGCCTGCTGCAGCCCCGACCCGGCAGAGCAAGCGGCTCTGCTACCT
CAGCCACGTAGCTGATGGCATCCTTCAGGTTCAGCTCGTGGAAGACATTCAGGAT
CCGGTCTCGAGACTTCTGGGCCGACCGTCTCATGAGGACCCTGCTGAGGAACTTC
CTGTCGAAGTGGGACCACCTGTAGGGACAGACCTTGGGTGTGAGCCTCAGGTGA
CAGGCGCCCTAGAGCCCGCCGGACGCGTGGCCCGGCCCCTTCTCTCCTGAATTTT
GTTTGCTATAGTGACCCTGTAGGCGCGTTTAAAATGAGGGAAGCAGCCCCTGCCA
CACGCCCAGGCCGTCCGCCGTTCTCCCGCCTGTCCTGTTGGATGGAGGCCGTTAG
ACGCATATGAAACTGCATGCCGCCTCCTCCAGAGGGTGGCTCAGGACACGGTGG
GTGTCAGGCCTGGTCAGGCAAGGGGGCTTTGGCCACATGGGGGGCACCTTCAGG
TGCACAGGAGGAAGGGCAGGGGCGGACAGACACCCTGAGCCCTTAGACTTGTGG
GAGCCAAGCTGACCAGAGTGAGGTTTTTTTTAGCCTAACGGAATTAGAGTATTCG
CTGGTTATCCGGATCAGAAGGGACGGTGGCCTGGCCGGACTTAGAGGAAACTCT
GGGGCACAAGGAGGTGATGCCTGTCACTTGGACATGGGTGCAGCCGCCAGAGCC
GCCCTCCAGGGCACAGGGTGGGCCCGGGTGAGCTTGTGTGCTCACACCTGGGCA
GGCCCCGCGGCAGCAATGGCAGCTCTCCTGTACAGGCTGAGTTTCAGCCACACCA
AGAAGTCAAAGCTAACCGAGGCTGTGCCTTCCGAGACCCCCGGGATGGCCCCTG
GGAGGCCAAGGAGTCGGGGACTGGGTACCCGGAGCAGAGTCACTGTGGCCACGG
AGAACCGCAGCTGAGCTTTATGAAGCCACGTGGCCACACCTCCCGGTGCCTCCAC
CCCAAGCAAACACAGATCGCTCAGAAAATGGGAACCCAGGGCAAATTGTATGTG
CTCCTTACTGGGTTTATTATAAGTGTCACATGTTTTTTATAATAAAACATAGGTGA
TTTCACCTTAAAAAAAAAAAAAAA
>EF177379 NCode human ncRNA array Probe: IVGNh23506 Primary
Accession: EF177379
(SEQ ID NO: 64)
GGAGTTAGCGACAGGGAGGGATGCGCGCCTGGGTGTAGTTGTGGGGGAGGAAGT
GGCTAGCTCAGGGCTTCAGGGGACAGACAGGGAGAGATGACTGAGTTAGATGAG
ACGAGGGGGCGGGCTGGGGGTGCGAGAAGGAAGCTTGGCAAGGAGACTAGGTC
TAGGGGGACCACAGTGGGGCAGGCTGCATGGAAAATATCCGCAGGGTCCCCCAG
GCAGAACAGCCACGCTCCAGGCCAGGCTGTCCCTACTGCCTGGTGGAGGGGGAA
CTTGACCTCTGGGAGGGCGCCGCTCTTGCATAGCTGAGCGAGCCCGGGTGCGCTG
GTCTGTGTGGAAGGAGGAAGGCAGGGAGAGGTAGAAGGGGTGGAGGAGTCAGG
AGGAATAGGCCGCAGCAGCCCTGGAAATGATCAGGAAGGCAGGCAGTGGGTGC
AGGGCTGCAGGAGGGCCGGGAGGGCTAATCTTCAACTTGTCCATGCCAGCAGCC
CCTTTTTTTCCAGACCAAGGGCTGTGAACCCGCCTGGGGATGAGGCCTGGTCTTG
TGGAACTGAACTTAGCTCGACGGGGCTGACCGCTCTGGCCCAGGGTGGTATGTA
ATTTTCGCTCGGCCTGGGACGGGGCCCAGGCCGGGCCCAGCCTGGTGGAGCGTC
CAGGTCTGGGTGCGAAGCCAGGCCCCTGGGCGGAGGTGAGGGGTGGTCTGAGGA
GTGATGTGGAGTTAAGGCGCCATCCTCACCGGTGACTGGTGCGGCACCTAGCATG
TTTGACAGGCGGGGACTGCGAGGCACGCTGCTCGGGTGTTGGGGACAACATTGA
CCAACGCTTTATTTTCCAGGTGGCAGTGCTCCTTTTGGACTTTTCTCTAGGTTTGG
CGCTAAACTCTTCTTGTGAGCTCACTCCACCCCTTCTTCCTCCCTTTAACTTATCC
ATTCACTTAAAACATTACCTGGTCATCTGGTAAGCCCGGGACAGTAAGCCGAGTG
GCTGTTGGAGTCGGTATTGTTGGTAATGGTGGAGGAAGAGAGGCCTTCCCGCTGA
GGCTGGGGTGGGGCGGATCGGTGTTGCTTGCCTGCAGAGAGGGTGGGGAGTGAA
TGTGCACCCTTGGGTGGGCCTGCAGCCATCCAGCTGAAAGTTACAAAAATGCTTC
ATGGACCGTGGTTTGTTACTATAGTGTTCCTCATGGCGAGCAGATGGAACCGGGA
GACATGGAGTCCCTGGCCAGTGTGAGTCCTAGCATTGCAGGAGGGGAGACCCTG
GAGGAGAGAGCCCGCCTCAATTGATGCCTGCAGATTGAATTTCCAGAGGCTTAG
GAGGAGGAAGTTCTCCAATGTTCTGTTTCCAGGCCTTGCTCAGGAAGCCCTGTAT
TCAGGAGGCTACCATTTAAAGTTTGCAGATGAGCTTATGGGGGGCAATCTTAAAA
AGTCCACAGCAGATGCATCCGGCTCGAGGGGCCATCAGCTTTGAATAAATGCTTG
TTCCAGAGCCCATGAATGCCAGCAGGCACCCCTCCTTTCCTGGGGTAAAGGTTTT
CAGATGCTGCATCTTCTAAATTGAGCCTCCGGTCATACTAGTTTTGTGCTTGGAAC
CTTGCTTCAAGAAGATCCCTAAGCTGTAGAACATTTTAACGTTGATGCCACAACG
CAGATTGATGCCTTGTAGATGGAGCTTGCAGATGGAGCCCCGTGACCTCTCACCT
ACCCACCTGTTTGCCTGCCTTCTTGTGCGTTTCTCGGAGAAGTTCTTAGCCTGATG
AAATAACTTGGGGCGTTGAAGAGCTGTTTAATTTTAAATGCCTTAGACTGGGGAT
ATATTAGAGGAAGCAGATTGTCAAATTAAGGGTGTCATTGTGTTGTGCTAAACGC
TGGGAGGGTACAAGTTGGTCATTCCTAAATCTGTGTGTGAGAAATGGCAGGTCTA
GTTTGGGCATTGTGATTGCATTGCAGATTACTAGGAGAAGGGAATGGTGGGTAC
ACCGGTAGTGCTCTTTTGTTCTTGCTTCGTTTTTTTAAACTTGAACTTTACTTCGTT
AGATTTCATAATACTTTCTTGGCATTCTAGTAAGAGGACCCTGAGGTGGGAGTTG
TGGGGGACGGGGAGAAGGGGACAGCTTGGCACCGGTCCCGTGGGCGTTGCAGTG
TGGGGGATGGGGGTATGCAGCTTGGCACTGGTACTGGGAGGGATGAGGGTGAAG
AAGGGGAGAGGGTTGGTTAGAGATACAGTGTGGGTGGTGGGGGTGGTAGGAAAT
GCAGGTTGAAGGGAATTCTCTGGGGCTTTGGGGAATTTAGTGCGTGGGTGAGCC
AAGAAAATACTAATTAATAATAGTAAGTTGTTAGTGTTGGTTAAGTTGTTGCTTG
GAAGTGAGAAGTTGCTTAGAAACTTTCCAAAGTGCTTAGAACTTTAAGTGCAAAC
AGACAAACTAACAAACAAAAATTGTTTTGCTTTGCTACAAGGTGGGGAAGACTG
AAGAAGTGTTAACTGAAAACAGGTGACACAGAGTCACCAGTTTTCCGAGAACCA
AAGGGAGGGGTGTGTGATGCCATCTCACAGGCAGGGGAAATGTCTTTACCAGCT
TCCTCCTGGTGGCCAAGACAGCCTGTTTCAGAGGGTTGTTTTGTTTGGGGTGTGG
GTGTTATCAAGTGAATTAGTCACTTGAAAGATGGGCGTCAGACTTGCATACGCAG
CAGATCAGCATCCTTCGCTGCCCCTTAGCAACTTAGGTGGTTGATTTGAAACTGT
GAAGGTGTGATTTTTTCAGGAGCTGGAAGTCTTAGAAAAGCCTTGTAAATGCCTA
TATTGTGGGCTTTTAACGTATTTAAGGGACCACTTAAGACGAGATTAGATGGGCT
CTTCTGGATTTGTTCCTCATTTGTCACAGGTGTCTTGTGATTGAAAATCATGAGCG
AAGTGAAATTGCATTGAATTTCAAGGGAATTTAGTATGTAAATCGTGCCTTAGAA
ACACATCTGTTGTCTTTTCTGTGTTTGGTCGATATTAATAATGGCAAAATTTTTGC
CTATCTAGTATCTTCAAATTGTAGTCTTTGTAACAACCAAATAACCTTTTGTGGTC
ACTGTAAAATTAATATTTGGTAGACAGAATCCATGTACCTTTGCTAAGGTTAGAA
TGAATAATTTATTGTATTTTTAATTTGAATGTTTGTGCTTTTTAAATGAGCCAAGA
CTAGAGGGGAAACTATCACCTAAAATCAGTTTGGAAAACAAGACCTAAAAAGGG
AAGGGGATGGGGATTGTGGGGAGAGAGTGGGCGAGGTGCCTTTACTACATGTGT
GATCTGAAAACCCTGCTTGGTTCTGAGCTGCGTCTATTGAATTGGTAAAGTAATA
CCAATGGCTTTTTATCATTTCCTTCTTCCCTTTAAGTTTCACTTGAAATTTTAAAAA
TCATGGTTATTTTTATCGTTGGGATCTTTCTGTCTTCTGGGTTCCATTTTTTAAATG
TTTAAAAATATGTTGACATGGTAGTTCAGTTCTTAACCAATGACTTGGGGATGAT
GCAAACAATTACTGTCGTTGGGATTTAGAGTGTATTAGTCACGCATGTATGGGGA
AGTAGTCTCGGGTATGCTGTTGTGAAATTGAAACTGTAAAAGTAGATGGTTGAAA
GTACTGGTATGTTGCTCTGTATGGTAAGAACTAATTCTGTTACGTCATGTACATA
ATTACTAATCACTTTTCTTCCCCTTTACAGCACAAATAAAGTTTGAGTTCTAAACT
CA
>uc001pyz NCode human ncRNA array Probe: IVGNh27660 Primary
Accession: uc001pyz
(SEQ ID NO: 65)
GAACAGCTATAGGATCTAAAGTTCCATTACAGCTTACTGTGAAAGAATTGACAA
GACTGGCCTCAGACAAGCTAATCATGGTGCGACTCTCTCCCTTCCTCATCCACCT
CTTTGGGGACAAGAGGATTACATCTCAGGCCAGCAAGATCAGCTGCTTGAAGCT
CTGTGTAAGAGCACTGCACTGACGGTTTGGAGACCTGAGCCTGGGTCCTGACTTT
TCCATTGACTAAGCTCTGTGGCCTTGGGCAAGTCACTCCCCCTCTCTGAGCTTCAG
TATCCTCCTGTCACAGGAGGGAGTTGGGCTAGATCATCTTTAAGGTAGGTTCTAG
CTTTGACATCATCTTGGGGGTTAGGCCAGAGGCTGGGAAGACTGGGTGGACTTTC
TCAATTGCTCTGCCAGGAGGGAACAAGCCCAGAGGCTGAAGCTTCCCAGTATTTA
GAGGTGTGGTAGGGCAGTGTCTGCATTCCCAGGAGACCCAGGGTGATTAAAATT
TATTCTTTAGGTGGCTAGGAGGGCTGGGGAGGCCCAGTGGAAGAGAGAGAGAGA
GAGAGAGAGAGAGAGAGAGAGAGAGAGAGAGATCGAGCTTGATGTATTGCTCA
GTATTCACTTAGAAGGGTTTCTTTCTCTTTGGCCTAGTTTGTGAAGGGATCTTCCT
TTGGACTTTCTCTAAGTTGGGAGAAGAACATTCTTTTCAATGGAGCTCATCTTCTA
TCTCTAGGGTCTGTTCAGCCTTTCATCTATCCATCCTTCCTCTTTATTGGTAGAAG
AAACAGTGGAGAGTAGCCACTTCTGGTTCTAGCACTTCTCTTTTGTTAAGATAGG
GTTTGGATTTAGTATGAAGCTTTGGCTAAAACCCTTGGGTTTGCCTTAGAACACT
GACACTAAGAACCTGGAATGACATGGGGAGGACAAAGAGAGCTCAAGAGGAAT
GCTTTGTGAGAAGTGGATTCTCTCCGTGTCCCTGCCCCCCACCCAAACTTGAACT
ATACCTATTACATTTCCAGGCAGTATCCCTAAGATGAGATCCTGGAGAAAGGACT
AGGGGAAGTATCTTTCTGGATGCTTGTGGTCCCAGAAGGGTACTTTCTGTGTCAT
ACCATGCCACTTCTTTAAGCTCTTCAGGGCAGCCAAAGCCAGCCCTTTTCTCCTAC
TGCCCCCAGGAGAAATAGCACTCTTCTCCCTTCCCCCAGATGGCAGGGCTCTGGC
CTCCCTACACCTCATACCCTGCCTGCCTCCTCCAGGAGGAATCTCCGGGGCCCCT
TCCTGACTCTCCCCACCTTCGCCACTTGTCTCTAGGCTATGGGACAATCATCCCAT
TCACCACTTGACATCCTTGACATCCTTGACTTTCATTCCCCCAACCTCCAGCAGGT
TGGCCCCAATCCTCTTCACCTCTGTGTTTTCTTCTAGAAGATGCATTTTGGGTCTG
AGAGGAGCATTTTCCTGGAAGGCCATCTTTTAAGGCCCCTGCTTGCTGTCATAGT
GCAGAGCAGAAACTTGCACACTATTTAGAGAGCTCCCTTCCCACCTCTCTGCCCA
GCCTTGTTACCTCACTTCTGCTCTGGCCATGGCTGTGAAGGGCCCAGCCAGCTCC
CTGTTTTGATGTTCTGTGCAACAGCTCCGGGGTCTTGTGACTGGAGATCCTCAAC
AGGCCCTGGAGCCAGGACTGGAGTCTTGGCAGCTGATGAGCAGCACCTTGCCGG
CCAGGAGGAGCTGATGCTGACGATCTCCCCAACATCTGAAGGCTTAAAGAACAT
TGTCGTTCTTCAGCCCTCCTTGCTTCTCTCAATACAATAAGACATTGCAGAAGCA
AAAGGGTGGCCTCTGCTCCAGGCAAGGCAGCTGGCTCTGTCTGGGGCATCGGCCT
GGGGCTTGGGTGCCACGTGCTGAGATTGCATAGTCAAAACAGCCATTTTTGCCAA
CAATAGCTTGTGGCTCCCCACATTTTCCTACCCTGCACTCAAGGGCCAGACCACT
CTCTGCATGGACCAGACCATCTTCCCAAACCCATGGTGCTTTTTCCCCAACTCAA
CCTAGACTCCAAGGTGGGGAGGGATGGGTCAGAGGCCATAGTGGCCCCTGGATA
ATCCTGACGTGGGGTGGAGTGGGGTGAGGCAGAGGGAGCAGCCCCAACACCTGC
ACTGGGCCATCTATGGGAAAGAACACGGGTCGAGTGCAGTCGAGTTGTCTGGCC
ATCTGTATTTGGATCTATAACTGTACTTTGCCTGGCGCTGTGCGCAAGGTCAGAA
AACTTACTGCTAGTACCTAGAAACACACAAGGCTGCCCAGCCAAATCTTAATGTA
AAGTAGCTAGAGCCATGGAAGTACAGTATGAATTAAAAAGAAAAAAGTATTGAA
CTACA
>uc002llc NCode human ncRNA array Probe: IVGNh31353 Primary
Accession: uc002llc
(SEQ ID NO: 66)
GCTGACTCTCTTTTCGGACTCAGCCCGCCTGCACCCAGGTGAAATAAACAGCCAT
GTTGCTCACACAAAGCCTGTTTGGTGGTCTCTTCACAGGGACACGGATGAAATTT
GGTGCCGTGACTCGGATCGGGGAACCTCCCTTAGGAGATCAATCCCCTGTACTCC
TTTTCTTTGCCCTGTGAGAAAGATCCACCTATGACCTCAGGTCCTCAGACCGACC
AGCCCAAGGAACATCTCACCAATTTTAAATCAGACCTTGAAGATTTGTTGTTCAA
GGAGAAACTGAAGAGCAAGAAGGAAAGTGAGAGCCAGCAATACCAGCAGAGCC
AGATCTGAGCTGGGAGAAGGGGAGAAAGTTTGTGAAGAGGAGATCGGTGACCTG
GGCTCCTTATGTGCCTGAAAGAGTTTGAGTTTCCTGTTAACTCCAAATCAACAGT
ATTTTCAACAAGAAATGTGCAATTGAAATCAAGTGCTGTTTAAGTGCAGCTAGGA
TTTCCACAGGAAGACACTTGCAGTGAACAGAGTTATGGAGCAGCAAAAACACAG
ATCTATTTGGAAAAAGAGAAAACATATGCGTTGTATTTTGCTTCAATTATAAAAT
ACCATCCTCTCAAAGGTGGTTCTAAATTACAAAGGACTTTGATTTCTAGGTAGAT
TCTGGGTAGAGACTTCCTTTCATATTGAGGCATTAATGACACCTTTTAACCTGGG
AAGCAATATGACTGGAGTTGTACTTTGAGAAGATTAATCAGGTTTGGTTGCAGAA
TGAAAGAGAAGATGAAGTCAAGAGATTGGTTTAGAGGCTCTAGCAGAAGCTTAG
TCATATTTCAAAATGATCAAATATCAAGAAAAATTCTGAGCTGCATAACTTGTAT
AAAGTAATTTTCAGTGATTTTTTTCATGGTTATGATAAAAGAACTGGATTAGCAG
AAACTTTTACCCTGAATCAAGATTTAATTTTTCTTTGAGCTCATCTTAAGGATATC
GGAACATAGGGAGCAAACGATGGTGTGGCTGCCTCAGTGCTTGATTTTTAACGGT
TTTGAAGAGAATAGTTACATTTCTTCTCCTAGTAAGAACTAATAAATACATTAAC
AGAAATGAATTCCCTATCCCTTTGTACACTGGTCTATTTCTTCAAAACATTAAATA
CTATTGATAAGAT
>LOC400958 Agilent Human SurePrint G3 Probe: A_19_P00800206
Primary Accession: NR_036586
(SEQ ID NO: 67)
GGTAACCTAGAGTTGAGAGATGGAGGAAGAGATACAGAATCTGGATGGCTATGC
TCTGATCCTGTAATCCGACTATGCCTGAATGTGGATCTACCTTCCAAAGGACTTCT
CCAGCTCCATTTACAGTCTGGCTCCTGGGCCTTTGGATCCCAGCAGTGTCCGAGC
AGGAGCTCAAAGGACAGCCCCACCATGGGGGATCAGCCCTAGAAGCTGTCACTA
CATCTCCAACGGACGCAACTATTTTCCAGGAACACCGGGGGAGAGAGCCAACAA
CAGCACAGTGGCCCCGGACCGTGACCCTTGGACTGAAGGAACCTACAGATGTGG
TTTTTTTTGGTGACATTTTACATGCAACTCCAGATTTCAAACTCTTTTGGAGAAGC
AGGCAATCTGGCAACAGTCGTTTTGGATTCTCAGAAGGCAATAAAGAACAGCTG
CCACCTTCCGAGGGGCACGGTGGATGCCCTGTTCTGCCAGAATTGCCAACACGTT
TATCGTCTTAGACTTGCCCAAGGTGTCGCAGTTAGAGACTGCCTCCCTTATTCAC
GCTCCTGCCTGGTGCCCGTGGGCTTGAATTTGCTCCCCTTGGAGTGGGGTGAGGC
TCTGCAGACACTTCTCATACACCTCCCCTGCAGACAGCAAGCTCCTGGAACACAA
GTCACATGCATTTCATTTCCTGCTCTCTTGCTACCACCCAACATGGGCTCTCAATA
CATGTTGAAAGCAAGGATCAATGAATAAATGGGCAACTATCAGCTGTAGACTTG
TATGTGCCAGGTATGGTGCTAGGCATGCTAGGCACCAAAAGGGCCACAGAGGTG
TTACATGCCAGGATATCAGGGAGTTCATGACATAGTGAGGGAAAGAAAAAGCTT
ATGCAGTGTGTGTGTGAAACTTTAAGCAAACATGGTGCATAACAATAACAGGAA
TGACTTTGCCTGCCCCTGATGAAACTTCAGCAGGGCTATGCCCTGTCTTGCCACCT
TTAGGAAACAGCAGTCTTATAGTCCTTTGCCCCTCTGAGTTACAACCACTGTCTCC
TTTCAGGAGAATGCCCAGTGTTATATCATAATCAAGGCTTTGAACTTGATGTGGC
ATTACATGTTCTTCCATCTCCCCAGCCACCTGAGAAGGGAGATGGGGTAGCTTTT
CTCTCTCACTCTCTCTCCCCCAACCCCTCCTTTTCCCACCGGCAGGTGAATGAGCT
TCCTGCCCATAGGAGAAAGGGTAAAATCACAAGGTGGTGCCCTTGTCTCCAAATC
TCAAGGTCCTCTGGATGGCAGGTGAGTAAAGGTGACTCTTGTGATTATGGGTGTT
TTGGGTGTTCCTCAGAGATCCCCCAAACTGGGGTCTTGTCCACCATTCCCAGGAC
TCTGCCATGTGGAGCCATGGGAATGTGAAGTTCACCTCACACTTCCTTTCAGCTG
AGGTCACCACACAGCCCCTACCAGCCCGGCTATATTGGGTGGGATTTCAGATGCC
CCCACAATGGCTGCCTTGGAGACTTTCCACTGGTCCTCAAGAAGCAACAACGCTC
CCCTTGCTCTGCCTTTGGTGGAGGGCAATTCCTCCTCTCTCTCTGCCTGGCCCCAG
GCTGCTTCCACTGTCTCAGAAACTGGTCCCCGGATTCCCCCAGTTACAGAGAACC
CTCATCAAGCTCTCAAGTGGCCACTGAAACCCAGGCTCTCTAGGCTCTGGAGTAT
GGAAGTGACAGCTCCATTTAATTTCTCCTTTCCTCTTGTAGGCTTACAGCATAGCA
CTCTCCCAAGAAATCATCCAAAAATTACCTCAACCATTCTATAGACCCCAAGCTG
ACCAGGGGAGGGAGGACCAAGAATCTTGAAACGTAAATACTACATTTGATGGTC
TCCTTCAGACTTATTTTGGGATCTGATATCTCTTTAACAAAAATTATAAAAATTGA
GGCAAAGAGAGCCCCATTTTTTATATACTGTTCTAATAAATAACAGGTACCCTTA
GAAGAATGCAGACAAACACTCCTATGGAAATTTAAAGGAGCATAAGACTTCTTG
CAGTATAGGGAGAGAACCAAGGAAGACTTCCTGGAGGAAATGGCCATTGAACTG
GGCCTTGGACATGTGGAGGTGAGGGATGAGAGTATTCCAGATGAAGAGTCCAGC
ATAGGGAAGGCCCACAGGAAGGAATTGTGCTGTATTAATGCTGTCTTAGAGGCA
TTTCCATTGCCAGACACAGATACTCAAATTACTTCAGAGAGAGAGAGAGAGAGT
ATTGAAAGGGTTTCTGTGAATACCTCCACAACTGTGGTTCTCAAAGTGTAATCCC
TGGGCCAGCAGCATCGGCATCACCTGGGAACTTGTTAGAAATGCAGATTCCCAG
GCTGGGTGCAGTGGCTCACGCCTGTAACCCTAGCACTTTGGGAGTCCGAGGTGGG
TGGATCACCTGAGGTTGGGAGTTTGAGACCAGCCTGACCAACATGGAGAAACCC
TGTCTCTACTAAAAATACAAAAAGCCAGGTGTGGTGGCGCATGCCTGTAATCCCA
GCTGCTTGGGAGGCTGAGGCAGGAGAATCGCTTGAACCCGGGAGGCAGAGGTTG
CGGTGAGCCGAGATTGCACCACTGCACTCCAGCCTGGGTAACAAGAGCGAAACT
CCACCGAAGAAAGAAGAGAGAGAGAGAGGGAGAAAGAAAGAAAGGAGAAAGA
GAGAAAGAAAGAAAAAGAAAGAAAGAAAAGAAAAGAAAAAAGAAAGGAAAAG
AAAAGGCAAATTCACCAGAGCCTTTGAATCAGAAAAGAACCCCCAGGGGCTGGT
GGTAGCAGTCCTTGCACAGGCCCTCCAGGTGATTCTCATCCAGGGAAGCCTGCGA
GCCCTTGAGGTGGAATATTCTCAGGAATCCCTGAGATATGTGAAGAACTGATGGC
ATAGGCTATTTCTAGGGAGGAAATGGGGCTGCTGGGTGCACAGATGAGGGGAGG
TGGGAGACCTCTGTAATTGTGTACCATGTGCATATATTACCTATTCAGAGAATAA
TAAAACAATGCGTTTAATCCCCAAAAAAAAAAAAAAAAAAAAAAAAAAAAAAA
AAAAAAA
>XLOC_005327 Agilent Human SurePrint G3 Probe: A_19_P00802433
Primary Accession: ENST00000448327
(SEQ ID NO: 68)
CCAGGCGGCACATACATGATCCCAGACACCGAAGTAACCTCTGTCTCACTCCTCC
ACTTCCAGCAAGGGATGGAAAACAAACTGAAACTGGCTCAAGTGAATGCTCACT
GGAAGGCTTACTGGAAAACTTACTGGAAGGATGTGAGGACATGTTCGGGAATCT
ATTTGCAGAAAACATATTCAGCCCTGTCCACCACAGCCAGCTGGCTGAAGAGCTC
AAAAGGCAAGAAATCAGCAAGAGAGAGAGATGAAGCATGAGAAATGAGCAAAA
AACACCCAGCACATCATAATCTTGGACAGTTTAGCAGTACATGAAAATAGATGG
TCCTCGCCCCAAGGGACTGCAGTAACCCTGAATAAACAGGATGTCTCTCACTTTT
AGCAGTTCTTTCTGTGCTAGTATTGGGGAAATATATTTTTGGCTGCATGCAAAAT
GGTAAAAGACATCTATTAAGAAAATGAAAACAATGCTTCTGTTTTAGACGAAGC
TTTTGAAGGTTTAAGGATCACCTATTTATTGACAAAATTGTTTCCGTGGCTTAAAA
ATAAAATACAAACAAATACTA
>LINC00340 Agilent Human SurePrint G3 Probe: A_19_P00809119
Primary Accession: NR_015410
(SEQ ID NO: 69)
GTCTGCTCCGGGACTTGGAACAAAAGGGGGAACTCTGATGAACTCTCTTTCCTCC
CCTCTCCCCCGGACGCCGGGGTATCTCCCTCTCGCAACTTTGCCGCCCCGACTTTC
TCTGCTGTCAGGCCGGGAAAAAGTGTCCGAACGCCTCGTGGACTGCAGCGGGGG
AAATGTCCCTTAAAAGTGCGACGAAGTGGGGAAGAAGGTGTAATTACTATTATC
AGCATCTAGAAAGCATCATGAATTTGCTGGAGTACTTCCTAGCACTGACCTCCTT
CATTCTGCGTTGTTCTTACTGGATCTTTCCATCAGCCAACAATATGGAAGTACCA
ATACAAGGTCAAATCATTCCTGGATTCATCTGGAGTTGCTTAAAAGTTAAATCAT
TGGAATTTTTGATGATACCTTTTCTATATGGATTACAATTTGATCGCTGGGAATTC
TCCACCTTAAAGAAGTACCCTCAGGTGACTACAGATGTGTTAACACCCAGCATGT
TCCGGTAGGAGACTTTCTGGATGGGGAAGATTTCCAGGAATTGGCAACAAGCTC
ATTTCACTGGTGGGTTTGCTGAAGCATTATCACAAGACAGTCAGAATGACTGATG
AGTGCTCTTCAGGTGTGAATCATGGCAATACAGTGAAAGACAGTGATTTACTGCT
TTTGAGGGCGTGCATGTATATGATTAACGGATGGAAGTGCAGGACTCCAAGATTT
ACTTCCTTCCCTTTCCAGCAGAATTACCTGAGACGAGTAAAATCTACTGGTGGAG
TCACTCCATTATTCTTATCTGTGGAGATCTAGATCTTGATTTGAAAGTTTCTGAGA
AAATCTTCAGCTCAGACTTGAGGGTCAACTTTACCAGCTGAAGGATCTGCATTTA
CTGCTCAACCACATCTAATTTGATGTCCTCTGCAGATTTAAAATGTGTGCCTTCTC
TTCCGTCACCAAGTCATCCCTGGGTTACTACTGAACATCCTTCTCAATTCCCCCCG
ACCCATGGATGGCTGTTCTCCATTGTCTGTTTCACCAGATGTCCTCAAAACAAAC
AGACAGAAGAAGGAAGTGGCTAATGGAGCTGTGGAGTCCAAGTGTGACTGCCAA
GAGGAATCCAGCAAAGCCAAAAAGCCCAAGCATGTAGCCCTGCCCGAAGCACGC
CACACGCATGGAAAACCCAGAGGAAATGAGTGAGGATCAATGGGAAGAAGAGA
GCCAGCCAGGAAGTTGAAGATTTGTCCAGGAGCAGATAGCTGAAGAGAGAGAG
AGAGAAGAGAGAACGGCTTACAGCTCAGGTCCTCTCTCCATGCTTAGGAACCAC
TACAAATGCTACTGCCTTGAGTCTCATTTTGTTTCCCTCTGGAAACCACATGTGTA
CCTTGTTTGCAACAGTATGGGCTCACAGGCAGAAGGAATTTTCCTTGTCTTGGAT
GAGACTTTTGACTTGGACTTTTGGGTTAAGTTCTGGAGACCAGAAGGCCAAAATC
AAAAGTATGGGCAGGCTTGATTTCTTTAGAAGACTCCAGCGGAGAACTGTGTCTC
CTTGCTTCTGATTCTACATCTCCATCCATGGGCCACTGTTTCAGCAACCTCAGCCA
GTGCAACACAACCTCAGCCAAGAAGAGTATGCAGAGAAAGGAGTCCCCTACCTG
CCACAAAACTGTTGTCTGAAAACTGTCTCATATTGTCTCAAGTTGTCATTCATTGT
GAATTAGACCTGTTTAACATGTAATCTGCAACATGCTTCACTGTCTAATTTTCCAG
AGCCCCTCATATAAGGAACTGTATTATTGGTATAATCATCATGGTGAAGAAGTTG
GTATGTGGGGGAGAGATGACAGAAACAGAGAGTAAGTCAGAGCTGGCTGCCTGA
CAGATAAAAAGGAAATGACCAAAAAAAAAAAAAAAAA
>XLOC_000495 Agilent Human SurePrint G3 Probe: A_21_P0001708
Primary Accession: TCONS_00002202
(SEQ ID NO: 70)
CATTCATGCTTCCAGAGCTTCCTTTCTGTTGATCAGGGATTACGGGAATGATGTTT
TAGAAATTTGGTCACCTGGTTTGTAAAGTAAGGTCTTCCTGGGCAGAGTTTTGCT
TTTCCCTTTGTCTTCTGAGGTAGATTTATAGGCAGCTCTTGTTATTTACACTAAAG
GAAGGAGGAGAGAGAGAGATTTTATAAAAGGCATGATCCATGAAAGAAAG
>XLOC_001699 Agilent Human SurePrint G3 Probe: A_21_P0001923
Primary Accession: ENST00000450667
(SEQ ID NO: 71)
GAGAAGGAAAAGGATGTTGTACATGGGCAGAAAGGTGAAAATTGTGGCCAGAG
GCAGATTGTGCTATCAGGTAAATAAGCCCAGGCTATTGTGCTGGATGATGAGAG
GTCACTGGAACACTGATGAGCCATAACAGCTGAGGCCATTCTAGAATAGGCAGC
TTTAGCCAACCAGGCAGCTGACTGCAGATACAGGCCTGCATCCAGATGAGTCCA
GAAAAGCAACCTAGACACTACTGGCAAAGACATTGAAAAGAATCACACTGCTAA
ACCAGTAGCAGCTTTTATCTTCTTCTCAGATTGTATACATGCAGGATAAAAGTGT
TTCTAACACATAGACTTCTATATTCCAGCATAAAGATGTATTAGGCGCATTTATA
CTTTTACATCGAAGCCTGTTTCAACATGAACGGAGCCCAGATATTTATGTAGGCC
AGTAATTGGAGTGTATTTCCCTGTTCAATTTCACATTGTCTATAAAAGGGTTTCTC
ATGATCGATGACTTTCTAGTTAAATTCAGAAGAAAACATCTTGCAGCTACTGCCC
TATTGCCATGGCCACACATCACTGCCTTCTAGACATGGGAAGTTGGTGACTTAAT
CCTCGTTATACCCTGATTTTGGACAAATTTTACAAGAAGAATAGGACTTCTACTT
CTCTATGTCTACATGCACCTGGAACAGTGCTAGGTCTATGCTTACAAATTGATTG
GTTCAGGTCACCGCCATGAGCCTCAGGACAGAAAATTGCCGGATATGAGAGAGA
GAGAGAGATTTGACTGGGCTAAGAAAGAAATGAACACGATTT
>XLOC_008559 Agilent Human SurePrint G3 Probe: A_21_P0007070
Primary Accession: TCONS_00018783
(SEQ ID NO: 72)
CTGCACTCCAGCCTGGGCGACAGACCAAGACTCTGTCTCAAAAAAAAAAAAAAG
TTATAGTTTAATTTTTAAGGTTAATTTATTATTGAAGAAAAATTTTTAATGAGTTT
AGTGTAGCCTAGGTGTACACTAGGTGTTTATAGAGTCTACGATAGTGTACAGTCA
TGTCCTAGGCCTTCACATTCACTCATCACTCACTAACTCACACAGAGCAACTTCT
GGTCCTGCAAACTCCATTCGTGTTGAGTGTCCTATGTAGGTGTATTACTTTATATC
TTTTGTACTATATTTTTACTGTATTTTTTCTTTGTTTAGAAATGTTTGGATACACAA
ACACTAGTGTGTTACAATTGCCTACAGTATTCATTCAGTACAGTAACATGCTGTT
GCAACCTAGAAGCAATAAGCTACACCATATAGCCTAGGTGTGCAGTAGGCTACA
CCATCTAGCTTTGTGTAAGTACATTCTTTGAAGCTTGCACGATGACAAAATTGCC
TAATGACACATTTCTCAGAACATAACTCCATCATTAAGCTACATAACTTAAACCC
CTGCTATGCAATGAAACTCAGGTAGCATATTAAAAAATAGATAACTCAAGCATT
GCATACAGAGAAGCCATTCTTGGAACACCAGACAATAAGCATTGCATTAGATCA
GAGCAGTTCTGGGCACATCTATGGTCAACAAGAAATATTCTCAAAGTCTGAACTT
TGAGCTATAGTAGACAGACAAACTAAGAATTCCTCAAAGTTAGTATTTCCAACCG
TGATGTAAGAGTCTATTCTGAGTGTTGTGACAAACTATCTCCAGATCTCGCTAGA
GTAACACAATAAAGGTTTGTTTCTCACCCATCACAGTCGGGTATGGCTGTATGAG
GGAGGCGTGAGGAAGGATCTGCTCCTCGCCCATCACAGTCGGGTATGGCTGTAT
GAGGGAGGCGTGAGGAAGGCTCTGCTCCATGCATTCATGGAAGTGGCCTTGATC
ACCAGCCTAGCACTTCACTGGCAGGGCTCAGTCAATGACATCTAGTGGCTGGGA
AGCTCGGAAATGAGCTTTCCTTTGTGCTCAGAAGTAGGACTTGGGCGAACACATA
GCAGTATCTCTGCTCCATCCACATAAACGGGCTCAGAACTTAAATGGAAAGAGA
CGCTGAAGAGGGCATCAAATATATGAGAACTGGAACAGGGAAAGGAACAAAGA
TCTGAACAGGATCAGATAGAGATATTTGCCTACAGACAAGTCCTTGGTTAAAAG
ACCGTGGAAATTGATTCTAGAACTATATATTATTTATGGCTTGTGGGACGCAGAA
ATGTGTTCTGGTTACCTGTGCAATAAACTGTACATACTTCTCATTTCAGAGTTGGA
GTCAATCACTCTCTGTTGGCCTTTTTTGCTGTCTTTACAAAGTCATGGGTTAACGA
ACCCTACTGGGTACTTCTAACATGAGGTGTCTGGGCTGGGAGAGTCTTACTGGCA
ATTGATGTCAAGATTCTTCGTCCAGAGGCACAGAGCAGAAAGGTTCTTGGTCCAC
AGACACCTTAAAACAAGGCCACCCTGGCCAGGTTTATTCCCGTCTGGCGGCCTAC
ACATTTCTTATATCCTGGAAAAACTGGTGAGCAAGCAAGTGTCGACCTCAGAGTC
TCTGACAGGGCTATTTTGAAACCACACACCATGAAAACTCTCAGGGAAGTTAAA
AAACAAACAATCATAACCAAGGCAGTTTAGCTGTTTTGAAAAGAGATGGAGCTT
CATTACTTCAAACCCAAATTTCTGCAAGCCTGACAACCACCTTACATCAAAATAA
ACGTCTACCTGCTAGCTGAAATGTTTAAAAACACAGTTACCATGTGAGGTAAGCA
GAGCTGACCTTGACTGGCATCTCTATCAGCAGCTCAGTGGGATTAAATGGCTTGC
CAATGTCACAAGAATGTGAGCTCCTTTCTTCATCTTTCTGCTCCAATGTAGCAACT
ACCAAGGGGCCACCTGACAGAACATGGCCGCTGCAGAGGAACCCTGCTACCTGC
AGTTGGTGACATGGCCTAGGTCCCAGAGGCCTCGTGGTGCCACACACACAAGAA
CAGGCACCAACAACCAGTGACATTTTGACAGTCAAATGGAACCTGTGACTGCCA
TCTGTAGATGTGCCAGCCAAGAATGTGACCCTGGGGAAAGCCCTTCACACAGGT
CTTTCCTTGGTGTATTTATATTTAGTTCCAGCGAAAAACTGCAGTTGTTTTTCTCA
GTGACAGGCATCAAACGATAACCGAAAAGAATGAGAAATAATTGTTCCCTTTCT
CCCTGTTAGGAGATTGTACTCTTTGAATTTGGGACCACAGCTCTCTGAACAGCTA
GCTCTCCCATGCCTGGCTCATGAGACATCATAAATGTTGATTGTATTAAAGACAA
TTTAGAGGGAAAGGACTTGAATTCTGGTTCTAAGCTATTAAAAATATTTCTACAT
TTTAATTTTTAAATTAAGAAAGATTTTGTACATATGGAAAGGTGCAGAATATAAA
ACAGACAACCATATGCTTACCATCCAGATTAAACAACTGTTAACGTTTTCTCGTA
TTTACTTCAGATCACTTGAAACAAAAGAAAGACAAAAAGATACGGCTAAAGCCT
TGGCCCCCTTCACTCACATCCCTCCCCTCCTCCCCTCTGCAGAGCAACTTCTGCCT
GAAGCTGGTGTGTGTCATTTCCATGCATGATCTTGTGCTTTCAGTACATATTTGTA
TATCCAAAACAATATTTACTATTGTTTTGTGTGCATTCTTAATTTACATAAATGGC
ATCATATTGTAAATTCTCTTGCAACTTGGCTTTTCTTACTCAACAGTACATTTTAG
GGACTTATTTATGTTGTGTGGATACAGTGTAGACCTAGTTCATTCATTTTAACTTA
ATTGTGAAATACCATAGTTTACTTATCCATTTCCCTATTGGGTAAAATTAGTTATT
GCTTTATTGTCGTTGTTGTTTATTGCAATGAACATGCCTGTGCATGCATCTTTGTG
CACGTGTTTGTTAGTGTAAATGCCCTGAAGTGAAATTGCTAATTAGTAGGAAATA
TACTTCTGCACCTTCCTTAGCAGAGACAAATTGTTCTCCCAAGTGGTTGTACCTAT
TTGAACTCATGCTAGATTAGAAATCCCTGTGTTCCTACATCCTTACCATCATTTGT
GAGGCTTTCAATTTTTCTTATCCAATAAGTACAAATGACATTTTATTTTTTTAATT
CACATCTCTCTAATTATTCATGAGCTTAAGCATTTTTACATGTTTACTAACCAGTT
GTGTATGTGCATGTGTGTGCATGTGAGAGAGAGAGAGAAATAGGTTTTAATCCTT
TGTTCTTTTCTTATAAATTTATAGTTGTATTTATTCTGAAGTTCTTATCTGAGTTGA
AAAGTGTTCTCACAAATGGTATCTTGCCTTTTAATTTTGTTTATGTCATGTTCTATT
ATAAATAGCTTTTTAATTTTCATGTAGTTAAATTTATATGTCTTTTCAAGGTTTGT
GGGCATTTGTCCCTTAGTTAATAAATCTGTTTCTAACTCTACATTCAAGATATTCT
CCCACATTGTTTTCTAAAAATTCTAAATTTTTTTTCCCTTCACATTTAAATTTTTGT
CCATCTGGAATTTACTTTTGCTTATGTGATGAGTAGGGATCTAATTTTATCTTTTT
CCAAGCAGAAAGTTAATTGTCAAGGATGATCCAGACTTTCCCGCTGTTTGAAATG
TCATTTCTGGTGTTTTTTTTTTTTTTTTTT
>XLOC_009911 Agilent Human SurePrint G3 Probe: A_21_P0007854
Primary Accession: TCONS_00021223
(SEQ ID NO: 73)
GAATGATGACAGAGAGCTGGCCTTGCAAAGATCCACAGGAAAAGAGTTCCTGGC
AGAGGGAACAGCAAGGGCAGAAGGCTCAGGAAACCGTCCATTTGGAGGTCTGG
AAACCGGCACAGAAATAAACACGGTAGAGCTAGACCAGAGACCAACAAAGTGA
ATCTGGAGCTTAGATGGAGAGAGAAGAGAGAGATTAATTGAGGCCCCAGGTACT
GCGGAATGCTTCCCCAGGAGTGGATGAGGCCGTCTGAAAGGAGACCTCCGAAGT
GTTTCTTGAGGAAATGTGGCTGCAGACCCTAGAAGAAGCTACACAGCACTTGCC
AGGGCTGGGATGATGTCCAGGCCATGGAAACACCGTGTACCTGGTCCCAGGAAG
ATGAAGTGTGGGCCCAGAGACTAATGGCTTGAGCATCTCAGGCTAAGGTTGCCG
AGAAGTAGACAGCACCTCTAGATCCTAGTCAACATCTCTACAGGCTTGAAGTCTC
CCCAGAGGGCAAGGTTGGAATAAATCTGAAGCCTGTGGCTTGCCTGGGAGCTGC
CC
>XLOC_012294 Agilent Human SurePrint G3 Probe: A_21_P0009268
Primary Accession: TCONS_00025474
(SEQ ID NO: 74)
CCAAGCATCAAGCCAAGGAGGCAGTGGGCTTCTAGGTGCCCAAAGGAGAGAGA
GAATAAACTTGAACATTCTGACTTTGAAGAACATGACCAGGCTAGCCCAGGAGA
AAGATGGAGCACATATGGAGCAGAGCTGCCCCAGCCAACCTGCTCTTGAGACCC
CAGCTTAGAGCATCCAACTCCCAGCTAACACCCAGAAGCATGAGTGACTCCATTT
AAGGTCAATAAAACCATCTAGCCGAGTCCAGTGAGATAAGCCAGCCCTTGGTTG
ATCACAGATGCATGAGCTAAATAAA
>RPS18 4 Agilent mis-annotated. It is BC039356 in chr1.
BC039356 Agilent Human SurePrint G3 Probe: A_21_P0010744 Primary
Accession: TCONS_11_00002326
(SEQ ID NO: 75)
GTCTTTAAAAGAAGAGGGAAATATGGACACAGACATAGACACAGAGGAAGATG
ATGTGAAGACACACAGGGAAAACATCATGTAAAGACAGGCTTGGAGTGGTGCAC
CTACAAGCCAACACAGAATCACAGCATCTCAGAGTTGGAAGGAATTCTTCATAT
GACCACATTGATTTTTTTTTTCCTGTTGGTCGGCATCAGATTTGTGAAGGCCCCTG
GAAGATTGGATGGTGCCTGCCTATACGGAGGGCGGATCTTCCCCTCCTCGTCCAC
TCAGACTCACATGCAAGTCTCCTCTAGAAACACCCTTGCAGACACACCCCAAAAT
GACACTTTTAGAGCCCCTAGAAGATGCCTTAGAGATGAAAAAAAAAACACACGC
ATTTCCTAATGAAGAGGCAGCCAGATGCAGCCTCTGAGCCCTGACTGCACAGTGT
GACAGTCACTCAACCCAACACAGCTCTCTTGCCTTTGCTGCAACCTCAACACCCT
GCGTCCTGCCAAATCTCTTCCCATTTCATCAGTCCATCTATGCTGGTGTCCAGCCA
TTCCAGCCCACCATGGCATTTAAAAATCTTTCCAGCTCTCTGTGGAAGATCTGAG
ACTTGAGAAAGAGACTGTTGCTCAGGGCTGGACAGGAAGGAAGTATGCATTCCT
GGCTCCCAGAACAGAACAGCAATGTGGGTGACCCTTCGTCCCCTCCCCAAGGCGT
CCCCTTGGGCCGACACAAAAATAGATTCTATCCTCCTTGGTTCGTCTCCACCTCCC
TCGGGAAAGAAGACACAGGCTTCGAGTGAGTCAACAGTATTATCGGGGCTTGAC
TGTCTTTCAGGAATGACCAGATGTTGGGAAGAGGATAATGTGCCATTTCCTTTAA
CAAATAGTCCGGGCATCTGTGCATTTCCTTTTGAGCCAGCTCTTCAGGAGACTGT
GCCGCTGTGACAGGGAAGGACGAATCACCCTGGTTTCTACTCTCACGGATACTAG
GGGGCTCCTCGAACCCTTTGGATTCCAGCCCTCCATTAAGAAAATATTTCTGTCCT
TTGTATGCATGAGTGGCACCACGAGAAGACAGCATAGGGAGTGGTTACAAGCAA
AGAATTTAGAGACAAAATAAATGCTCTAAGGGAAAAAGACAAGTAGCCAAGGA
ACGCTGGGAGAGGGCTTGGAGGAAGCAAATTGTTCATCCATTCCCCCAAATCAG
TGGTTCTCAGTAGAAAACCAACATGAGTAACATTTGCCTGGGAACTTGTCTACCA
CCCCAGCCCTACTGAAACTCCAGGGGTGAAGCCCAGCAATCTCCTTTGACAAGCC
TTCCAGGAGATTCTGATGTGAGCTCAAGATTGAGAACTACTGATCCAGATAGATC
TTAGCTGGTCCTGGGGCTTCCCAGAAAGCATTTTTAAAAAAGCAGAGATTCTCCT
CCACAGGAGGCCTACATGCTGCCACCTCTGTGGCCACCATGTCTCTAGTGATCCC
TGAAGAGTTCCAGCATATTCTGCGAGTACTCAACACCAGCATCGGTGGGCGGTG
GAAAAAAAGCCTTTGCCATCACTGCCATTTAGGCTGTGGGTCGAAGATATGCTCA
TGCGGTGTTGAGGAAAGCAGACTTTGACCACACCAAGAGGGCAGGAGAACTCAC
TGAGGATGAGGTGCAACGTGTGATCACCATTATACAGGATCCATGCCAGTACAA
GATCCCGGACTGGTTCTTGAACAGACAGAAGGATGTAAAGTCTGGAAAATACAG
CCAGATCCCAGCCAATGGACAACAAGTTCTGTGACGACCTGGAGTGATTGAAGA
AGTTTCAGGCCCATAGAGGGCTGCGCCACCTCTGGGGCCTTCGTGTCTTGAGGCC
AGCACAGCAAGACCACTGGCTGCCATGGCTGTACTACGGGTGTGTCCAAGAAGG
AATAAGTCTGTAGGCCTTGTCTGTTAATAAATAGTTTATATACCAAAAAAAAAAA
AAAAA
>XLOC_12_008560 Agilent Human SurePrint G3 Probe: A_21_P0012112
Primary Accession: TCONS_12_00016171
(SEQ ID NO: 76)
CCGTTGCTCCCTTTCCCCTGGCTGGCAGCGCGGAAGCCGCACGATGCCTGGAGTT
CCTGTAAACCACGTGAACCAGCGGGACTTCGTCAGAGCTCTGGCAGCCTTTCTCA
AAAAGTCCGGGAAGCTGAAAGTCCCCGAATGGGTGGACACCGTCAAGCTGGCCA
AGCACAAAGAGCTTCCTCCCTACGTTGAGAACTGGTTCTACACACGAGCCGGTGG
CAGCTGCCAACAAGAAGCATTGGAACAAACCATGCTGGGTTAATACAT

What is claimed is:

1. A method for accessing the progression of prostate cancer in a subject who is undergoing treatment for prostate cancer, which method comprises:
(i) assessing the expression level of a long noncoding RNA in a biological sample obtained from the subject;
(ii) comparing the expression level of the long noncoding RNA in the sample to a reference derived from the expression level of the long noncoding RNA in samples obtained from healthy subjects and determining the current condition of the subject; and
(iii) for the subject determined to suffer from prostate cancer periodically repeating steps (i) and (ii) during treatment as a basis to determine the efficacy of said treatment by assessing whether the expression level of the long noncoding RNA in the subject is up-regulated or down-regulated, wherein a down-regulation in the expression level of the long noncoding RNA correlates to an improvement in the subject's condition.

2. The method of claim 1, wherein the long noncoding RNA is selected from the group consisting of SEQ ID NOs: 2-76.

3. The method of claim 2, further comprising assessing the expression level of SPRY4-IT1 (SEQ ID NO: 1).

4. The method of any one of claims 1-3, wherein the expression level of the long noncoding RNA is assessed by evaluating the amount of the long noncoding RNA using a probe.

5. The method of claim 4, wherein the biological sample comprises a tissue sample.

6. The method of claim 5, wherein the tissue sample is a prostatic adenocarcinoma tissue sample.

7. The method of claim 1, wherein the prostate cancer is early stage prostate cancer.

8. The method of claim 1, wherein the long noncoding RNA is XLOC_007697 (SEQ ID NO: 2).

9. The method of claim 1, wherein the long noncoding RNA is XLOC_009911 (SEQ ID NO: 3).

10. The method of claim 1, wherein the long noncoding RNA is XLOC_008559 (SEQ ID NO: 4).

11. The method of claim 1, wherein the long noncoding RNA is XLOC_005327 (SEQ ID NO: 5).

12. The method of claim 1, wherein the long noncoding RNA is LOC100287482 (SEQ ID NO: 6).

13. A method for treating prostate cancer in a patient diagnosed as having prostate cancer comprising administering to the patient an effective amount of a therapeutic agent that reduces or down-regulates the expression level of a long noncoding RNA.

14. The method of claim 13, wherein the long noncoding RNA is selected from the group consisting of SEQ ID NOs: 2-76.

15. The method of claim 13 or claim 14, wherein the long noncoding RNA expression is reduced or down-regulated in prostate cancer cells.

16. The method of claim 15, wherein the long noncoding RNA expression is reduced by at least about 50%, 60%, 70%, 80% or 90%.

17. The method of any one of claims 13-16, wherein the therapeutic agent is an siRNA.

18. The method of any one of claims 13-17, wherein the therapeutic agent is contained within a liposome.

19. The method of claim 13, wherein the long noncoding RNA is XLOC_007697 (SEQ ID NO: 2).

20. The method of claim 13, wherein the long noncoding RNA is XLOC_009911 (SEQ ID NO: 3).

21. The method of claim 13, wherein the long noncoding RNA is XLOC_008559 (SEQ ID NO: 4).

22. The method of claim 13, wherein the long noncoding RNA is XLOC_005327 (SEQ ID NO: 5).

23. The method of claim 13, wherein the long noncoding RNA is LOC100287482 (SEQ ID NO: 6).

24. A method for determining a treatment regimen for a patient with prostate cancer which method comprises:
identifying whether said cancer is aggressive or indolent by identifying one or more of markers for aggressive prostate cancer said marker is one or more of PSA isoforms, kallikreins, GSTP1, AMACR, ERG, gene fusions involving ETS-related genes, PCA3, or a combination thereof;
treating said cancer with a regimen consistent with whether the cancer is aggressive or indolent.

25. The method of claim 24, wherein the progress of said treatment regimen is monitored by further evaluating the presence and quantity of one or more of said markers in said patient and optionally adjusting the treatment protocol based on said evaluation.

26. The method of claim 24 and claim 25, wherein said treatment regimen is one or more of open prostatectomy, minimally invasive laparoscopic robotic surgery, intensity modulated radiation therapy (IMIRT), proton therapy, brachytherapy, cryotherapy, molecular-targeted therapy, vaccine therapy and gene therapy, hormone therapy, active surveillance, or a combination thereof.

27. A method for detecting prostate cancer in a patient suspected of having prostate cancer, which method comprises:
(i) assessing the expression level of a long noncoding RNA in a biological sample obtained from said patient;
(ii) comparing the expression level of the long noncoding RNA in the sample to a reference derived from the expression level of the long noncoding RNA in samples obtained from healthy subjects;
(iii) identifying said patient as having prostate cancer when the expression level of the long noncoding RNA in said patient is greater than the reference or identifying said patient as not having prostate cancer when the expression level of the long noncoding RNA is equal or less than the reference.

28. The method of claim 27, wherein said patient is suspected of prostate cancer based on the patient's prostate specific antigen (PSA) Score, the Myriad Prolaris Assay (MPA) Score, the Oncotype DX Genomic Prostate Score (GPS), or the Cancer of the Prostate Risk Assessment (CAPRA) Score.

29. A method for differentiating indolent and aggressive prostate cancer, which method comprises:
identifying the aggressive prostate cancer based on the expression of one or more of aggressive tumor-predictive genes associated with the aggressive prostate cancer; and
identifying the indolent prostate cancer based on the lack of the expression or the low expression of one or more of aggressive tumor-predictive genes associated, and wherein the expression of aggressive tumor-predictive genes is determined by one or more of prostate specific antigen (PSA) Score, the Myriad Prolaris Assay (MPA) Score, the Oncotype DX Genomic Prostate Score (GPS), the Cancer of the Prostate Risk Assessment (CAPRA) Score, or a combination thereof.

30. A kit comprising a composition comprising a long noncoding RNA, and instructions for use, wherein the long noncoding RNA is selected from the group consisting of SEQ ID NOs: 2-76.
